SEQUENCE LISTING (137 sequences)

SEQ ID NO: 1 - protein, 640 aa, Artificial
  Synthetic Sequence
NYIKGDPYFAEYATKLSFILNPSDANNPSGETANHNDEACNCNESGISSVGQAQTSGPSSNKTCITHSSIKTNKKKECKDVKLGVRENDKDLKICVIEDTSLSGVDNCCCQDLLGILQENCSDNKRGSSSNDSCDNKNQDECQKKLEKVFASLTNGYKCDKCKSGTSRSKKKWIWKKSSGNEEGLQEEYANTIGLPPRTQSLYLGNLPKLENVCEDVKDINFDTKEKFLAGCLIVSFHEGKNLKKRYPQNKNSGNKENLCKALEYSFADYGDLIKGTSIWDNEYTKDLELNLQNNFGKLFGKYIKKNNTAEQDTSYSSLDELRESWWNTNKKYIWTAMKHGAEMNITTCNADGSVTGSGSSCDDIPTIDLIPQYLRFLQEWVENFCEQRQAKVKDVITNCKSCKESGNKCKTECKTKCKDECEKYKKFIEACGTAGGGIGTAGSPWSKRWDQIYKRYSKHIEDAKRNRKAGTKNCGTSSTTNAAASTDENKCVQSDIDSFFKHLIDIGLTTPSSYLSNVLDDNICGADKAPWTTYTTYTTTEKCNKERDKSKSQSSDTLVVVNVPSPLGNTPYRYKYACQCKIPTNEETCDDRKEYMNQWSCGSARTMKRGYKNDNYELCKYNGVDVKPTTVRSNSSKLD

SEQ ID NO: 2 - protein, 341 aa, Plasmodium falciparum
KCDKCKSGTSRSRKIWTWRKSSGNKEGLQEEYANTIGLSPRTQLLYLGNLRKLENVCEDVTDINFDTKEKFLAGCLIAAFHEGKNLKKRYLEKKKGDNNSKLCKDLKYSFADYGDLIKGTSIWDNDFTKDLELNLQQIFGKLFRKYIKKKNISTEQDTSYSSLDELRESWWNTNKKYIWLAMKHGAGMNSTTCSCSGDSSSGENQTNSCDDIPTIDLIPQYLRFLQEWVEHFCEQRQAKVKDVITNCNSCKESGGTCNSDCEKKCKNKCDAYKTFIEDCKGVGGTGTAGSSWVKRWYQIYMRYSKYIEDAKRNRKAGTKSCGTSSTTNVSVSTDENKCVQS

SEQ ID NO: 3 - protein, 656 aa, Artificial
  Synthetic Sequence
DYIKGDPYFAEYATKLSFILNSSDANNPSGETANHNDEVCNPNESEISSVGQAQTSDPSSNKTCNTHSSIKANKKKVCKHVKLGINNNDKVLRVCVIEDTSLSGVENCCFKDLLGILQENCSDNKSGSSSNGSCNNKNQEACEKNLEKVLASLTNCYKCDKCKSGTSTVNKNWIWKKSSGNKEGLQKEYANTIGLPPRTHSLYLGNLPKLENVCEDVKDINFDTKEKFLAGCLIAAFHEGKNLKKRYPQNKNDDNNSKLCKALEYSFADYGDLIKGTSIWDNEYTKDLELNLQQIFGKLFRKYIKKNISTEQDTLYSSLDELRESWWNTNKKYIWLAMKHGAGMNITTCCGDGSVTGSGSSCDDIPTIDLIPQYLRFLQEWVEHFCKQRQEKVKDVINSCNSCKNTSSKTKLGDTCNSDCEKKCKIECEKYKKFIEECRTAVGGTAGSSWSKRWDQIYKMYSKHIEDAKRNRKAGTKNCGITTGTISGESSGANSGVTTTENKCVQSDIDSFFKHLIDIGLTTPSSYLSIVLDDNICGDDKAPWTTYTTYTTYTTTEKCNKERDKSKSQQSNTSVVVNVPSPLGNTPHGYKYACQCKIPTNEETCDDRKEYMNQWISDTSKNPKGSGSTNNDYELYTYNGVKETKLPKKLNSPKLD

SEQ ID NO: 4 - protein, 643 aa, Artificial
  Synthetic Sequence
DYIKDDPYSKEYTTKLSFILNSSDANTSSGETANHNDEACNCNESEISSVGQAQTSGPSSNKTCITHSFIKANKKKVCKDVKLGVRENDKVLRVCVIEDTSLSGVDNCCCQDLLGILQENCSDNKRGSSSNGSCNNKNQDECQKKLEKVFVSLTNGYKCDKCKSGTSTVNKKWIWKKSSGNEKGLQKEYANTIGLPPRTQSLYLGNLPKLGNVCEDVTDINFDTKEKFLAGCLIAAFHEGKNLKISHEKKKGDNGKKLCKALEYSFADYGDLIKGTSIWDNEYTKDLELNLQKAFGKLFGKYIKKNIASDENTSYSSLDELRESWWNTNKKYIWTAMKHGAEMNSTMCNADGSVTGSGSSCDDIPTTDFIPQYLRFLQEWVEHFCKQRQEKVNAVIENCNSCKNTSGERKIGGTCNGDCKTECKNKCEAYKNFIEDCKGGDGTAGSSWVKRWDQIYKRYSKHIEDAKRNRKAGTKSCGPSSITNASVSTDENKCVQSDIDSFFKHLIDIGLTTPSSYLSIVLDENNCGEDNAPWTTYTTYTTTEKCNKDKKKSKSQSCNTAVVVNVPSPLGNTPHEYKYACQCKIPTTEETCDDRKEYMNQWISDTSKKQKGSGSTNNDYELYTYTGVKETKLPKKLNSPKLD

SEQ ID NO: 5 - protein, 640 aa, Artificial
  Synthetic Sequence
SYVKNDPYSKEYVTKLSFILNPSDANNPSGETANHNDEACNPNESEIASVGQAQTSDRLSQKACITHSFIGANKKIVCKDVKLGVREKDKDLKICVIEDDSLRGVENCCFKDLLGILQENCSDNKSGSSSNGSCNNKNQDECQKKLDEALASLHNGYKCDKCKSGTSRSKKIWTWRKFPGNGEGLQKEYANTIGLPPRTQSLYLGNLRKLENVCKGVTDINFDTKEKFLAGCLIAAFHEGKNLKISNKKKNDDNGKKLCKDLKYSFADYGDLIKGTSIWDNEYTKDLELNLQKIFGKLFRKYIKKNIASDENTLYSSLDELRESWWNTNKKYIWLAMKHGTTCSSGSGDNGDGSVTGSGSSCDDMSTIDLIPQYLRFLQEWVEHFCKQRQEKVKDVIENCKSCKNTSGERIIGGTCGSDCKTKCKGECDAYKNFIEECKRGDGTAGSPWSKRWDQIYMRYSKYIEDAKRNRKAGTKNCGTSSTTNAAENKCVQSDIDSFFKHLIDIGLTTPSSYLSIVLDENICGDDKAPWTTYTTYTTTEKCNKETDKSKSQSCNTAVVVNVPSPLGNTPHGYKYACECKIPTTEETCDDRKEYMNQWISDTSKKPKGGRSTNNDYELYTYNGVKETKLPKKSSSSKLD

SEQ ID NO: 6 - protein, 358 aa, Plasmodium falciparum
KCEKCKSEQSKKNNNIWIWRKFPGNGEGLQKEYANTIGLPPRTHSLYLGNLPKLENVCKDVKDINFDTKEKFLAGCLIAAFHEGKNLKTTYPQNKNADNNSKLCKDLKYSFADYGDLIKGTSIWDNDFTKDLELNLQKIFGKLFRKYIKKNIASDENTLYSSLDELRESWWNTNKKYIWLAMKHGAEMNSTMCNGDGSVTGSSDSGSTTCSGDNGSISCDDIPTIDLIPQYLRFLQEWVEHFCKQRQEKVKPVIENCKSCKNTSGERIIGGTCGSDCEKKCKGECDAYKKFIEECKGGGGGTGTAGSPWSKRWDQIYKRYSKYIEDAKRNRKAGTKSCGPSSTTNAAASTTESKCVQS

SEQ ID NO: 7 - protein, 333 aa, Plasmodium falciparum
KCDKCKSEQSKKNNKNWIWKQFPGNGEGLQKEYANTIGLPPRTHSLYLGNLPKLENVCKGVTDINFDTKEKFLAGCLIAAFHEGKNLKTSHEKKKGDNGKKLCKDLKYSFADYGDLIKGTSIWDNDFTKDLELNLQQIFGKLFRKYIKKNISAEQDTSYSSLDELRESWWNTNKKYIWLAMKHGTTCSSGSGDNGDGSVTGSGSSCDDMPTTDFIPQYLRFLQEWVEHFCKQRQEKVNAVITNCKSCKESGGTCNSDCEKKCKDECEKYKKFIEECRTAADGTAGSSWSKRWDQIYKMYSKHIEDAKRNRKAGTKNCGTSSTTNAAENKCVQS

SEQ ID NO: 8 - protein, 269 aa, Plasmodium falciparum
DYIKDDPYSKEYTTKLSFILNSSDANTSSGETANHNDEACNCNESEIASVEQASISDRSSQKAYITHSSIKTNKKKVCKYVKLGINNNDKVLRVCVIEDTSLSGVENCCFKDLLGILQENCSDNKRGSSFNDSCNNNNEEACQKKLEKVLASLTNGYKCEKCKSGTSRSKKKWIWKKSSGKEGGLQKEYANTIGLPPRTQSLYLGNLPKLENVCKGVTDINFDTKEKFLAGCLIAAFHEGKNLKPSHQNKNDDNNSKLCKDLKYSFADY

SEQ ID NO: 9 - protein, 333 aa, Plasmodium falciparum
KCDKCKSGTSRSKKKWTWRKSSGNKEGLQKEYANTIGLPPRTHSLYLGNLRKLENVCEDVTDINFDTKEKFLAGCLIAAFHEGKNLKTTYPQNKNDDNNSKLCKALKYSFADYGDLIKGTSIWDNDFTKDLELNLQKIFGKLFRKYIKKNISTEQHTSYSSLDELRESWWNTNKKYIWLAMKHGAEMNGTTCSCSGDSSDDIPTIDLIPQYLRFLQEWVEHFCKQRQAKVNAVINSCNSCKNTSGERKLGGTCGSECKTECKNKCDAYKEFIDGTGSGGGTGTAGSSWVKRWDQIYKRYSKYIEDAKRNRKAGSKNCGTSSTTNAAESKCVQS

SEQ ID NO: 10 - protein, 650 aa, Artificial
  Synthetic Sequence
SYVKNNPYSAEYVTKLSFILNSSDANTSSETPSKYYDEVCNCNESEISSVGQAQTSGPSSNKTCITHSSIKTNKKKVCKDVKLGINNNDKVLRVCVIEDTSLSGVDNCCCQDLLGILQENCSDKNQSGSSSNGSCNNKNQDECQKKLEKVFASLTNGYKCDKCKSGTSRSKKKWIWRKSSGNEEGLQKEYANTIGLPPRTQSLYLGNLRKLENVCKGVTDINFDTKEKFLAGCLIAAFHEGKNLKTTYPQNKKKLCKDLKYSFADYGDLIKGTSIWDNEYTKDLELNLQKAFGKLFRKYIKKNISTEQHTLYSSLDELRESWWNTNKKYIWLAMKHGAGMNSTTCCGDGSVTGSGSSCDDIPTIDLIPQYLRFLQEWVEHFCKQRQEKVNAVIENCNSCKECGDTCNGECKTECEKKCKIECEKYKTFIEECVTAVGGTSGSPWSKRWDQIYKRYSKYIEDAKRNRKAGTKNCGITTGTISGESSGANSGVTTTENKCVQSDIDSFFKHLIDIGLTTPSSYLSIVLDDNICGADNAPWTTYTTYTTYTTTKNCDIKKKTPKSQPINTSVVVNVPSPLGNTPHGYKYACQCKIPTTEESCDDRKEYMNQWIIDTSKKQKGSGSTNNDYELYTYNGVKETKLPKKSSSSKLD

SEQ ID NO: 11 - protein, 643 aa, Artificial
  Synthetic Sequence
SYVKDDPYSAEYVTKLSFILNSSDANTSSETPSKYYDEVCNCNESEISSVGQAQTSGPSSNKTCITHSSIKTNKKKVCKDVKLGINNNDKVLRVCVIEDTSLSGVDNCCCQDLLGILQENCSDKNQSGSSSNGSCNNKNQDECQKKLEKVFASLTNGYKCDKCKSGTSRSKKKWIWRKSSGNEEGLQKEYANTIGLPPRTQSLYLGNLPKLENVCKGVTDIIYDTKEKFLSGCLIAAFHEGKNLKTSHEKKNDDNGKKLCKALEYSFADYGDLIKGTSIWDNDFTKDLELNLQKIFGKLFRKYIKKNNTAEQDTSYSSLDELRESWWNTNKKYIWTAMKHGAGMNSTTCSGDGSVTGSGSSCDDMPTIDLIPQYLRFLQEWVEHFCKQRQEKVKDVITNCNSCKECGDTCNGECKTECKTKCKGECEKYKNFIEECNGTADGGTSGSSWSKRWDQIYKRYSKYIEDAKRNRKAGTKNCGTSSTTNAAASTTENKCVQSDIDSFFKHLIDIGLTTPSSYLSNVLDDNICGEDKAPWTTYTTYTTKNCDIQKKTPKPQSCDTLVVVNVPSPLGNTPHGYKYVCECKIPTTEETCDDRKEYMNQWIIDTSKKQKGSGSTNNDYELYTYNGVQIKQAAGTLKNSKLD

SEQ ID NO: 12 - protein, 269 aa, Plasmodium falciparum
NYIKGDPYSAEYATKLSFILNSSDTENASEKIQKNNDEVCNCNESEIASVEQAPISDRSSQKACITHSSIKANKKKVCKHVKLGVRENDKDLKICVIEDTSLSGVDNCCCQDLLGILQENCSDNKSGSSSNGSCNNNNEEICQKKLEKVLASLTNGYKCDKCKSGTSTVNKNWIWKKYSGKEGGLQEEYANTIGLPPRTQSLYLGNLPKLENVCEDVKDINFDTKEKFLAGCLIAAFHEGKNLKTSNKKKNDDNNSKLCKALKYSFADY

SEQ ID NO: 13 - protein, 344 aa, Plasmodium falciparum
KCDKCKSGTSTVNKKWIWKKYSGTEGGLQEEYANTIALPPRTQSLYLGNLPKLENVCKDVTDINFDTKEKFLAGCLIAAFHEGKNLKTTYLEKKKGDNGKKNDDNNSKLCKALKYSFADYGDLIKGTSIWDNDFTKDLELNLQQIFGKLFRKYIKKNIASDENTLYSSLDELRESWWNTNKKYIWLAMKHGAGMNSTMCNADGSVTGSGSSCDDIPTIDLIPQYLRFLQEWVEHFCKQRQAKVKDVITNCNSCKECGGTCNGECKTECEKKCKGECDAYKKFIEECKGKADEGTSGSSWSKRWDQIYKRYSKYIEDAKRNRKAGTKNCGPSSTTSTAESKCVQS

SEQ ID NO: 14 - protein, 334 aa, Plasmodium falciparum
KCDKCKSEQSKKNNNIWIWKKSSGTEGGLQKEYANTIALPPRTQSLYLGNLRKLENVCEDVKDINFDTKEKFLAGCLIAAFHEGKNLKKRYLEKKNGDNNSKLCKALKYSFADYGDLIKGTSIWDNEYTKDLELNLQKIFGKLFRKYIKKNNTAEQHTSYSSLDELRESWWNTNKKYIWLAMKHGTTCSSGSGDNGSISCDDIPTIDLIPQYLRFLQEWVEHFCEQRQGKVNAVIENCNSCKNTSSKTKLGGTCNGECKTECKGECDAYKEFIEKCKGTAAEGTSGSSWVKRWYQIYMRYSKYIEDAKRNRKAGTKNCGTSSTTSTAESKCVQS

SEQ ID NO: 15 - protein, 332 aa, Plasmodium falciparum
KCDKCKSEQSKKNNNIWIWKKSSGTEGGLQKEYANTIALPPRTQSLYLGNLRKLENVCEDVKDINFDTKEKFLAGCLIAAFHEGKNLKKRYLEKKNGDNNSKLCKALKYSFADYGDLIKGTSIWDNEYTKDLELNLQKIFGKLFRKYIKKNNTAEQDTSYSSLDELRESWWNTNKKYIWTAMKHGTTCSSGSGDNGSISCDDIPTIDLIPQYLRFLQEWVEHFCEQRQEKVKDVIKNCNSCKECGGTCNGECKTECKNKCKDECDAYKKFIEECEGKAAEGTSGSSWSKRWDQIYKRYSKYIEDAKRNRKAGTKNCGTSSTTSTAENKCVQS

SEQ ID NO: 16 - protein, 267 aa, Plasmodium falciparum
NYIKDDPYSAEYTTKLSFILNSSDTENASEKIQKNNDEVCNPNESGIACVELAQTSGSSSNKTCNTHSFIKANKKKVCKDVKLGINKKDKDLKICVIEDDSLRGVDNCCCQDLLGILQENCSDKNQSGSSSNGSCNNKNQEACQKKLENVFASLTNGYKCEKCKSEQSKKNNKNWIWKKYSVKEEGLQKEYANTIALPPRTQSLYLGNLPKLGNVCKGVTDINFDTKEKFLAGCLIAAFHEGKNLKTTYLQNKKKLCKALKYSFADY

SEQ ID NO: 17 - protein, 263 aa, Plasmodium falciparum
DYIKGDPYFAEYATKLSFILNSSDANTSSGETANHNDEACNPNESEIASVEQASISDRSSQKACNTHSSIKANKKKECKHVKLGVRENDKDLKICVIEDTSLSGVDNCCCQDLLGILQENCSDNKRGSSSNGSCDKNSEEICQKKLDEALASLHNGYKNQKCKSEQSKKNKNKWIWKKSSGNEKGLQKEYANTIGLPPRTQSLYLGNLPKLENVCEDVTDINFDTKEKFLAGCLIAAFHEGKNLKTTYPQNKNDDNGKKLCKD

SEQ ID NO: 18 - protein, 338 aa, Plasmodium falciparum
KCDKCKSEQSKKNNNIWIWKKSSGNKKGLQKEYANTIGLPPRTQSLYLGNLPKLENVCKDVTDINFDTKEKFLAGCLIAAFHEGKNLKISNEKKNDDNGKKLCKDLKYSFADYGDLIKGTSIWDNEYTKDLELNLQNNFGKLFRKYIKKNNTAEQHTLYSSLDELRESWWNTNKKYIWLAMKHGTTCSSGSGDNGDGSVTGSGSSCDDMSTIDLIPQYLRFLQEWVEHFCKQRQEKVNAVIENCNSCKNTSSKTKLGGTCNGECKTECEKKCKDECEKYKEFIEECKRGDGTAGSPWVKRWDQIYMRYSKYIEDAKRNRKAGTKSCGTSAAENKCVQS

SEQ ID NO: 19 - protein, 341 aa, Plasmodium falciparum
KCDKCKSEQSKKNNNIWIWKKSSGDEKGLQKEYANTIALPPRTQSLYLGNLPKLENVCKDVTDINFDTKEKFLAGCLIAAFHEGKNLKTSHQNKNADNGKKNDDNGKKLCKALKYSFADYGDLIKGTSIWDNEYTKDLELNLQQIFGKLFRKYIKRNNTAEQHTLYSSLDELRESWWNTNKKYIWLAMKHGTTCSSGSGDNGDGSVTGSGSSCDDMSTIDLIPQYLRFLQEWVEHFCKQRQEKVKDVITNCNSCKECGGTCGSDCKTKCEAYKKFIEECNGTADGGTSGSSWSKRWDQIYKRYSKYIEDAKRNRKAGTKNCGPSSGANSGVTTTENKCVQS

SEQ ID NO: 20 - protein, 352 aa, Plasmodium falciparum
KCEKCESEQSKKNNKYWIWKKSSGNGEGLQEEYANTIALPPRTHSLCLVCLHEKEGKKTQELKNIRTNSELLKERIIAAFHEGKNLKTSPQNKNDNGKKLCKDLKYSFADYGDLIKGTSIWDNEYTKDLELNLQKIFGKLFRKYIKKNNTAEQHTLYSSLDELRESWWNTNKKYIWLAMKHGAGMNSTMCNADGSVTGSSDSGSTTCCGDNGSISCDDMPTIDLIPQYLRFLQEWVEHFCEQRQEKVNAVITNCKSCKECGGTCNSDCEKKCKAYKEFIEKCKGGGTEGTSGSSWSKRWDQIYKRHSKHIEDAKRNRKAGTKNCGITTGTISGESSGANSGVTTTENKCVQS

SEQ ID NO: 21 - protein, 344 aa, Plasmodium falciparum
KCDKCKSGTSRSRKIWTWRKFRGNGEGLQKEYANTIGLSPRTQLLYLVCLHEKGKKTQELKNISTNSELLKEWIIAAFHEGKNLKTTYPQKKNDDNGKKLCKALKYSFADYGDLIKGTSIWDNDFTKDLELNLQKIFGKLFRKYIKKNIASDENTSYSSLDELRESWWNTNKKYIWTAMKHGAGMNGTTCCGDGSVTGSSDSGSTTCCGDGSVTGSGSSCDDIPTIDLIPQYLRFLQEWVEHFCEQRQEKVKDVITNCKSCKESEKKCKNKCDAYKEFIDGTGSGGGTGTAGSSWSKRWDQIYMRYSKYIEDAKRNRKAGTKNCGTSSGANSGVTTTENKCVQS

SEQ ID NO: 22 - protein, 350 aa, Plasmodium falciparum
KCEKCKSEQSKKNNKIWTWRKFPGNGEGLQKEYANTIGLSPRTQLLYLVCLHEKGKKTQHKTISTNSELLKEWIIAAFHEGKNLKKRYLEKKKGDNNSKLCKDLKYSFADYGDLIKGTSIWDNDFTKDLELNLQQIFGKLFRKYIKKNIASDENTSYSSLDELRESWWNTNKKYIWTAMKHGAGMNSTMCNGDGSVTGSSDSGSTTCSGDNGSISCDDIPTIDLIPQYLRFLQEWVEHFCEQRQEKVKDVIKNCNSCKECGGTCNGECKTECKNKCKDECEKYKNFIEVCTGGDGTAGSPWSKRWYQIYMRYSKYIEDAKRNRKAGTKSCGTSSGANSGVTTTESKCVQS

SEQ ID NO: 23 - protein, 359 aa, Plasmodium falciparum
KCEKCKSEQSKKNNKNWIWRKFPGNGEGLQKEYANTIGLPPRTHSLYLVCLHEKGKKTQELKNIRTNSELLKEWIIAAFHEGKNLKKRYHQNNNSGNKKKLCKALEYSFADYGDLIKGTSIWDNEYTKDLELNLQQIFGKLFRKYIKKNISTEQDTLYSSLDELRESWWNTNKKYIWLAMKHGAGMNSTTCCGDGSVTGSSDSGSTTCSGDNGSISCDDMPTIDLIPQYLRFLQEWVEHFCEQRQEKVKDVIENCKSCKNTSGERIIGGTCNGECKTECEKKCKAACEAYKTFIEECEGKAAEGTSGSSWSKRWYQIYMRYSKYIEDAKRNRKAGTKNCGKSSGANSGVTTTENKCVQS

SEQ ID NO: 24 - protein, 270 aa, Plasmodium falciparum
NYIKDDPYSKEYVTKLSFIPNSSDANNPSGETANHNDEVCNPNESEISSVEHAQTSVLLSQKAYITHSSIKANKKKVCKYVKLGVRENDKDLKICVIEDDSLRGVENCCFKDFLRILQENCSDNKRESSSNGSCNNNNEEACEKNLDEALASLTNCYKNQKCKSGTSTVNNNKWIWKKSSGKEGGLQKEYANTIGLPPRTQSLCLVVCLDEKEGKTQELKNIRTNSELLKEWIIAAFHEGKNLKKRYHQNKNDDNNSKLCKALKYSFADY

SEQ ID NO: 25 - protein, 334 aa, Plasmodium falciparum
KCDKCKSEQSKKNNKYWIWKKYSVKEGGLQKEYANTIALPPRTQSLCLVVCLDEKEGKTQELKNIRTNSELLKERIIAAFHEGKNLKTYHEKKKGDDGKKLCKDLKYSFADYGDLIKGTSIWDNDFTKDLELNLQKIFGKLFRKYIKKNNTAEQHTSYSSLDELRESWWNTNKKYIWTAMKHGAEMNGTTCSCSGDSSNDIPTIDLIPQYLRFLQEWVEHFCEQRQAKVNAVIKNCKSCKECGGTCNGECKTECKTKCKGECEKYKEFIEKCEGQAAEGTSGSSWSKRWYQIYMRYSKYIEDAKRNRKAGTKNCGTSSGANSGVTTTENKCVQS

SEQ ID NO: 26 - protein, 351 aa, Plasmodium falciparum
KCDKCKSEQSKKNNKNWIWKKYSGTEGGLQKEYANTIALPPRTQSLYLVCLHEKEEKTQELKNISTNSELLKEWIIAAFHEGKNLKISPQNKNDNGKNLCKDLKYSFADYGDLIKGTSIWDNDFTKDLELNLQQIFGKLFRKYIKKNNTAEQDTLYSSLDELRESWWNTNKKYIWTAMKHGAGMNGTTCCGDGSVTGSSDSGSTTCCGDGSVTGSGSSCDDIPTIDLIPQYLRFLQEWVEHFCEQRQAKVKDVIKNCNSCKECGGTCNGECKTECEKKCKGECEAYKKFIEKCNGGGGEGTSGSSWSKRWDQIYMRYSKYIEDAKRNRKAGTKNCGTSSTTNAAENKCVQS

SEQ ID NO: 27 - protein, 353 aa, Plasmodium falciparum
KCDKCKSGTSTVNKKWIWKKFPGKEGGLQEEYANTIALPPRTQSLCLVVCLDEKEGKTQHKTISTNSELLKEWIIAAFHEGKNLKISNKKKNDENNSKLCKDLKYSFADYGDLIKGTSIWDNDFTKDLELNLQKIFGKLFRKYIKKNNTAEQDTSYSSLDELRESWWNTNKKYIWLAMKHGTTCSSGSGDNGDGSVTGSSDSGSTTCCGDGSVTGSGSSCDDIPTIDLIPQYLRFLQEWVEHFCKQRQAKVKDVIENCKSCKNTSSKTKLGDTCNSDCKTKCKVACEKYKEFIEKCVSAAGGTSGSSWVKRWDQIYMRYSKYIEDAKRNRKAGTKNCGPSSTTSTAESKCVQS

SEQ ID NO: 28 - protein, 327 aa, Plasmodium falciparum
KCDKCKSGTSTVNKKWIWKKYSGKEGGLQKEYANTIGLPPRTQSLCLVCLHEKEGKTQELKNISTNSELLKEWIIAAFHEGKNLKISNKKKNDDNGKKLCKDLKYSFADYGDLIKGTSIWDNDFTKDLELNLQKIFGKLFRKYIKKNNTAEQDTLYSSLDELRESWWNTNKKYIWTAMKHGAGMNSTTCSCSGDSSNDIPTIDLIPQYLRFLQEWVEHFCKQRQEKVNAVITNCKSCKESGGTCNSDCEKKCKIECEKYKNFIEKCVTAAGGTSGSSWSKRWDQIYKMYSKYIEDAKRNRKAGTKNCGPSSTTNAAASTDENKCVQS

SEQ ID NO: 29 - protein, 628 aa, Artificial
  Synthetic Sequence
NYIKGDPYFAEYATKLSFILNSSDTENASETPSKYYDEACNCNESEIASVGQAQTSGPSSNKTCITHSSIKTNKKKECKDVKLGINNNDKVLRVCVIEDTSLSGVDNCCCQDLLGILQENCSDNKRGSSSNGSCDKNSEEICQKKLEKVFASLTNGYKCDKCKSGTSRSKKKWIWKKSSGNEEGLQKEYANTIGLPPRTQSLCLVCLHEKEGKTQHKTISTNSELLKEWIIAAFHEGKNLKTSHEKKNDDNGKKLCKALEYSFADYGDLIKGTSIWDNEYTKDLELNLQKIFGKLFRKYIKKNNTAEQHTSYSSLDELRESWWNTNKKYIWTAMKHGAGMNGTTCSCSGDSSNDMPTIDLIPQYLRFLQEWVEHFCKQRQEKVNAVIENCNSCKESGGTCNSDCKTECKNKCEAYKEFIEDCKGGGTGTAGSPWSKRWDQIYKRYSKHIEDAKRNRKAGTKNCGTSSTTNAAASTDENKCVQSDVDSFFKHLIDIGLTTPSSYLSNVLDDNICGADKAPWTTYTTYTTTKNCDIQKKTPKSQSCDTLVVVNVPSPLGNTPHEYKYACECKIPTTEETCDDRKEYMNQWSCGSAQTVRGRSGKDDYELYTYNGVKETKPLGTLKNSKLD

SEQ ID NO: 30 - protein, 350 aa, Plasmodium falciparum
KCEKCKSEQSKKNNKNWIWRKFRGTEGGLQEEYANTIGLPPRTQSLCLVVCLDEKGKKTQELKNIRTNSELLKEWIIAAFHEGKNLKPSHQNKNSGNKENLCKALKYSFADYGDLIKGTSIWDNDFTKDLELNLQKIFGKLFRKYIKKNNTAEQHTSYSSLDELRESWWNTNKKYIWTAMKHGAEMNGTTCNADGSVTGSSDSGSTTCSGDNGSISCDDIPTIDLIPQYLRFLQEWVEHFCKQRQEKVNAVINSCNSCKNTSSKTKLGDTCNSDCKTKCKIECEKYKTFIEKCVTAAGGTSGSPWSKRWDQIYKRYSKYIEDAKRNRKAGTKNCGPSSTTSTAESKCVQS

SEQ ID NO: 31 - protein, 330 aa, Plasmodium falciparum
KCDKCKSEQSKKNNKNWIWRKYSGNGEGLQKEYANTIGLPPRTHSLYLVCLHEKEGKTQELKNIRTNSELLKEWIIAAFHEGKNLKTTYLENKNDENKKKLCKALKYSFADYGDLIKGTSIWDNDFTKDLELNLQKIFGKLFRKYIKKNIASDENTLYSSLDELRESWWNTNKKYIWTAMKHGAEMNGTTCSSGSGDNGSISCDDIPTIDLIPQYLRFLQEWVGHFCKQRQEKVNAVITNCNSCKESGGTCNSDCEKKCKIECEKYKKFIEECRTAAGGTSGSPWSKRWDQIYKMYSKYIEDAKRNRKAGTKNCGPSSTTSTAESKCVQS

SEQ ID NO: 32 - protein, 334 aa, Plasmodium falciparum
KCDKCKSEQSKKNNKNWIWRKYSGNGEGLQKEYANTIGLPPRTHSLYLVCLHEKEGKTQHKTISTNSELLKEWIIAAFHEGKNLKKRYPQNNNSGNKKKLCKDLKYSFADYGDLIKGTSIWDNEYTKDLELNLQKAFGKLFRKYIKKNIASDENTLYSSLDELRESWWNTNKKYIWLAMKHGAEMNGTMCNADGSVTGSGSSCDDMSTIDLIPQYLRFLQEWVEHFCEQRQAKVKDVINSCKSCKESGDTCNSDCEKKCKNKCDAYKTFIEEFCTADGGTAGSPWSKRWDQIYKRYSKYIEDAKRNRKAGTKNCGTSSGANSGVTTTENKCVQS

SEQ ID NO: 33 - protein, 350 aa, Plasmodium falciparum
KCDKCKSGTSTVNKNWIWKKYSGKEEGLQKEYANTIALPPRTHSLYLVCLHEKGKKTQELKNIRTNSELLKEWIIAAFHEGKNLKTSPQNNNSGNKKKLCKALKYSFADYGDLIKGTSIWDNDFTKDLELNLQKIFGKLFRKYIKKNNTAEQHTSYSSLDELRESWWNTNKKYIWLAMKHGAEMNGTTCCGDGSVTGSSDSGSTTCSGDNGSISCDDMPTTDFIPQYLRFLQEWVEHFCKQRQEKVKHVMESCKSCKECGDTCNGECKTECEKKCKNKCEAYKTFIEKCVSADGGTSGSSWSKRWDQIYMRYSKYIEDAKRNRKAGTKNCGTSSTTNAAASTAENKCVQS

SEQ ID NO: 34 - protein, 647 aa, Artificial
  Synthetic Sequence
DYIKDDPYSAEYATKLSFILNPSDANTSSGETANHNDEVCNCNESEIASVELAPISDSSSNKTCITHSFIGANKKKECKDVKLGVREKDKDLKICVIEDDSLRGVENCCCQDLLGILQENCSDNKSGSSSNGSCDKNSEDECQKKLENVFASLKNGYKCDKCKSGTSTVNKKWIWRKYSGNGEGLQKEYANTIGLPPRTHSLYLVCLHEKEGKTQHKTISTNSELLKEWIIAAFHEGKNLKTSHQNNNSGNKKKLCKALKYSFADYGDLIKGTSIWDNDFTKDLELNLQKIFGKLFRKYIKKNIASDENTSYSSLDELRESWWNTNKKYIWLAMKHGAEMNSTMCNGDGSVTGSSDSGSTTCSGDNGSISCDDIPTIDLIPQYLRFLQEWVEHFCKQRQEKVKDVITNCKSCKESGDTCNSDCEKKCKNKCEAYKKFIEERRTAAQGTAESSWVKRWDQIYMRYSKYIEDAKRNRKAGTKSCGPSSTTNAAASTAENKCVQSDIDSFFKHLIDIGLTTPSSYLSIVLDDNICGADNAPWTTYTTYTTTKNCDIKKKTPKPQSCDTLVVVNVPSPLGNTPHEYKYACQCRTPNKQESCDDRKEYMNQWSSGSAQTVRGRSTNNDYELYTYNGVKETKPLGTLKNSKLD

SEQ ID NO: 35 - protein, 341 aa, Plasmodium falciparum
KCDKCKSGTSTVNKKWIWRKSSGNKEGLQKEYANTIGLPPRTQSLYLGNLPKLENVCEDVKDINFDTKEKFLAGCLIVSFHEGKNLKTSHEKKNDDNGKKLCKALEYSFADYGDLIKGTSIWDNEYTKDLELNLQKIFGKLFRKYIKKNNTAEQDTSYSSLDELRESWWNTNKKYIWTAMKHGAGMNITTCCGDGSSGENQTNSCDDIPTIDLIPQYLRFLQEWVEHFCKQRQEKVNAVVTNCKSCKESGGTCNGECKTKCKNKCEVYKTFIDNVGDGTAGSPWVKRWDQIYKRYSKHIEDAKRNRKAGTKNCGITTGTISGESSGATSGVTTTENKCVQS

SEQ ID NO: 36 - protein, 632 aa, Artificial
  Synthetic Sequence
NYIKDDPYSKEYVTKLSFIPNSSDANTSSEKIQKNNDEVCNPNESGISSVEQAQTSGPSSNKTCITHSSIKANKKKECKDVKLGVRENDKDLKICVIEDTSLSGVDNCCCQDLLGILQENCSDNKRGSSSNDSCDNKNQDECQKKLDEALESLHNGYKNQKCKSGTSTVNKKWIWKKSSGNKEGLQKEYANTIGLPPRTQSLYLGNLPKLENVSKGVTDIIYDTKEKFLAGCLIVSFHEGKNLKTSHEKKNDDNGKKLCKALEYSFADYGDLIKGTSIWDNEYTKDLELNLQKAFGKLFRKYIKKNISAEQDTSYSSLDELRESWWNTNKKYIWIAMKHGAGMNGTTCCGDGSSGENQTNSCDDIPTIDLIPQYLRFLQEWVEHFCEQRQAKVKDVITNCKSCKNTSGERKIGGTCNGECKTKCKNKCEAYKTFIEHCKGGDGTAGSSWVKRWDQIYKRYSKHIEDAKRNRKAGTKSCGTSTAENKCVQSDIDSFFKHLIDIGLTTPSSYLSIVLDENNCGEDKAPWTTYTTTKNCDIQKDKSKSQSSDTLVVVNVPSPLGNTPHGYKYACQCKIPTTEETCDDRKEYMNQWSCGSARTMKRGYKNDNYELCKYNGVDVKPTTVRSSSTKLD

SEQ ID NO: 37 - protein, 639 aa, Artificial
  Synthetic Sequence
DYIKGDPYSAEYVTKLSFIPNSSDANNPSEKIQKNNDEVCNCNESEISSVGQASISDPSSNKTCNTHSSIKANKKKVCKDVKLGVRENDKVLKICVIEHTSLRGVDNCCFKDLLGILQEPRIDKNQSGSSSNGSCDKNSEEACEKNLEKVLASLTNGYKCDKCKSGTSRSKKKWIWKKYSGKEGGLQEEYANTIGLPPRTQSLCLVVCLDEKEGKTQELKNISTNSELLKEWIIAAFPEGKNLKPSPEKKKGDNGKKLCKDLKYSFADYGDLIKGTSIWDNEYTKDLELNLQKIFGKLFRKYIKKNIASDENTLYSSLDELRESWWNTNKKYIWLAMKHGAGMNSTMCNADGSVTGSGSSCDDMPTIDLIPQYLRFLQEWVEHFCKQRQEKVKPVIENCNSCKNTSSERKIGGTCNSDCKTECKNKCEVYKKFIEDCKGGDGTAGSSWSKRWDQIYKRYSKYIEDAKRNRKAGTKNCGPSSTTNAAENKCVQSDIDSFFKHLIDIGLTTPSSYLSTVLDDNICGEDNAPWTTYTTYTTTKNCDKDKKKSKSQSCDTLVVVNVPSPLGNTPHEYKYACECRTPNKQESCDDRKEYMNQWISDNTKNPKGSGSGKDYYELYTYNGVDVKPTTVRSSSTKLD

SEQ ID NO: 38 - protein, 655 aa, Artificial
  Synthetic Sequence
DYIKGDPYFAEYATKLSFILNSSDANTSSGETANHNDEACNCNESEISSVEHASISDPSSNKTCNTHSSIKANKKKVCKHVKLGVRENDKDLRVCVIEHTSLSGVENCCFKDFLRILQENCSDNKSGSSSNGSCDKNNEEACEKNLEKVFASLTNCYKCEKCKSEQSKKNNKKWTWRKSSGNKGGLQEEYANTIGLPPRTQSLCLVVCLDEKEGKKTQELKNIRTNSELLKEWIIAAFHEGKNLKPSHEKKNDDNGKKNDDNNSKLCKDLKYSFADYGDLIKGTSIWDNEYTKDLELNLQKIFGKLFRKYIKKNIASDENTLYSSLDELRESWWNTNKKYIWLAMKHGAEMNGTTCNADGSVTGSGSSCDDIPTIDLIPQYLRFLQEWVEHFCKQRQAKVKDVIENCKSCKESGNKCKTECKNKCEAYKKFIENCKGGDGTAGSSWVKRWDQIYMRYSKYIEDAKRNRKAGTKNCGPSSITNVSASTDENKCVQSDIDSFFKHLIDIGLTTPSSYLSIVLDDNICGDDKAPWTTYTTYTTYTTYTTYTTYTTYTTTKNCDKERDKSKSQSCNTAVVVNVPSPLGNTPHEYKYACECRTPSNKELCDDRKEYMNQWSSGSAQTVRDRSGKDYYELYTYNGVKETKLPKKLNSSKLD

SEQ ID NO: 39 - protein, 347 aa, Plasmodium falciparum
KCDKCKSEQSKKNNKYWIWKKSSVKEEGLQKEYANTIALPPRTHSLCLVVCLDEKGKKTQELKNISTNSELLKERIIAAFHEGKNLKTTYLEKKNADNNSKLCKALKYSFADYGDLIKGTSIWDNEYTKDLELNLQQIFGKLFRKYIKKNNTAEQHTLYSSLDELRESWWNTNKKYIWLAMKHGAGMNGTTCCGDGSVTGSSDSGSTTCSGDNGSISCDDMPTTDFIPQYLRFLQEWVEHFCKQRQEKVKDVIENCNSCKNNLGKTEINEKCKTECKNKCEAYKNFIEKFCTADGGTSGSPWSKRWDQIYKRYSKYIEDAKRNRKAGTKNCGTSSTTSTAENKCVQS

SEQ ID NO: 40 - protein, 335 aa, Plasmodium falciparum
KCEKCKSGTSTVNKYWIWRKSSGNKEGLQKEYANTIALPPRTHSLCLVVCLDEKEGKTQELKNISTNSELLKERIIAAFHEGENLKTSHEKKKGDDGKKNADNNSKLCKALKYSFADYGDLIKGTSIWDNEYTKDLELNLQKIFGKLFRKYIKKNIASDENTSYSSLDELRESWWNTNKKYIWLAMKHGAGMNGTTCSCSGDSSDDMPTTDFIPQYLRFLQEWVEHFCKQRQENVNAVIENCNSCKECGGTCNSDCEKKCKTECKNKCEAYKNFIEKFCTADGGTSGYSWSKRWDQIYKRYSKYIEDAKRNRKAGTKSCGTSSTTSTAESKCVQS

SEQ ID NO: 41 - protein, 667 aa, Artificial
  Synthetic Sequence
SYVKNNPYSKEYVTKLSFILNPSDANNPSETPSKYYDEVCNCNESGIACVGQAQTSGPSSNKTCITHSFIGANKKKVCKDVKLGVREKDKDLKICVIEDTYLSGVDNCCFKDFLGMLQENCSDNKSGSSSNGSCNNKNQDECEKNLDEALASLTNGYKCEKCKSGTSTVNKYWIWRKSSGNKEGLQKEYANTIALPPRTHSLCLVVCLDEKEGKTQHKTISTNSELLKEWIIAAFHEGKNLKTSHEKKKGDDGKKNADNNSKLCKALKYSFADYGDLIKGTSIWDNDFTKDLELNLQKIFGKLFRKYIKKNIASDENTSYSSLDELRESWWNTNKKYIWLAMKHGAGMNSTTCCGDGSVTGSSDSGSTTCCGDGSVTGSGSSCDDMPTTDFIPQYLRFLQEWVEHFCKQRQENVNAVIENCNSCKECGGTCNSDCEKKCKTECKGECDAYKEFIEKCNGGAAEGTSGSSWSKRWDQIYKRYSKYIEDAKRNRKAGTKNCGTSSTTSTAESKCVQSDIDSFFKHLIDIGLTTPSSYLSIVLDENICGADNAPWTTYTTYTTYTTYTTTEKCNKETDKSKLQQCNTSVVVNVPSPLGNTPHGYKYVCECRTPNKQETCDDRKEYMNQWISDNTKNPKGSRSTNNDYELYTYNGVQIKPTTVRSNSTKLD

SEQ ID NO: 42 - protein, 348 aa, Plasmodium falciparum
KCDKCKSEQSKKNNKNWIWKKSSGNEKGLQKEYANTIGLPPRTQSLCLVVCLDEKEGKTQELKNIRTNSELLKEWIIAAFHEGKNLKTSHEKKKGDNNSKLCKDLKYSFADYGDLIKGTSIWDNEYTKDLELNLQNNFGKLFRKYIKKNIASDENTSYSSLDELRESWWNTNKKYIWLAMKHGAGMNSTTCSSGSGSTTCSSGSGSTTCSSGSGDSCDDMPTIDLIPQYLRFLQEWVEHFCKQRQEKVNAVIKNCNSCKESGGTCNGECKTECKNKCEAYKTFIEEFCTADGGTSGSPWSKRWDQIYKMYSKHIEDAKRNRKAGTKNCGPSSTTNVSVSTDENKCVQS

SEQ ID NO: 43 - protein, 652 aa, Artificial
  Synthetic Sequence
DYIKDDPYFAEYVTKLSFILNSSDANNPSGETANHNDEVCNPNESGIASVEQAQTSDPSSNKTCNTHSSIKANKKKVCKHVKLGVRENDKDLKICVIEHTSLSGVENCCCQDFLRILQENCSDNKSGSSSNGSCNNKNQEACEKNLEKVLASLTNCYKCDKCKSEQSKKNNKNWIWKKSSGNEKGLQKEYANTIGLPPRTQSLCLVVCLDEKEGKTQELKNIRTNSELLKEWIIAAFHEGKNLKKRYPQNKNDDNNSKLCKDLKYSFADYGDLIKGTSIWDNEYTKDLELNLQNNFGKLFRKYIKKNISTEQDTLYSSLDELRESWWNTNKKYIWLAMKHGAGMNSTTCSSGSGSTTCSSGSGSTTCSSGSGDSCDDMPTTDFIPQYLRFLQEWVEHFCKQRQEKVNAVIKNCNSCKESGGTCNGECKTECKNKCEAYKTFIEEFCTADGGTSGSPWSKRWDQIYKMYSKHIEDAKRNRKAGTKNCGPSSTTNVSVSTDENKCVQSDIDSFFKHLIDIGLTTPSSYLSIVLDDNICGEDKAPWTTYTTYTTTKKCNKETDKSKSQSCNTAVVVNVPSPLGNTPHGYKYACECKIPTTEETCDDRKEYMNQWIIDTSKKQKGSGSGKDDYELYTYNGVDVKPTTVRSNSTKLD

SEQ ID NO: 44 - protein, 628 aa, Artificial
  Synthetic Sequence
DYIKDDPYSAQYTTKLSFILNPSDANTSSEKIQKNNDEACNCNESGISSVGQAQTSGPSSNKTCITHSSIKANKKKVCKDVKLGINNNDKVLRVCVIEDTSLSGVDNCCCQDLLGILQENCSDNKRGSSSNGSCNNNNEEACEKNLDEAPASLHNGYKNQKCKSGTSRSKKKWIWKKSSGNEKGLQEEYANTIGLPPRTQSLCLVCLHEKEGKTQHKTISTNSELLKEWIIAAFHEGKNLKTSHEKKNDDNGKKLCKALEYSFADYGDLIKGTSIWDNEYTKDLELNLQKAFGKLFRKYIKKNNTAEQDTSYSSLDELRESWWNTNKKYIWIAMKHGAGMNGTTCSCSGDSSNDMPTIDLIPQYLRFLQEWVEHFCEQRQAKVKDVITNCKSCKESGNKCKTECKTKCKDECEKYKTFIEDCNGGGTGTAGSSWVKRWDQIYKRYSKHIEDAKRNRKAGTKNCGPSSITNAAASTDENKCVQSDIDSFFKHLIDIGLTTPSSYLSNVLDENSCGDDKAPWTTYTTYTTTKNCDIQKDKSKSQPINTSVVVNVPSPLGNTPYRYKYACECKIPTTEESCDDRKEYMNQWSCGSARTMKRGYKNDNYELCKYNGVDVKPTTVRSNSSKLD

SEQ ID NO: 45 - protein, 653 aa, Artificial
  Synthetic Sequence
DYIKGDPYFAEYATKLSFILNPSDTENASETPSKYYDEACNPNESEIASVEQAQTSGPSSNKTCITHSSIKTNKKKECKDVKLGVRENDKDLKICVIEDTSLSGVDNCCFKDLLGILQENCSDNKRGSSSNDSCNNNNEEACEKNLDEALASLTNGYKCDKCKSGTSTVNKKWTWRKSSGNEEGLQKEYANTIGLPPRTQSLCLVCLHEKEGKTKHKTISTNSELLKEWIIAAFHEGKNLKTSHEKKNDDNGKKLCKALEYSFADYGDLIKGTSIWDNEYTKDLELNLQKAFGKLFRKYIKKNNTAEQDTSYSSLDELRESWWNTNKKYIWTAMKHGAEMNGTTCSSGSGDNGDSSITGSSDSGSTTCSGDNGSISCDDIPTTDFIPQYLRFLQEWVEHFCEQRQAKVKDVINSCNSCNESGGTCNGECKTKCKDECEKYKKFIEDCNGGDGTAGSSWVKRWDQIYKRYSKHIEDAKRNRKAGTKNCGPSSITNAAASTDENKCVQSDVDSFFKHLIDIGLTTPSSYLSIVLDENSCGDDKAPWTTYTTYTTTEKCNKERDKSKSQSSDTLVVVNVPSPLGNTPHEYKYACECKIPTNEETCDDRKDYMNQWISDTSKKQKGSGSGKDYYELYTYNGVQIKQAAGRSSSTKLD

SEQ ID NO: 46 - protein, 490 aa, Plasmodium falciparum
KCDKCKSEQSKKNNNKWIWKKYSGNGEGLQKEYANTIGLPPRTQSLCLVCLHEKEGKTQHKTISTNSELLKEWIIAAFHEGKNLKKRYPQNKNDDNNSKLCKALEYSFADYGDLIKGTSIWDNEYTKDLELNLQKAFGKLFRKYIKKNNTAEQDTSYSSLDELRESWWNTNKKYIWTAMKHGAEMNGTTCSSGSGDNGDSSCDDIPTIDLIPQYLRFLQEWVEHFCKQRQAKVKDVINSCNSCKNTSGERKIGGTCNSDCEKKCKVACDAYKTFIEECRTAVGGTAGSSWVKRWDQIYKRYSKHIEDAKRNRKAGTKNCGPSSTTNAAENKCVQSDIDSFFKHLIDIGLTTPSSYLSNVLDENSCGADKAPWTTYTTYTTYTTYTTYTTTEKCNKERDKSKSQQSNTSVVVNVPSPLGNTPHEYKYACECKIPTTEETCDDRKEYMNQWIIDNTKNPKGSGSTDNDYELYTYNGVQIKQAAGRSSSTKLD

SEQ ID NO: 47 - protein, 335 aa, Plasmodium falciparum
KCEKCKSGTSTVNNKWIWRKSSGKEGGLQKEYANTIGLPPRTQSLYLGNLPKLENVCKGVTDIIYDTKEKFLSGCLIAAFHEGKNLKTTYLEKKNDDNGKKLCKALEYSFADYGDLIKGTSIWDNEYTKDLELNLQKIFGKLFRKYIKKNNTAEQDTSYSSLDELRESWWNTNKKYIWIAMKHGAGMNGTTCSSGSGDSSNDIPTTDFIPQYLRFLQEWVENFCEQRQAKVKPVIENCNSCKESGGTCNGECKTKCKVACDAYKKFIDGTGSGGGSRPTGIAGSSWSKRWDQIYKRYSKHIEDAKRNRKAGTKNCGPSSITNVSVSTDENKCVQS

SEQ ID NO: 48 - protein, 637 aa, Artificial
  Synthetic Sequence
NYIKDDPYSKEYVTKLSFIPNSSDANTSSEKIQKNNDEVCNPNESGISSVEQAQTSDPSSNKTCITHSSIKANKKKECKDVKLGVRENDKDLKICVIEHTSLSGVDNCCFKDFLRMLQEPRIDKNQRGSSSNGSCDKNSEEACEKNLDEALASLTNGYKCDKCKSEQSKKNNNKWIWKKFPGKEGGLQEEYANTIGLPPRTQYLCLVVCLDEKEGKTQELKNIRTNSELLKEWIIAAFHEGKNLKTTYPQKKNDDNGKKLCKDLKYSFADYGDLIKGTSIWDNEYTKNVELNLQNNFGKLFRKYIKKNNTAEQDTSYSSLDELRESWWNTNKKYIWLAMKHGAEMNSTTCCGDGSVTGSGSSCDDIPTIDLIPQYLRFLQEWVEHFCKQRQAKVKDVITNCNSCKESGNKCKTECKNKCKDECEKYKKFIEACGTAVGGTGTAGSPWSKRWDQIYKRYSKHIEDAKRNRKAGTKNCGPSSTTNAAENKCVQSDIDSFFKHLIDIGLTTPSSYLSIVLDDNICGADKAPWTTYTTYTTENCDIQKKTPKSQSCDTLVVVNVPSPLGNTPHGYKYACQCRTPNKQESCDDRKEYMNQWIIDNTKNPKGSGSGKDYYELCKYNGVKETKPLGTLKNSKLD

SEQ ID NO: 49 - protein, 330 aa, Plasmodium falciparum
KCDKCKSEQSKKNNNKWIWRKFPGKEGGLQKEYANTIGLPPRTQSLCLVCLHEKEGKTQHKTISTNSELLKEWIIAAFHEGKNLKTTYLEKKNAENKKKLCKALKYSFADYGDLIKGTSIWDNEYTKDLELNLQKIFGKLFRKYIKKNNTAEQDTSYSSLDELRESWWNTNKKYIWTAMKHGAGMNGTMCNADGSVTGSGSSCDDMPTTDFIPQYLRFLQEWVEHFCKQRQAKVKDVIENCKSCKESGNKCKTECKNKCDAYKTFIEECGTAVGGTAGSSWVKRWDQIYKRYSKHIEDAKRNRKAGTKNCGTSSTTNAAASTAENKCVQS

SEQ ID NO: 50 - protein, 269 aa, Plasmodium falciparum
NYIKDDPYSKEYVTKLSFILNSSDAENASETPSKYYDEACNCNESGISSVEQASISDRSSQKACNTHSFIGANKKKVCKHVKLGVRENDKDLKICVIEDDSLRGVENCCFKDFLRMLQEPRIDKNQRGSSSNDSCNNNNEEACEKNLDEALASLHNGYKNQKCKSEQSKKNNNKWIWKKSSGKEGGLQKEYANTIGLPPRTQSLCLVCLHEKEGKTQHKTISTNSELLKEWIIDAFHEGKNLKTTYLEKKKGDNGKKLCKALKYSFADY

SEQ ID NO: 51 - protein, 347 aa, Plasmodium falciparum
KCDKCKSEQSKKNNKNWIWKKSSGKEGGLQKEYANTIALPPRTQSLCLVVCLHEKEGKTQHKTISTNSELLKEWIIDAFHEGKNLKTTYLEKQNADNGKKNADNNSKLCKDLKYSFADYGDLIKGTSIWDNEYTKDLELNLQQIFGKLFRKYIKKNIASDENTLYSSLDELRESWWNTNKKYIWTAMKHGAEMNGTTCSSGSGDSSSGENQTNSCDDIPTIDLIPQYLRFLQEWVEHFCEQRQAKVKDVITNCKSCKESGGTCNSDCKTKCKGECEKYKKFIEKCKGGGTEGTSGSSWVKRWYQIYMRYSKYIEDAKRNRKAGTKSCGTSSGANSGVTTTESKCVQS

SEQ ID NO: 52 - protein, 269 aa, Plasmodium falciparum
DYIKDDPYSKEYTTKLSFILNSSDANTSSEKIQKNNDEVCNPNESEISSVEQAQTSRPSSNKTCITHSSIKANKKKVCKDVKLGVRENDKVLRVCVIEHTSLSGVENCCCQDLLGILQENCSDNKRGSSSNGSCDKNSEEACEKNLDEALASLTNCYKNQKCKSEQSKKNNNKWIWKKSSGNEKGLQKEYANTIGLPPRTQSLCLVCLHEKEGKTQELKNISTNSELLKEWIIAAFHEGKNLKTTYPQNKNDDNGKKLFKDLKYSFADY

SEQ ID NO: 53 - protein, 646 aa, Artificial
  Synthetic Sequence
DYIKDDPYSKEYTTKLSFILNSSDANTSSEKIQKNNDEVCNPNESEISSVEQAQTSRPSSNKTCITHSSIKANKKKVCKDVKLGVRENDKVLRVCVIEHTSLSGVENCCCQDLLGILQENCSDNKRGSSSNGSCDKNSEEACEKNLDEALASLTNCYKNQKCKSEQSKKNNNKWIWKKSSGKEGGLQKEYANTIGLPPRTQSLYLGNLPKLENVCKGVTDINFDTKEKFLAGCLIAAFHEGKNLKTTYLEKKNDDNGKKLCKALEYSFADYGDLIKGTSIWDNEYTKDLELNLQKAFGKLFRKYIKKNNTAEQDTSYSSLDELRESWWNTNKKYIWTAMKHGAGMNGTTCSSGSGDSSNDIPTTDFIPQYLRFLQEWVENFCEQRQAKVKDVIENCNSCKNTSGERKIGDTCNSDCEKKCKDECEKYKKFIEDCKGGDGTAGSSWVKRWDQIYKRYSKHIEDAKRNRKAGTKNCGITTGTISGESSGATSGVTTTENKCVQSDIDSFFKHLIDIGLTTPSSYLSNVLDDNICGEDNAPWTTYTTYTTEKCNKETDKSKSQQSNTAVVVNVPSPLGNTPHGYKYACECKIPTTEETCDDRKEYMNQWSCGSAQTVRDRSGKDDYELCKYNGVQIKQAAGTLKNSKLD

SEQ ID NO: 54 - protein, 632 aa, Plasmodium falciparum
NYIKGDPYFAEYATKLSFILNSSDANNPSEKIQKNNDEVCNCNESGIASVEQEQISDPSSNKTCITHSSIKANKKKVCKHVKLGVRENDKDLRVCVIEHTSLSGVENCCCQDFLRILQENCSDNKSGSSSNGSCNNKNQEACEKNLEKVLASLTNCYKCDKCKSEQSKKNNKNWIWKKSSGKEGGLQKEYANTIGLPPRTQSLCLVVCLDEKGKKTQELKNIRTNSELLKEWIIAAFHEGKNLKPSHEKKNDDNGKKLCKALEYSFADYGDLIKGTSIWDNEYTKDLELNLQKIFGKLFRKYIKKNNTAEQDTSYSSLDELRESWWNTNKKYIWLAMKHGAGMNSTTCCGDGSVTGSGSSCDDIPTIDLIPQYLRFLQEWVEHFCKQRQEKVKPVIENCKSCKESGGTCNGECKTECKNKCEVYKKFIEDCKGGDGTAGSSWVKRWDQIYKRYSKYIEDAKRNRKAGTKNCGPSSTTNAAENKCVQSDIDSFFKHLIDIGLTTPSSYLSIVLDDNICGADKAPWTTYTTYTTTEKCNKETDKSKLQQCNTAVVVNVPSPLGNTPHGYKYACQCKIPTNEETCDDRKEYMNQWSCGSARTMKRGYKNDNYELCKYNGVDVKPTTVRSNSSKLD

SEQ ID NO: 55 - protein, 2730 aa, Plasmodium falciparum
MDKSSIANKIEAYLGAKSDDSKIDQSLKADPSEVQYYGSGGDGYYLRKNICKITVNHSDSGTNDPCDRIPPPYGDNDQWKCAIILSKVSEKPENVFVPPRRQRMCINNLEKLNVDKIRDKHAFLADVLLTARNEGERIVQNHPDTNSSNVCNALERSFADIADIIRGTDLWKGTNSNLEQNLKQMFAKIRENDKVLQDKYPKDQNYRKLREDWWNANRQKVWEVITCGARSNDLLIKRGWRTSGKSNGDNKLELCRKCGHYEEKVPTKLDYVPQFLRWLTEWIEDFYREKQNLIDDMERHREECTSEDHKSKEGTSYCSTCKDKCKKYCECVKKWKSEWENQKNKYTELYQQNKNETSQKNTSRYDDYVKDFFKKLEANYSSLENYIKGDPYFAEYATKLSFILNSSDANNPSEKIQKNNDEVCNCNESGIASVEQEQISDPSSNKTCITHSSIKANKKKVCKHVKLGVRENDKDLRVCVIEHTSLSGVENCCCQDFLRILQENCSDNKSGSSSNGSCNNKNQEACEKNLEKVLASLTNCYKCDKCKSEQSKKNNKNWIWKKSSGKEGGLQKEYANTIGLPPRTQSLCLVVCLDEKGKKTQELKNIRTNSELLKEWIIAAFHEGKNLKPSHEKKNDDNGKKLCKALEYSFADYGDLIKGTSIWDNEYTKDLELNLQKIFGKLFRKYIKKNNTAEQDTSYSSLDELRESWWNTNKKYIWLAMKHGAGMNSTTCCGDGSVTGSGSSCDDIPTIDLIPQYLRFLQEWVEHFCKQRQEKVKPVIENCKSCKESGGTCNGECKTECKNKCEVYKKFIEDCKGGDGTAGSSWVKRWDQIYKRYSKYIEDAKRNRKAGTKNCGPSSTTNAAENKCVQSDIDSFFKHLIDIGLTTPSSYLSIVLDDNICGADKAPWTTYTTYTTTEKCNKETDKSKLQQCNTAVVVNVPSPLGNTPHGYKYACQCKIPTNEETCDDRKEYMNQWSCGSARTMKRGYKNDNYELCKYNGVDVKPTTVRSNSSKLDDKDVTFFNLFEQWNKEIQYQIEQYMTNTKISCNNEKNVLSRVSDEAAQPKFSDNERDRNSITHEDKNCKEKCKCYSLWIEKINDQWDKQKDNYNKFQRKQIYDANKGSQNKKVVSLSNFLFFSCWEEYIQKYFNGDWSKIKNIGSDTFEFLIKKCGNDSGDGETIFSEKLNNAEKKCKENESTNNKMKSSETSCDCSEPIYIRGCQPKIYDGKIFPGKGGEKQWICKDTIIHGDTNGACIPPRTQNLCVGELWDKRYGGRSNIKNDTKESLKQKIKNAIQKETELLYEYHDKGTAIISRNPMKGQKEKEEKNNDSNGLPKGFCHAVQRSFIDYKNMILGTSVNIYEYIGKLQEDIKKIIEKGTTKQNGKTVGSGAENVNAWWKGIEGEMWDAVRCAITKINKKQKKNGTFSIDECGIFPPTGNDEDQSVSWFKEWSEQFCIERLQYEKNIRDACTNNGQGDKIQGDCKRKCEEYKKYISEKKQEWDKQKTKYENKYVGKSASDLLKENYPECISANFDFIFNDNIEYKTYYPYGDYSSICSCEQVKYYEYNNAEKKNNKSLCHEKGNDRTWSKKYIKKLENGRTLEGVYVPPRRQQLCLYELFPIIIKNKNDITNAKKELLETLQIVAEREAYYLWKQYHAHNDTTYLAHKKACCAIRGSFYDLEDIIKGNDLVHDEYTKYIDSKLNEIFDSSNKNDIETKRARTDWWENEAIAVPNITGANKSDPKTIRQLVWDAMQSGVRKAIDEEKEKKKPNENFPPCMGVQHIGIAKPQFIRWLEEWTNEFCEKYTKYFEDMKSNCNLRKGADDCDDNSNIECKKACANYTNWLNPKRIEWNGMSNYYNKIYRKSNKESEDGKDYSMIMEPTVIDYLNKRCNGEINGNYICCSCKNIGENSTSGTVNKKLQKKETQCEDNKGPLDLMNKVLNKMDPKYSEHKMKCTEVYLEHVEEQLKEIDNAIKDYKLYPLDRCFDDKSKMKVCDLIGDAIGCKHKTKLDELDEWNDVDMRDPYNKYKGVLIPPRRRQLCFSRIVRGPANLRNLKEFKEEILKGAQSEGKFLGNYYNEDKDKEKALEAMKNSFYDYEYIIKGSDMLTNIQFKDIKRKLDRLLEKETNNTEKVDDWWETNKKSIWNAMLCGYKKSGNKIIDPSWCTIPTTETPPQFLRWIKEWGTNVCIQKEEHKEYVKSKCSNVTNLGAQESESKNCTSEIKKYQEWSRKRSIQWEAISEGYKKYKGMDEFKNTFKNIKEPDANEPNANEYLKKHCSKCPCGFNDMQEITKYTNIGNEAFKQIKEQVDIPAELEDVIYRLKHHEYDKGNDYICNKYKNINVNMKKNNDDTWTDLVKNSSDINKGVLLPPRRKNLFLKIDESDICKYKRDPKLFKDFIYSSAISEVERLKKVYGEAKTKVVHAMKYSFADIGSIIKGDDMMENNSSDKIGKILGDGVGQNEKRKKWWDMNKYHIWESMLCGYKHAYGNISENDRKMLDIPNNDDEHQFLRWFQEWTENFCTKRNELYENMVTACNSAKCNTSNGSVDKKECTEACKNYSNFILIKKKEYQSLNSQYDMNYKETKAEKKESPEYFKDKCNGECSCLSEYFKDETRWKNPYETLDDTEVKNNCMCKPPPPASNNTSDILQKTIPFGIALALGSIAFLFMKKKPKTPVDLLRVLDIPKGDYGIPTPKSSNRYIPYASDRYKGKTYIYMEGDTSGDDDKYIWDL

SEQ ID NO: 56 - protein, 2734 aa, Artificial
  Synthetic Sequence
MDSTSTIANKIEEYLGAKSDDSKIDELLKADPSEVEYYRSGGDGDYLKNNICKITVNHSDSGKYDPCEKKLPPYDDNDQWKCQQNSSDGSGKPENICVPPRRERLCTYNLENLKFDKIRDNNAFLADVLLTARNEGEKIVQNHPDTNSSNVCNALERSFADLADIIRGTDQWKGTNSNLEKNLKQMFAKIRENDKVLQDKYPKDQKYTKLREAWWNANRQKVWEVITCGARSNDLLIKRGWRTSGKSDRKKNFELCRKCGHYEKEVPTKLDYVPQFLRWLTEWIEDFYREKQNLIDDMERHREECTREDHKSKEGTSYCSTCKDKCKKYCECVKKWKTEWENQENKYKDLYEQNKNKTSQKNTSRYDDYVKDFFEKLEANYSSLENYIKGDPYFAEYATKLSFILNPSDANNPSGETANHNDEACNCNESGISSVGQAQTSGPSSNKTCITHSSIKTNKKKECKDVKLGVRENDKDLKICVIEDTSLSGVDNCCCQDLLGILQENCSDNKRGSSSNDSCDNKNQDECQKKLEKVFASLTNGYKCDKCKSGTSRSKKKWIWKKSSGNEEGLQEEYANTIGLPPRTQSLYLGNLPKLENVCEDVKDINFDTKEKFLAGCLIVSFHEGKNLKKRYPQNKNSGNKENLCKALEYSFADYGDLIKGTSIWDNEYTKDLELNLQNNFGKLFGKYIKKNNTAEQDTSYSSLDELRESWWNTNKKYIWTAMKHGAEMNITTCNADGSVTGSGSSCDDIPTIDLIPQYLRFLQEWVENFCEQRQAKVKDVITNCKSCKESGNKCKTECKTKCKDECEKYKKFIEACGTAGGGIGTAGSPWSKRWDQIYKRYSKHIEDAKRNRKAGTKNCGTSSTTNAAASTDENKCVQSDIDSFFKHLIDIGLTTPSSYLSNVLDDNICGADKAPWTTYTTYTTTEKCNKERDKSKSQSSDTLVVVNVPSPLGNTPYRYKYACQCKIPTNEETCDDRKEYMNQWSCGSARTMKRGYKNDNYELCKYNGVDVKPTTVRSNSSKLDGNDVTFFNLFEQWNKEIQYQIEQYMTNANISCIDEKEVLDSVSDEGTPKVRGGYEDGRNNNTDQGTNCKEKCKCYKLWIEKINDQWGKQKDNYNKFRSKQIYDANKGSQNKKVVSLSNFLFFSCWEEYIQKYFNGDWSKIKNIGSDTFEFLIKKCGNNSAHGEEIFNEKLKNAEKKCKENESTDTNINKSETSCDLNATNYIRGCQSKTYDGKIFPGKGGEKQWICKDTIIHGDTNGACIPPRTQNLCVGELWDKSYGGRSNIKNDTKELLKEKIKNAIHKETELLYEYHDTGTAIISKNDKKGQKGKNDPNGLPKGFCHAVQRSFIDYKNMILGTSVNIYEHIGKLQEDIKKIIEKGTPQQKDKIGGVGSSTENVNAWWKGIEREMWDAVRCAITKINKKNNNSIFNGDECGVSPPTGNDEDQSVSWFKEWGEQFCIERLRYEQNIREACTINGKNEKKCINSKSGQGDKIQGACKRKCEKYKKYISEKKQEWDKQKTKYENKYVGKSASDLLKENYPECISANFDFIFNDNIEYKTYYPYGDYSSICSCEQVKYYKYNNAEKKNNKSLCYEKDNDMTWSKKYIKKLENGRSLEGVYVPPRRQQLCLYELFPIIIKNEEGMEKAKEELLETLQIVAEREAYYLWKQYNPTGKGIDDANKKACCAIRGSFYDLEDIIKGNDLVHDEYTKYIDSKLNEIFGSSDTNDIDTKRARTDWWENETITNGTDRKTIRQLVWDAMQSGVRYAVEEKNENFPLCMGVEHIGIAKPQFIRWLEEWTNEFCEKYTKYFEDMKSKCDPPKRADTCGDNSNIECKKACANYTNWLNPKRIEWNGMSNYYNKIYRKSNKESEGGKDYSMIMAPTVIDYLNKRCHGEINGNYICCSCKNIGAYNTTSGTVNKKLQKKETECEEEKGPLDLMNEVLNKMDKKYSAHKMKCTEVYLEHVEEQLNEIDNAIKDYKLYPLDRCFDDQTKMKVCDLIADAIGCKDKTKLDELDEWNDMDLRGTYNKHKGVLIPPRRRQLCFSRIVRGPANLRSLNEFKEEILKGAQSEGKFLGNYYKEHKDKEKALEAMKNSFYDYEDIIKGTDMLTNIEFKDIKIKLDRLLEKETNNTKKAEDWWKTNKKSIWNAMLCGYKKSGNKIIDPSWCTIPTTETPPQFLRWIKEWGTNVCIQKQEHKEYVKSKCSNVTNLGAQASESNNCTSEIKKYQEWSRKRSIRWETISKRYKKYKRMDILKDVKEPDANTYLREHCSKCPCGFNDMEEMNNNEDNEKEAFKQIKEQVKIPAELEDVIYRIKHHEYDKGNDYICNKYKNIHDRMKKNNGNFVTDNFVKKSWEISNGVLIPPRRKNLFLYIDPSKICEYKKDPKLFKDFIYWSAFTEVERLKKAYGGARAKVVHAMKYSFTDIGSIIKGDDMMEKNSSDKIGKILGDTDGQNEKRKKWWDMNKYHIWESMLCGYREAEGDTETNENCRFPDIESVPQFLRWFQEWSENFCDRRQKLYDKLNSECISAECTNGSVDNSKCTHACVNYKNYILTKKTEYEIQTNKYDNEFKNKNSNDKDAPDYLKEKCNDNKCECLNKHIDDKNKTWKNPYETLEDTFKSKCDCPKPLPSPIKPDDLPPQADEPFDPTILQTTIPFGIALALGSIAFLFMKVIYIYIYVCCICMYVCMYVCMYVCMYVCMYVCMHVCMLCVYVIYVFKICIYIEKEKRKK

SEQ ID NO: 57 - protein, 58 aa, Artificial [sequence omitted]

SEQ ID NO: 58 - protein, 324 aa, Artificial
  Synthetic Sequence
RHRQPRGWEQLEQCGYPVQRLVALYLAARLSWNQVDQVIRNALASPGSGGDLGEAIREQPEQARLALTLAAAESERFVRQGTGNDEAGAANGPADSGDALLERNYPTGAEFLGDGGDISFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTSLTLAAPEAAGEVERLIGHPLPLRLDAITGPEEEGGRLETILGWPLAERTVVIPSAIPTDPRNVGGDLDPSSIPDKEQAISALPDYASQPGKPPRKDEL

SEQ ID NO: 59 - protein, 231 aa, Artificial
  Synthetic Sequence
RHRQPRGWEQLYPTGAEFLGDGGDISFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTSLTLAAPEAAGEVERLIGHPLPLRLDAITGPEEEGGRLETILGWPLAERTVVIPSAIPTDPRNVGGDLDPSSIPDKEQAISALPDYASQPGKPPRKDEL

SEQ ID NO: 60 - protein, 929 aa, Artificial
  Synthetic Sequence
RPDFCLEPPYTGPCKARIIRYFYNAKAGLCQTFVYGGCRAKRNNFKSAEDCMRTCGGANYIKGDPYFAEYATKLSFILNPSDANNPSGETANHNDEACNCNESGISSVGQAQTSGPSSNKTCITHSSIKTNKKKECKDVKLGVRENDKDLKICVIEDTSLSGVDNCCCQDLLGILQENCSDNKRGSSSNDSCDNKNQDECQKKLEKVFASLTNGYKCDKCKSGTSRSKKKWIWKKSSGNEEGLQEEYANTIGLPPRTQSLYLGNLPKLENVCEDVKDINFDTKEKFLAGCLIVSFHEGKNLKKRYPQNKNSGNKENLCKALEYSFADYGDLIKGTSIWDNEYTKDLELNLQNNFGKLFGKYIKKNNTAEQDTSYSSLDELRESWWNTNKKYIWTAMKHGAEMNITTCNADGSVTGSGSSCDDIPTIDLIPQYLRFLQEWVENFCEQRQAKVKDVITNCKSCKESGNKCKTECKTKCKDECEKYKKFIEACGTAGGGIGTAGSPWSKRWDQIYKRYSKHIEDAKRNRKAGTKNCGTSSTTNAAASTDENKCVQSDIDSFFKHLIDIGLTTPSSYLSNVLDDNICGADKAPWTTYTTYTTTEKCNKERDKSKSQSSDTLVVVNVPSPLGNTPYRYKYACQCKIPTNEETCDDRKEYMNQWSCGSARTMKRGYKNDNYELCKYNGVDVKPTTVRSNSSKLDRHRQPRGWEQLYPTGAEFLGDGGDISFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTSLTLAAPEAAGEVERLIGHPLPLRLDAITGPEEEGGRLETILGWPLAERTVVIPSAIPTDPRNVGGDLDPSSIPDKEQAISALPDYASQPGKPPRKDEL

SEQ ID NO: 61 - protein, 1045 aa, Artificial
  Synthetic Sequence
RPDFCLEPPYTGPCKARIIRYFYNAKAGLCQTFVYGGCRAKRNNFKSAEDCMRTCGGANYIKGDPYFAEYATKLSFILNPSDANNPSGETANHNDEACNCNESGISSVGQAQTSGPSSNKTCITHSSIKTNKKKECKDVKLGVRENDKDLKICVIEDTSLSGVDNCCCQDLLGILQENCSDNKRGSSSNDSCDNKNQDECQKKLEKVFASLTNGYKCDKCKSGTSRSKKKWIWKKSSGNEEGLQEEYANTIGLPPRTQSLYLGNLPKLENVCEDVKDINFDTKEKFLAGCLIVSFHEGKNLKKRYPQNKNSGNKENLCKALEYSFADYGDLIKGTSIWDNEYTKDLELNLQNNFGKLFGKYIKKNNTAEQDTSYSSLDELRESWWNTNKKYIWTAMKHGAEMNITTCNADGSVTGSGSSCDDIPTIDLIPQYLRFLQEWVENFCEQRQAKVKDVITNCKSCKESGNKCKTECKTKCKDECEKYKKFIEACGTAGGGIGTAGSPWSKRWDQIYKRYSKHIEDAKRNRKAGTKNCGTSSTTNAAASTDENKCVQSDIDSFFKHLIDIGLTTPSSYLSNVLDDNICGADKAPWTTYTTYTTTEKCNKERDKSKSQSSDTLVVVNVPSPLGNTPYRYKYACQCKIPTNEETCDDRKEYMNQWSCGSARTMKRGYKNDNYELCKYNGVDVKPTTVRSNSSKLDPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAANGPADSGDALLERNYPTGAEFLGDGGDISFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTSLTLAAPEAAGEVERLIGHPLPLRLDAITGPEEEGGRLETILGWPLAERTVVIPSAIPTDPRNVGGDLDPSSIPDKEQAISALPDYASQPGKPPRKDEL

SEQ ID NO: 62 - protein, 987 aa, Artificial
  Synthetic Sequence
NYIKGDPYFAEYATKLSFILNPSDANNPSGETANHNDEACNCNESGISSVGQAQTSGPSSNKTCITHSSIKTNKKKECKDVKLGVRENDKDLKICVIEDTSLSGVDNCCCQDLLGILQENCSDNKRGSSSNDSCDNKNQDECQKKLEKVFASLTNGYKCDKCKSGTSRSKKKWIWKKSSGNEEGLQEEYANTIGLPPRTQSLYLGNLPKLENVCEDVKDINFDTKEKFLAGCLIVSFHEGKNLKKRYPQNKNSGNKENLCKALEYSFADYGDLIKGTSIWDNEYTKDLELNLQNNFGKLFGKYIKKNNTAEQDTSYSSLDELRESWWNTNKKYIWTAMKHGAEMNITTCNADGSVTGSGSSCDDIPTIDLIPQYLRFLQEWVENFCEQRQAKVKDVITNCKSCKESGNKCKTECKTKCKDECEKYKKFIEACGTAGGGIGTAGSPWSKRWDQIYKRYSKHIEDAKRNRKAGTKNCGTSSTTNAAASTDENKCVQSDIDSFFKHLIDIGLTTPSSYLSNVLDDNICGADKAPWTTYTTYTTTEKCNKERDKSKSQSSDTLVVVNVPSPLGNTPYRYKYACQCKIPTNEETCDDRKEYMNQWSCGSARTMKRGYKNDNYELCKYNGVDVKPTTVRSNSSKLDPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAANGPADSGDALLERNYPTGAEFLGDGGDISFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTSLTLAAPEAAGEVERLIGHPLPLRLDAITGPEEEGGRLETILGWPLAERTVVIPSAIPTDPRNVGGDLDPSSIPDKEQAISALPDYASQPGKPPRKDEL

SEQ ID NO: 63 - protein, 871 aa, Artificial
  Synthetic Sequence
NYIKGDPYFAEYATKLSFILNPSDANNPSGETANHNDEACNCNESGISSVGQAQTSGPSSNKTCITHSSIKTNKKKECKDVKLGVRENDKDLKICVIEDTSLSGVDNCCCQDLLGILQENCSDNKRGSSSNDSCDNKNQDECQKKLEKVFASLTNGYKCDKCKSGTSRSKKKWIWKKSSGNEEGLQEEYANTIGLPPRTQSLYLGNLPKLENVCEDVKDINFDTKEKFLAGCLIVSFHEGKNLKKRYPQNKNSGNKENLCKALEYSFADYGDLIKGTSIWDNEYTKDLELNLQNNFGKLFGKYIKKNNTAEQDTSYSSLDELRESWWNTNKKYIWTAMKHGAEMNITTCNADGSVTGSGSSCDDIPTIDLIPQYLRFLQEWVENFCEQRQAKVKDVITNCKSCKESGNKCKTECKTKCKDECEKYKKFIEACGTAGGGIGTAGSPWSKRWDQIYKRYSKHIEDAKRNRKAGTKNCGTSSTTNAAASTDENKCVQSDIDSFFKHLIDIGLTTPSSYLSNVLDDNICGADKAPWTTYTTYTTTEKCNKERDKSKSQSSDTLVVVNVPSPLGNTPYRYKYACQCKIPTNEETCDDRKEYMNQWSCGSARTMKRGYKNDNYELCKYNGVDVKPTTVRSNSSKLDRHRQPRGWEQLYPTGAEFLGDGGDISFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTSLTLAAPEAAGEVERLIGHPLPLRLDAITGPEEEGGRLETILGWPLAERTVVIPSAIPTDPRNVGGDLDPSSIPDKEQAISALPDYASQPGKPPRKDEL

SEQ ID NO: 64 - protein, 1258 aa, Artificial
  Synthetic Sequence
RPDFCLEPPYTGPCKARIIRYFYNAKAGLCQTFVYGGCRAKRNNFKSAEDCMRTCGGANHSDSGKYDPCEKKLPPYDDNDQWKCQQNSSDGSGKPENICVPPRRERLCTYNLENLKFDKIRDNNAFLADVLLTARNEGEKIVQNHPDTNSSNVCNALERSFADLADIIRGTDQWKGTNSNLEKNLKQMFAKIRENDKVLQDKYPKDQKYTKLREAWWNANRQKVWEVITCGARSNDLLIKRGWRTSGKSDRKKNFELCRKCGHYEKEVPTKLDYVPQFLRWLTEWIEDFYREKQNLIDDMERHREECTREDHKSKEGTSYCSTCKDKCKKYCECVKKWKTEWENQENKYKDLYEQNKNKTSQKNTSRYDDYVKDFFEKLEANYSSLENYIKGDPYFAEYATKLSFILNPSDANNPSGETANHNDEACNCNESGISSVGQAQTSGPSSNKTCITHSSIKTNKKKECKDVKLGVRENDKDLKICVIEDTSLSGVDNCCCQDLLGILQENCSDNKRGSSSNDSCDNKNQDECQKKLEKVFASLTNGYKCDKCKSGTSRSKKKWIWKKSSGNEEGLQEEYANTIGLPPRTQSLYLGNLPKLENVCEDVKDINFDTKEKFLAGCLIVSFHEGKNLKKRYPQNKNSGNKENLCKALEYSFADYGDLIKGTSIWDNEYTKDLELNLQNNFGKLFGKYIKKNNTAEQDTSYSSLDELRESWWNTNKKYIWTAMKHGAEMNITTCNADGSVTGSGSSCDDIPTIDLIPQYLRFLQEWVENFCEQRQAKVKDVITNCKSCKESGNKCKTECKTKCKDECEKYKKFIEACGTAGGGIGTAGSPWSKRWDQIYKRYSKHIEDAKRNRKAGTKNCGTSSTTNAAASTDENKCVQSDIDSFFKHLIDIGLTTPSSYLSNVLDDNICGADKAPWTTYTTYTTTEKCNKERDKSKSQSSDTLVVVNVPSPLGNTPYRYKYACQCKIPTNEETCDDRKEYMNQWSCGSARTMKRGYKNDNYELCKYNGVDVKPTTVRSNSSKLDRHRQPRGWEQLYPTGAEFLGDGGDISFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTSLTLAAPEAAGEVERLIGHPLPLRLDAITGPEEEGGRLETILGWPLAERTVVIPSAIPTDPRNVGGDLDPSSIPDKEQAISALPDYASQPGKPPRKDEL

SEQ ID NO: 65 - protein, 1374 aa, Artificial
  Synthetic Sequence
RPDFCLEPPYTGPCKARIIRYFYNAKAGLCQTFVYGGCRAKRNNFKSAEDCMRTCGGANHSDSGKYDPCEKKLPPYDDNDQWKCQQNSSDGSGKPENICVPPRRERLCTYNLENLKFDKIRDNNAFLADVLLTARNEGEKIVQNHPDTNSSNVCNALERSFADLADIIRGTDQWKGTNSNLEKNLKQMFAKIRENDKVLQDKYPKDQKYTKLREAWWNANRQKVWEVITCGARSNDLLIKRGWRTSGKSDRKKNFELCRKCGHYEKEVPTKLDYVPQFLRWLTEWIEDFYREKQNLIDDMERHREECTREDHKSKEGTSYCSTCKDKCKKYCECVKKWKTEWENQENKYKDLYEQNKNKTSQKNTSRYDDYVKDFFEKLEANYSSLENYIKGDPYFAEYATKLSFILNPSDANNPSGETANHNDEACNCNESGISSVGQAQTSGPSSNKTCITHSSIKTNKKKECKDVKLGVRENDKDLKICVIEDTSLSGVDNCCCQDLLGILQENCSDNKRGSSSNDSCDNKNQDECQKKLEKVFASLTNGYKCDKCKSGTSRSKKKWIWKKSSGNEEGLQEEYANTIGLPPRTQSLYLGNLPKLENVCEDVKDINFDTKEKFLAGCLIVSFHEGKNLKKRYPQNKNSGNKENLCKALEYSFADYGDLIKGTSIWDNEYTKDLELNLQNNFGKLFGKYIKKNNTAEQDTSYSSLDELRESWWNTNKKYIWTAMKHGAEMNITTCNADGSVTGSGSSCDDIPTIDLIPQYLRFLQEWVENFCEQRQAKVKDVITNCKSCKESGNKCKTECKTKCKDECEKYKKFIEACGTAGGGIGTAGSPWSKRWDQIYKRYSKHIEDAKRNRKAGTKNCGTSSTTNAAASTDENKCVQSDIDSFFKHLIDIGLTTPSSYLSNVLDDNICGADKAPWTTYTTYTTTEKCNKERDKSKSQSSDTLVVVNVPSPLGNTPYRYKYACQCKIPTNEETCDDRKEYMNQWSCGSARTMKRGYKNDNYELCKYNGVDVKPTTVRSNSSKLDPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAANGPADSGDALLERNYPTGAEFLGDGGDISFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTSLTLAAPEAAGEVERLIGHPLPLRLDAITGPEEEGGRLETILGWPLAERTVVIPSAIPTDPRNVGGDLDPSSIPDKEQAISALPDYASQPGKPPRKDEL

SEQ ID NO: 66 - protein, 1200 aa, Artificial
  Synthetic Sequence
NHSDSGKYDPCEKKLPPYDDNDQWKCQQNSSDGSGKPENICVPPRRERLCTYNLENLKFDKIRDNNAFLADVLLTARNEGEKIVQNHPDTNSSNVCNALERSFADLADIIRGTDQWKGTNSNLEKNLKQMFAKIRENDKVLQDKYPKDQKYTKLREAWWNANRQKVWEVITCGARSNDLLIKRGWRTSGKSDRKKNFELCRKCGHYEKEVPTKLDYVPQFLRWLTEWIEDFYREKQNLIDDMERHREECTREDHKSKEGTSYCSTCKDKCKKYCECVKKWKTEWENQENKYKDLYEQNKNKTSQKNTSRYDDYVKDFFEKLEANYSSLENYIKGDPYFAEYATKLSFILNPSDANNPSGETANHNDEACNCNESGISSVGQAQTSGPSSNKTCITHSSIKTNKKKECKDVKLGVRENDKDLKICVIEDTSLSGVDNCCCQDLLGILQENCSDNKRGSSSNDSCDNKNQDECQKKLEKVFASLTNGYKCDKCKSGTSRSKKKWIWKKSSGNEEGLQEEYANTIGLPPRTQSLYLGNLPKLENVCEDVKDINFDTKEKFLAGCLIVSFHEGKNLKKRYPQNKNSGNKENLCKALEYSFADYGDLIKGTSIWDNEYTKDLELNLQNNFGKLFGKYIKKNNTAEQDTSYSSLDELRESWWNTNKKYIWTAMKHGAEMNITTCNADGSVTGSGSSCDDIPTIDLIPQYLRFLQEWVENFCEQRQAKVKDVITNCKSCKESGNKCKTECKTKCKDECEKYKKFIEACGTAGGGIGTAGSPWSKRWDQIYKRYSKHIEDAKRNRKAGTKNCGTSSTTNAAASTDENKCVQSDIDSFFKHLIDIGLTTPSSYLSNVLDDNICGADKAPWTTYTTYTTTEKCNKERDKSKSQSSDTLVVVNVPSPLGNTPYRYKYACQCKIPTNEETCDDRKEYMNQWSCGSARTMKRGYKNDNYELCKYNGVDVKPTTVRSNSSKLDRHRQPRGWEQLYPTGAEFLGDGGDISFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTSLTLAAPEAAGEVERLIGHPLPLRLDAITGPEEEGGRLETILGWPLAERTVVIPSAIPTDPRNVGGDLDPSSIPDKEQAISALPDYASQPGKPPRKDEL

SEQ ID NO: 67 - protein, 1316 aa, Artificial
  Synthetic Sequence
NHSDSGKYDPCEKKLPPYDDNDQWKCQQNSSDGSGKPENICVPPRRERLCTYNLENLKFDKIRDNNAFLADVLLTARNEGEKIVQNHPDTNSSNVCNALERSFADLADIIRGTDQWKGTNSNLEKNLKQMFAKIRENDKVLQDKYPKDQKYTKLREAWWNANRQKVWEVITCGARSNDLLIKRGWRTSGKSDRKKNFELCRKCGHYEKEVPTKLDYVPQFLRWLTEWIEDFYREKQNLIDDMERHREECTREDHKSKEGTSYCSTCKDKCKKYCECVKKWKTEWENQENKYKDLYEQNKNKTSQKNTSRYDDYVKDFFEKLEANYSSLENYIKGDPYFAEYATKLSFILNPSDANNPSGETANHNDEACNCNESGISSVGQAQTSGPSSNKTCITHSSIKTNKKKECKDVKLGVRENDKDLKICVIEDTSLSGVDNCCCQDLLGILQENCSDNKRGSSSNDSCDNKNQDECQKKLEKVFASLTNGYKCDKCKSGTSRSKKKWIWKKSSGNEEGLQEEYANTIGLPPRTQSLYLGNLPKLENVCEDVKDINFDTKEKFLAGCLIVSFHEGKNLKKRYPQNKNSGNKENLCKALEYSFADYGDLIKGTSIWDNEYTKDLELNLQNNFGKLFGKYIKKNNTAEQDTSYSSLDELRESWWNTNKKYIWTAMKHGAEMNITTCNADGSVTGSGSSCDDIPTIDLIPQYLRFLQEWVENFCEQRQAKVKDVITNCKSCKESGNKCKTECKTKCKDECEKYKKFIEACGTAGGGIGTAGSPWSKRWDQIYKRYSKHIEDAKRNRKAGTKNCGTSSTTNAAASTDENKCVQSDIDSFFKHLIDIGLTTPSSYLSNVLDDNICGADKAPWTTYTTYTTTEKCNKERDKSKSQSSDTLVVVNVPSPLGNTPYRYKYACQCKIPTNEETCDDRKEYMNQWSCGSARTMKRGYKNDNYELCKYNGVDVKPTTVRSNSSKLDPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAANGPADSGDALLERNYPTGAEFLGDGGDISFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTSLTLAAPEAAGEVERLIGHPLPLRLDAITGPEEEGGRLETILGWPLAERTVVIPSAIPTDPRNVGGDLDPSSIPDKEQAISALPDYASQPGKPPRKDEL

SEQ ID NO: 68 - protein, 964 aa, Artificial
  Synthetic Sequence
LSFILNSSDANNPSEKIQKNNDEVCNCNESGIASVEQEQISDPSSNKTCITHSSIKANKKKVCKHVKLGVRENDKDLRVCVIEHTSLSGVENCCCQDFLRILQENCSDNKSGSSSNGSCNNKNQEACEKNLEKVLASLTNCYKCDKCKSEQSKKNNKNWIWKKSSGKEGGLQKEYANTIGLPPRTQSLCLVVCLDEKGKKTQELKNIRTNSELLKEWIIAAFHEGKNLKPSHEKKNDDNGKKLCKALEYSFADYGDLIKGTSIWDNEYTKDLELNLQKIFGKLFRKYIKKNNTAEQDTSYSSLDELRESWWNTNKKYIWLAMKHGAGMNSTTCCGDGSVTGSGSSCDDIPTIDLIPQYLRFLQEWVEHFCKQRQEKVKPVIENCKSCKESGGTCNGECKTECKNKCEVYKKFIEDCKGGDGTAGSSWVKRWDQIYKRYSKYIEDAKRNRKAGTKNCGPSSTTNAAENKCVQSDIDSFFKHLIDIGLTTPSSYLSIVLDDNICGADKAPWTTYTTYTTTEKCNKETDKSKLQQCNTAVVVNVPSPLGNTPHGYKYACQCKIPTNEETCDDRKEYMNQWSCGSARTMKRGYKNDNYELCKYNGVDVKPTTVRSNSSKLDPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAANGPADSGDALLERNYPTGAEFLGDGGDISFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTSLTLAAPEAAGEVERLIGHPLPLRLDAITGPEEEGGRLETILGWPLAERTVVIPSAIPTDPRNVGGDLDPSSIPDKEQAISALPDYASQPGKPPRKDEL

SEQ ID NO: 69 - protein, 848 aa, Artificial
  Synthetic Sequence
LSFILNSSDANNPSEKIQKNNDEVCNCNESGIASVEQEQISDPSSNKTCITHSSIKANKKKVCKHVKLGVRENDKDLRVCVIEHTSLSGVENCCCQDFLRILQENCSDNKSGSSSNGSCNNKNQEACEKNLEKVLASLTNCYKCDKCKSEQSKKNNKNWIWKKSSGKEGGLQKEYANTIGLPPRTQSLCLVVCLDEKGKKTQELKNIRTNSELLKEWIIAAFHEGKNLKPSHEKKNDDNGKKLCKALEYSFADYGDLIKGTSIWDNEYTKDLELNLQKIFGKLFRKYIKKNNTAEQDTSYSSLDELRESWWNTNKKYIWLAMKHGAGMNSTTCCGDGSVTGSGSSCDDIPTIDLIPQYLRFLQEWVEHFCKQRQEKVKPVIENCKSCKESGGTCNGECKTECKNKCEVYKKFIEDCKGGDGTAGSSWVKRWDQIYKRYSKYIEDAKRNRKAGTKNCGPSSTTNAAENKCVQSDIDSFFKHLIDIGLTTPSSYLSIVLDDNICGADKAPWTTYTTYTTTEKCNKETDKSKLQQCNTAVVVNVPSPLGNTPHGYKYACQCKIPTNEETCDDRKEYMNQWSCGSARTMKRGYKNDNYELCKYNGVDVKPTTVRSNSSKLDRHRQPRGWEQLYPTGAEFLGDGGDISFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTSLTLAAPEAAGEVERLIGHPLPLRLDAITGPEEEGGRLETILGWPLAERTVVIPSAIPTDPRNVGGDLDPSSIPDKEQAISALPDYASQPGKPPRKDEL

SEQ ID NO: 70 - protein, 808 aa, Artificial
  Synthetic Sequence
NYIKGDPYFAEYATKLSFILNPSDANNPSGETANHNDEACNCNESGISSVGQAQTSGPSSNKTCITHSSIKTNKKKECKDVKLGVRENDKDLKICVIEDTSLSGVDNCCCQDLLGILQENCSDNKRGSSSNDSCDNKNQDECQKKLEKVFASLTNGYKCDKCKSGTSRSKKKWIWKKSSGNEEGLQEEYANTIGLPPRTQSLYLGNLPKLENVCEDVKDINFDTKEKFLAGCLIVSFHEGKNLKKRYPQNKNSGNKENLCKALEYSFADYGDLIKGTSIWDNEYTKDLELNLQNNFGKLFGKYIKKNNTAEQDTSYSSLDELRESWWNTNKKYIWTAMKHGAEMNITTCNADGSVTGSGSSCDDIPTIDLIPQYLRFLQEWVENFCEQRQAKVKDVITNCKSCKESGNKCKTECKTKCKDECEKYKKFIEACGTAGGGIGTAGSPWSKRWDQIYKRYSKHIEDAKRNRKAGTKNCGTSSTTNAAASTDENKCVQSDIDSFFKHLIDIGLTTPSSYLSNVLDDNICGADKAPWTTYTTYTTTEKCNKERDKSKSQSSDTLVVVNVPSPLGNTPYRYKYRHRQPRGWEQLYPTGAEFLGDGGDISFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTSLTLAAPEAAGEVERLIGHPLPLRLDAITGPEEEGGRLETILGWPLAERTVVIPSAIPTDPRNVGGDLDPSSIPDKEQAISALPDYASQPGKPPRKDEL

SEQ ID NO: 71 - protein, 393 aa, Artificial
  Synthetic Sequence
MGADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPMHEF

SEQ ID NO: 72 - protein, 1356 aa, Artificial
  Synthetic Sequence
MGADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPMHEFHSDSGTNDPCDRIPPPYGDNDQWKCAIILSKVSEKPENVFVPPRRQRMCINNLEKLNVDKIRDKHAFLADVLLTARNEGERIVQNHPDTNSSNVCNALERSFADIADIIRGTDLWKGTNSNLEQNLKQMFAKIRENDKVLQDKYPKDQNYRKLREDWWNANRQKVWEVITCGARSNDLLIKRGWRTSGKSNGDNKLELCRKCGHYEEKVPTKLDYVPQFLRWLTEWIEDFYREKQNLIDDMERHREECTSEDHKSKEGTSYCSTCKDKCKKYCECVKKWKSEWENQKNKYTELYQQNKNETSQKNTSRYDDYVKDFFKKLEANYSSLENYIKGDPYFAEYATKLSFILNSSDANNPSEKIQKNNDEVCNCNESGIASVEQEQISDPSSNKTCITHSSIKANKKKVCKHVKLGVRENDKDLRVCVIEHTSLSGVENCCCQDFLRILQENCSDNKSGSSSNGSCNNKNQEACEKNLEKVLASLTNCYKCDKCKSEQSKKNNKNWIWKKSSGKEGGLQKEYANTIGLPPRTQSLCLVVCLDEKGKKTQELKNIRTNSELLKEWIIAAFHEGKNLKPSHEKKNDDNGKKLCKALEYSFADYGDLIKGTSIWDNEYTKDLELNLQKIFGKLFRKYIKKNNTAEQDTSYSSLDELRESWWNTNKKYIWLAMKHGAGMNSTTCCGDGSVTGSGSSCDDIPTIDLIPQYLRFLQEWVEHFCKQRQEKVKPVIENCKSCKESGGTCNGECKTECKNKCEVYKKFIEDCKGGDGTAGSSWVKRWDQIYKRYSKYIEDAKRNRKAGTKNCGPSSTTNAAENKCVQSDIDSFFKHLIDIGLTTPSSYLSIVLDDNICGADKAPWTTYTTYTTTEKCNKETDKSKLQQCNTAVVVNVPSPLGNTPHGYKYACQCKIPTNEETCDDRKEYMNQWSCGSARTMKRGYKNDNYELCKYNGVDVKPTTVRSNSSKLDSGR

SEQ ID NO: 73 - protein, 1364 aa, Artificial
  Synthetic Sequence
MGADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPMHEFHSDSGKYDPCEKKLPPYDDNDQWKCQQNSSDGSGKPENICVPPRRERLCTYNLENLKFDKIRDNNAFLADVLLTARNEGEKIVQNHPDTNSSNVCNALERSFADLADIIRGTDQWKGTNSNLEKNLKQMFAKIRENDKVLQDKYPKDQKYTKLREAWWNANRQKVWEVITCGARSNDLLIKRGWRTSGKSDRKKNFELCRKCGHYEKEVPTKLDYVPQFLRWLTEWIEDFYREKQNLIDDMERHREECTREDHKSKEGTSYCSTCKDKCKKYCECVKKWKTEWENQENKYKDLYEQNKNKTSQKNTSRYDDYVKDFFEKLEANYSSLENYIKGDPYFAEYATKLSFILNPSDANNPSGETANHNDEACNCNESGISSVGQAQTSGPSSNKTCITHSSIKTNKKKECKDVKLGVRENDKDLKICVIEDTSLSGVDNCCCQDLLGILQENCSDNKRGSSSNDSCDNKNQDECQKKLEKVFASLTNGYKCDKCKSGTSRSKKKWIWKKSSGNEEGLQEEYANTIGLPPRTQSLYLGNLPKLENVCEDVKDINFDTKEKFLAGCLIVSFHEGKNLKKRYPQNKNSGNKENLCKALEYSFADYGDLIKGTSIWDNEYTKDLELNLQNNFGKLFGKYIKKNNTAEQDTSYSSLDELRESWWNTNKKYIWTAMKHGAEMNITTCNADGSVTGSGSSCDDIPTIDLIPQYLRFLQEWVENFCEQRQAKVKDVITNCKSCKESGNKCKTECKTKCKDECEKYKKFIEACGTAGGGIGTAGSPWSKRWDQIYKRYSKHIEDAKRNRKAGTKNCGTSSTTNAAASTDENKCVQSDIDSFFKHLIDIGLTTPSSYLSNVLDDNICGADKAPWTTYTTYTTTEKCNKERDKSKSQSSDTLVVVNVPSPLGNTPYRYKYACQCKIPTNEETCDDRKEYMNQWSCGSARTMKRGYKNDNYELCKYNGVDVKPTTVRSNSSKLDSGR

SEQ ID NO: 74 - protein, 1013 aa, Artificial
  Synthetic Sequence
MGADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPMHEFLSFILNSSDANNPSEKIQKNNDEVCNCNESGIASVEQEQISDPSSNKTCITHSSIKANKKKVCKHVKLGVRENDKDLRVCVIEHTSLSGVENCCCQDFLRILQENCSDNKSGSSSNGSCNNKNQEACEKNLEKVLASLTNCYKCDKCKSEQSKKNNKNWIWKKSSGKEGGLQKEYANTIGLPPRTQSLCLVVCLDEKGKKTQELKNIRTNSELLKEWIIAAFHEGKNLKPSHEKKNDDNGKKLCKALEYSFADYGDLIKGTSIWDNEYTKDLELNLQKIFGKLFRKYIKKNNTAEQDTSYSSLDELRESWWNTNKKYIWLAMKHGAGMNSTTCCGDGSVTGSGSSCDDIPTIDLIPQYLRFLQEWVEHFCKQRQEKVKPVIENCKSCKESGGTCNGECKTECKNKCEVYKKFIEDCKGGDGTAGSSWVKRWDQIYKRYSKYIEDAKRNRKAGTKNCGPSSTTNAAENKCVQSDIDSFFKHLIDIGLTTPSSYLSIVLDDNICGADKAPWTTYTTYTTTEKCNKETDKSKLQQCNTAVVVNVPSPLGNTPHGYKYACQCKIPTNEETCDDRKEYMNQWSCGSARTMKRGYKNDNYELCKYNGVDVKPTTVRSNSSKLDSGR

SEQ ID NO: 75 - protein, 1036 aa, Artificial
  Synthetic Sequence
MGADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPMHEFNYIKGDPYFAEYATKLSFILNPSDANNPSGETANHNDEACNCNESGISSVGQAQTSGPSSNKTCITHSSIKTNKKKECKDVKLGVRENDKDLKICVIEDTSLSGVDNCCCQDLLGILQENCSDNKRGSSSNDSCDNKNQDECQKKLEKVFASLTNGYKCDKCKSGTSRSKKKWIWKKSSGNEEGLQEEYANTIGLPPRTQSLYLGNLPKLENVCEDVKDINFDTKEKFLAGCLIVSFHEGKNLKKRYPQNKNSGNKENLCKALEYSFADYGDLIKGTSIWDNEYTKDLELNLQNNFGKLFGKYIKKNNTAEQDTSYSSLDELRESWWNTNKKYIWTAMKHGAEMNITTCNADGSVTGSGSSCDDIPTIDLIPQYLRFLQEWVENFCEQRQAKVKDVITNCKSCKESGNKCKTECKTKCKDECEKYKKFIEACGTAGGGIGTAGSPWSKRWDQIYKRYSKHIEDAKRNRKAGTKNCGTSSTTNAAASTDENKCVQSDIDSFFKHLIDIGLTTPSSYLSNVLDDNICGADKAPWTTYTTYTTTEKCNKERDKSKSQSSDTLVVVNVPSPLGNTPYRYKYACQCKIPTNEETCDDRKEYMNQWSCGSARTMKRGYKNDNYELCKYNGVDVKPTTVRSNSSKLDSGR

SEQ ID NO: 76 - DNA, 18 bp, Artificial
  Synthetic Sequence
aactacatcaagggcgac

SEQ ID NO: 77 - DNA, 21 bp, Artificial
  Synthetic Sequence
cttgttgatattggtgtcggt

SEQ ID NO: 78 - DNA, 18 bp, Artificial
  Synthetic Sequence
cacagcgatagcggcaag

SEQ ID NO: 79 - DNA, 18 bp, Artificial
  Synthetic Sequence
gtccagcttgctggagtt

SEQ ID NO: 80 - DNA, 18 bp, Artificial
  Synthetic Sequence
aactacatcaagggcgac

SEQ ID NO: 81 - DNA, 18 bp, Artificial
  Synthetic Sequence
gtccagcttgctggagtt

SEQ ID NO: 82 - DNA, 18 bp, Artificial
  Synthetic Sequence
aactacatcaagggcgac

SEQ ID NO: 83 - DNA, 18 bp, Artificial
  Synthetic Sequence
agcggcgttggtggtgga

SEQ ID NO: 84 - DNA, 18 bp, Artificial
  Synthetic Sequence
aactacatcaagggcgac

SEQ ID NO: 85 - DNA, 18 bp, Artificial
  Synthetic Sequence
gtacttgtaccggtaggg

SEQ ID NO: 86 - DNA, 18 bp, Artificial
  Synthetic Sequence
cacagcgatagcggcaag

SEQ ID NO: 87 - DNA, 18 bp, Artificial
  Synthetic Sequence
gtacttgtaccggtaggg

SEQ ID NO: 88 - DNA, 18 bp, Artificial
  Synthetic Sequence
ctgaccaactgctacaag

SEQ ID NO: 89 - DNA, 19 bp, Artificial
  Synthetic Sequence
ggtccagagggtacagctt

SEQ ID NO: 90 - DNA, 18 bp, Artificial
  Synthetic Sequence
ctgtccttcatcctgaac

SEQ ID NO: 91 - DNA, 21 bp, Artificial
  Synthetic Sequence
ttcagcgttgttgtactcgta

SEQ ID NO: 92 - DNA, 18 bp, Artificial
  Synthetic Sequence
ctgtccttcatcctgaac

SEQ ID NO: 93 - DNA, 18 bp, Artificial
  Synthetic Sequence
gtccagagggtacagctt

SEQ ID NO: 94 - DNA, 18 bp, Artificial
  Synthetic Sequence
cactctgactctggcacc

SEQ ID NO: 95 - DNA, 24 bp, Artificial
  Synthetic Sequence
agaggacttcatcttgttgttggt

SEQ ID NO: 96 - DNA, 18 bp, Artificial
  Synthetic Sequence
ctgtccttcatcctgaac

SEQ ID NO: 97 - DNA, 24 bp, Artificial
  Synthetic Sequence
agaggacttcatcttgttgttggt

SEQ ID NO: 98 - DNA, 18 bp, Artificial
  Synthetic Sequence
cactctgactctggcacc

SEQ ID NO: 99 - DNA, 18 bp, Artificial
  Synthetic Sequence
gtccagcttagaggagtt

SEQ ID NO: 100 - DNA, 18 bp, Artificial
  Synthetic Sequence
ctgtccttcatcctgaac

SEQ ID NO: 101 - DNA, 18 bp, Artificial
  Synthetic Sequence
gtccagcttagaggagtt

SEQ ID NO: 102 - DNA, 18 bp, Artificial
  Synthetic Sequence
cactctgactctggcacc

SEQ ID NO: 103 - DNA, 18 bp, Artificial
  Synthetic Sequence
ggcggcgttggtggtaga

SEQ ID NO: 104 - DNA, 18 bp, Artificial
  Synthetic Sequence
ctgtccttcatcctgaac

SEQ ID NO: 105 - DNA, 18 bp, Artificial
  Synthetic Sequence
ggcggcgttggtggtaga

SEQ ID NO: 106 - DNA, 18 bp, Artificial
  Synthetic Sequence
cactctgactctggcacc

SEQ ID NO: 107 - DNA, 18 bp, Artificial
  Synthetic Sequence
gtacttgtatccgtgggg

SEQ ID NO: 108 - DNA, 18 bp, Artificial
  Synthetic Sequence
ctgtccttcatcctgaac

SEQ ID NO: 109 - DNA, 18 bp, Artificial
  Synthetic Sequence
gtacttgtatccgtgggg

SEQ ID NO: 110 - DNA, 18 bp, Artificial
  Synthetic Sequence
cacagcgatagcggcaag

SEQ ID NO: 111 - DNA, 36 bp, Artificial
  Synthetic Sequence
ggtgtcgaagttgatgtcgggcagattgcccaggta

SEQ ID NO: 112 - DNA, 18 bp, Artificial
  Synthetic Sequence
cacagcgatagcggcaag

SEQ ID NO: 113 - DNA, 36 bp, Artificial
  Synthetic Sequence
agctgcggccagattagcgccctcgtggaaggacac

SEQ ID NO: 114 - DNA, 18 bp, Artificial
  Synthetic Sequence
cacagcgatagcggcaag

SEQ ID NO: 115 - DNA, 36 bp, Artificial
  Synthetic Sequence
agcgcattcagctgcggcgttggtcttgatggagct

SEQ ID NO: 116 - DNA, 18 bp, Artificial
  Synthetic Sequence
cacagcgatagcggcaag

SEQ ID NO: 117 - DNA, 18 bp, Artificial
  Synthetic Sequence
gtccagcttgctggagtt

SEQ ID NO: 118 - DNA, 36 bp, Artificial
  Synthetic Sequence
gctaatctggccgcagcttacccccagaataagaac

SEQ ID NO: 119 - DNA, 18 bp, Artificial
  Synthetic Sequence
gtccagcttgctggagtt

SEQ ID NO: 120 - DNA, 36 bp, Artificial
  Synthetic Sequence
gccgcagctgaatgcgctgacgtgaagctgggcgtg

SEQ ID NO: 121 - DNA, 18 bp, Artificial
  Synthetic Sequence
gtccagcttgctggagtt

SEQ ID NO: 122 - DNA, 18 bp, Artificial
  Synthetic Sequence
cacagcgatagcggcaag

SEQ ID NO: 123 - DNA, 18 bp, Artificial
  Synthetic Sequence
gtccagcttgctggagtt

SEQ ID NO: 124 - DNA, 18 bp, Artificial
  Synthetic Sequence
cacagcgatagcggcaag

SEQ ID NO: 125 - DNA, 18 bp, Artificial
  Synthetic Sequence
gtccagcttgctggagtt

SEQ ID NO: 126 - DNA, 18 bp, Artificial
  Synthetic Sequence
cacagcgatagcggcaag

SEQ ID NO: 127 - DNA, 18 bp, Artificial
  Synthetic Sequence
gtccagcttgctggagtt

SEQ ID NO: 128 - protein, 640 aa, Artificial Sequence
  Synthetic Sequence
NYIKGDPYFAEYATKLSFILNPSDANNPSGETANHNDEACNCNESGISSVGQAQTSGPSSQKTCITHSSIKTNKKKECKDVKLGVRENDKDLKICVIEDTSLSGVDNCCCQDLLGILQENCSDNKRGSSSNDSCDNKNQDECQKKLEKVFASLTNGYKCDKCKSGTSRSKKKWIWKKSSGNEEGLQEEYANTIGLPPRTQSLYLGNLPKLENVCEDVKDINFDTKEKFLAGCLIVSFHEGKNLKKRYPQNKNSGNKENLCKALEYSFADYGDLIKGTSIWDNEYTKDLELNLQNNFGKLFGKYIKKQNTAEQDTSYSSLDELRESWWNTNKKYIWTAMKHGAEMQITTCNADGSVTGSGSSCDDIPTIDLIPQYLRFLQEWVENFCEQRQAKVKDVITNCKSCKESGNKCKTECKTKCKDECEKYKKFIEACGTAGGGIGTAGSPWSKRWDQIYKRYSKHIEDAKRNRKAGTKNCGTSSTTNAAASTDENKCVQSDIDSFFKHLIDIGLTTPSSYLSNVLDDNICGADKAPWTTYTTYTTTEKCNKERDKSKSQSSDTLVVVNVPSPLGNTPYRYKYACQCKIPTNEETCDDRKEYMNQWSCGSARTMKRGYKNDNYELCKYNGVDVKPTTVRSNSSKLD

SEQ ID NO: 129 - protein, 116 aa, Artificial Sequence
  Synthetic Sequence
GAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHI

SEQ ID NO: 130 - protein, 13 aa, Artificial Sequence
  Synthetic Spytag sequence
AHIVMVDAYKPTK

SEQ ID NO: 131 - protein, 8 aa, Artificial Sequence
  Synthetic Spytag sequence
AHIVMVDA

SEQ ID NO: 132 - protein, 10 aa, Artificial Sequence
  The beta-strand of CnaB2
ATHIKFSKRD

SEQ ID NO: 133 - protein, 104 aa, Artificial Sequence
  Synthetic SpyLigase sequence
HHHHHHDYDGQSGDGKELAGATMELRDSSGKTISTWISDGQVKDFYLYPGKYTFVETAAPDGYEVATAITFTVNEQGQVTVNGKATKGGSGGSGGSGEDSATHI

SEQ ID NO: 134 - protein, 16 aa, Artificial Sequence
  Synthetic isopeptide Spy0128 sequence
TDKDMTITFTNKKDAE

SEQ ID NO: 135 - protein, 282 aa, Artificial Sequence
  Synthetic Split-Spy0128 sequence
ATTVHGETVVNGAKLTVTKNLDLVNSNALIPNTDFTFKIEPDTTVNEDGNKFKGVALNTPMTKVTYTNSDKGGSNTKTAEFDFSEVTFEKPGVYYYKVTEEKIDKVPGVSYDTTSYTVQVHVLWNEEQQKPVATYIVGYKEGSKVPIQFKNSLDSTTLTVKKKVSGTGGDRSKDFNFGLTLKANQYYKASEKVMIEKTTKGGQAPVQTEASIDQLYHFTLKDGESIKVTNLPVGVDYVVTEDDYKSEKYTTNVEVSPQDGAVKNIAGNSTEQETSTDKDMTI

SEQ ID NO: 136 - protein, 93 aa, Artificial Sequence
  Synthetic SpyCatcher-delta-N sequence
EDSATHIKFSKRDEDGKELAGATMELRDSSGKTISTWISDGQVKDFYLYPGKYTFVETAAPDGYEVATAITFTVNEQGQVTVNGKATKGDAHI

SEQ ID NO: 137 - protein, 13 aa, Artificial Sequence
  Synthetic inversed spytaq sequence
KTPKYADVMVIHA

(19) United States
(12) Patent Application Publication
(10) Pub. No.: US 2018/0298098 A1
(43) Pub. Date: Oct. 18, 2018

(54) IMMUNOTHERAPEUTIC TARGETING OF PLACENTAL-LIKE CHONDROITIN SULFATE USING CHIMERIC ANTIGEN RECEPTORS (CARS) AND IMMUNOTHERAPEUTIC TARGETING OF CANCER USING CARS WITH SPLIT-PROTEIN BINDING SYSTEMS

(71) Applicant: VAR2 Pharmaceuticals ApS, Copenhagen N (DK)

(72) Inventor: Mai-Britt Zocca, Copenhagen K (DK)

(73) Assignee: VAR2 Pharmaceutical ApS, Copenhagen N (DK)

(21) Appl. No.: 15/553,783

(22) PCT Filed: Feb. 26, 2016
(86) PCT No.: PCT/EP2016/054084

§ 371 Date: Aug. 25, 2017

(30) Foreign Application Priority Data
Feb. 26, 2015 (EP) 15156736.9

(51) Int. Cl. C07K 16/28; C07K 14/445; C07K 14/725; C07K 14/705

(52) U.S. Cl. CPC C07K 16/2809; C07K 14/445; C07K 14/7051; A61P 35/00; C07K 14/70521; C07K 2319/03; C07K 14/70517

(57) ABSTRACT

The present invention relates to polypeptides comprising conditionally active chimeric antigen receptor (CAR) and a split-protein binding system, a VAR2CSA polypeptide, or any other targeting agent. The present invention further relates to poly-peptides comprising VAR2CSA polypeptides or other targeting agent, and a split-protein binding system.

### FIELD OF THE INVENTION

The present invention provides compositions and methods for immunotherapeutic treatment of cancer using a chimeric antigen receptor (CAR) fused to VAR2CSA (VAR2-CAR) or a split-protein binding system (Spy-CAR), vectors encoding these constructs, and recombinant T-cells comprising the CARs.

### BACKGROUND OF THE INVENTION

In cell-based adoptive immunotherapy, immune cells isolated from a patient can be modified to express synthetic proteins that enable the cells to perform new therapeutic functions after they are subsequently transferred back into the patient. An example of such a synthetic protein is a chimeric antigen receptor (CAR). An example of a currently used CAR is a fusion of an extracellular target recognition domain (e.g., an antigen-binding ScFv chain), a transmembrane domain (e.g. from CD8 or CD28), and one or more intracellular signaling domains including CD28, OX40/4-1BB and CD3zeta. Upon antigen engagement, the intracellular signaling portion of the CAR can initiate an activation-related response in an immune cell; such are release of cytolytic molecules to induce tumor cell death, etc. Chimeric antigen receptors (CARs) are described in Porter et al., 2011, N. Engl. J. Med. 365:725-733; and Kalos et al., 2011, Sci. Transl. Med. 3:95ra73.

The split-protein binding system (SPBS) is characterized by the ability of two split-fragments of a protein to re-constitute into a stable covalently locked structure. An example of such a SPBS is the Spy-tag-Spy-Catcher system, which is comprised of the reactive sub-unit protein partners (Spy-Tag and Spy-Catcher) from the second immunoglobulin-like collagen adhesin domain (CnaB2) of the fibronectin-binding protein (FbaB) of Streptococcus pyogenes (PMID: 20235501). The SpyTag and SpyCatcher interact via a spontaneous isopeptide bond formation and is an example of a split-protein binding system (Zakeri, B. et al. PNAS. 2012). If one protein contain the SpyTag and another the SpyCather the two proteins will be spontaneously linked through a covalent interaction between the SpyTag and the SpyCather which ensures a high binding strength and a one-to-one interaction between the SpyTag and SpyCatcher linked proteins. The Spytag-SpyCatcher system, together with other split-protein binding systems, is described in Veggiani et al., 2014, Trends Biotechnol. October; 32(10):506-12. The SpyCatcher protein-sequence (116 aa) displays some degree of immunogenicity in vivo, which can be reduced by truncating the N-terminal part of the protein (aa 1-24) (PMID: 25434527). While non-immunogenic, the ΔN-SpyCatcher sequence retains full binding capacity to the 13 aa Spytag sequence (PMID: 25434527) and can therefore be used for immuno-sensitive applications such as vaccine production or chimeric immune receptors.

Chondroitin sulfate A (CSA) belongs to the family of glycosaminoglycans (GAGs), which are linear polymers of alternating amino sugars and hexuronic acid residues, attached to proteoglycans. There are several types of GAGs including; chondroitin sulfate (CS), dermatan sulfate (DS or CSB), heparan sulfate (HS) and heparin. While the polysaccharide backbone of these GAGs is simple, considerable diversity arises in modifications such as sulfation and uronate epimerization. This is the basis for the wide variety in domain structure and biological activities of different GAGs. Abnormal or inappropriate expression of CSA has been reported in various human diseases including cancer. A CSA is chain is defined by being predominantly Chondroitin Sulfate A, however a CSA chain can also have a minority DS or CSC along the CSA chain.

The malaria parasite Plasmodium falciparum utilizes host cell proteoglycans in almost all stages of its complex life cycle. The sporozoite infects hepatocytes in the liver through surface-expressed circumsporozoite protein interacting with highly sulfated heparan sulfate proteoglycans (HSPG). Merozoite infection of the erythrocytes is mediated by EBA-175 binding to sialic acid on glycophorin A. In addition, a number of Plasmodium falciparum Erythrocyte Membrane Protein 1 (PfEMP1) proteins, mediating host endothelial adhesion, have been described as glycan-binding. One of these is VAR2CSA, which is a unique member of the PfEMP1 protein family. VAR2CSA binds with high affinity to a specific type of chondroitin sulfate A (CSA) attached to proteoglycans, so called Chondroitin Sulfate Proteoglycan (CSPG), in the intervillous spaces of the placenta. VAR2CSA is a large multi-domain protein (350 kDa) expressed on the surface of P. falciparum-infected erythrocytes (IEs), and the VAR2CSA-CSA interaction is responsible for placenta specific sequestration in placental malaria (PM). Importantly, recombinant full-length VAR2CSA ecto-domain from FCR3 and 3D7 type parasites has shown affinity for CSA in the low nano-molar range.

### OBJECT OF THE INVENTION

It is an object of embodiments of the invention to provide compositions and methods for immunotherapeutic treatment of diseases using a chimeric antigen receptor (CAR) fused with VAR2CSA (VAR2-CAR) or with a split-protein binding system (Spy-CAR), vectors encoding the same, and recombinant T-cells comprising the CAR. In some embodiments, the scFv portion of the CAR is substituted with one portion of a split-protein binding system such as SpyTag (SEQ ID NO:130), SpyCatcher (SEQ ID NO:129), or SpyCatcher-AN (SEQ ID NO:136). In these embodiments, disease antigen-recognition molecules such as antibodies, scFv single chains, nanobodies or recombinant proteins, including VAR2CSA peptides or fragments hereof mono- or bi-specific, are attached to a CAR through the split-protein binding system. The invention also relates to CARs specific to chondroitin sulfate A (CSA) modifications using VAR2CSA. In some embodiments, the scFv portion of the CAR is substituted with VAR2CSA variants comprising at least the minimal CSA binding domain ID1-ID2a. Other objects of embodiments of the invention are to provide methods for deploying split-protein binding enabled CARs for treating conditions associated with inappropriate expression of an antigen, such as a disease-associated antigen, such as disease-associated CSA. Other objects of embodiments of the invention are to provide methods wherein VAR2CSA chimeric constructs, polypeptides or fragments thereof are used to target tissue or cells in humans having an expression, such as inappropriate expression of CSA. The invention also includes VAR2CSA-based bi-specific fusion proteins (bi-specific VAR2) able to engraft an immune response to cells and tissues, such as fusion proteins between VAR2CSA and immune cell-binding domains, such as a scFv anti-CD3 domain.

### SUMMARY OF THE INVENTION

It has been found by the present inventors that recombinant VAR2CSA binds with high affinity and specificity to placental-like CSA chains on a limited repertoire of disease associated proteoglycans. More importantly, the present inventors have found that placental-like CSA chains are uniquely re-expressed in virtually all types of human tumors and that recombinant VAR2CSA proteins bind with high and specific affinity to placental-like CSA on cancer cells and within primary cancer tissues. This function of VAR2SCA can be used to provide constructs suitable for immunotherapy, which constructs comprise elements or domains of a CAR, such as including at least a CD28 and a CD3zeta domain.

Accordingly, the present inventors suggest using this specific and high affinity binding between VAR2CSA and CSA for grafting an immune response to human tumors and metastasis with high or otherwise inappropriate expression of placental-like CSA-modified proteoglycans. This can be obtained through a direct genetic fusion between the CAR and VAR2CSA (VAR2-CAR). It can also be obtained by fusing on fragment of a split-protein binding system, such as the SpyTag or SpyCatcher, to the CAR (Spy-CAR) and subsequently attach a recombinant VAR2CSA protein fused to the corresponding SpyTag or SpyCatcher in vitro or in vivo. The Spy-CAR may be used in other scenarios where the attached targeting molecule is different from VAR2CSA, such as an antibody or a fragment of an antibody. Engraftment of immune cells to tumor tissue or other tissue with high or inappropriate expression of placental-like CSA may also be obtained by using a bi-specific VAR2CSA protein with dual specificity to placental-like CSA and immune cells, such as CD3 positive cells. This is obtained by fusing VAR2CSA with a single chain antibody (such as scFv anti-CD3) specific to a certain population of immune cells (such as CD3-positive cells). By combining the CAR technology with the Split protein system, it is possible to make a universal CAR that can be used as a personalized treatment by further treating the patient with a cancer targeting protein fused to the other part of the split protein. Dosing can be controlled, treatment can be stopped and treatment can be changed during the course of a treatment regimen. In particular by making a Split system CAR and a corresponding VAR2CSA split protein allows for perfect control of monitoring of VAR2CSA protein fold, binding affinity, binding specificity and secondary modifications during Good Manufacturing Practices prior to treatment of a patient.

So, in a first aspect the present invention relates to a polypeptide comprising i) a first polypeptide domain being a transmembrane domain and an endodomain of a chimeric antigen receptor (CAR), and ii) a second extracellular polypeptide domain selected from a VAR2CSA polypeptide or a peptide part of a split-protein binding system.

In a second aspect the present invention relates to a polypeptide comprising i) a first polypeptide domain being a VAR2CSA polypeptide or other tumor targeting agent, such as an single chain scFv, and ii) a second polypeptide domain selected from one or more of a peptide part of a split-protein binding system, a single-domain antibody, such as an scFv or nanobody.

It is to be understood that any of the constructs described herein including CARs and peptide parts of a split-protein binding system may be made in any orientation, forward or reverse translated in the chimeric construct. Accordingly and as an example a spytaq sequence may be AHIVMVDAYKPTK (SEQ ID NO: 130) or the inversed KTPKYADVMVIHA (SEQ ID NO: 137).

In some embodiments the isolated protein fragment of VAR2CSA according to the present invention comprises ID2a.

In a third aspect the present invention relates to an isolated nucleic acid molecule comprising a nucleotide sequences encoding a polypeptide of the invention.

In a further aspect the present invention relates to an expression vector comprising an isolated nucleic acid molecule comprising a nucleotide sequences encoding a polypeptide of the invention.

In a further aspect the present invention relates to a cell comprising a vector of the invention. In some embodiments, the cell is selected from the group consisting of an αβT cell, γδT cell, a Natural Killer (NK) cell, a cytotoxic T lymphocyte (CTL), a regulatory T cell, or any combination thereof. In a further aspect the present invention relates to a two-component therapeutic comprising
a) a first component being a polypeptide comprising i) a first polypeptide domain being a transmembrane domain and an endodomain of a chimeric antigen receptor (CAR), and ii) a second polypeptide domain being a peptide part of a split-protein binding system attached to the extracellular part of said CAR; and
b) a second component being a polypeptide comprising i) a first polypeptide domain being a VAR2CSA polypeptide or any other tumor targeting agent, such as an antibody or scFv, and ii) a second polypeptide domain being a peptide part of a split-protein binding system; optionally further comprising iii) a single-domain antibody, such as an scFv or nanobody,

wherein said peptide parts of a split-protein binding system defined by components a) and b) spontaneously form an irreversible isopeptide bond.

In a further aspect the present invention relates to a method of treating or preventing the occurrence of any indication or condition associated with expression, such as inappropriate expression of CSA, such as in cancer in a subject; the method comprising: (a) culturing a population of cytotoxic lymphocytes under conditions promoting activation; (b) transfecting the lymphocyte population of (a) with a vector encoding a polypeptide comprising i) a first polypeptide domain being the transmembrane domain and the endodomain of a chimeric antigen receptor (CAR), and ii) a second polypeptide domain selected from a VAR2CSA polypeptide, or any other targeting agent, such as an antibody; (c) administering a therapeutically or prophylactically effective number of the transfected lymphocytes of (b) to a subject having or in risk at said indication; wherein the polypeptide comprising the domains of a CAR has binding specificity for the targeted moiety or can be bound by the targeted moiety; thereby treating or preventing said indication in said subject.

In a further aspect the present invention relates to a method of treating or preventing the occurrence of any indication or condition associated with expression, such as inappropriate expression of CSA, such as in cancer, arthritis, arthrosis, multiple sclerosis, pathological conditions caused by neural damage, conditions of the cartilage and scar tissue, such as in rheumatism, cartilage repair or wound healing, or in psoriasis in a subject; the method comprising: (a) culturing a population of cytotoxic lymphocytes under conditions promoting activation; (b) transfecting the lymphocyte population of (a) with a vector encoding a polypeptide comprising i) a first polypeptide domain being the transmembrane domain and the endodomain of a chimeric antigen receptor (CAR), and ii) a second polypeptide domain a peptide part of a split-protein binding system attached to the extracellular part of said CAR; (c) administering a therapeutically or prophylactically effective number of the transfected lymphocytes of (b) to a subject having or in risk at said indication; and (d) administering a polypeptide comprising i) a first polypeptide domain being a VAR2CSA polypeptide or other targeting agent, such as an antibody, and ii) a second polypeptide domain being a peptide part of a split-protein binding system, wherein said peptide parts of a split-protein binding system defined in the two different polypeptides used spontaneously form an irreversible isopeptide bond; thereby treating or preventing said indication in said subject.

In a further aspect the present invention relates to a polypeptide according to the invention, or two-component therapeutic according to the invention for use as a medicament.

In a further aspect the present invention relates to a pharmaceutical composition comprising the polypeptide according to the invention, or two-component therapeutic according to the invention.

In a further aspect the present invention relates to a use of a polypeptide according to the invention, or two-component therapeutic according to the invention; for the preparation of a medicament.

In a further aspect the present invention relates to a polypeptide according to the invention, or two-component therapeutic according to the invention, or pharmaceutical composition according to the invention for the treatment of any indications associated with a condition involving expression, such as inappropriate expression of CSA, such as in cancer, arthritis, arthrosis, multiple sclerosis and healing after neural damage, cartilage repair, wound healing, and in psoriasis.

In a further aspect the present invention relates to a polypeptide according to the invention, or two-component therapeutic according to the invention, or pharmaceutical composition according to the invention in combination with any other suitable anticancer agent.

In some embodiments the cancer is selected from CSA-expressing malignancies including carcinomas (including but not limited to Breast carcinoma, Pancreatic carcinoma, Ovarian carcinoma, Endometrial carcinoma, Hepatocellular carcinoma, Lung carcinoma, Colon carcinoma, Prostate carcinoma, Cervix carcinoma, Testis carcinoma, Basal cell skin carcinoma, Clear cell renal cell carcinoma, Head and neck squamous cell carcinoma, Skin squamous cell carcinoma, Vulvar kreatinized squamous cell carcinoma and Vulvar basal cell carcinoma), sarcomas (including but not limited to Fibrosarcoma, Dedifferentiated chondro- and liposarcoma, Leiomyosarcoma, Liposarcoma, Myxoid liposarcoma, Uterine corpus leiomyosarcoma, Osteosarcoma, Ewing sarcoma and Rhabdomyosarcoma, Synovial sarcoma, Solitary Fibrous tumor), hematopoietic cancers (including but not limited to Chronic lymphatic leukaemia (CLL), Acute lymphatic leukaemia (ALL), Acute myeloid leukaemia (AML), B-cell, T-cell and large granular lymphoma), tumours of neuroepithelial tissue, such but not limited to Astrocytomas (Pleomorphic Xanthoastrocytoma, Fibrillary Astrocytomas, Anaplastic astrocytoma, Glioblastoma Multiforme), Oligodrendroglioma, Ependymoma, Choroid plexus turmor, Oligoastrocytoma, gliosarcoma, Ganglioglioma, Retinoblastoma, Neurocytoma, Neuroblastomas (Esthesioneuroblastoma and Ganglioneuroblastoma), Medulloblastoma, Atypical Teratoid Rhabdoid tumors and all types of neuroendocrine cancer, and any other CSA-expressing cancer.

It is to be understood that for a fusion or chimeric protein comprising a VAR2CSA polypeptide, any VAR2CSA sequences and polypeptides as defined herein may be used. Accordingly, this aspect is not limited to the use of minimal binding fragments. This applies whenever the term VAR2CSA sequence or polypeptide is used and is accordingly equally relevant when used for medical treatment or targeting.

### DETAILED DISCLOSURE OF THE INVENTION

In some embodiments, the polypeptides of the invention comprise the scFv portion of the CAR and a VAR2CSA peptide. In other embodiments, recombinant VAR2CSA peptides are attached to a CAR using a spit-protein binding system. In other embodiments, disease-targeting units such as antibodies, single chain binders (scFv) or recombinant peptides are attached to a CAR using a split-protein binding system. The invention also includes VAR2CSA-based fusion proteins able to recruit an immune response to cells and tissues expressing placental-like CSA through dual binding to placental-like CSA and immune cell-specific receptors (bi-specific VAR2).

Mono- or Bi-Specific VAR2-CARs:

The polypeptide constructs according to the present invention may comprise VAR2CSA in conjunction to a transmembrane domain, and the endodomain of a CAR. In some aspects of the present invention, immune cells of a host are modified to express said polypeptide constructs according to the present invention comprising a CAR. Genetic engineering of human T lymphocytes to express tumor-directed chimeric antigen receptors (CAR) can produce anti-tumor effector cells that bypass tumor immune escape mechanisms that are due to abnormalities in protein-antigen processing and presentation. Moreover, these transgenic receptors can be directed to tumor-associated antigens that are not protein-derived. In certain embodiments of the disclosure there are CTLs that are modified to comprise at least a CAR.

In particular cases, the cytotoxic T lymphocytes (CTLs) include a receptor that is chimeric, non-natural and engineered at least in part by the hand of man. In particular cases, the polypeptide constructs according to the present invention, in addition to the CAR further comprises one, two, three, four, or more components, and in some embodiments the one or more components facilitate targeting or binding of the T lymphocyte to the tumor antigen-comprising cancer cell, such as a VAR2CSA polypeptide. In specific embodiments, the polypeptide constructs according to the present invention comprises a CAR, as part or all of a cytoplasmic signaling domain, and/or part or all of one or more co-stimulatory molecules, for example endodomains of co-stimulatory molecules, and a VAR2CSA polypeptide alone (mono-specific) or fused with an antibody, single chain scFV or recombinant peptide specific for a tumor antigen (bi-specific VAR2-CAR). In certain aspects the antibody is directed at target antigens on the cell surface of cancer cells that are treatable by Temozolomide (TMZ), for example. In certain embodiments, a cytoplasmic signaling domain, such as those derived from the T cell receptor ζ-chain, is employed as at least part of the chimeric receptor in order to produce stimulatory signals for T lymphocyte proliferation and effector function following engagement of the chimeric receptor with the target antigen. Examples would include, but are not limited to, endodomains from co-stimulatory molecules such as CD27, CD28, 4-IBB, and OX40 or the signaling components of cytokine receptors such as IL7 and IL15. In particular embodiments, co-stimulatory molecules are employed to enhance the activation, proliferation, and cytotoxicity of T cells produced by the CAR after antigen engagement. In specific embodiments, the co-stimulatory molecules are CD28, OX40, and 4-1BB and cytokine and the cytokine receptors are IL7 and IL15.

The CAR may be first generation, second generation, or third generation (CAR in which signaling is provided by CD3ζ together with co-stimulation provided by CD28 and a tumor necrosis factor receptor (TNFr) domain, such as 4-1BB or OX40) or any other CAR comprised of at least one of these domains. The polypeptide construct according to the present invention VAR2-CAR may also contains a signal peptide (SP) so that the CAR part of the construct can be glycosylated and anchored in the cell membrane of the immune effector cell. The transmembrane domain (TD) connects the ectodomain to the endodomain and resides within the cell membrane when expressed by a cell and may be derived either from a natural or from a synthetic source. Where the source is natural, the domain may be derived from any membrane-bound or transmembrane protein. For example, the transmembrane region may be derived from (i.e. comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, or CD154. Alternatively the TD may be synthetic, in which case it will comprise predominantly hydrophobic residues such as leucine and valine. In some cases, a triplet of phenylalanine, tryptophan and valine will be found at each end of a synthetic TD.

A CAR may further comprise a hinge sequence. A hinge sequence is a short sequence of amino acids that facilitates protein flexibility (see, e.g., Woof et al, Nat. Rev. Immunol, 4(2): 89-99 (2004)). The hinge sequence may be positioned between the antigen recognition moiety and the TD. The hinge sequence can be any suitable sequence derived or obtained from any suitable molecule. In some embodiments, for example, the hinge sequence is derived from a CD8a molecule or a CD28 molecule, or derived from an IgG, such as the IgG1 hinge region.

The polypeptide constructs according to the present invention comprising a VAR2-CAR may have dual specificity to other disease-associated epitopes including, but not limited to, CD 19, CD20, CD22, Kappa or light chain, CD30, CD33, CD 123, CD38, ROR1, HER2/ErbB2, ErbB3/4, EGFR, EGFRvIII, EphA2, FAP, carcinoembryonic antigen, EGP2, EGP40, mesothelin, TAG72, PSMA, NKG2D ligands, B7-H6, IL-13 receptor cc2, IL-11 receptor a, MUC1, MUC16, CA9, GD2, GD3, HMW-MAA, CD171, Lewis Y, G250/CAIX, HLA-AI MAGE AI, HLA-A2 NY-ESO-1, PSC1, folate receptor-a, CD44v7/8, 8H9, NCAM, VEGF receptors, 5T4, Fetal AchR, NKG2D ligands, CD44v6, TEM1, TEM8, viral-associated antigens expressed by the tumor, or other tumor-associated antigens that are identified through genomic analysis and or differential expression studies of tumors.

The polypeptide constructs according to the present invention comprising the transmembrane domain, and the endodomain of a chimeric antigen receptor (CAR) may further comprise other regulatory elements or suicide gene systems, such as inducible caspase-9 (iCASP9) Clackson T. et al. (1998). Proc. Natl. Acad. Sci. U.S.A. 95 10437-10442; Straathof K. C. et al. (2005). Blood 105 4247-4254, or Sleeping Beauty (SB) transposon system Maiti S N et al, 3 Immunother. 2013 February; 36(2):112-23.

The Split-Protein Binding System (SPBS):

In the present invention, the SpyTag-SpyCatcher SPBS may be combined with CAR technology. Substituting the scFv domain of a CAR construct with a fragment of a SPBS, such as the SpyCatcher sequence, will allow for posttranslational attachment of a targeting unit (such as an scFv, and antibody or a recombinant protein) fused to the second half of the SPBS to the CAR. The interaction between SpyTag and SpyCatcher occurs when the unprotonated amine of Lys31 nucleophilically attacks the carbonyl carbon of Asp117, catalyzed by the neighboring Glu77. The minimal peptide to mediate this binding is AHIVMVDA (SEQ ID NO:131) whereas a c-terminal extension giving the sequence: AHIVMVDAYKPTK (SEQ ID NO: 130) provides the most optimal region, designated “SpyTag” (Zakeri et al PNAS 2012).

SpyCatcher is a part of the CnaB2 domain from the FbaB protein from Streptococcus pyogenes and binds SpyTag consisting of another part of the CnaB2 domain. When these two polypeptides of the CnaB2 domain are mixed, they will spontaneously form an irreversible isopeptide bond thereby completing the formation of the CnaB2 domain.

A K-Tag/SpyTag/SpyLigase system may also be used in the present invention. The CnaB2 domain from Streptococcus pyogenes can be used to generate a covalent peptide-peptide ligation system (Fierer J O. et al. 2014). This is done by splitting the CnaB2 into three parts a) the 13 amino acid SpyTag (SEQ ID NO: 130), b) the β-strand of CnaB2 (SEQ ID NO 132)) named K-Tag, and c) the SpyLigase (SEQ ID NO: 133) constructed from the remaining SpyCatcher polypeptide. By expressing the targeting protein with the small K-Tag fused at the C- or N-terminus and mixing that fusion protein with SpyTag-displaying T-Cells together with the SpyLigase, the K-Tag-fusion antigen will be attached to the SpyTag-VLPs by covalent ligation of the SpyTag with the K-Tag facilitated by the SpyLigase. Conversely, the K-Tag may also be inserted genetically into a CAR whereby the targeting antigen should then be fused to the SpyTag at the C- or N-terminus.

As part of a similar strategy for covalent coupling of targeting-antigens at the surface of T-cells, another pair of split-protein binding partners may be used in the present invention. The major pilin protein, Spy0128, from Streptococcus pyogenes can be split into two fragments (split-Spy0128 (residues 18-299 of Spy0128) (SEQ ID NO 135) and isopeptide (residues 293-308 of Spy0128 (TDKDMTITFTNKKDAE))) (SEQ ID NO 134), which together are capable of forming an intermolecular covalent complex (Zakeri, B. et al. 2010). In line with the described SpyTag-SpyCatcher strategy, the Spy0128 isopeptide can be inserted into the either the TCR or the targeting antigen and the same for the split-Spy0128 binding partner. Again simple mixing, or pre-injection of tagged targeting protein will result in a covalent interaction between the tagged CAR on a T-cell and the targeting antigen.

The Spy-CAR may also be used to construct bi- and multi-specific CARs by attaching K-tagged antibodies, K-tag-linked antibodies, K-tag-linked scFv chains, K-tagged recombinant proteins, or combinations hereof to the Spy-CAR obtaining bi- or multi-specific CAR constructs.

It is to be understood that the SpyTag may be replaced by a Spy0128 isopeptide, and the SpyCatcher replaced by split-Spy0128.

Currently used CARs are not easily pharmacologically controlled. The use of a split-protein binding system may facilitate CARs able to be activated and deactivated against a specific antigen in vivo.

One important aspect of the present invention is to i) collect, ii) modify and iii) infuse a population of T-cells from a subject with high or inappropriate expression of placental-like CSA, such as a cancer patient. In the collection step, T-cells are purified from a patient blood sample. In the modification step, the T-cell population is transfected with a vector encoding a CAR consisting of at least an extracellular split-protein binding domain (such as a spyTag or a SpyCatcher), a transmembrane domain (such as CD8 or CD28), and an activation domain (such as CD3ζ). An antigen binding molecule such as an antibody, single chain scFv, or a recombinant VAR2CSA polypeptide tagged, conjugated or fused to the other part of split-protein binding system is subsequently administered intravenously. This molecule will bind to the corresponding part of the split protein presented by the CAR, which will enable the CAR to bind the target of interest and activate. In the infusion step, recombinant T-cells expressing the CARs are injected into the patient establishing a population of T-cells able to react against the target.

This principle may be applied to any tumor binding molecule (including but not limited to antibodies, scFv single chains, and recombinant proteins) and will have the advantage that:

a) it is possible to target many different tumor antigens or targets with the same CAR infusion.

b) it is possible to revert the treatment by suspending infusion of spyTagged (or similar peptide) tumor-target molecules.

c) it is possible to use complex targeting agents such as recombinant VAR2SCA that are not easily made chimeric as part of a CAR construct.

Bi-Specific VAR2 Proteins:

As VARCSA can be produced as a single chain recombinant protein, it can be fused with other single chain proteins able to recognize specific disease targets. For example, fusing VAR2CSA to a scFv chain targeting CD3 will be able to recruit CD3-expressing immune cells to cells and tissues expressing placental-like CSA. Accordingly, such a dual specificity VAR2CSA-based molecule may be used to graft a T-cell response to cells and tissues with expression of placental-like CSA. In some embodiments, VAR2CSA may be genetically fused to a single chain antibody such as an scFv chain or nanobody creating a bi-specific VAR2CSA protein. In some embodiments, such a bi-specific VAR2CSA protein may contain a split-protein binding tag, such as a SpyTag or a K-Tag. In some embodiments, a bi-specific VAR2CSA protein may be attached to a CAR through a split binding protein system.

VAR2CSA Polypeptides

The present invention is based on the fact that a part of a malaria protein, the so-called VAR2CSA, can bind to a cancer specific CSA and extra-cellular CSA-conjugated CSPG with very high specificity and very high binding strength. This part of the technology is described in WO2013117705 A1.

CSA interacts with many important factors such as growth hormones, cytokines, chemokines, and adhesion molecules and is thought to be involved in structural stabilization, cytokinesis, cell proliferation, differentiation, cell migration, tissue morphogenesis, organogenesis, infection, and wound repair. CS chains are composed of alternating units of N-acetyl-D-galactosamine (GalNAc) and glucuronic acid residues. Glucuronic acid can be sulfated at its C2 position and GalNAc can be sulfated at C4 and/or C6, giving rise to various disaccharide units. Varying modifications of the sugar backbone allows structural and functional heterogeneity of the CS chains. Placenta adhering P. falciparum parasites specifically associate with CS chains that are predominantly C4 sulfated.

Recombinant VAR2CSA protein has been shown to bind with unprecedented high affinity and specificity to CSA. This may be due to an interaction with CSA that is not only dependent on the charged sulfates but also on the CS backbone. The inventors of the present invention have demonstrated that CSA present in the placenta is very similar to the CSA presented on various cancer cells including cancer stem cells. This is substantiated by the fact that VAR2CSA expressing malaria parasites adhere specifically to CSA on C32 melanoma cells and to human brain cancer cells.

Accordingly, the current invention relies on the high affinity and specificity between VAR2CSA recombinant proteins and placental-like CSA. By tagging this protein the invention can be used in a wide range of applications including the tracking of metastases in vivo and to diagnose metastatic disease. By coupling VAR2CSA to an apoptotic or cytotoxic reagent the invention can be used to specifically target and eliminate cancer cells and cancer stem cells. By simple therapy using VAR2CSA recombinant protein it will be possible to neutralize the activity of CSA thereby inhibiting tumorigenesis and/or metastasis of CSA-expressing cancer cells. CSA can be present on a number of protein backbones, e.g. CSPG4, CD44, biglycan, decorin, versican, aggrecan (the major CSPG in cartilage), perlecan, syndecan.

The present invention is envisioned to be particularly relevant to malignant melanoma cancer including cutaneous, ocular and conjuctival melanoma having CSPG4 with CSA chains on the surface of the melanoma cells. This GAG chain is believed to be involved in mitoses and metastases. However, CSPG4 is not only specific to melanoma. Micro- and tissue array analyses, performed by the inventors on data from large panels of human tissue and cell lines, suggest that CSPG4 and other types of CSA-containing proteoglycans may be present on a wide range of cancer types originating from all three cellular germ layers. These cancer types include carcinomas (Breast carcinoma, Pancreatic carcinoma, Ovarian carcinoma, Endometrial carcinoma, Hepatocellular carcinoma, Lung carcinoma, Colon carcinoma, Prostate carcinoma, Cervix carcinoma, Testis carcinoma, Basal cell skin carcinoma, Clear cell renal cell carcinoma, Kreatinized head and neck squamous cell carcinoma, Skin squamous cell carcinoma, Vulvar kreatinized squamous cell carcinoma and Vulvar basal cell carcinoma), sarcomas (Breast liposarcoma, Fibrosarcoma, Dedifferentiated chondro- and liposarcoma, Leiomyosarcoma, Liposarcoma, Myxoid liposarcoma, Uterine corpus leiomyosarcoma, Osteosarcoma, Ewing sarcoma and Rhabdomyosarcoma), hematopoietic cancers (Chronic lymphatic leukaemia (CLL), Acute lymphatic leukaemia (ALL), Acute myeloid leukaemia (AML), B-cell, T-cell and large granular lymphoma), tumours of neuroepithelial tissue, such as Astrocytomas (Pleomorphic Xanthoastrocytoma, Fibrillary Astrocytomas, Anaplastic astrocytoma, Glioblastoma Multiforme), Oligodrendroglioma, Ependymoma, Choroid plexus turmor, Oligoastrocytoma, gliosarcoma, Ganglioglioma, Retinoblastoma, Neurocytoma, Neuroblastomas (Esthesioneuroblastoma and Ganglioneuroblastoma), Medulloblastoma, Atypical Teratoid Rhabdoid tumors and all types of neuroendocrine cancer.

The inventors of the present invention have identified a malaria protein, VAR2CSA, which binds CSA in the intervillous spaces of the placenta with an affinity below 10 nM. Smaller recombinant parts of VAR2CSA have been produced at high yields that bind CSA with characteristics similar to that of the full-length and native VAR2CSA protein. The recombinant VAR2CSA protein does not bind other types of CS such as chondroitin sulfate C (C6S) or highly sulfated GAGs such as heparan sulfate (HS). Recombinant proteins can be produced to bind with high affinity to CSA in various expression systems, here among S2 cells, T.Ni cells, CHO cells and E. coli strains including BL21 and Shuffle cells (tm New England Biolabs).

The inventors of the present invention have also identified a single small (75 kDa) antigen that binds CSA with very high affinity (nM) and high specificity. Table 3 (See example 2) lists the CSA affinity of all the analyzed VAR2CSA proteins using biosensor technology.

The inventors of the present invention have shown that this VAR2CSA recombinant protein binds strongly at low concentrations to a wide range of cancer cell lines including cutaneous Melanoma (C32, MeWo), Lung carcinoma (A549), Breast carcinoma (HCC1395), Osterosarcoma (U2OS, MNNG/HOS), Rhabdomyosarcoma (RH30) and cutaneous T-cell lymphoma (Table 4 and 5). As a control molecule another VAR2CSA protein was used, which is identical to the minimal binding VAR2CSA construct except for a 151 amino acids truncation in the C-terminal part of the molecule. This truncation removes the CSA binding. Recombinant VAR2CSA binds all CSPG4 expressing cell lines and cancer cell lines expressing other CSPG molecules having CSA chains (e.g. T-cell lymphoma). Recombinant VAR2CSA protein fails to interact with human red blood cells and peripheral blood mononuclear cells (PBMC) (Table 4).

The inventors of the present invention have shown herein that malaria parasites adhere to cancer cells and primary human tumors, through a specific interaction between VAR2CSA and a placental-like CSA chain attached to a given proteoglycan. Thus, it is envisioned that recombinant VAR2CSA and conjugates thereof may be used to direct an immune response to the tumor environment.

The advantages of targeting CSA on tumors with VAR2CSA over other current therapies in development are numerous:

1) The interaction between VAR2CSA and CSA is of unprecedented high affinity and highly specific.

2) VAR2CSA is an evolutionary refined malaria protein and it is thus unlikely that therapy will break tolerance and cause autoimmune reactions in the patient.

3) VAR2CSA is a stable protein that is well characterized and can be highly expressed in organisms compatible with large-scale protein production.

4) VAR2CSA is a polymorphic protein with a number of serovariants. Repeated therapy could be offered by different serovariants to avoid issues with neutralizing antibodies.

5) VAR2CSA is naturally exposed extracellularly on the P. falciparum-infected the red blood cell and is thus by nature a stable protein in human serum and has been shown to be highly protease resistant.

The advantages of using a bi-specific VAR2CSA-based molecule, such as an VAR2CSA-scFv-CD3 fusion protein, are numerous:

1) VAR2CSA binds a common cancer-associated CSA modification expressed by multiple malignant disease indications, thus a bi-specific VAR2SCA fusion protein can graft a T-cell response directly to the CSA-positive tumor environment.

2) As the malignancy-associated CSA modification is present on several proteoglycans expressed in a given malignant disease, it is unlikely that a given cancer will develop resistance towards a bi-specific VAR2CSA fusion protein by down regulating a specific proteoglycan.

3) As the VAR2CSA sequence has been naturally optimized in evolution by the P. falciparum malaria parasite to evade human immune defense mechanisms and serum proteases, it is unlikely that a patient will develop immunological (or otherwise) resistance towards a bi-specific VAR2CSA-fusion protein.

4) Evident by the fact that VAR2CSA-expressing malaria parasites only sequester to the placenta (and cannot be found elsewhere in the human body/vasculature), a bi-specific VAR2CSA-fusion protein will be predicted to have minimum adverse effects in a treatment context.

The advantages of using VAR2CSA-CARs over scFv-based CARs are numerous:

1) VAR2CSA binds a common cancer-associated CSA modification expressed by multiple malignant disease indications, thus a VAR2-CAR has a broad but cancer specific targeting range and may be used in several cancer indications.

2) As the malignancy-associated CSA modification is present on several proteoglycans expressed in a given malignant disease, it is unlikely that a given cancer will develop resistance towards a VAR2CSA-CAR by down regulating a specific proteoglycan.

3) As the VAR2CSA sequence has been naturally optimized in evolution by the P. falciparum malaria parasite to evade human immune defense mechanisms and serum proteases, it is unlikely that a patient will develop immunological (or otherwise) resistance towards a VAR2CSA-based CAR.

4) Evident by the fact that VAR2CSA-expressing malaria parasites only sequester to the placenta (and cannot be found elsewhere in the human body/vasculature), a VAR2CSA-CAR will be predicted to have minimum adverse effects in a treatment context.

The advantages of using CARs with a split-protein binding system (Spy-CAR) over traditional first, second, and third generation CARs are numerous:

1) A Spy-CAR does not by itself pose a targeting sequence and will not by itself react against a human antigen.

2) A Spy-CAR can be ‘armed’ with VAR2CSA-Spy protein, which will enable the VAR2CSA-Spy-Spy-CAR to react against a selected disease target.

3) A Spy-CAR can be ‘armed’ with any given targeting molecule that is fused with, or conjugated to, a corresponding fragment of the split-protein binding system, which will enable the Targeting sequence-Spy-Spy-CAR to react against a selected disease target.

4) A Spy-CAR transduced T-cell can be ‘armed’ with VAR2CSA-Spy protein in vitro, which will enable the VAR2CSA-Spy-Spy-CAR to react against a selected disease target.

5) A Spy-CAR transduced T-cell can be ‘armed’ with any given targeting molecule that is fused with, or conjugated to, a corresponding fragment of the split-protein binding system, in vitro, which will enable the targeting sequence-Spy-Spy-CAR to react against a selected disease target.

6) A Spy-CAR transduced T-cell can be ‘armed’ with any given polymeric complex of targeting molecules attached to each other through the K-tag in vitro, which will enable the targeting sequence-Spy-Spy-CAR to react against a selected disease target.

7) A Spy-CAR transduced T-cell can be ‘armed’ with VAR2CSA-Spy protein in vivo by chasing Spy-CAR transduced T-cells with a targeting sequence fused with, or conjugated to, a corresponding fragment of the split-protein binding system, which will enable the VAR2CSA-Spy-Spy-CAR to react against a selected disease target.

8) A Spy-CAR transduced T-cell can be ‘armed’ with any given targeting molecule that is fused with, or conjugated to, a corresponding fragment of the split-protein binding system, in vivo by chasing Spy-CAR transduced T-cells with a targeting sequence fused with, or conjugated to, a corresponding fragment of the split-protein binding system, which will enable the targeting sequence-Spy-Spy-CAR to react against a selected disease target.

9) A Spy-CAR transduced T-cell can be ‘armed’ with any given polymeric complex of targeting molecules attached to each other through the K-tag, in vitro or in vivo by chasing Spy-CAR transduced T-cells with a targeting sequence fused with, or conjugated to, a corresponding fragment of the split-protein binding system, which will enable the targeting sequence-Spy-Spy-CAR to react against a selected disease target.

10) As activated cytotoxic T-cells have a limited lifespan, VAR2CSA-Spy-Spy-CAR transduced T-cells, or T-cells armed with any given targeting molecule that is fused with, or conjugated to, a corresponding fragment of the split-protein binding system, will de cleared from the organism over time, while maintaining ‘un-armed’ Spy-CAR-expressing memory T-cells. As such, the VAR2CSA-Spy-Spy-CAR ‘armed’ T-cell population will be eliminated over time by natural clearance in the spleen. In case of disease recurrence, persistent memory T-cells expressing only the Spy-CAR can then be ‘re-armed’ in vivo by chasing with the same or a new disease targeting sequence fused to the corresponding fragment the split-protein binding system.

11) A spy-CAR can be armed with any cancer-targeting moiety that cannot be produced in the recombinant T-cell as a chimeric protein

12) During a CAR treatment period a spy-CAR can be ‘armed’ first with one spy-tagged protein and as resistance occurs, or if the cancer changes phenotype, or an immune response to the spy-tagged protein is induced, the spy-CAR can be armed with another second-line spy-tagged cancer targeting molecule.

### Definitions

The term “SpyTag” refers to a part of the CnaB2 domain from the FbaB protein from Streptococcus pyogenes optimized to bind SpyCatcher consisting of another part of the CnaB2 domain (SEQ ID NO: 130 and 131). The interaction occurs when the unprotonated amine of Lys31 nucleophilically attacks the carbonyl carbon of Asp117, catalyzed by the neighboring Glu77. The minimal peptide to mediate this binding is AHIVMVDA whereas a c-terminal extension giving the sequence: AHIVMVDAYKPTK provides the most optimal region, designated “SpyTag” (Zakeri, B. et al. PNAS. 2012).

The term “SpyCatcher” refers to a part of the CnaB2 domain from the FbaB protein from Streptococcus pyogenes optimized to bind SpyTag consisting of another part of the CnaB2 domain (SEQ ID NO: 129). SpyCatcher can be residue number 1-113 of CnaB2 and binding can be optimized by the following two mutations: I34E and M69Y (Zakeri, B. et al. PNAS, 2012).

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The terms “antibodies” and “immunoglobulin” include antibodies or immunoglobulins of any isotype, fragments of antibodies which retain specific binding to antigen, including, but not limited to, Fab, Fv, scFv, and Fd fragments, chimeric antibodies, humanized antibodies, single-chain antibodies, and fusion proteins comprising an antigen-binding portion of an antibody and a non-antibody protein.

“Antibody fragments” comprise a portion of an intact antibody, for example, the antigen binding or variable region of the intact antibody. Examples of antibody fragments include Fab, Fab′, F(ab′)2, and Fv fragments; diabodies; linear antibodies (Zapata et al., Protein Eng. 8(10): 1057-1062 (1995)); single-chain antibody molecules; and multispecific antibodies formed from antibody fragments. Papain digestion of antibodies produces two identical antigen-binding fragments, called “Fab” fragments, each with a single antigen-binding site, and a residual “Fe” fragment, a designation reflecting the ability to crystallize readily.

The term “VAR2CSA polypeptide” as used herein refers to the extracellular part of a specific Erythrocyte Membrane Protein 1 (PfEMP1) protein expressed by Plasmodium falciparum interacting with chondroitin sulfate proteoglycans (CSPG) and characterized by having a sequence of SEQ ID NO:55 or SEQ ID NO:56 or fragments or variants thereof with the ability to bind chondroitin sulfate A (CSA) that could be presented on a proteoglycans (CSPG).

In some embodiments, the VAR2CSA polypeptide according to the present invention at least comprises the protein fragment of VAR2CSA, which fragment consist of a sequential amino acid sequence of a) ID1, and b) DBL2Xb.

In some embodiments, the VAR2CSA polypeptide according to the present invention at least comprises the protein fragment of VAR2CSA, which fragment consist of a sequential amino acid sequence of a) ID1, and b) DBL2Xb, and c) ID2a.

Included within the definition of a VAR2CSA polypeptide is polypetides described in Salanti A. et al Mol. Micro 2003 July; 49(1):179-91, in Khunrae P. et al, J Mol Biol. 2010 Apr. 2; 397(3):826-34, in Srivastava A. et al, Proc Natl Acad Sci USA. 2010 Mar. 16; 107(11):4884-9, in Dahlbäck M. et al, J Biol Chem. 2011 May 6; 286(18):15908-17, or in Srivastava A. et al, PLoS One. 2011; 6(5):e20270.

The term “ID1” as used herein refers to a domain of VAR2CSA characterized by having an amino acid sequence with at least 70% sequence identity to an amino acid sequence identified by 1-152 of SEQ ID NO:1.

The term “DBL2Xb” as used herein refers to a domain of VAR2CSA characterized by having an amino acid sequence with at least 70% sequence identity with to amino acid sequence identified by 153-577 of SEQ ID NO:1.

The term “ID2a” as used herein refers to a domain of VAR2CSA characterized by having an amino acid sequence of at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, or at least 62, such as the 63 consecutive amino acids from the N-terminal of amino acids 578-640 of SEQ ID NO:1 and with at least 70% sequence identity to such a sequence of consecutive amino acids.

In some embodiments an amino acid sequence identity referred to herein of at least 70% of any one sequence identified by SEQ ID NO:1-75 or a fragment thereof, refers to a sequence with at least 71, 72, 73, 74, 75, 76, 77, 78, 79, 80, 81, 8, 83, 84, 85, 86, 87, 88, 89, 90, 91, 92, 93, 94, 95, 96, 97, 98, or 99% sequence identity to this sequence.

The terms “variant” or “variants”, as used herein, refers to a VAR2CSA polypeptide having an amino acid sequence of SEQ ID NO:55 or SEQ ID NO:56 or a fragments a VAR2CSA polypeptide comprising an amino acid sequence of SEQ ID NO:1-54, which fragments or variants retain the ability to bind chondroitin sulfate A (CSA) on proteoglycans (CSPG), wherein one or more amino acids have been substituted by another amino acid and/or wherein one or more amino acids have been deleted and/or wherein one or more amino acids have been inserted in the polypeptide and/or wherein one or more amino acids have been added to the polypeptide. Such addition can take place either at the N-terminal end or at the C-terminal end or both. The “variant” or “variants” within this definition still have functional activity in terms of being able to bind chondroitin sulfate A (CSA). In some embodiment a variant has at least 70%, such as at least 71, 72, 73, 74, 75, 76, 77, 78, 79, 80, 81, 8, 83, 84, 85, 86, 87, 88, 89, 90, 91, 92, 93, 94, 95, 96, 97, 98, or 99% sequence identity with the sequence of SEQ ID NO:1-75, and 128, such as the sequence of SEQ ID NO:1, 3-5, 10, 11, 29, 34, 36-38, 41, 43-45, 48, 53-56, 60-70, 72-75, and 128.

The phrases “functional variant”, “functional fragment”, and “functional derivatives” as used herein refers to variants, fragments, truncated versions, as well as derivatives of SEQ ID NO:55 or SEQ ID NO:56, such as any one of SEQ ID NO:1, 3-5, 10, 11, 29, 34, 36-38, 41, 43-45, 48, 53-56, 60-70, 72-75, and 128, which polypeptides comprises essential binding sequence parts of SEQ ID NO:55 or SEQ ID NO:56 and at least possess the ability to bind chondroitin sulfate A (CSA). It is to be understood that a VAR2CSA functional variant or functional fragment may have two or three features selected from being a both a variant, and/or a fragment and/or a derivative.

A functional variant or fragment of a VAR2CSA polypeptide encompass those that exhibit at least about 25%, such as at least about 50%, such as at least about 75%, such as at least about 90% of the binding affinity of wild-type VAR2CSA polypeptide that has been produced in the same cell type, when tested in the assays as described herein.

The term “immunologic fragment” as used herein refers to fragment of an amino acid sequence that possess essentially the same functional activities and the same spatial orientation to be recognized by an antibody. Accordingly a specific antibody will bind both the polypeptide and immunologic fragments thereof.

The term “another amino acid” as used herein means one amino acid that is different from that amino acid naturally present at that position. This includes but is not limited to amino acids that can be encoded by a polynucleotide. In some embodiments the different amino acid is in natural L-form and can be encoded by a polynucleotide.

The term “derivative” as used herein, is intended to designate a VAR2CSA polypeptide exhibiting substantially the same or improved biological activity relative to wild-type VAR2CSA identified by SEQ ID NO:55 or SEQ ID NO:56, or a fragment thereof, in which one or more of the amino acids of the parent peptide have been chemically modified, e.g. by alkylation, PEGylation, acylation, ester formation or amide formation or the like.

The term “construct” is intended to indicate a polynucleotide segment which may be based on a complete or partial naturally occurring nucleotide sequence encoding the polypeptide of interest. The construct may optionally contain other polynucleotide segments. In a similar way, the term “amino acids which can be encoded by polynucleotide constructs” covers amino acids which can be encoded by the polynucleotide constructs defined above, i.e. amino acids such as Ala, Val, Leu, Ile, Met, Phe, Trp, Pro, Gly, Ser, Thr, Cys, Tyr, Asn, Glu, Lys, Arg, His, Asp and Gln.

The term “vector”, as used herein, means any nucleic acid entity capable of the amplification in a host cell. Thus, the vector may be an autonomously replicating vector, i.e. a vector, which exists as an extra-chromosomal entity, the replication of which is independent of chromosomal replication, e.g. a plasmid. Alternatively, the vector may be one which, when introduced into a host cell, is integrated into the host cell genome and replicated together with the chromosome(s) into which it has been integrated. The choice of vector will often depend on the host cell into which it is to be introduced. Vectors include, but are not limited to plasmid vectors, phage vectors, viruses or cosmid vectors. Vectors usually contain a replication origin and at least one selectable gene, i.e., a gene which encodes a product which is readily detectable or the presence of which is essential for cell growth.

As used herein the term “appropriate growth medium” means a medium containing nutrients and other components required for the growth of cells and the expression of the nucleic acid sequence encoding the VAR2CSA polypeptide of the invention.

In the present context, the term “treatment” is meant to include prevention, curing and alleviating the symptoms of a disease, disorder or condition involving expression, such as inappropriate expression of CSA, such as in cancer. Prophylactic and therapeutic administration of VAR2CSA polypeptide, conjugate or derivative according to the invention is thus included in the term “treatment”.

The term “subject” as used herein means any animal, in particular mammals, such as humans, and may, where appropriate, be used interchangeably with the term “patient”.

The term “sequence identity” as known in the art, refers to a relationship between the sequences of two or more polypeptide molecules or two or more nucleic acid molecules, as determined by comparing the sequences. In the art, “identity” also means the degree of sequence relatedness between nucleic acid molecules or between polypeptides, as the case may be, as determined by the number of matches between strings of two or more nucleotide residues or two or more amino acid residues. “Identity” measures the percent of identical matches between the smaller of two or more sequences with gap alignments (if any) addressed by a particular mathematical model or computer program (i.e., “algorithms”).

The term “similarity” is a related concept, but in contrast to “identity”, refers to a sequence relationship that includes both identical matches and conservative substitution matches. If two polypeptide sequences have, for example, (fraction ( 10/20)) identical amino acids, and the remainder are all non-conservative substitutions, then the percent identity and similarity would both be 50%. If, in the same example, there are 5 more positions where there are conservative substitutions, then the percent identity remains 50%, but the percent similarity would be 75% ((fraction ( 15/20))). Therefore, in cases where there are conservative substitutions, the degree of similarity between two polypeptides will be higher than the percent identity between those two polypeptides.

Conservative modifications to the amino acid sequence of SEQ ID NO: 1-56, 60-70, and 72-75, 128 (and the corresponding modifications to the encoding nucleotides) will produce VAR2CSA polypeptides having functional and chemical characteristics similar to those of naturally occurring VAR2CSA polypeptides. In contrast, substantial modifications in the functional and/or chemical characteristics of a VAR2CSA polypeptide may be accomplished by selecting substitutions in the amino acid sequence of SEQ ID NO: 1-56, 60-70, 72-75, and 128 that differ significantly in their effect on maintaining (a) the structure of the molecular backbone in the area of the substitution, for example, as a sheet or helical conformation, (b) the charge or hydrophobicity of the molecule at the target site, or (c) the bulk of the side chain.

For example, a “conservative amino acid substitution” may involve a substitution of a native amino acid residue with a nonnative residue such that there is little or no effect on the polarity or charge of the amino acid residue at that position. Furthermore, any native residue in the polypeptide may also be substituted with alanine, as has been previously described for “alanine scanning mutagenesis” (see, for example, MacLennan et al., 1998, Acta Physiol. Scand. Suppl. 643:55-67; Sasaki et al., 1998, Adv. Biophys. 35:1-24, which discuss alanine scanning mutagenesis).

Desired amino acid substitutions (whether conservative or non-conservative) can be determined by those skilled in the art at the time such substitutions are desired. For example, amino acid substitutions can be used to identify important residues of a polypeptide according to the present invention preferably containing VAR2CSA, or to increase or decrease the affinity of a polypeptide described herein.

Naturally occurring residues may be divided into classes based on common side chain properties:
1) hydrophobic: norleucine, Met, Ala, Val, Leu, Ile;
2) neutral hydrophilic: Cys, Ser, Thr, Asn, Gln;
3) acidic: Asp, Glu;
4) basic: His, Lys, Arg;
5) residues that influence chain orientation: Gly, Pro; and
6) aromatic: Trp, Tyr, Phe.

For example, non-conservative substitutions may involve the exchange of a member of one of these classes for a member from another class. Such substituted residues may be introduced into regions of the Plasmodium falciparum VAR2CSA polypeptide that are homologous with non-Plasmodium falciparum VAR2CSA polypeptides, or into the non-homologous regions of the molecule.

In making such changes, the hydropathic index of amino acids may be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is understood in the art. Kyte et al., 3. Mol. Biol., 157:105-131 (1982). It is known that certain amino acids may be substituted for other amino acids having a similar hydropathic index or score and still retain a similar biological activity. In making changes based upon the hydropathic index, the substitution of amino acids whose hydropathic indexes are within ±2 is preferred, those that are within ±1 are particularly preferred, and those within ±0.5 are even more particularly preferred.

It is also understood in the art that the substitution of like amino acids can be made effectively on the basis of hydrophilicity, particularly where the biologically functionally equivalent protein or peptide thereby created is intended for use in immunological embodiments, as in the present case. The greatest local average hydrophilicity of a protein, as governed by the hydrophilicity of its adjacent amino acids, correlates with its immunogenicity and antigenicity, i.e., with a biological property of the protein.

The following hydrophilicity values have been assigned to amino acid residues: arginine (+3.0); lysine (′3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). In making changes based upon similar hydrophilicity values, the substitution of amino acids whose hydrophilicity values are within ±2 is preferred, those that are within ±1 are particularly preferred, and those within ±0.5 are even more particularly preferred. One may also identify epitopes from primary amino acid sequences on the basis of hydrophilicity. These regions are also referred to as “epitopic core regions.”

A skilled artisan will be able to determine suitable variants of the polypeptide as set forth in SEQ ID NO:1-75, and 128 using well known techniques. For identifying suitable areas of the molecule that may be changed without destroying activity, one skilled in the art may target areas not believed to be important for activity. For example, when similar polypeptides with similar activities from the same species or from other species are known, one skilled in the art may compare the amino acid sequence of a VAR2CSA polypeptide to such similar polypeptides. With such a comparison, one can identify residues and portions of the molecules that are conserved among similar polypeptides. It will be appreciated that changes in areas of a VAR2CSA polypeptide that are not conserved relative to such similar polypeptides would be less likely to adversely affect the biological activity and/or structure of the VAR2CSA polypeptide. One skilled in the art would also know that, even in relatively conserved regions, one may substitute chemically similar amino acids for the naturally occurring residues while retaining activity (conservative amino acid residue substitutions). Therefore, even areas that may be important for biological activity or for structure may be subject to conservative amino acid substitutions without destroying the biological activity or without adversely affecting the polypeptide structure.

Additionally, one skilled in the art can review structure-function studies identifying residues in similar polypeptides that are important for activity or structure. In view of such a comparison, one can predict the importance of amino acid residues in a VAR2CSA polypeptide that correspond to amino acid residues that are important for activity or structure in similar polypeptides. One skilled in the art may opt for chemically similar amino acid substitutions for such predicted important amino acid residues of VAR2CSA polypeptides and other polypeptides of the invention.

One skilled in the art can also analyze the three-dimensional structure and amino acid sequence in relation to that structure in similar polypeptides. In view of that information, one skilled in the art may predict the alignment of amino acid residues of a polypeptide with respect to its three dimensional structure. One skilled in the art may choose not to make radical changes to amino acid residues predicted to be on the surface of the protein, since such residues may be involved in important interactions with other molecules. Moreover, one skilled in the art may generate test variants containing a single amino acid substitution at each desired amino acid residue. The variants can then be screened using activity assays as described herein. Such variants could be used to gather information about suitable variants. For example, if one discovered that a change to a particular amino acid residue resulted in destroyed, undesirably reduced, or unsuitable activity, variants with such a change would be avoided. In other words, based on information gathered from such routine experiments, one skilled in the art can readily determine the amino acids where further substitutions should be avoided either alone or in combination with other mutations.

A number of scientific publications have been devoted to the prediction of secondary structure. See Moult J., Curr. Op. in Biotech., 7(4):422-427 (1996), Chou et al., Biochemistry, 13(2):222-245 (1974); Chou et al., Biochemistry, 113(2):211-222 (1974); Chou et al., Adv. Enzymol. Relat. Areas Mol. Biol, 47:45-148 (1978); Chou et al., Ann. Rev. Biochem., 47:251-276 and Chou et al., Biophys. J., 26:367-384 (1979). Moreover, computer programs are currently available to assist with predicting secondary structure. One method of predicting secondary structure is based upon homology modeling. For example, two polypeptides or proteins, which have a sequence identity of greater than 30%, or similarity greater than 40% often have similar structural topologies. The recent growth of the protein structural data base (PDB) has provided enhanced predictability of secondary structure, including the potential number of folds within a polypeptide's or protein's structure. See Holm et al., Nucl. Acid. Res., 27(1):244-247 (1999). It has been suggested (Brenner et al., Curr. Op. Struct. Biol., 7(3):369-376 (1997)) that there are a limited number of folds in a given polypeptide or protein and that once a critical number of structures have been resolved, structural prediction will gain dramatically in accuracy.

Additional methods of predicting secondary structure include “threading” (Jones, D., Curr. Opin. Struct. Biol., 7(3):377-87 (1997); Sippl et al., Structure, 4(1):15-9 (1996)), “profile analysis” (Bowie et al., Science, 253:164-170 (1991); Gribskov et al., Meth. Enzymol., 183:146-159 (1990); Gribskov et al., Proc. Nat. Acad. Sci., 84(13):4355-4358 (1987)), and “evolutionary linkage” (See Home, supra, and Brenner, supra).

Identity and similarity of related polypeptides can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math., 48:1073 (1988).

Preferred methods to determine identity and/or similarity are designed to give the largest match between the sequences tested. Methods to determine identity and similarity are described in publicly available computer programs. Preferred computer program methods to determine identity and similarity between two sequences include, but are not limited to, the GCG program package, including GAP (Devereux et al., Nucl. Acid. Res., 12:387 (1984); Genetics Computer Group, University of Wisconsin, Madison, Wis.), BLASTP, BLASTN, and FASTA (Altschul et al., 3. Mol. Biol., 215:403-410 (1990)). The BLASTX program is publicly available from the National Center for Biotechnology Information (NCBI) and other sources (BLAST Manual, Altschul et al. NCB/NLM/NIH Bethesda, Md. 20894; Altschul et al., supra). The well-known Smith Waterman algorithm may also be used to determine identity.

Certain alignment schemes for aligning two amino acid sequences may result in the matching of only a short region of the two sequences, and this small aligned region may have very high sequence identity even though there is no significant relationship between the two full length sequences. Accordingly, in a preferred embodiment, the selected alignment method (GAP program) will result in an alignment that spans at least 50 contiguous amino acids of the target polypeptide.

For example, using the computer algorithm GAP (Genetics Computer Group, University of Wisconsin, Madison, Wis.), two polypeptides for which the percent sequence identity is to be determined are aligned for optimal matching of their respective amino acids (the “matched span”, as determined by the algorithm). A gap opening penalty (which is calculated as 3 times the average diagonal; the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. A standard comparison matrix (see Dayhoff et al., Atlas of Protein Sequence and Structure, vol. 5, supp. 3 (1978) for the PAM 250 comparison matrix; Henikoff et al., Proc. Natl. Acad. Sci USA, 89:10915-10919 (1992) for the BLOSUM 62 comparison matrix) is also used by the algorithm.

Preferred parameters for a polypeptide sequence comparison include the following:

Algorithm: Needleman et al., 3. Mol. Biol, 48:443-453 (1970); Comparison matrix: BLOSUM 62 from Henikoff et al., Proc. Natl. Acad. Sci. USA, 89:10915-10919 (1992); Gap Penalty: 12, Gap Length Penalty: 4, Threshold of Similarity: 0.

The GAP program is useful with the above parameters. The aforementioned parameters are the default parameters for polypeptide comparisons (along with no penalty for end gaps) using the GAP algorithm.

Preferred parameters for nucleic acid molecule sequence comparisons include the following: Algorithm: Needleman et al., 3. Mol Biol., 48:443-453 (1970); Comparison matrix: matches=+10, mismatch=0, Gap Penalty: 50, Gap Length Penalty: 3.

The GAP program is also useful with the above parameters. The aforementioned parameters are the default parameters for nucleic acid molecule comparisons.

Other exemplary algorithms, gap opening penalties, gap extension penalties, comparison matrices, thresholds of similarity, etc. may be used, including those set forth in the Program Manual, Wisconsin Package, Version 9, September, 1997. The particular choices to be made will be apparent to those of skill in the art and will depend on the specific comparison to be made, such as DNA to DNA, protein to protein, protein to DNA; and additionally, whether the comparison is between given pairs of sequences (in which case GAP or BestFit are generally preferred) or between one sequence and a large database of sequences (in which case FASTA or BLASTA are preferred).

The inventors of the present invention has now addressed and found the answers to the following key questions related to the molecular mechanism behind placental adhesion in PM: 1) is the described differential CSA adhesion related to the VAR2CSA sequence 2) what are the exact minimal structural requirements for VAR2CSA binding to CSA 3) what type of chemical interaction exists between VAR2CSA and CSA and finally 4) can this information be used to design an optimal vaccine antigen?

By expressing identical FCR3 and 3d7 VAR2CSA truncations, the present inventors showed that VAR2CSA bind CSA with similar affinity and specificity, regardless of parasite strain origin. These two sequences has a sequence identity of 79.6%. The present inventors further demonstrate that the high CSA binding-affinity is retained in several shorter fragments, and that DBL2X, including small regions from the flanking interdomains, form a compact core that contains the high affinity CSA binding site. In silico the present inventors defined putative GAG binding sites in VAR2CSA and by deletion and substitution the present inventors showed that mutations in these sites have no effect on CSPG binding. Using the theory of polyelectrolyte-protein interactions the present inventors have shown that the VAR2CSA-CSA interaction may not, solely, be dependent on ionic interactions. Finally, the present inventors have shown that several short VAR2CSA fragments are capable of inducing the production of adhesion-blocking antibodies and that the anti-adhesive antibodies target the proposed CSA binding region. These data provide the first detailed insight into the biochemical nature of the interaction between a PfEMP1 molecule and its ligand.

Preparation of Polypeptides of the Invention

The VAR2CSA polypeptides and other polypeptides of the invention described herein may be produced by means of recombinant nucleic acid techniques and as described in WO2013/117705. In general, a cloned wild-type VAR2CSA nucleic acid sequence is modified to encode the desired protein. This modified sequence is then inserted into an expression vector, which is in turn transformed or transfected into host cells. Higher eukaryotic cells, in particular cultured mammalian cells, may be used as host cells. Procaryotic cells such as Lactococcus lactis or E. coli can also be used to express the polypeptides as long as these prokaryotes are able to produce disulfide bonds or the protein is or may be refolded correctly. In addition, Yeast strains can also be used to express the protein, here among Saccharomyces cerevisiae and P. Pichia.

The amino acid sequence alterations may be accomplished by a variety of techniques. Modification of the nucleic acid sequence may be by site-specific mutagenesis. Techniques for site-specific mutagenesis are well known in the art and are described in, for example, Zoller and Smith (DNA 3:479-488, 1984) or “Splicing by extension overlap”, Horton et al., Gene 77, 1989, pp. 61-68. Thus, using the nucleotide and amino acid sequences of VAR2CSA, one may introduce the alteration(s) of choice. Likewise, procedures for preparing a DNA construct using polymerase chain reaction using specific primers are well known to per-sons skilled in the art (cf. PCR Protocols, 1990, Academic Press, San Diego, Calif., USA).

The polypeptides of the present invention can also comprise non-naturally occurring amino acid residues. Non-naturally occurring amino acids include, without limitation, beta-alanine, desaminohistidine, trans-3-methylproline, 2,4-methanoproline, cis-4-hydroxyproline, trans-4-hydroxyproline, N-methylglycine, allo-threonine, methylthreonine, hydroxyethylcys-teine, hydroxyethylhomocysteine, nitroglutamine, homoglutamine, pipecolic acid, thiazolidine carboxylic acid, dehydroproline, 3- and 4-methylproline, 3,3-dimethylproline, tert-leucine, nor-valine, 2-azaphenylalanine, 3-azaphenylalanine, 4-azaphenylalanine, and 4-fluorophenylalanine. Several methods are known in the art for incorporating non-naturally occurring amino acid residues into polypeptides. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell-free system comprising an E. coli S30 extract and commercially available enzymes and other reagents. Polypeptides are purified by chromatography. See, for example, Robertson et al., J. Am. Chem. Soc. 113:2722, 1991; Ellman et al., Methods Enzymol. 202:301, 1991; Chung et al., Science 259:806-9, 1993; and Chung et al., Proc. Natl. Acad. Sci. USA 90:10145-9, 1993). In a second method, translation is carried out in Xenopus oocytes by microinjection of mutated mRNA and chemically aminoacylated suppressor tRNAs (Turcatti et al., J. Biol. Chem. 271:19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the polypeptide in place of its natural counterpart. See, Koide et al., Biochem. 33:7470-6, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993).

The nucleic acid construct encoding the VAR2CSA polypeptides and other polypeptides of the invention of the invention may suitably be of genomic or cDNA origin, for instance obtained by preparing a genomic or cDNA library and screening for DNA sequences coding for all or part of the polypeptide by hybridization using synthetic oligonucleotide probes in accordance with standard techniques (cf. Sambrook et al., Molecular Cloning: A Laboratory Manual, 2nd. Ed. Cold Spring Harbor Labora-tory, Cold Spring Harbor, N.Y., 1989).

The nucleic acid construct encoding a VAR2CSA polypeptide may also be prepared synthetically by established standard methods, e.g. the phosphoamidite method described by Beaucage and Caruthers, Tetrahedron Letters 22 (1981), 1859-1869, or the method described by Matthes et al., EMBO Journal 3 (1984), 801-805. According to the phosphoamidite method, oligonucleotides are synthesised, e.g. in an automatic DNA synthesiser, purified, annealed, ligated and cloned in suitable vectors. The DNA sequences encoding the Plasmodium falciparum VAR2CSA polypeptides and other polypeptides of the invention may also be prepared by polymerase chain reaction using specific primers, for instance as described in U.S. Pat. No. 4,683,202, Saiki et al., Science 239 (1988), 487-491, or Sambrook et al., supra.

Furthermore, the nucleic acid construct may be of mixed synthetic and genomic, mixed synthetic and cDNA or mixed genomic and cDNA origin prepared by ligating fragments of syn-thetic, genomic or cDNA origin (as appropriate), the fragments corresponding to various parts of the entire nucleic acid construct, in accordance with standard techniques.

The nucleic acid construct is preferably a DNA construct. DNA sequences for use in producing VAR2CSA polypeptides and other polypeptides according to the present invention will typically encode a pre-pro polypeptide at the amino-terminus of VAR2CSA to obtain proper posttranslational processing and secretion from the host cell.

The DNA sequences encoding the Plasmodium falciparum VAR2CSA polypeptides and other polypeptides according to the present invention are usually inserted into a recombinant vector which may be any vector, which may conveniently be subjected to recombinant DNA procedures, and the choice of vector will often depend on the host cell into which it is to be introduced. Thus, the vector may be an autonomously replicating vector, i.e. a vector, which exists as an extrachromosomal entity, the replication of which is independent of chromosomal replication, e.g. a plasmid. Alternatively, the vector may be one which, when introduced into a host cell, is integrated into the host cell genome and replicated together with the chromosome(s) into which it has been integrated.

The vector is preferably an expression vector in which the DNA sequence encoding the Plasmodium falciparum VAR2CSA polypeptides and other polypeptides according to the present invention is operably linked to additional segments required for transcription of the DNA. In general, the expression vector is derived from plasmid or viral DNA, or may contain elements of both. The term, “operably linked” indicates that the segments are arranged so that they function in concert for their intended purposes, e.g. transcription initiates in a promoter and proceeds through the DNA sequence coding for the polypeptide.

Expression vectors for use in expressing VAR2CSA polypeptides and other polypeptides according to the present invention will comprise a promoter capable of directing the transcription of a cloned gene or cDNA. The promoter may be any DNA sequence, which shows transcriptional activity in the host cell of choice and may be derived from genes encoding proteins either homologous or heterologous to the host cell.

Examples of suitable promoters for directing the transcription of the DNA encoding the Plasmodium falciparum VAR2CSA polypeptide in mammalian cells are the SV40 promoter (Subramani et al., Mol. Cell Biol. 1 (1981), 854-864), the MT-1 (metallothionein gene) promoter (Palmiter et al., Science 222 (1983), 809-814), the CMV promoter (Boshart et al., Cell 41:521-530, 1985) or the adenovirus 2 major late promoter (Kaufman and Sharp, Mol. Cell. Biol, 2:1304-1319, 1982).

An example of a suitable promoter for use in insect cells is the polyhedrin promoter (U.S. Pat. No. 4,745,051; Vasuvedan et al., FEBS Lett. 311, (1992) 7-11), the P10 promoter (J. M. Vlak et al., J. Gen. Virology 69, 1988, pp. 765-776), the Autographa californica polyhedrosis virus basic protein promoter (EP 397 485), the baculovirus immediate early gene 1 promoter (U.S. Pat. No. 5,155,037; U.S. Pat. No. 5,162,222), or the baculovirus 39K delayed-early gene promoter (U.S. Pat. No. 5,155,037; U.S. Pat. No. 5,162,222).

Examples of suitable promoters for use in yeast host cells include promoters from yeast glycolytic genes (Hitzeman et al., 3. Biol. Chem. 255 (1980), 12073-12080; Alber and Kawasaki, 3. Mol. Appl. Gen. 1 (1982), 419-434) or alcohol dehydrogenase genes (Young et al., in Genetic Engineering of Microorganisms for Chemicals (Hollaender et al, eds.), Plenum Press, New York, 1982), or the TPI1 (U.S. Pat. No. 4,599,311) or ADH2-4c (Russell et al., Nature 304 (1983), 652-654) promoters.

Examples of suitable promoters for use in filamentous fungus host cells are, for instance, the ADH3 promoter (McKnight et al., The EMBO J. 4 (1985), 2093-2099) or the tpiA promoter. Examples of other useful promoters are those derived from the gene encoding A. oryzae TAKA amylase, Rhizomucor miehei aspartic proteinase, A. niger neutral alpha-amylase, A. niger acid stable alpha-amylase, A. niger or A. awamori glucoamylase (gluA), Rhizomucor miehei lipase, A. oryzae alkaline protease, A. oryzae triose phosphate isomerase or A. nidulans acetamidase. Preferred are the TAKA-amylase and gluA promoters. Suitable promoters are mentioned in, e.g. EP 238 023 and EP 383 779.

The DNA sequences encoding the Plasmodium falciparum VAR2CSA polypeptides and other polypeptides according to the present invention may also, if necessary, be operably connected to a suitable terminator, such as the human growth hormone terminator (Palmiter et al., Science 222, 1983, pp. 809-814) or the TPI1 (Alber and Kawasaki, 3. Mol. Appl. Gen. 1, 1982, pp. 419-434) or ADH3 (McKnight et al., The EMBO J. 4, 1985, pp. 2093-2099) terminators. Expression vectors may also contain a set of RNA splice sites located downstream from the promoter and upstream from the insertion site for the VAR2CSA sequence itself. Preferred RNA splice sites may be obtained from adenovirus and/or immunoglobulin genes. Also contained in the expression vectors is a polyadenylation signal located downstream of the insertion site. Particularly preferred polyadenylation signals include the early or late polyadenylation signal from SV40 (Kaufman and Sharp, ibid.), the polyadenylation signal from the adenovirus 5 Elb region, the human growth hormone gene terminator (DeNoto et al. Nucl. Acids Res. 9:3719-3730, 1981) or the polyadenylation signal from Plasmodium falciparum, human or bovine genes. The expression vectors may also include a noncoding viral leader sequence, such as the adenovirus 2 tripartite leader, located between the promoter and the RNA splice sites; and enhancer sequences, such as the SV40 enhancer.

To direct the Plasmodium falciparum VAR2CSA polypeptides and other polypeptides of the present invention into the secretory pathway of the host cells, a secretory signal sequence (also known as a leader sequence, prepro sequence or pre sequence) may be provided in the recombinant vector. The secretory signal sequence is joined to the DNA sequences encoding the Plasmodium falciparum VAR2CSA polypeptides and other polypeptides according to the present invention in the correct reading frame. Secretory signal sequences are commonly positioned 5′ to the DNA sequence encoding the peptide. The secretory signal sequence may be that, normally associated with the protein or may be from a gene encoding another secreted protein.

For secretion from yeast cells, the secretory signal sequence may encode any signal peptide, which ensures efficient direction of the expressed Plasmodium falciparum VAR2CSA polypeptides and other polypeptides according to the present invention into the secretory pathway of the cell. The signal peptide may be naturally occurring signal peptide, or a functional part thereof, or it may be a synthetic peptide. Suitable signal peptides have been found to be the alpha-factor signal peptide (cf. U.S. Pat. No. 4,870,008), the signal peptide of mouse salivary amylase (cf. O. Hagenbuchle et al., Nature 289, 1981, pp. 643-646), a modified carboxypeptidase signal peptide (cf. L. A. Valls et al., Cell 48, 1987, pp. 887-897), the yeast BAR1 signal peptide (cf. WO 87/02670), or the yeast aspartic protease 3 (YAP3) signal peptide (cf. M. Egel-Mitani et al., Yeast 6, 1990, pp. 127-137).

For efficient secretion in yeast, a sequence encoding a leader peptide may also be inserted downstream of the signal sequence and upstream of the DNA sequence encoding the Plasmodium falciparum VAR2CSA polypeptides and other polypeptides according to the present invention. The function of the leader peptide is to allow the expressed peptide to be directed from the endoplasmic reticulum to the Golgi apparatus and further to a secretory vesicle for secretion into the culture medium (i.e. exportation of the Plasmodium falciparum VAR2CSA polypeptides and other polypeptides according to the present invention across the cell wall or at least through the cellular membrane into the periplasmic space of the yeast cell). The leader peptide may be the yeast alpha-factor leader (the use of which is described in e.g. U.S. Pat. No. 4,546,082, U.S. Pat. No. 4,870,008, EP 16 201, EP 123 294, EP 123 544 and EP 163 529). Alternatively, the leader peptide may be a synthetic leader peptide, which is to say a leader peptide not found in nature. Synthetic leader peptides may, for instance, be constructed as described in WO 89/02463 or WO 92/11378.

For use in filamentous fungi, the signal peptide may conveniently be derived from a gene encoding an Aspergillus sp. amylase or glucoamylase, a gene encoding a Rhizomucor miehei lipase or protease or a Humicola lanuginosa lipase. The signal peptide is preferably derived from a gene encoding A. oryzae TAKA amylase, A. niger neutral alpha-amylase, A. niger acid-stable amylase, or A. niger glucoamylase. Suitable signal peptides are disclosed in, e.g. EP 238 023 and EP 215 594.

For use in insect cells, the signal peptide may conveniently be derived from an insect gene (cf. WO 90/05783), such as the lepidopteran Manduca sexta adipokinetic hormone precursor signal peptide (cf. U.S. Pat. No. 5,023,328).

The procedures used to ligate the DNA sequences coding for the Plasmodium falciparum VAR2CSA polypeptides and other polypeptides according to the present invention, the promoter and optionally the terminator and/or secretory signal sequence, respectively, and to insert them into suitable vectors containing the information necessary for replication, are well known to persons skilled in the art (cf., for instance, Sambrook et al., Molecular Cloning: A Laboratory Manual, Cold Spring Harbor, N.Y., 1989).

Methods of transfecting mammalian cells and expressing DNA sequences introduced in the cells are described in e.g. Kaufman and Sharp, 3. Mol. Biol. 159 (1982), 601-621; Southern and Berg, 3. Mol. Appl. Genet. 1 (1982), 327-341; Loyter et al., Proc. Natl. Acad. Sci. USA 79 (1982), 422-426; Wigler et al., Cell 14 (1978), 725; Corsaro and Pearson, Somatic Cell Genetics 7 (1981), 603, Graham and van der Eb, Virology 52 (1973), 456; and Neumann et al., EMBO J. 1 (1982), 841-845.

Cloned DNA sequences are introduced into cultured mammalian cells by, for example, calcium phosphate-mediated transfection (Wigler et al., Cell 14:725-732, 1978; Corsaro and Pearson, Somatic Cell Genetics 7:603-616, 1981; Graham and Van der Eb, Virology 52d:456-467, 1973) or electroporation (Neumann et al., EMBO J. 1:841-845, 1982). To identify and select cells that express the exogenous DNA, a gene that confers a selectable phenotype (a selectable marker) is generally introduced into cells along with the gene or cDNA of interest. Preferred selectable markers include genes that confer resistance to drugs such as neomycin, hygromycin, and methotrexate. The selectable marker may be an amplifiable selectable marker. A preferred amplifiable selectable marker is a dihydrofolate reductase (DHFR) sequence. Selectable markers are reviewed by Thilly (Mammalian Cell Technology, Butterworth Publishers, Stoneham, Mass., incorporated herein by reference). The person skilled in the art will easily be able to choose suitable selectable markers.

Selectable markers may be introduced into the cell on a separate plasmid at the same time as the gene of interest, or they may be introduced on the same plasmid. If on the same plasmid, the selectable marker and the gene of interest may be under the control of different promoters or the same promoter, the latter arrangement producing a dicistronic message. Constructs of this type are known in the art (for example, Levinson and Simonsen, U.S. Pat. No. 4,713,339). It may also be advantageous to add additional DNA, known as “carrier DNA,” to the mixture that is introduced into the cells.

After the cells have taken up the DNA, they are grown in an appropriate growth medium, typically 1-2 days, to begin expressing the gene of interest. As used herein the term “appropriate growth medium” means a medium containing nutrients and other components required for the growth of cells and the expression of the Plasmodium falciparum VAR2CSA polypeptide of interest. Media generally include a carbon source, a nitrogen source, essential amino acids, essential sugars, vitamins, salts, phospholipids, protein and growth factors. Drug selection is then applied to select for the growth of cells that are expressing the selectable marker in a stable fashion. For cells that have been transfected with an amplifiable selectable marker the drug concentration may be increased to select for an increased copy number of the cloned sequences, thereby in-creasing expression levels. Clones of stably transfected cells are then screened for expression of the Plasmodium falciparum VAR2CSA polypeptide of interest.

The host cell into which the DNA sequences encoding the Plasmodium falciparum VAR2CSA polypeptides and other polypeptides according to the present invention is introduced may be any cell, which is capable of producing the posttranslational modified polypeptides and includes yeast, fungi and higher eukaryotic cells.

Examples of mammalian cell lines for use in the present invention are the COS-1 (ATCC CRL 1650), baby hamster kidney (BHK) and 293 (ATCC CRL 1573; Graham et al., J. Gen. Virol. 36:59-72, 1977) cell lines. A preferred BHK cell line is the tk-ts13 BHK cell line (Waechter and Baserga, Proc. Natl. Acad. Sci. USA 79:1106-1110, 1982, incorporated herein by reference), hereinafter referred to as BHK 570 cells. The BHK 570 cell line has been deposited with the American Type Culture Collection, 12301 Parklawn Dr., Rockville, Md. 20852, under ATCC accession number CRL 10314. A tk-ts13 BHK cell line is also available from the ATCC under accession number CRL 1632. In addition, a number of other cell lines may be used within the present invention, including Rat Hep I (Rat hepatoma; ATCC CRL 1600), Rat Hep II (Rat hepatoma; ATCC CRL 1548), TCMK (ATCC CCL 139), Human lung (ATCC HB 8065), NCTC 1469 (ATCC CCL 9.1), CHO (ATCC CCL 61) and DUKX cells (Urlaub and Chasin, Proc. Natl. Acad. Sci. USA 77:4216-4220, 1980).

Examples of suitable yeasts cells include cells of Saccharomyces spp. or Schizosaccharomyces spp., in particular strains of Saccharomyces cerevisiae or Saccharomyces kluyveri. Methods for transforming yeast cells with heterologous DNA and producing heterologous poly-peptides there from are described, e.g. in U.S. Pat. No. 4,599,311, U.S. Pat. No. 4,931,373, U.S. Pat. Nos. 4,870,008, 5,037,743, and U.S. Pat. No. 4,845,075, all of which are hereby incorporated by reference. Transformed cells are selected by a phenotype determined by a selectable marker, commonly drug resistance or the ability to grow in the absence of a particular nutrient, e.g. leucine. A preferred vector for use in yeast is the POT1 vector disclosed in U.S. Pat. No. 4,931,373. The DNA sequences encoding the Plasmodium falciparum VAR2CSA polypeptides and other polypeptides according to the present invention may be preceded by a signal sequence and optionally a leader sequence, e.g. as described above. Further examples of suitable yeast cells are strains of Kluyveromyces, such as K. lactis, Hansenula, e.g. H. polymorpha, or Pichia, e.g. P. pastoris (cf. Gleeson et al., J. Gen. Microbiol. 132, 1986, pp. 3459-3465; U.S. Pat. No. 4,882,279).

Examples of other fungal cells are cells of filamentous fungi, e.g. Aspergillus spp., Neurospora spp., Fusarium spp. or Trichoderma spp., in particular strains of A. oryzae, A. nidulans or A. niger. The use of Aspergillus spp. for the expression of proteins is described in, e.g., EP 272 277, EP 238 023, EP 184 438 The transformation of F. oxysporum may, for instance, be carried out as described by Malardier et al., 1989, Gene 78: 147-156. The transformation of Trichoderma spp. may be performed for instance as described in EP 244 234.

When a filamentous fungus is used as the host cell, it may be transformed with the DNA construct of the invention, conveniently by integrating the DNA construct in the host chromosome to obtain a recombinant host cell. This integration is generally considered to be an advantage as the DNA sequence is more likely to be stably maintained in the cell. Integration of the DNA constructs into the host chromosome may be performed according to conventional methods, e.g. by homologous or heterologous recombination.

Transformation of insect cells and production of heterologous polypeptides therein may be performed as described in U.S. Pat. No. 4,745,051; U.S. Pat. No. 4,879,236; U.S. Pat. Nos. 5,155,037; 5,162,222; EP 397,485) all of which are incorporated herein by reference. The insect cell line used as the host may suitably be a Lepidoptera cell line, such as Spodoptera frugiperda cells or Trichoplusia ni cells (cf. U.S. Pat. No. 5,077,214). Culture conditions may suitably be as described in, for instance, WO 89/01029 or WO 89/01028, or any of the aforementioned references.

The transformed or transfected host cell described above is then cultured in a suitable nutrient medium under conditions permitting expression of the Plasmodium falciparum VAR2CSA polypeptide after which all or part of the resulting peptide may be recovered from the culture. The medium used to culture the cells may be any conventional medium suitable for growing the host cells, such as minimal or complex media containing appropriate supplements. Suitable media are available from commercial suppliers or may be prepared according to published recipes (e.g. in catalogues of the American Type Culture Collection).

The Plasmodium falciparum VAR2CSA polypeptide produced by the cells may then be recovered from the culture medium by conventional procedures including separating the host cells from the medium by centrifugation or filtration, precipitating the proteinaqueous components of the supernatant or filtrate by means of a salt, e.g. ammonium sulfate, purification by a variety of chromatographic procedures, e.g. ion exchange chromatography, gelfiltration chromatography, affinity chromatography, or the like, dependent on the type of polypeptide in question.

Transgenic animal technology may be employed to produce the VAR2CSA polypeptides and other polypeptides of the invention. It is preferred to produce the proteins within the mammary glands of a host female mammal. Expression in the mammary gland and subsequent secretion of the protein of interest into the milk overcomes many difficulties encountered in isolating proteins from other sources. Milk is readily collected, available in large quantities, and biochemically well characterized. Furthermore, the major milk proteins are present in milk at high concentrations (typically from about 1 to 15 g/I).

From a commercial point of view, it is clearly preferable to use as the host a species that has a large milk yield. While smaller animals such as mice and rats can be used (and are preferred at the proof of principle stage), it is preferred to use livestock mammals including, but not limited to, pigs, goats, sheep and cattle. Sheep are particularly preferred due to such factors as the previous history of transgenesis in this species, milk yield, cost and the ready availability of equipment for collecting sheep milk (see, for example, WO 88/00239 for a comparison of factors influencing the choice of host species). It is generally desirable to select a breed of host animal that has been bred for dairy use, such as East Friesland sheep, or to introduce dairy stock by breeding of the transgenic line at a later date. In any event, animals of known, good health status should be used.

To obtain expression in the mammary gland, a transcription promoter from a milk protein gene is used. Milk protein genes include those genes encoding caseins (see U.S. Pat. No. 5,304,489), beta lactoglobulin, a lactalbumin, and whey acidic protein. The beta lactoglobulin (BLG) promoter is preferred. In the case of the ovine beta lactoglobulin gene, a region of at least the proximal 406 bp of 5′ flanking sequence of the gene will generally be used, although larger portions of the 5′ flanking sequence, up to about 5 kbp, are preferred, such as a ˜4.25 kbp DNA segment encompassing the 5′ flanking promoter and non-coding portion of the beta lactoglobulin gene (see Whitelaw et al., Biochem. J. 286: 31 39 (1992)). Similar fragments of promoter DNA from other species are also suitable.

Other regions of the beta lactoglobulin gene may also be incorporated in constructs, as may genomic regions of the gene to be expressed. It is generally accepted in the art that constructs lacking introns, for example, express poorly in comparison with those that contain such DNA sequences (see Brinster et al., Proc. Natl. Acad. Sci. USA 85: 836 840 (1988); Palmiter et al., Proc. Natl. Acad. Sci. USA 88: 478 482 (1991); Whitelaw et al., Transgenic Res. 1: 3 13 (1991); WO 89/01343; and WO 91/02318, each of which is incorporated herein by reference). In this regard, it is generally preferred, where possible, to use genomic sequences containing all or some of the native introns of a gene encoding the protein or polypeptide of interest, thus the further inclusion of at least some introns from, e.g, the beta lactoglobulin gene, is preferred. One such region is a DNA segment that provides for intron splicing and RNA polyadenylation from the 3′ non-coding region of the ovine beta lactoglobulin gene. When substituted for the natural 3′ non-coding sequences of a gene, this ovine beta lactoglobulin segment can both enhance and stabilize expression levels of the protein or polypeptide of interest. Within other embodiments, the region surrounding the initiation ATG of the VAR2CSA sequence is replaced with corresponding sequences from a milk specific protein gene. Such replacement provides a putative tissue specific initiation environment to enhance expression. It is convenient to replace the entire VAR2CSA pre pro and 5′ non-coding sequences with those of, for example, the BLG gene, although smaller regions may be replaced.

For expression of VAR2CSA polypeptides and other polypeptides according to the present invention in transgenic animals, a DNA segment encoding VAR2CSA is operably linked to additional DNA segments required for its expression to produce expression units. Such additional segments include the above mentioned promoter, as well as sequences that provide for termination of transcription and polyadenylation of mRNA. The expression units will further include a DNA segment encoding a secretory signal sequence operably linked to the segment encoding modified VAR2CSA. The secretory signal sequence may be a native secretory signal sequence or may be that of another protein, such as a milk protein (see, for example, von Heijne, Nucl. Acids Res. 14: 4683 4690 (1986); and Meade et al., U.S. Pat. No. 4,873,316, which are incorporated herein by reference).

Construction of expression units for use in transgenic animals is carried out by inserting a VAR2CSA sequence into a plasmid or phage vector containing the additional DNA segments, although the expression unit may be constructed by essentially any sequence of ligations. It is particularly convenient to provide a vector containing a DNA segment encoding a milk protein and to replace the coding sequence for the milk protein with that of a VAR2CSA variant; thereby creating a gene fusion that includes the expression control sequences of the milk protein gene. In any event, cloning of the expression units in plasmids or other vectors facilitates the amplification of the VAR2CSA sequence. Amplification is conveniently carried out in bacterial (e.g. E. coli) host cells, thus the vectors will typically include an origin of replication and a selectable marker functional in bacterial host cells. The expression unit is then introduced into fertilized eggs (including early stage embryos) of the chosen host species. Introduction of heterologous DNA can be accomplished by one of several routes, including microinjection (e.g. U.S. Pat. No. 4,873,191), retroviral infection (Jaenisch, Science 240: 1468 1474 (1988)) or site directed integration using embryonic stem (ES) cells (reviewed by Bradley et al., Bio/Technology 10: 534 539 (1992)). The eggs are then implanted into the oviducts or uteri of pseudopregnant females and allowed to develop to term. Offspring carrying the introduced DNA in their germ line can pass the DNA on to their progeny in the normal, Mendelian fashion, allowing the development of transgenic herds. General procedures for producing transgenic animals are known in the art (see, for example, Hogan et al., Manipulating the Mouse Embryo: A Laboratory Manual, Cold Spring Harbor Laboratory, 1986; Simons et al., Bio/Technology 6: 179 183 (1988); Wall et al., Biol. Reprod. 32: 645 651 (1985); Buhler et al., Bio/Technology 8: 140 143 (1990); Ebert et al., Bio/Technology 9: 835 838 (1991); Krimpenfort et al., Bio/Technology 9: 844 847 (1991); Wall et al., 3. Cell. Biochem. 49: 113 120 (1992); U.S. Pat. No. 4,873,191; U.S. Pat. No. 4,873,316; WO 88/00239, WO 90/05188, WO 92/11757; and GB 87/00458). Techniques for introducing foreign DNA sequences into mammals and their germ cells were originally developed in the mouse (see, e.g., Gordon et al., Proc. Natl. Acad. Sci. USA 77: 7380 7384 (1980); Gordon and Ruddle, Science 214: 1244 1246 (1981); Palmiter and Brinster, Cell 41: 343 345 (1985); Brinster et al., Proc. Natl. Acad. Sci. USA 82: 4438 4442 (1985); and Hogan et al. (ibid.)). These techniques were subsequently adapted for use with larger animals, including livestock species (see, e.g., WO 88/00239, WO 90/05188, and WO 92/11757; and Simons et al., Bio/Technology 6: 179 183 (1988)). To summarize, in the most efficient route used to date in the generation of transgenic mice or livestock, several hundred linear molecules of the DNA of interest are injected into one of the pro nuclei of a fertilized egg according to established techniques. Injection of DNA into the cytoplasm of a zygote can also be employed.

Production in transgenic plants may also be employed. Expression may be generalised or directed to a particular organ, such as a tuber (see, Hiatt, Nature 344:469 479 (1990); Edelbaum et al., 3. Interferon Res. 12:449 453 (1992); Sijmons et al., Bio/Technology 8:217 221 (1990); and EP 0 255 378).

Administration and Pharmaceutical Compositions

Combination Treatments

The polypeptide, derivative, or conjugate as defined in the present specification preferably including VAR2CSA may be administered simultaneously or sequentially with one or more other cancer agent, and/or be used in a combination treatment with other known therapies. The factors may be supplied in single-dosage form wherein the single-dosage form contains both compounds, or in the form of a kit-of-parts comprising a preparation of a VAR2CSA polypeptide as a first unit dosage form and a preparation of the one or more other compound as a second unit dosage form. Whenever a first or second or third, etc., unit dose is mentioned throughout this specification this does not indicate the preferred order of administration, but is merely done for convenience purposes.

Suitable other cancer agents or therapies that may be used in combination with a VAR2CSA polypeptide includes antibodies already on the market or in development, including Vemurafenib (Hoffmann-La Roche), human monoclonal antibodies against MCSP, Therapeutical (Micromet Inc) anti-MCSP using BITE antibody platform technology, and Adoptive transfer of cytotoxic T cells with specificity for MCSP.

By “simultaneous” dosing of a preparation of a VAR2CSA polypeptide and a preparation of one or more other compound is meant administration of the compounds in single-dosage form, or administration of a first agent followed by administration of a second agent with a time separation of no more than 15 minutes, preferably 10, more preferred 5, more preferred 2 minutes. Either factor may be administered first.

By “sequential” dosing is meant administration of a first agent followed by administration of a second agent with a time separation of more than 15 minutes. Either of the two unit dosage form may be administered first. Preferably, both products are injected through the same intravenous access.

Another object of the present invention is to provide a pharmaceutical formulation comprising a polypeptide according to the present invention preferably containing VAR2CSA which is present in a serum/plasma concentration from 0 mg/ml to 1 mg/ml, and wherein the formulation has a pH from 2.0 to 10.0. The formulation may further comprise a buffer system, preservative(s), tonicity agent(s), chelating agent(s), stabilizers and surfactants. In some embodiments of the invention the pharmaceutical formulation is an aqueous formulation, i.e. formulation comprising water. Such formulation is typically a solution or a suspension. In a further embodiment of the invention the pharmaceutical formulation is an aqueous solution. The term “aqueous formulation” is defined as a formulation comprising at least 50% w/w water. Likewise, the term “aqueous solution” is defined as a solution comprising at least 50% w/w water, and the term “aqueous suspension” is defined as a suspension comprising at least 50% w/w water.

In other embodiments the pharmaceutical formulation is a freeze-dried formulation, whereto the physician or the patient adds solvents and/or diluents prior to use.

In other embodiments the pharmaceutical formulation is a dried formulation (e.g. freeze-dried or spray-dried) ready for use without any prior dissolution.

In a further aspect the invention relates to a pharmaceutical formulation comprising an aqueous solution of a VAR2CSA containing polypeptide, and a buffer, wherein the VAR2CSA containing polypeptide is present in a serum/plasma concentration from 0-1 mg/ml or above, and wherein the formulation has a pH from about 2.0 to about 10.0.

In a other embodiments of the invention the pH of the formulation is selected from the list consisting of 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, and 10.0.

In a further embodiment of the invention the buffer is selected from the group consisting of sodium acetate, sodium carbonate, citrate, glycylglycine, histidine, glycine, lysine, arginine, sodium dihydrogen phosphate, disodium hydrogen phosphate, sodium phosphate, and tris(hydroxymethyl)-aminomethan, bicine, tricine, malic acid, succinate, maleic acid, fumaric acid, tartaric acid, aspartic acid or mixtures thereof. Each one of these specific buffers constitutes an alternative embodiment of the invention.

In a further embodiment of the invention the formulation further comprises a pharmaceutically acceptable preservative. In a further embodiment of the invention the preservative is selected from the group consisting of phenol, o-cresol, m-cresol, p-cresol, methyl p-hydroxybenzoate, propyl p-hydroxybenzoate, 2-phenoxyethanol, butyl p-hydroxybenzoate, 2-phenylethanol, benzyl alcohol, chlorobutanol, and thiomerosal, bronopol, benzoic acid, imidurea, chlorohexidine, sodium dehydroacetate, chlorocresol, ethyl p-hydroxybenzoate, benzethonium chloride, chlorphenesine (3p-chlorphenoxypropane-1,2-diol) or mixtures thereof. In a further embodiment of the invention the preservative is present in a concentration from 0.1 mg/ml to 20 mg/ml. In a further embodiment of the invention the preservative is present in a concentration from 0.1 mg/ml to 5 mg/ml. In a further embodiment of the invention the preservative is present in a concentration from 5 mg/ml to 10 mg/ml. In a further embodiment of the invention the preservative is present in a concentration from 10 mg/ml to 20 mg/ml. Each one of these specific preservatives constitutes an alternative embodiment of the invention. The use of a preservative in pharmaceutical compositions is well-known to the skilled person. For convenience reference is made to Remington: The Science and Practice of Pharmacy, 19^{th} edition, 1995.

In a further embodiment of the invention the formulation further comprises an isotonic agent. In a further embodiment of the invention the isotonic agent is selected from the group consisting of a salt (e.g. sodium chloride), a sugar or sugar alcohol, an amino acid (e.g. L-glycine, L-histidine, arginine, lysine, isoleucine, aspartic acid, tryptophan, threonine), an alditol (e.g. glycerol (glycerine), 1,2-propanediol (propyleneglycol), 1,3-propanediol, 1,3-butanediol) polyethyleneglycol (e.g. PEG400), or mixtures thereof. Any sugar such as mono-, di-, or polysaccharides, or water-soluble glucans, including for example fructose, glucose, mannose, sorbose, xylose, maltose, lactose, sucrose, trehalose, dextran, pullulan, dextrin, cyclodextrin, soluble starch, hydroxyethyl starch and carboxymethylcellulose-Na may be used. In some embodiments the sugar additive is sucrose. Sugar alcohol is defined as a C4-C8 hydrocarbon having at least one —OH group and includes, for example, mannitol, sorbitol, inositol, galactitol, dulcitol, xylitol, and arabitol. In some embodiments the sugar alcohol additive is mannitol. The sugars or sugar alcohols mentioned above may be used individually or in combination. There is no fixed limit to the amount used, as long as the sugar or sugar alcohol is soluble in the liquid preparation and does not adversely affect the stabilizing effects achieved using the methods of the invention. In some embodiments, the sugar or sugar alcohol concentration is between about 1 mg/ml and about 150 mg/ml. In a further embodiment of the invention the isotonic agent is present in a concentration from 1 mg/ml to 50 mg/ml. In a further embodiment of the invention the isotonic agent is present in a concentration from 1 mg/ml to 7 mg/ml. In a further embodiment of the invention the isotonic agent is present in a concentration from 8 mg/ml to 24 mg/ml. In a further embodiment of the invention the isotonic agent is present in a concentration from 25 mg/ml to 50 mg/ml. Each one of these specific isotonic agents constitutes an alternative embodiment of the invention. The use of an isotonic agent in pharmaceutical compositions is well known to the skilled person. For convenience reference is made to Remington: The Science and Practice of Pharmacy, 19^{th} edition, 1995.

In a further embodiment of the invention the formulation further comprises a chelating agent. In a further embodiment of the invention the chelating agent is selected from salts of ethylenediaminetetraacetic acid (EDTA), citric acid, and aspartic acid, and mixtures thereof. In a further embodiment of the invention the chelating agent is present in a concentration from 0.1 mg/ml to 5 mg/ml. In a further embodiment of the invention the chelating agent is present in a concentration from 0.1 mg/ml to 2 mg/ml. In a further embodiment of the invention the chelating agent is present in a concentration from 2 mg/ml to 5 mg/ml. Each one of these specific chelating agents constitutes an alternative embodiment of the invention. The use of a chelating agent in pharmaceutical compositions is well-known to the skilled person. For convenience reference is made to Remington: The Science and Practice of Pharmacy, 19^{th} edition, 1995.

In a further embodiment of the invention the formulation further comprises a stabilizer. The use of a stabilizer in pharmaceutical compositions is well-known to the skilled person. For convenience reference is made to Remington: The Science and Practice of Pharmacy, 19^{th} edition, 1995.

More particularly, compositions of the invention are stabilized liquid pharmaceutical compositions whose therapeutically active components include a polypeptide that possibly exhibits aggregate formation during storage in liquid pharmaceutical formulations. By “aggregate formation” is intended a physical interaction between the polypeptide molecules that results in formation of oligomers, which may remain soluble, or large visible aggregates that precipitate from the solution. By “during storage” is intended a liquid pharmaceutical composition or formulation once prepared, is not immediately administered to a subject. Rather, following preparation, it is packaged for storage, either in a liquid form, in a frozen state, or in a dried form for later reconstitution into a liquid form or other form suitable for administration to a subject. By “dried form” is intended the liquid pharmaceutical composition or formulation is dried either by freeze drying (i.e., lyophilization; see, for example, Williams and Polli (1984) J. Parenteral Sci. Technol. 38:48-59), spray drying (see Masters (1991) in Spray-Drying Handbook (5th ed; Longman Scientific and Technical, Essez, U.K.), pp. 491-676; Broadhead et al. (1992) Drug Devel. Ind. Pharm. 18:1169-1206; and Mumenthaler et al. (1994) Pharm. Res. 11:12-20), or air drying (Carpenter and Crowe (1988) Cryobiology 25:459-470; and Roser (1991) Biopharm. 4:47-53). Aggregate formation by a polypeptide during storage of a liquid pharmaceutical composition can adversely affect biological activity of that polypeptide, resulting in loss of therapeutic efficacy of the pharmaceutical composition. Furthermore, aggregate formation may cause other problems such as blockage of tubing, membranes, or pumps when the polypeptide-containing pharmaceutical composition is administered using an infusion system.

The pharmaceutical compositions of the invention may further comprise an amount of an amino acid base sufficient to decrease aggregate formation by the polypeptide during storage of the composition. By “amino acid base” is intended an amino acid or a combination of amino acids, where any given amino acid is present either in its free base form or in its salt form. Where a combination of amino acids is used, all of the amino acids may be present in their free base forms, all may be present in their salt forms, or some may be present in their free base forms while others are present in their salt forms. In some embodiments, amino acids to use in preparing the compositions of the invention are those carrying a charged side chain, such as arginine, lysine, aspartic acid, and glutamic acid. Any stereoisomer (i.e., L, D, or DL isomer) of a particular amino acid (e.g. glycine, methionine, histidine, imidazole, arginine, lysine, isoleucine, aspartic acid, tryptophan, threonine and mixtures thereof) or combinations of these stereoisomers, may be present in the pharmaceutical compositions of the invention so long as the particular amino acid is present either in its free base form or its salt form. In some embodiments the L-stereoisomer is used. Compositions of the invention may also be formulated with analogues of these amino acids. By “amino acid analogue” is intended a derivative of the naturally occurring amino acid that brings about the desired effect of decreasing aggregate formation by the polypeptide during storage of the liquid pharmaceutical compositions of the invention. Suitable arginine analogues include, for example, aminoguanidine, ornithine and N-monoethyl L-arginine, suitable methionine analogues include ethionine and buthionine and suitable cysteine analogues include S-methyl-L cysteine. As with the other amino acids, the amino acid analogues are incorporated into the compositions in either their free base form or their salt form. In a further embodiment of the invention the amino acids or amino acid analogues are used in a concentration, which is sufficient to prevent or delay aggregation of the protein.

In a further embodiment of the invention methionine (or other sulphuric amino acids or amino acid analogous) may be added to inhibit oxidation of methionine residues to methionine sulfoxide when the polypeptide acting as the therapeutic agent is a polypeptide comprising at least one methionine residue susceptible to such oxidation. By “inhibit” is intended minimal accumulation of methionine oxidized species over time. Inhibiting methionine oxidation results in greater retention of the polypeptide in its proper molecular form. Any stereoisomer of methionine (L, D, or DL isomer) or combinations thereof can be used. The amount to be added should be an amount sufficient to inhibit oxidation of the methionine residues such that the amount of methionine sulfoxide is acceptable to regulatory agencies. Typically, this means that the composition contains no more than about 10% to about 30% methionine sulfoxide. Generally, this can be achieved by adding methionine such that the ratio of methionine added to methionine residues ranges from about 1:1 to about 1000:1, such as 10:1 to about 100:1.

In a further embodiment of the invention the formulation further comprises a stabilizer selected from the group of high molecular weight polymers or low molecular compounds. In a further embodiment of the invention the stabilizer is selected from polyethylene glycol (e.g. PEG 3350), polyvinyl alcohol (PVA), polyvinylpyrrolidone, carboxy-/hydroxycellulose or derivates thereof (e.g. HPC, HPC-SL, HPC-L and HPMC), cyclodextrins, sulphur-containing substances as monothioglycerol, thioglycolic acid and 2-methylthioethanol, and different salts (e.g. sodium chloride). Each one of these specific stabilizers constitutes an alternative embodiment of the invention.

The pharmaceutical compositions may also comprise additional stabilizing agents, which further enhance stability of a therapeutically active polypeptide therein. Stabilizing agents of particular interest to the present invention include, but are not limited to, methionine and EDTA, which protect the polypeptide against methionine oxidation, and a nonionic surfactant, which protects the polypeptide against aggregation associated with freeze-thawing or mechanical shearing.

In a further embodiment of the invention the formulation further comprises a surfactant. In a further embodiment of the invention the surfactant is selected from a detergent, ethoxylated castor oil, polyglycolyzed glycerides, acetylated monoglycerides, sorbitan fatty acid esters, polyoxypropylene-polyoxyethylene block polymers (eg. poloxamers such as Pluronic F68, poloxamer 188 and 407, Triton X-100), polyoxyethylene sorbitan fatty acid esters, polyoxyethylene and polyethylene derivatives such as alkylated and alkoxylated derivatives (tweens, e.g. Tween-20, Tween-40, Tween-80 and Brij-35), monoglycerides or ethoxylated derivatives thereof, diglycerides or polyoxyethylene derivatives thereof, alcohols, glycerol, lectins and phospholipids (eg. phosphatidyl serine, phosphatidyl choline, phosphatidyl ethanolamine, phosphatidyl inositol, diphosphatidyl glycerol and sphingomyelin), derivates of phospholipids (eg. dipalmitoyl phosphatidic acid) and lysophospholipids (eg. palmitoyl lysophosphatidyl-L-serine and 1-acyl-sn-glycero-3-phosphate esters of ethanolamine, choline, serine or threonine) and alkyl, alkoxyl (alkyl ester), alkoxy (alkyl ether)-derivatives of lysophosphatidyl and phosphatidylcholines, e.g. lauroyl and myristoyl derivatives of lysophosphatidylcholine, dipalmitoylphosphatidylcholine, and modifications of the polar head group, that is cholines, ethanolamines, phosphatidic acid, serines, threonines, glycerol, inositol, and the positively charged DODAC, DOTMA, DCP, BISHOP, lysophosphatidylserine and lysophosphatidylthreonine, and glycerophospholipids (eg. cephalins), glyceroglycolipids (eg. galactopyransoide), sphingoglycolipids (eg. ceramides, gangliosides), dodecylphosphocholine, hen egg lysolecithin, fusidic acid derivatives—(e.g. sodium tauro-dihydrofusidate etc.), long-chain fatty acids and salts thereof C6-C12 (eg. oleic acid and caprylic acid), acylcarnitines and derivatives, N^{α}-acylated derivatives of lysine, arginine or histidine, or side-chain acylated derivatives of lysine or arginine, N^{α}-acylated derivatives of dipeptides comprising any combination of lysine, arginine or histidine and a neutral or acidic amino acid, N^{α}-acylated derivative of a tripeptide comprising any combination of a neutral amino acid and two charged amino acids, DSS (docusate sodium, CAS registry no [577-11-7]), docusate calcium, CAS registry no [128-49-4]), docusate potassium, CAS registry no [7491-09-0]), SDS (sodium dodecyl sulfate or sodium lauryl sulfate), sodium caprylate, cholic acid or derivatives thereof, bile acids and salts thereof and glycine or taurine conjugates, ursodeoxycholic acid, sodium cholate, sodium deoxycholate, sodium taurocholate, sodium glycocholate, N-Hexadecyl-N,N-dimethyl-3-ammonio-1-propanesulfonate, anionic (alkyl-aryl-sulphonates) monovalent surfactants, zwitterionic surfactants (e.g. N-alkyl-N,N-dimethylammonio-1-propanesulfonates, 3-cholamido-1-propyldimethylammonio-1-propanesulfonate, cationic surfactants (quaternary ammonium bases) (e.g. cetyl-trimethylammonium bromide, cetylpyridinium chloride), non-ionic surfactants (eg. Dodecyl β-D-glucopyranoside), poloxamines (eg. Tetronic's), which are tetrafunctional block copolymers derived from sequential addition of propylene oxide and ethylene oxide to ethylenediamine, or the surfactant may be selected from the group of imidazoline derivatives, or mixtures thereof. Each one of these specific surfactants constitutes an alternative embodiment of the invention.

The use of a surfactant in pharmaceutical compositions is well known to the skilled person. For convenience reference is made to Remington: The Science and Practice of Pharmacy, 19^{th} edition, 1995.

It is possible that other ingredients may be present in the peptide pharmaceutical formulation of the present invention. Such additional ingredients may include wetting agents, emulsifiers, antioxidants, bulking agents, tonicity modifiers, chelating agents, metal ions, oleaginous vehicles, proteins (e.g., human serum albumin, gelatine or proteins) and a zwitterion (e.g., an amino acid such as betaine, taurine, arginine, glycine, lysine and histidine). Such additional ingredients, of course, should not adversely affect the overall stability of the pharmaceutical formulation of the present invention.

Pharmaceutical compositions containing a polypeptides according to the present invention may be administered to a patient in need of such treatment at several sites, for example, at topical sites, for example, skin and mucosal sites, at sites which bypass absorption, for example, administration in an artery, in a vein, in the heart, and at sites which involve absorption, for example, administration in the skin, under the skin, in a muscle or in the abdomen.

Topical administration may be a particular advantage in the treatment of conditions associated with local inflammation, such as in the treatment of inflammation associated with burn or other conditions associated with the skin. Accordingly, in some embodiments administration is by topical administration.

In some particular embodiments, eye droplets may be used in conditions associated with the eye, such as keratitis, such as diffuse lamellar keratitis (DLK).

Administration of pharmaceutical compositions according to the invention may be through several routes of administration, for example, lingual, sublingual, buccal, in the mouth, oral, in the stomach and intestine, nasal, pulmonary, for example, through the bronchioles and alveoli or a combination thereof, epidermal, dermal, transdermal, vaginal, rectal, ocular, for examples through the conjunctiva, uretal, and parenteral to patients in need of such a treatment.

Compositions of the current invention may be administered in several dosage forms, for example, as solutions, suspensions, emulsions, microemulsions, multiple emulsion, foams, salves, pastes, plasters, ointments, tablets, coated tablets, rinses, capsules, for example, hard gelatine capsules and soft gelatine capsules, suppositories, rectal capsules, drops, gels, sprays, powder, aerosols, inhalants, eye drops, ophthalmic ointments, ophthalmic rinses, vaginal pessaries, vaginal rings, vaginal ointments, injection solution, in situ transforming solutions, for example in situ gelling, in situ setting, in situ precipitating, in situ crystallization, infusion solution, and implants.

Compositions of the invention may further be compounded in, or attached to, for example through covalent, hydrophobic and electrostatic interactions, a drug carrier, drug delivery system and advanced drug delivery system in order to further enhance stability of the polypeptides, increase bioavailability, increase solubility, decrease adverse effects, achieve chronotherapy well known to those skilled in the art, and increase patient compliance or any combination thereof. Examples of carriers, drug delivery systems and advanced drug delivery systems include, but are not limited to, polymers, for example cellulose and derivatives, polysaccharides, for example dextran and derivatives, starch and derivatives, poly(vinyl alcohol), acrylate and methacrylate polymers, polylactic and polyglycolic acid and block copolymers thereof, polyethylene glycols, carrier proteins, for example albumin, gels, for example, thermogelling systems, for example block co-polymeric systems well known to those skilled in the art, micelles, liposomes, microspheres, nanoparticulates, virus like particles, bacteria like particles, liquid crystals and dispersions thereof, L2 phase and dispersions there of, well known to those skilled in the art of phase behaviour in lipid-water systems, polymeric micelles, multiple emulsions, self-emulsifying, self-microemulsifying, cyclodextrins and derivatives thereof, and dendrimers.

Compositions of the current invention are useful in the formulation of solids, semisolids, powder and solutions for pulmonary administration of the VAR2CSA containing polypeptide, using, for example a metered dose inhaler, dry powder inhaler and a nebulizer, all being devices well known to those skilled in the art.

Compositions of the current invention are specifically useful in the formulation of controlled, sustained, protracting, retarded, and slow release drug delivery systems. More specifically, but not limited to, compositions are useful in formulation of parenteral controlled release and sustained release systems (both systems leading to a many-fold reduction in number of administrations), well known to those skilled in the art. Even more preferably, are controlled release and sustained release systems administered subcutaneous. Without limiting the scope of the invention, examples of useful controlled release system and compositions are hydrogels, oleaginous gels, liquid crystals, polymeric micelles, microspheres, nanoparticles,

Methods to produce controlled release systems useful for compositions of the current invention include, but are not limited to, crystallization, condensation, co-crystallization, precipitation, co-precipitation, emulsification, dispersion, high pressure homogenisation, encapsulation, spray drying, microencapsulating, coacervation, phase separation, solvent evaporation to produce microspheres, extrusion and supercritical fluid processes. General reference is made to Handbook of Pharmaceutical Controlled Release (Wise, D. L., ed. Marcel Dekker, New York, 2000) and Drug and the Pharmaceutical Sciences vol. 99: Protein Formulation and Delivery (MacNally, E. J., ed. Marcel Dekker, New York, 2000).

Parenteral administration may be performed by subcutaneous, intramuscular, intraperitoneal or intravenous injection by means of a syringe, optionally a pen-like syringe. Alternatively, parenteral administration can be performed by means of an infusion pump. A further option is a composition, which may be a solution or suspension for the administration of the polypeptide according to the present invention preferably containing VAR2CSA in the form of a nasal or pulmonal spray. As a still further option, the pharmaceutical compositions containing the polypeptide of the invention can also be adapted to transdermal administration, e.g. by needle-free injection or from a patch, optionally an iontophoretic patch, or transmucosal, e.g. buccal, administration.

The term “stabilized formulation” refers to a formulation with increased physical stability, increased chemical stability or increased physical and chemical stability.

The term “physical stability” of the protein formulation as used herein refers to the tendency of the protein to form biologically inactive and/or insoluble aggregates of the protein as a result of exposure of the protein to thermo-mechanical stresses and/or interaction with interfaces and surfaces that are destabilizing, such as hydrophobic surfaces and interfaces. Physical stability of the aqueous protein formulations is evaluated by means of visual inspection and/or turbidity measurements after exposing the formulation filled in suitable containers (e.g. cartridges or vials) to mechanical/physical stress (e.g. agitation) at different temperatures for various time periods. Visual inspection of the formulations is performed in a sharp focused light with a dark background. The turbidity of the formulation is characterized by a visual score ranking the degree of turbidity for instance on a scale from 0 to 3 (a formulation showing no turbidity corresponds to a visual score 0, and a formulation showing visual turbidity in daylight corresponds to visual score 3). A formulation is classified physical unstable with respect to protein aggregation, when it shows visual turbidity in daylight. Alternatively, the turbidity of the formulation can be evaluated by simple turbidity measurements well known to the skilled person. Physical stability of the aqueous protein formulations can also be evaluated by using a spectroscopic agent or probe of the conformational status of the protein. The probe is preferably a small molecule that preferentially binds to a non-native conformer of the protein. One example of a small molecular spectroscopic probe of protein structure is Thioflavin T. Thioflavin T is a fluorescent dye that has been widely used for the detection of amyloid fibrils. In the presence of fibrils, and perhaps other protein configurations as well, Thioflavin T gives rise to a new excitation maximum at about 450 nm and enhanced emission at about 482 nm when bound to a fibril protein form. Unbound Thioflavin T is essentially non-fluorescent at the wavelengths.

Other small molecules can be used as probes of the changes in protein structure from native to non-native states. For instance the “hydrophobic patch” probes that bind preferentially to exposed hydrophobic patches of a protein. The hydrophobic patches are generally buried within the tertiary structure of a protein in its native state, but become exposed as a protein begins to unfold or denature. Examples of these small molecular, spectroscopic probes are aromatic, hydrophobic dyes, such as antrhacene, acridine, phenanthroline or the like. Other spectroscopic probes are metal-amino acid complexes, such as cobalt metal complexes of hydrophobic amino acids, such as phenylalanine, leucine, isoleucine, methionine, and valine, or the like.

The term “chemical stability” of the protein formulation as used herein refers to chemical covalent changes in the protein structure leading to formation of chemical degradation products with potential less biological potency and/or potential increased immunogenic properties compared to the native protein structure. Various chemical degradation products can be formed depending on the type and nature of the native protein and the environment to which the protein is exposed. Elimination of chemical degradation can most probably not be completely avoided and increasing amounts of chemical degradation products is often seen during storage and use of the protein formulation as well-known by the person skilled in the art. Most proteins are prone to deamidation, a process in which the side chain amide group in glutaminyl or asparaginyl residues is hydrolysed to form a free carboxylic acid. Other degradations pathways involves formation of high molecular weight transformation products where two or more protein molecules are covalently bound to each other through transamidation and/or disulfide interactions leading to formation of covalently bound dimer, oligomer and polymer degradation products (Stability of Protein Pharmaceuticals, Ahern. T. J. & Manning M. C., Plenum Press, New York 1992). Oxidation (of for instance methionine residues) can be mentioned as another variant of chemical degradation. The chemical stability of the protein formulation can be evaluated by measuring the amount of the chemical degradation products at various time-points after exposure to different environmental conditions (the formation of degradation products can often be accelerated by for instance increasing temperature). The amount of each individual degradation product is often determined by separation of the degradation products depending on molecule size and/or charge using various chromatography techniques (e.g. SEC-HPLC and/or RP-HPLC).

Hence, as outlined above, a “stabilized formulation” refers to a formulation with increased physical stability, increased chemical stability or increased physical and chemical stability. In general, a formulation must be stable during use and storage (in compliance with recommended use and storage conditions) until the expiration date is reached.

In some embodiments of the invention the pharmaceutical formulation comprising the VAR2CSA polypeptide is stable for more than 6 weeks of usage and for more than 3 years of storage. In other embodiments of the invention the pharmaceutical formulation comprising the VAR2CSA polypeptide is stable for more than 4 weeks of usage and for more than 3 years of storage. In a further embodiment of the invention the pharmaceutical formulation comprising the VAR2CSA polypeptide is stable for more than 4 weeks of usage and for more than two years of storage. In an even further embodiment of the invention the pharmaceutical formulation comprising the VAR2CSA polypeptide is stable for more than 2 weeks of usage and for more than two years of storage.

Indications for Use of Polypeptides According to the Invention

The polypeptides according to the present invention preferably containing VAR2CSAs may be used in a wide range of indications associated with expression, such as inappropriate expression of CSA, such as in various cancers, such as metastatic cancers including melanomas, such as C32 melanoma, sarcomas, lung carcinomas, oligodendrocytomas, human brain tumours including gliomas, leukaemia, such as lymphoblastic leukemia and acute myeloid leukemia, and carcinoma, such as squamous cell carcinomas and breast carcinomas, renal cell carcinomas, chondrosarcomas, and pancreatic cell carcinomas. The polypeptides may also be used for cancer stem cells and accordingly target the cells before development into a cancer. Other conditions associated with expression, such as inappropriate expression of CSA are conditions of the cartilage and/or the development of scar tissue.

### SPECIFIC EMBODIMENTS OF THE INVENTION

As described herein the present invention relates to a polypeptide comprising i) a first polypeptide domain being a transmembrane domain and an endodomain of a chimeric antigen receptor (CAR), and ii) a second extracellular polypeptide domain selected from a VAR2CSA polypeptide or a peptide part of a split-protein binding system, and a polypeptide comprising i) a first polypeptide domain being a VAR2CSA polypeptide or other tumor targeting agent, such as an single chain scFv, and ii) a second polypeptide domain selected from one or more of a peptide part of a split-protein binding system, a single-domain antibody, such as an scFv or nanobody.

In some embodiments the polypeptide comprising said CAR and a peptide part of a split-protein binding system may be combined with a split-protein tagged tumor targeting agent in vitro or in vivo, such as an antibody.

In some embodiments the one peptide part of a split-protein binding system is selected from K-Tag (SEQ ID NO:132), SpyCatcher (SEQ ID NO:129), SpyCatcher-ΔN (SEQ ID NO:136), SpyTag (SEQ ID NO:130), Minimal Spytag sequence (SEQ ID NO:131), split-Spy0128 (SEQ ID NO:135), isopeptide Spy0128 (SEQ ID NO:134), or any inverse sequence thereof, or a variant thereof with sequence identity of at least about 80%, such as at least about 82, 84, 86, 88, 90, 92, 94, 96, 98, or 99%.

In some embodiments the polypeptide further comprises one or more linker or hinge sequences, such as a GC linker.

In some embodiments the VAR2CSA polypeptide consist of SEQ ID NO:55 or SEQ ID NO:56 or fragments or variants thereof with the ability to bind chondroitin sulfate A (CSA) that could be presented on a proteoglycans (CSPG).

In some embodiments the VAR2CSA polypeptide is a fragment of VAR2CSA that consist of a sequential amino acid sequence of

a) ID1, and

b) DBL2Xb, and optionally

c) ID2a.

In some embodiments the VAR2CSA polypeptide binds chondroitin sulfate A (CSA) on proteoglycans (CSPG) with an affinity as measured by a KD lower than 100 nM, such as lower than 80 nM, such as lower than 70 nM, such as lower than 60 nM, such as lower than 50 nM, such as lower than 40 nM, such as lower than 30 nM, such as lower than 26 nM, such as lower than 24 nM, such as lower than 22 nM, such as lower than 20 nM, such as lower than 18 nM, such as lower than 16 nM, such as lower than 14 nM, such as lower than 12 nM, such as lower than 10 nM, such as lower than 9 nM, such as lower than 8 nM, such as lower than 7 nM, such as lower than 6 nM, or lower than 4 nM.

In some embodiments the VAR2CSA polypeptide comprises an amino acid sequence having at least 70% sequence identity with any one amino acid sequence of 1-577 of SEQ ID NO:1, 1-592 of SEQ ID NO:3, 1-579 of SEQ ID NO:4, 1-576 of SEQ ID NO:5, 1-586 of SEQ ID NO:10, 1-579 of SEQ ID NO:11, 1-565 of SEQ ID NO:29, 1-584 of SEQ ID NO:34, 1-569 of SEQ ID NO:36, 1-575 of SEQ ID NO:37, 1-592 of SEQ ID NO:38, 1-603 of SEQ ID NO:41, 1-588 of SEQ ID NO:43, 1-565 of SEQ ID NO:44, 1-589 of SEQ ID NO:45, 1-573 of SEQ ID NO:48, 1-583 of SEQ ID NO:53, 1-569 of SEQ ID NO:54, or 1-640 of SEQ ID NO:128.

In some embodiments the VAR2CSA polypeptide comprises an amino acid sequence having at least 70% sequence identity with an amino acid sequence of 578-640 of SEQ ID NO:1, 593-656 of SEQ ID NO:3, 580-643 of SEQ ID NO:4, 577-640 of SEQ ID NO:5, 587-650 of SEQ ID NO:10, 580-643 of SEQ ID NO:11, 566-628 of SEQ ID NO:29, 585-647 of SEQ ID NO:34, 570-632 of SEQ ID NO:36, 576-639 of SEQ ID NO:37, 593-655 of SEQ ID NO:38, 604-667 of SEQ ID NO:41, 589-652 of SEQ ID NO:43, 566-628 of SEQ ID NO:44, 590-653 of SEQ ID NO:45, 574-637 of SEQ ID NO:48, 584-646 of SEQ ID NO:53, or 570-632 of SEQ ID NO:54.

In some embodiments the VAR2CSA polypeptide comprises an amino acid sequence having at least 70% sequence identity with an amino acid sequence of SEQ ID NO:2, 6, 8, 9, 12, 13, 14, 15, 16, 17, 18, 19, 20, 21, 22, 23, 24, 25, 26, 27, 28, 30, 31, 32, 33, 35, 39, 40, 42, 46, 47, 49, 50, 51, 52, or 128.

In some embodiments the VAR2CSA polypeptide consists of an amino acid sequence having at least 70% sequence identity with any one amino acid sequence of 1-577 of SEQ ID NO:1, 1-592 of SEQ ID NO:3, 1-579 of SEQ ID NO:4, 1-576 of SEQ ID NO:5, 1-586 of SEQ ID NO:10, 1-579 of SEQ ID NO:11, 1-565 of SEQ ID NO:29, 1-584 of SEQ ID NO:34, 1-569 of SEQ ID NO:36, 1-575 of SEQ ID NO:37, 1-592 of SEQ ID NO:38, 1-603 of SEQ ID NO:41, 1-588 of SEQ ID NO:43, 1-565 of SEQ ID NO:44, 1-589 of SEQ ID NO:45, 1-573 of SEQ ID NO:48, 1-583 of SEQ ID NO:53, 1-569 of SEQ ID NO:54, or 1-640 of SEQ ID NO:128.

In some embodiments the VAR2CSA polypeptide consists of an amino acid sequence selected from the list consisting of SEQ ID NO:1, 3-5, 10, 11, 29, 34, 36-38, 41, 43-45, 48, 53, 54, and 128.

In some embodiments the VAR2CSA polypeptide consists of an amino acid sequence having a length of less than 700 amino acids, such as less than 690 amino acids, such as less than 680 amino acids, such as less than 670 amino acids, such as less than 660 amino acids, such as less than 650 amino acids, such as less than 640 amino acids, such as less than 630 amino acids, such as less than 620 amino acids, such as less than 610 amino acids, such as less than 600 amino acids, such as less than 590 amino acids, such as less than 580 amino acids, such as less than 570 amino acids.

In some embodiments the VAR2CSA polypeptide has a molecular mass of less than about 100 kDa under non-reducing conditions on an SDS-PAGE.

In some embodiments the VAR2CSA polypeptide is non-glycosylated.

In some embodiments the transmembrane domain and/or endodomain of a chimeric antigen receptor (CAR) is derived from a from a 1st, 2nd, or 3rd generation CAR.

In some embodiments the polypeptide comprising a first polypeptide domain being a transmembrane domain and an endodomain of a chimeric antigen receptor (CAR), further comprises other regulatory elements or suicide gene systems, such as inducible Caspase-9 (iCASP9) or Sleeping Beauty (SB) transposon system.

In some embodiments the transmembrane domain and/or endodomain of a chimeric antigen receptor (CAR) comprising a CD28 and/or CD3 zeta signaling domain.

In some embodiments the transmembrane domain and/or endodomain of a chimeric antigen receptor (CAR) is as described in any one of WO2014127261, WO13126729, WO13126733, WO14055442, WO14055771, WO2014039523, WO14179759 and WO14138704.

Sequences, Including Sequences of VAR2CSA Polypeptides:

>fcr3 745 amino acids I 640 aa; underlined sequence corresponds to the ID1
domain of FCR3, Sequence in bold corresponds to DBL2Xb domain of FCR3.
Remaining sequence is ID2a
(SEQ ID NO: 1)
NYIKGDPYFAEYATKLSFILNPSDANNPSGETANHNDEACNCNESGISSVGQAQTSGPSSNKTCITHSSIK
TNKKKECKDVKLGVRENDKDLKICVIEDTSLSGVDNCCCQDLLGILQENCSDNKRGSSSNDSCDNKNQ
DECQKKLEKVFASLTNGYKCDKCKSGTSRSKKKWIWKKSSGNEEGLQEEYANTIGLPPRTQSLY
LGNLPKLENVCEDVKDINFDTKEKFLAGCLIVSFHEGKNLKKRYPQNKNSGNKENLCKALEYSF
ADYGDLIKGTSIWDNEYTKDLELNLQNNFGKLFGKYIKKNNTAEQDTSYSSLDELRESWWNT
NKKYIWTAMKHGAEMNITTCNADGSVTGSGSSCDDIPTIDLIPQYLRFLQEWVENFCEQRQA
KVKDVITNCKSCKESGNKCKTECKTKCKDECEKYKKFIEACGTAGGGIGTAGSPWSKRWDQIY
KRYSKHIEDAKRNRKAGTKNCGTSSTTNAAASTDENKCVQSDIDSFFKHLIDIGLTTPSSYLSN
VLDDNICGADKAPWTTYTTYTTTEKCNKERDKSKSQSSDTLVVVNVPSPLGNTPYRYKYACQC
KIPTNEETCDDRKEYMNQWSCGSARTMKRGYKNDNYELCKYNGVDVKPTTVRSNSSKLD
>gi|254952610|gb|ACT97135.1| VAR2CSA [Plasmodium falciparum] | 341 aa
(SEQ ID NO: 2)
KCDKCKSGTSRSRKIWTWRKSSGNKEGLQEEYANTIGLSPRTQLLYLGNLRKLENVCEDVTDINFDTKEK
FLAGCLIAAFHEGKNLKKRYLEKKKGDNNSKLCKDLKYSFADYGDLIKGTSIWDNDFTKDLELNLQQIFGK
LFRKYIKKKNISTEQDTSYSSLDELRESWWNTNKKYIWLAMKHGAGMNSTTCSCSGDSSSGENQTNSC
DDIPTIDLIPQYLRFLQEWVEHFCEQRQAKVKDVITNCNSCKESGGTCNSDCEKKCKNKCDAYKTFIEDC
KGVGGTGTAGSSWVKRWYQIYMRYSKYIEDAKRNRKAGTKSCGTSSTTNVSVSTDENKCVQS-
>M24 745 amino acids | 656 aa
(SEQ ID NO: 3)
DYIKGDPYFAEYATKLSFILNSSDANNPSGETANHNDEVCNPNESEISSVGQAQTSDPSSNKT
CNTHSSIKANKKKVCKHVKLGINNNDKVLRVCVIEDTSLSGVENCCFKDLLGILQENCSDNKS
GSSSNGSCNNKNQEACEKNLEKVLASLTNCYKCDKCKSGTSTVNKNWIWKKSSGNKEGLQKE
YANTIGLPPRTHSLYLGNLPKLENVCEDVKDINFDTKEKFLAGCLIAAFHEGKNLKKRYPQNKN
DDNNSKLCKALEYSFADYGDLIKGTSIWDNEYTKDLELNLQQIFGKLFRKYIKKNISTEQDTLY
SSLDELRESWWNTNKKYIWLAMKHGAGMNITTCCGDGSVTGSGSSCDDIPTIDLIPQYLRFL
QEWVEHFCKQRQEKVKDVINSCNSCKNTSSKTKLGDTCNSDCEKKCKIECEKYKKFIEECRTA
VGGTAGSSWSKRWDQIYKMYSKHIEDAKRNRKAGTKNCGITTGTISGESSGANSGVTTTENK
CVQSDIDSFFKHLIDIGLTTPSSYLSIVLDDNICGDDKAPWTTYTTYTTYTTTEKCNKERDKSK
SQQSNTSVVVNVPSPLGNTPHGYKYACQCKIPTNEETCDDRKEYMNQWISDTSKNPKGSGSTNNDY
ELYTYNGVKETKLPKKLNSPKLD
>KMWII 745 amino acids | 643 aa
(SEQ ID NO: 4)
DYIKDDPYSKEYTTKLSFILNSSDANTSSGETANHNDEACNCNESEISSVGQAQTSGPSSNKTC
ITHSFIKANKKKVCKDVKLGVRENDKVLRVCVIEDTSLSGVDNCCCQDLLGILQENCSDNKRG
SSSNGSCNNKNQDECQKKLEKVFVSLTNGYKCDKCKSGTSTVNKKWIWKKSSGNEKGLQKEY
ANTIGLPPRTQSLYLGNLPKLGNVCEDVTDINFDTKEKFLAGCLIAAFHEGKNLKISHEKKKGD
NGKKLCKALEYSFADYGDLIKGTSIWDNEYTKDLELNLQKAFGKLFGKYIKKNIASDENTSYSS
LDELRESWWNTNKKYIWTAMKHGAEMNSTMCNADGSVTGSGSSCDDIPTTDFIPQYLRFLQ
EWVEHFCKQRQEKVNAVIENCNSCKNTSGERKIGGTCNGDCKTECKNKCEAYKNFIEDCKGG
DGTAGSSWVKRWDQIYKRYSKHIEDAKRNRKAGTKSCGPSSITNASVSTDENKCVQSDIDSF
FKHLIDIGLTTPSSYLSIVLDENNCGEDNAPWTTYTTYTTTEKCNKDKKKSKSQSCNTAVVVNV
PSPLGNTPHEYKYACQCKIPTTEETCDDRKEYMNQWISDTSKKQKGSGSTNNDYELYTYTGVKETKLP
KKLNSPKLD
>1248 745 amino acids | 640 aa
(SEQ ID NO: 5)
SYVKNDPYSKEYVTKLSFILNPSDANNPSGETANHNDEACNPNESEIASVGQAQTSDRLSQKA
CITHSFIGANKKIVCKDVKLGVREKDKDLKICVIEDDSLRGVENCCFKDLLGILQENCSDNKSG
SSSNGSCNNKNQDECQKKLDEALASLHNGYKCDKCKSGTSRSKKIWTWRKFPGNGEGLQKE
YANTIGLPPRTQSLYLGNLRKLENVCKGVTDINFDTKEKFLAGCLIAAFHEGKNLKISNKKKND
DNGKKLCKDLKYSFADYGDLIKGTSIWDNEYTKDLELNLQKIFGKLFRKYIKKNIASDENTLYS
SLDELRESWWNTNKKYIWLAMKHGTTCSSGSGDNGDGSVTGSGSSCDDMSTIDLIPQYLRFL
QEWVEHFCKQRQEKVKDVIENCKSCKNTSGERIIGGTCGSDCKTKCKGECDAYKNFIEECKRG
DGTAGSPWSKRWDQIYMRYSKYIEDAKRNRKAGTKNCGTSSTTNAAENKCVQSDIDSFFKHL
IDIGLTTPSSYLSIVLDENICGDDKAPWTTYTTYTTTEKCNKETDKSKSQSCNTAVVVNVPSPL
GNTPHGYKYACECKIPTTEETCDDRKEYMNQWISDTSKKPKGGRSTNNDYELYTYNGVKETKLPKKSS
SSKLD
>gi|254952618|gb|ACT97139.1| VAR2CSA [Plasmodium falciparum] | 358 aa
(SEQ ID NO: 6)
KCEKCKSEQSKKNNNIWIWRKFPGNGEGLQKEYANTIGLPPRTHSLYLGNLPKLENVCKDVKDINFDTKE
KFLAGCLIAAFHEGKNLKTTYPQNKNADNNSKLCKDLKYSFADYGDLIKGTSIWDNDFTKDLELNLQKIFG
KLFRKYIKKNIASDENTLYSSLDELRESWWNTNKKYIWLAMKHGAEMNSTMCNGDGSVTGSSDSGSTT
CSGDNGSISCDDIPTIDLIPQYLRFLQEWVEHFCKQRQEKVKPVIENCKSCKNTSGERIIGGTCGSDCEK
KCKGECDAYKKFIEECKGGGGGTGTAGSPWSKRWDQIYKRYSKYIEDAKRNRKAGTKSCGPSSTTNAA
ASTTESKCVQS
>gi|254952592|gb|ACT97126.1| VAR2CSA [Plasmodium falciparum] | 333 aa
(SEQ ID NO: 7)
KCDKCKSEQSKKNNKNWIWKQFPGNGEGLQKEYANTIGLPPRTHSLYLGNLPKLENVCKGVTDINFDTK
EKFLAGCLIAAFHEGKNLKTSHEKKKGDNGKKLCKDLKYSFADYGDLIKGTSIWDNDFTKDLELNLQQIF
GKLFRKYIKKNISAEQDTSYSSLDELRESWWNTNKKYIWLAMKHGTTCSSGSGDNGDGSVTGSGSSCD
DMPTTDFIPQYLRFLQEWVEHFCKQRQEKVNAVITNCKSCKESGGTCNSDCEKKCKDECEKYKKFIEECR
TAADGTAGSSWSKRWDQIYKMYSKHIEDAKRNRKAGTKNCGTSSTTNAAENKCVQS
>gi|90193467|gb|ABD92329.1| erythrocyte membrane protein 1 [Plasmodium falciparum] |
269 aa
(SEQ ID NO: 8)
DYIKDDPYSKEYTTKLSFILNSSDANTSSGETANHNDEACNCNESEIASVEQASISDRSSQKAYITHSSIK
TNKKKVCKYVKLGINNNDKVLRVCVIEDTSLSGVENCCFKDLLGILQENCSDNKRGSSFNDSCNNNNEE
ACQKKLEKVLASLTNGYKCEKCKSGTSRSKKKWIWKKSSGKEGGLQKEYANTIGLPPRTQSLYLGNLPKL
ENVCKGVTDINFDTKEKFLAGCLIAAFHEGKNLKPSHQNKNDDNNSKLCKDLKYSFADY
>gi|254952616|gb|ACT97138.1| VAR2CSA [Plasmodium falciparum] | 333 aa
(SEQ ID NO: 9)
KCDKCKSGTSRSKKKWTWRKSSGNKEGLQKEYANTIGLPPRTHSLYLGNLRKLENVCEDVTDINFDTKE
KFLAGCLIAAFHEGKNLKTTYPQNKNDDNNSKLCKALKYSFADYGDLIKGTSIWDNDFTKDLELNLQKIFG
KLFRKYIKKNISTEQHTSYSSLDELRESWWNTNKKYIWLAMKHGAEMNGTTCSCSGDSSDDIPTIDLIPQ
YLRFLQEWVEHFCKQRQAKVNAVINSCNSCKNTSGERKLGGTCGSECKTECKNKCDAYKEFIDGTGSGG
GTGTAGSSWVKRWDQIYKRYSKYIEDAKRNRKAGSKNCGTSSTTNAAESKCVQS
>hb31 745 amino acids | 650 aa
(SEQ ID NO: 10)
SYVKNNPYSAEYVTKLSFILNSSDANTSSETPSKYYDEVCNCNESEISSVGQAQTSGPSSNKTC
ITHSSIKTNKKKVCKDVKLGINNNDKVLRVCVIEDTSLSGVDNCCCQDLLGILQENCSDKNQS
GSSSNGSCNNKNQDECQKKLEKVFASLTNGYKCDKCKSGTSRSKKKWIWRKSSGNEEGLQKE
YANTIGLPPRTQSLYLGNLRKLENVCKGVTDINFDTKEKFLAGCLIAAFHEGKNLKTTYPQNKK
KLCKDLKYSFADYGDLIKGTSIWDNEYTKDLELNLQKAFGKLFRKYIKKNISTEQHTLYSSLDE
LRESWWNTNKKYIWLAMKHGAGMNSTTCCGDGSVTGSGSSCDDIPTIDLIPQYLRFLQEWV
EHFCKQRQEKVNAVIENCNSCKECGDTCNGECKTECEKKCKIECEKYKTFIEECVTAVGGTSGS
PWSKRWDQIYKRYSKYIEDAKRNRKAGTKNCGITTGTISGESSGANSGVTTTENKCVQSDID
SFFKHLIDIGLTTPSSYLSIVLDDNICGADNAPWTTYTTYTTYTTTKNCDIKKKTPKSQPINTSV
VVNVPSPLGNTPHGYKYACQCKIPTTEESCDDRKEYMNQWIIDTSKKQKGSGSTNNDYELYTYNGVK
ETKLPKKSSSSKLD
>hb32 745 amino acids | 643 aa
(SEQ ID NO: 11)
SYVKDDPYSAEYVTKLSFILNSSDANTSSETPSKYYDEVCNCNESEISSVGQAQTSGPSSNKTC
ITHSSIKTNKKKVCKDVKLGINNNDKVLRVCVIEDTSLSGVDNCCCQDLLGILQENCSDKNQS
GSSSNGSCNNKNQDECQKKLEKVFASLTNGYKCDKCKSGTSRSKKKWIWRKSSGNEEGLQKE
YANTIGLPPRTQSLYLGNLPKLENVCKGVTDIIYDTKEKFLSGCLIAAFHEGKNLKTSHEKKND
DNGKKLCKALEYSFADYGDLIKGTSIWDNDFTKDLELNLQKIFGKLFRKYIKKNNTAEQDTSY
SSLDELRESWWNTNKKYIWTAMKHGAGMNSTTCSGDGSVTGSGSSCDDMPTIDLIPQYLRFL
QEWVEHFCKQRQEKVKDVITNCNSCKECGDTCNGECKTECKTKCKGECEKYKNFIEECNGTAD
GGTSGSSWSKRWDQIYKRYSKYIEDAKRNRKAGTKNCGTSSTTNAAASTTENKCVQSDIDSF
FKHLIDIGLTTPSSYLSNVLDDNICGEDKAPWTTYTTYTTKNCDIQKKTPKPQSCDTLVVVNVP
SPLGNTPHGYKYVCECKIPTTEETCDDRKEYMNQWIIDTSKKQKGSGSTNNDYELYTYNGVQIKQAAG
TLKNSKLD
>gi|90193475|gb|ABD92333.1| erythrocyte membrane protein 1 [Plasmodium falciparum] |
269 aa
(SEQ ID NO: 12)
NYIKGDPYSAEYATKLSFILNSSDTENASEKIQKNNDEVCNCNESEIASVEQAPISDRSSQKACITHSSIK
ANKKKVCKHVKLGVRENDKDLKICVIEDTSLSGVDNCCCQDLLGILQENCSDNKSGSSSNGSCNNNNEE
ICQKKLEKVLASLTNGYKCDKCKSGTSTVNKNWIWKKYSGKEGGLQEEYANTIGLPPRTQSLYLGNLPKL
ENVCEDVKDINFDTKEKFLAGCLIAAFHEGKNLKTSNKKKNDDNNSKLCKALKYSFADY
>gi|254952600|gb|ACT97130.1| VAR2CSA [Plasmodium falciparum] | 344 aa
(SEQ ID NO: 13)
KCDKCKSGTSTVNKKWIWKKYSGTEGGLQEEYANTIALPPRTQSLYLGNLPKLENVCKDVTDINFDTKEK
FLAGCLIAAFHEGKNLKTTYLEKKKGDNGKKNDDNNSKLCKALKYSFADYGDLIKGTSIWDNDFTKDLEL
NLQQIFGKLFRKYIKKNIASDENTLYSSLDELRESWWNTNKKYIWLAMKHGAGMNSTMCNADGSVTGS
GSSCDDIPTIDLIPQYLRFLQEWVEHFCKQRQAKVKDVITNCNSCKECGGTCNGECKTECEKKCKGECD
AYKKFIEECKGKADEGTSGSSWSKRWDQIYKRYSKYIEDAKRNRKAGTKNCGPSSTTSTAESKCVQS
>gi|254952598|gb|ACT97129.1| VAR2CSA [Plasmodium falciparum] | 334 aa
(SEQ ID NO: 14)
KCDKCKSEQSKKNNNIWIWKKSSGTEGGLQKEYANTIALPPRTQSLYLGNLRKLENVCEDVKDINFDTKE
KFLAGCLIAAFHEGKNLKKRYLEKKNGDNNSKLCKALKYSFADYGDLIKGTSIWDNEYTKDLELNLQKIFG
KLFRKYIKKNNTAEQHTSYSSLDELRESWWNTNKKYIWLAMKHGTTCSSGSGDNGSISCDDIPTIDLIPQ
YLRFLQEWVEHFCEQRQGKVNAVIENCNSCKNTSSKTKLGGTCNGECKTECKGECDAYKEFIEKCKGTA
AEGTSGSSWVKRWYQIYMRYSKYIEDAKRNRKAGTKNCGTSSTTSTAESKCVQS
>gi|254952596|gb|ACT97128.1| VAR2CSA [Plasmodium falciparum] | 332 aa
(SEQ ID NO: 15)
KCDKCKSEQSKKNNNIWIWKKSSGTEGGLQKEYANTIALPPRTQSLYLGNLRKLENVCEDVKDINFDTKE
KFLAGCLIAAFHEGKNLKKRYLEKKNGDNNSKLCKALKYSFADYGDLIKGTSIWDNEYTKDLELNLQKIFG
KLFRKYIKKNNTAEQDTSYSSLDELRESWWNTNKKYIWTAMKHGTTCSSGSGDNGSISCDDIPTIDLIPQ
YLRFLQEWVEHFCEQRQEKVKDVIKNCNSCKECGGTCNGECKTECKNKCKDECDAYKKFIEECEGKAAE
GTSGSSWSKRWDQIYKRYSKYIEDAKRNRKAGTKNCGTSSTTSTAENKCVQS
>gi|90193465|gb|ABD92328.1| erythrocyte membrane protein 1 [Plasmodium falciparum] |
267 aa
(SEQ ID NO: 16)
NYIKDDPYSAEYTTKLSFILNSSDTENASEKIQKNNDEVCNPNESGIACVELAQTSGSSSNKTCNTHSFIK
ANKKKVCKDVKLGINKKDKDLKICVIEDDSLRGVDNCCCQDLLGILQENCSDKNQSGSSSNGSCNNKN
QEACQKKLENVFASLTNGYKCEKCKSEQSKKNNKNWIWKKYSVKEEGLQKEYANTIALPPRTQSLYLGNL
PKLGNVCKGVTDINFDTKEKFLAGCLIAAFHEGKNLKTTYLQNKKKLCKALKYSFADY
>gi|90193477|gb|ABD92334.1| erythrocyte membrane protein 1 [Plasmodium falciparum] |
263 aa
(SEQ ID NO: 17)
DYIKGDPYFAEYATKLSFILNSSDANTSSGETANHNDEACNPNESEIASVEQASISDRSSQKACNTHSSIK
ANKKKECKHVKLGVRENDKDLKICVIEDTSLSGVDNCCCQDLLGILQENCSDNKRGSSSNGSCDKNSEE
ICQKKLDEALASLHNGYKNQKCKSEQSKKNKNKWIWKKSSGNEKGLQKEYANTIGLPPRTQSLYLGNLP
KLENVCEDVTDINFDTKEKFLAGCLIAAFHEGKNLKTTYPQNKNDDNGKKLCKD
>gi|254952594|gb|ACT97127.1| VAR2CSA [Plasmodium falciparum] | 338 aa
(SEQ ID NO: 18)
KCDKCKSEQSKKNNNIWIWKKSSGNKKGLQKEYANTIGLPPRTQSLYLGNLPKLENVCKDVTDINFDTKE
KFLAGCLIAAFHEGKNLKISNEKKNDDNGKKLCKDLKYSFADYGDLIKGTSIWDNEYTKDLELNLQNNFG
KLFRKYIKKNNTAEQHTLYSSLDELRESWWNTNKKYIWLAMKHGTTCSSGSGDNGDGSVTGSGSSCDD
MSTIDLIPQYLRFLQEWVEHFCKQRQEKVNAVIENCNSCKNTSSKTKLGGTCNGECKTECEKKCKDECEK
YKEFIEECKRGDGTAGSPWVKRWDQIYMRYSKYIEDAKRNRKAGTKSCGTSAAENKCVQS
>gi|254952602|gb|ACT97131.1| VAR2CSA [Plasmodium falciparum] | 341 aa
(SEQ ID NO: 19)
KCDKCKSEQSKKNNNIWIWKKSSGDEKGLQKEYANTIALPPRTQSLYLGNLPKLENVCKDVTDINFDTKE
KFLAGCLIAAFHEGKNLKTSHQNKNADNGKKNDDNGKKLCKALKYSFADYGDLIKGTSIWDNEYTKDLE
LNLQQIFGKLFRKYIKRNNTAEQHTLYSSLDELRESWWNTNKKYIWLAMKHGTTCSSGSGDNGDGSVTG
SGSSCDDMSTIDLIPQYLRFLQEWVEHFCKQRQEKVKDVITNCNSCKECGGTCGSDCKTKCEAYKKFIEE
CNGTADGGTSGSSWSKRWDQIYKRYSKYIEDAKRNRKAGTKNCGPSSGANSGVTTTENKCVQS
>gi|254952660|gb|ACT97160.1| VAR2CSA [Plasmodium falciparum] | 352 aa
(SEQ ID NO: 20)
KCEKCESEQSKKNNKYWIWKKSSGNGEGLQEEYANTIALPPRTHSLCLVCLHEKEGKKTQELKNIRTNSE
LLKERIIAAFHEGKNLKTSPQNKNDNGKKLCKDLKYSFADYGDLIKGTSIWDNEYTKDLELNLQKIFGKLF
RKYIKKNNTAEQHTLYSSLDELRESWWNTNKKYIWLAMKHGAGMNSTMCNADGSVTGSSDSGSTTCC
GDNGSISCDDMPTIDLIPQYLRFLQEWVEHFCEQRQEKVNAVITNCKSCKECGGTCNSDCEKKCKAYKE
FIEKCKGGGTEGTSGSSWSKRWDQIYKRHSKHIEDAKRNRKAGTKNCGITTGTISGESSGANSGVTTTE
NKCVQS
>gi|254952652|gb|ACT97156.1| VAR2CSA [Plasmodium falciparum] | 344 aa
(SEQ ID NO: 21)
KCDKCKSGTSRSRKIWTWRKFRGNGEGLQKEYANTIGLSPRTQLLYLVCLHEKGKKTQELKNISTNSELL
KEWIIAAFHEGKNLKTTYPQKKNDDNGKKLCKALKYSFADYGDLIKGTSIWDNDFTKDLELNLQKIFGKLF
RKYIKKNIASDENTSYSSLDELRESWWNTNKKYIWTAMKHGAGMNGTTCCGDGSVTGSSDSGSTTCCG
DGSVTGSGSSCDDIPTIDLIPQYLRFLQEWVEHFCEQRQEKVKDVITNCKSCKESEKKCKNKCDAYKEFI
DGTGSGGGTGTAGSSWSKRWDQIYMRYSKYIEDAKRNRKAGTKNCGTSSGANSGVTTTENKCVQS
>gi|254952622|gb|ACT97141.1| VAR2CSA [Plasmodium falciparum] | 350 aa
(SEQ ID NO: 22)
KCEKCKSEQSKKNNKIWTWRKFPGNGEGLQKEYANTIGLSPRTQLLYLVCLHEKGKKTQHKTISTNSELL
KEWIIAAFHEGKNLKKRYLEKKKGDNNSKLCKDLKYSFADYGDLIKGTSIWDNDFTKDLELNLQQIFGKLF
RKYIKKNIASDENTSYSSLDELRESWWNTNKKYIWTAMKHGAGMNSTMCNGDGSVTGSSDSGSTTCS
GDNGSISCDDIPTIDLIPQYLRFLQEWVEHFCEQRQEKVKDVIKNCNSCKECGGTCNGECKTECKNKCK
DECEKYKNFIEVCTGGDGTAGSPWSKRWYQIYMRYSKYIEDAKRNRKAGTKSCGTSSGANSGVTTTESK
CVQS
>gi|254952626|gb|ACT97143.1| VAR2CSA [Plasmodium falciparum] | 359 aa
(SEQ ID NO: 23)
KCEKCKSEQSKKNNKNWIWRKFPGNGEGLQKEYANTIGLPPRTHSLYLVCLHEKGKKTQELKNIRTNSEL
LKEWIIAAFHEGKNLKKRYHQNNNSGNKKKLCKALEYSFADYGDLIKGTSIWDNEYTKDLELNLQQIFGK
LFRKYIKKNISTEQDTLYSSLDELRESWWNTNKKYIWLAMKHGAGMNSTTCCGDGSVTGSSDSGSTTCS
GDNGSISCDDMPTIDLIPQYLRFLQEWVEHFCEQRQEKVKDVIENCKSCKNTSGERIIGGTCNGECKTEC
EKKCKAACEAYKTFIEECEGKAAEGTSGSSWSKRWYQIYMRYSKYIEDAKRNRKAGTKNCGKSSGANSG
VTTTENKCVQS
>gi|90193469|gb|ABD92330.1| erythrocyte membrane protein 1 [Plasmodium falciparum] |
270 aa
(SEQ ID NO: 24)
NYIKDDPYSKEYVTKLSFIPNSSDANNPSGETANHNDEVCNPNESEISSVEHAQTSVLLSQKAYITHSSIK
ANKKKVCKYVKLGVRENDKDLKICVIEDDSLRGVENCCFKDFLRILQENCSDNKRESSSNGSCNNNNEE
ACEKNLDEALASLTNCYKNQKCKSGTSTVNNNKWIWKKSSGKEGGLQKEYANTIGLPPRTQSLCLVVCL
DEKEGKTQELKNIRTNSELLKEWIIAAFHEGKNLKKRYHQNKNDDNNSKLCKALKYSFADY
>gi|254952644|gb|ACT97152.1| VAR2CSA [Plasmodium falciparum] | 334 aa
(SEQ ID NO: 25)
KCDKCKSEQSKKNNKYWIWKKYSVKEGGLQKEYANTIALPPRTQSLCLVVCLDEKEGKTQELKNIRTNSE
LLKERIIAAFHEGKNLKTYHEKKKGDDGKKLCKDLKYSFADYGDLIKGTSIWDNDFTKDLELNLQKIFGKL
FRKYIKKNNTAEQHTSYSSLDELRESWWNTNKKYIWTAMKHGAEMNGTTCSCSGDSSNDIPTIDLIPQY
LRFLQEWVEHFCEQRQAKVNAVIKNCKSCKECGGTCNGECKTECKTKCKGECEKYKEFIEKCEGQAAEG
TSGSSWSKRWYQIYMRYSKYIEDAKRNRKAGTKNCGTSSGANSGVTTTENKCVQS
>gi|254952642|gb|ACT97151.1| VAR2CSA [Plasmodium falciparum] | 351 aa
(SEQ ID NO: 26)
KCDKCKSEQSKKNNKNWIWKKYSGTEGGLQKEYANTIALPPRTQSLYLVCLHEKEEKTQELKNISTNSEL
LKEWIIAAFHEGKNLKISPQNKNDNGKNLCKDLKYSFADYGDLIKGTSIWDNDFTKDLELNLQQIFGKLFR
KYIKKNNTAEQDTLYSSLDELRESWWNTNKKYIWTAMKHGAGMNGTTCCGDGSVTGSSDSGSTTCCG
DGSVTGSGSSCDDIPTIDLIPQYLRFLQEWVEHFCEQRQAKVKDVIKNCNSCKECGGTCNGECKTECEK
KCKGECEAYKKFIEKCNGGGGEGTSGSSWSKRWDQIYMRYSKYIEDAKRNRKAGTKNCGTSSTTNAAE
NKCVQS
>gi|254952658|gb|ACT97159.1| VAR2CSA [Plasmodium falciparum] | 353 aa
(SEQ ID NO: 27)
KCDKCKSGTSTVNKKWIWKKFPGKEGGLQEEYANTIALPPRTQSLCLVVCLDEKEGKTQHKTISTNSELL
KEWIIAAFHEGKNLKISNKKKNDENNSKLCKDLKYSFADYGDLIKGTSIWDNDFTKDLELNLQKIFGKLFR
KYIKKNNTAEQDTSYSSLDELRESWWNTNKKYIWLAMKHGTTCSSGSGDNGDGSVTGSSDSGSTTCC
GDGSVTGSGSSCDDIPTIDLIPQYLRFLQEWVEHFCKQRQAKVKDVIENCKSCKNTSSKTKLGDTCNSD
CKTKCKVACEKYKEFIEKCVSAAGGTSGSSWVKRWDQIYMRYSKYIEDAKRNRKAGTKNCGPSSTTSTA
ESKCVQS
>gi|254952640|gb|ACT97150.1| VAR2CSA [Plasmodium falciparum] | 327 aa
(SEQ ID NO: 28)
KCDKCKSGTSTVNKKWIWKKYSGKEGGLQKEYANTIGLPPRTQSLCLVCLHEKEGKTQELKNISTNSELL
KEWIIAAFHEGKNLKISNKKKNDDNGKKLCKDLKYSFADYGDLIKGTSIWDNDFTKDLELNLQKIFGKLF
RKYIKKNNTAEQDTLYSSLDELRESWWNTNKKYIWTAMKHGAGMNSTTCSCSGDSSNDIPTIDLIPQYL
RFLQEWVEHFCKQRQEKVNAVITNCKSCKESGGTCNSDCEKKCKIECEKYKNFIEKCVTAAGGTSGSSW
SKRWDQIYKMYSKYIEDAKRNRKAGTKNCGPSSTTNAAASTDENKCVQS
>dd2full 745 amino acids | 628 aa
(SEQ ID NO: 29)
NYIKGDPYFAEYATKLSFILNSSDTENASETPSKYYDEACNCNESEIASVGQAQTSGPSSNKTC
ITHSSIKTNKKKECKDVKLGINNNDKVLRVCVIEDTSLSGVDNCCCQDLLGILQENCSDNKRG
SSSNGSCDKNSEEICQKKLEKVFASLTNGYKCDKCKSGTSRSKKKWIWKKSSGNEEGLQKEY
ANTIGLPPRTQSLCLVCLHEKEGKTQHKTISTNSELLKEWIIAAFHEGKNLKTSHEKKNDDNGK
KLCKALEYSFADYGDLIKGTSIWDNEYTKDLELNLQKIFGKLFRKYIKKNNTAEQHTSYSSLDE
LRESWWNTNKKYIWTAMKHGAGMNGTTCSCSGDSSNDMPTIDLIPQYLRFLQEWVEHFCKQ
RQEKVNAVIENCNSCKESGGTCNSDCKTECKNKCEAYKEFIEDCKGGGTGTAGSPWSKRWDQ
IYKRYSKHIEDAKRNRKAGTKNCGTSSTTNAAASTDENKCVQSDVDSFFKHLIDIGLTTPSSYL
SNVLDDNICGADKAPWTTYTTYTTTKNCDIQKKTPKSQSCDTLVVVNVPSPLGNTPHEYKYAC
ECKIPTTEETCDDRKEYMNQWSCGSAQTVRGRSGKDDYELYTYNGVKETKPLGTLKNSKLD
>gi|254952636|gb|ACT97148.1| VAR2CSA [Plasmodium falciparum] | 350 aa
(SEQ ID NO: 30)
KCEKCKSEQSKKNNKNWIWRKFRGTEGGLQEEYANTIGLPPRTQSLCLVVCLDEKGKKTQELKNIRTNSE
LLKEWIIAAFHEGKNLKPSHQNKNSGNKENLCKALKYSFADYGDLIKGTSIWDNDFTKDLELNLQKIFGKL
FRKYIKKNNTAEQHTSYSSLDELRESWWNTNKKYIWTAMKHGAEMNGTTCNADGSVTGSSDSGSTTCS
GDNGSISCDDIPTIDLIPQYLRFLQEWVEHFCKQRQEKVNAVINSCNSCKNTSSKTKLGDTCNSDCKTKC
KIECEKYKTFIEKCVTAAGGTSGSPWSKRWDQIYKRYSKYIEDAKRNRKAGTKNCGPSSTTSTAESKCVQ
S
>gi|254952638|gb|ACT97149.1| VAR2CSA [Plasmodium falciparum] | 330 aa
(SEQ ID NO: 31)
KCDKCKSEQSKKNNKNWIWRKYSGNGEGLQKEYANTIGLPPRTHSLYLVCLHEKEGKTQELKNIRTNSEL
LKEWIIAAFHEGKNLKTTYLENKNDENKKKLCKALKYSFADYGDLIKGTSIWDNDFTKDLELNLQKIFGKL
FRKYIKKNIASDENTLYSSLDELRESWWNTNKKYIWTAMKHGAEMNGTTCSSGSGDNGSISCDDIPTID
LIPQYLRFLQEWVGHFCKQRQEKVNAVITNCNSCKESGGTCNSDCEKKCKIECEKYKKFIEECRTAAGGT
SGSPWSKRWDQIYKMYSKYIEDAKRNRKAGTKNCGPSSTTSTAESKCVQS
>gi|254952628|gb|ACT97144.1| VAR2CSA [Plasmodium falciparum] | 334 aa
(SEQ ID NO: 32)
KCDKCKSEQSKKNNKNWIWRKYSGNGEGLQKEYANTIGLPPRTHSLYLVCLHEKEGKTQHKTISTNSELL
KEWIIAAFHEGKNLKKRYPQNNNSGNKKKLCKDLKYSFADYGDLIKGTSIWDNEYTKDLELNLQKAFGKL
FRKYIKKNIASDENTLYSSLDELRESWWNTNKKYIWLAMKHGAEMNGTMCNADGSVTGSGSSCDDMST
IDLIPQYLRFLQEWVEHFCEQRQAKVKDVINSCKSCKESGDTCNSDCEKKCKNKCDAYKTFIEEFCTADG
GTAGSPWSKRWDQIYKRYSKYIEDAKRNRKAGTKNCGTSSGANSGVTTTENKCVQS
>gi|254952630|gb|ACT97145.1| VAR2CSA [Plasmodium falciparum] | 350 aa
(SEQ ID NO: 33)
KCDKCKSGTSTVNKNWIWKKYSGKEEGLQKEYANTIALPPRTHSLYLVCLHEKGKKTQELKNIRTNSELL
KEWIIAAFHEGKNLKTSPQNNNSGNKKKLCKALKYSFADYGDLIKGTSIWDNDFTKDLELNLQKIFGKLF
RKYIKKNNTAEQHTSYSSLDELRESWWNTNKKYIWLAMKHGAEM NGTTCCGDGSVTGSSDSGSTTCS
GDNGSISCDDMPTTDFIPQYLRFLQEWVEHFCKQRQEKVKHVMESCKSCKECGDTCNGECKTECEKKC
KNKCEAYKTFIEKCVSADGGTSGSSWSKRWDQIYMRYSKYIEDAKRNRKAGTKNCGTSSTTNAAASTAE
NKCVQS
>P13 745 amino acids | 647 aa
(SEQ ID NO: 34)
DYIKDDPYSAEYATKLSFILNPSDANTSSGETANHNDEVCNCNESEIASVELAPISDSSSNKTC
ITHSFIGANKKKECKDVKLGVREKDKDLKICVIEDDSLRGVENCCCQDLLGILQENCSDNKSGS
SSNGSCDKNSEDECQKKLENVFASLKNGYKCDKCKSGTSTVNKKWIWRKYSGNGEGLQKEYA
NTIGLPPRTHSLYLVCLHEKEGKTQHKTISTNSELLKEWIIAAFHEGKNLKTSHQNNNSGNKK
KLCKALKYSFADYGDLIKGTSIWDNDFTKDLELNLQKIFGKLFRKYIKKNIASDENTSYSSLDE
LRESWWNTNKKYIWLAMKHGAEMNSTMCNGDGSVTGSSDSGSTTCSGDNGSISCDDIPTID
LIPQYLRFLQEWVEHFCKQRQEKVKDVITNCKSCKESGDTCNSDCEKKCKNKCEAYKKFIEER
RTAAQGTAESSWVKRWDQIYMRYSKYIEDAKRNRKAGTKSCGPSSTTNAAASTAENKCVQS
DIDSFFKHLIDIGLTTPSSYLSIVLDDNICGADNAPWTTYTTYTTTKNCDIKKKTPKPQSCDTL
VVVNVPSPLGNTPHEYKYACQCRTPNKQESCDDRKEYMNQWSSGSAQTVRGRSTNNDYELYTYNGV
KETKPLGTLKNSKLD
>gi|254952608|gb|ACT97134.1| VAR2CSA [Plasmodium falciparum] | 341 aa
(SEQ ID NO: 35)
KCDKCKSGTSTVNKKWIWRKSSGNKEGLQKEYANTIGLPPRTQSLYLGNLPKLENVCEDVKDINFDTKEK
FLAGCLIVSFHEGKNLKTSHEKKNDDNGKKLCKALEYSFADYGDLIKGTSIWDNEYTKDLELNLQKIFGKL
FRKYIKKNNTAEQDTSYSSLDELRESWWNTNKKYIWTAMKHGAGMNITTCCGDGSSGENQTNSCDDIP
TIDLIPQYLRFLQEWVEHFCKQRQEKVNAVVTNCKSCKESGGTCNGECKTKCKNKCEVYKTFIDNVGDG
TAGSPWVKRWDQIYKRYSKHIEDAKRNRKAGTKNCGITTGTISGESSGATSGVTTTENKCVQS
>7g8 745 amino acids | 632 aa
(SEQ ID NO: 36)
NYIKDDPYSKEYVTKLSFIPNSSDANTSSEKIQKNNDEVCNPNESGISSVEQAQTSGPSSNKT
CITHSSIKANKKKECKDVKLGVRENDKDLKICVIEDTSLSGVDNCCCQDLLGILQENCSDNKRG
SSSNDSCDNKNQDECQKKLDEALESLHNGYKNQKCKSGTSTVNKKWIWKKSSGNKEGLQKE
YANTIGLPPRTQSLYLGNLPKLENVSKGVTDIIYDTKEKFLAGCLIVSFHEGKNLKTSHEKKND
DNGKKLCKALEYSFADYGDLIKGTSIWDNEYTKDLELNLQKAFGKLFRKYIKKNISAEQDTSYS
SLDELRESWWNTNKKYIWIAMKHGAGMNGTTCCGDGSSGENQTNSCDDIPTIDLIPQYLRFL
QEWVEHFCEQRQAKVKDVITNCKSCKNTSGERKIGGTCNGECKTKCKNKCEAYKTFIEHCKGG
DGTAGSSWVKRWDQIYKRYSKHIEDAKRNRKAGTKSCGTSTAENKCVQSDIDSFFKHLIDIG
LTTPSSYLSIVLDENNCGEDKAPWTTYTTTKNCDIQKDKSKSQSSDTLVVVNVPSPLGNTPHG
YKYACQCKIPTTEETCDDRKEYMNQWSCGSARTMKRGYKNDNYELCKYNGVDVKPTTVRSSSTKLD
>Indo 745 amino acids | 639 aa
(SEQ ID NO: 37)
DYIKGDPYSAEYVTKLSFIPNSSDANNPSEKIQKNNDEVCNCNESEISSVGQASISDPSSNKTC
NTHSSIKANKKKVCKDVKLGVRENDKVLKICVIEHTSLRGVDNCCFKDLLGILQEPRIDKNQS
GSSSNGSCDKNSEEACEKNLEKVLASLTNGYKCDKCKSGTSRSKKKWIWKKYSGKEGGLQEE
YANTIGLPPRTQSLCLVVCLDEKEGKTQELKNISTNSELLKEWIIAAFPEGKNLKPSPEKKKGD
NGKKLCKDLKYSFADYGDLIKGTSIWDNEYTKDLELNLQKIFGKLFRKYIKKNIASDENTLYSS
LDELRESWWNTNKKYIWLAMKHGAGMNSTMCNADGSVTGSGSSCDDMPTIDLIPQYLRFLQ
EWVEHFCKQRQEKVKPVIENCNSCKNTSSERKIGGTCNSDCKTECKNKCEVYKKFIEDCKGGD
GTAGSSWSKRWDQIYKRYSKYIEDAKRNRKAGTKNCGPSSTTNAAENKCVQSDIDSFFKHLI
DIGLTTPSSYLSTVLDDNICGEDNAPWTTYTTYTTTKNCDKDKKKSKSQSCDTLVVVNVPSPL
GNTPHEYKYACECRTPNKQESCDDRKEYMNQWISDNTKNPKGSGSGKDYYELYTYNGVDVKPTTVRS
SSTKLD
>MC 745 amino acids | 655 aa
(SEQ ID NO: 38)
DYIKGDPYFAEYATKLSFILNSSDANTSSGETANHNDEACNCNESEISSVEHASISDPSSNKTC
NTHSSIKANKKKVCKHVKLGVRENDKDLRVCVIEHTSLSGVENCCFKDFLRILQENCSDNKSG
SSSNGSCDKNNEEACEKNLEKVFASLTNCYKCEKCKSEQSKKNNKKWTWRKSSGNKGGLQEE
YANTIGLPPRTQSLCLVVCLDEKEGKKTQELKNIRTNSELLKEWIIAAFHEGKNLKPSHEKKND
DNGKKNDDNNSKLCKDLKYSFADYGDLIKGTSIWDNEYTKDLELNLQKIFGKLFRKYIKKNIA
SDENTLYSSLDELRESWWNTNKKYIWLAMKHGAEMNGTTCNADGSVTGSGSSCDDIPTIDLI
PQYLRFLQEWVEHFCKQRQAKVKDVIENCKSCKESGNKCKTECKNKCEAYKKFIENCKGGDG
TAGSSWVKRWDQIYMRYSKYIEDAKRNRKAGTKNCGPSSITNVSASTDENKCVQSDIDSFFK
HLIDIGLTTPSSYLSIVLDDNICGDDKAPWTTYTTYTTYTTYTTYTTYTTYTTTKNCDKERDKSK
SQSCNTAVVVNVPSPLGNTPHEYKYACECRTPSNKELCDDRKEYMNQWSSGSAQTVRDRSGKDYY
ELYTYNGVKETKLPKKLNSSKLD
>gi|254952650|gb|ACT97155.1| VAR2CSA [Plasmodium falciparum] | 347 aa
(SEQ ID NO: 39)
KCDKCKSEQSKKNNKYWIWKKSSVKEEGLQKEYANTIALPPRTHSLCLVVCLDEKGKKTQELKNISTNSE
LLKERIIAAFHEGKNLKTTYLEKKNADNNSKLCKALKYSFADYGDLIKGTSIWDNEYTKDLELNLQQIFGKL
FRKYIKKNNTAEQHTLYSSLDELRESWWNTNKKYIWLAMKHGAGMNGTTCCGDGSVTGSSDSGSTTCS
GDNGSISCDDMPTTDFIPQYLRFLQEWVEHFCKQRQEKVKDVIENCNSCKNNLGKTEINEKCKTECKNK
CEAYKNFIEKFCTADGGTSGSPWSKRWDQIYKRYSKYIEDAKRNRKAGTKNCGTSSTTSTAENKCVQS
>gi|254952648|gb|ACT97154.1| VAR2CSA [Plasmodium falciparum] | 335 aa
(SEQ ID NO: 40)
KCEKCKSGTSTVNKYWIWRKSSGNKEGLQKEYANTIALPPRTHSLCLVVCLDEKEGKTQELKNISTNSEL
LKERIIAAFHEGENLKTSHEKKKGDDGKKNADNNSKLCKALKYSFADYGDLIKGTSIWDNEYTKDLELNL
QKIFGKLFRKYIKKNIASDENTSYSSLDELRESWWNTNKKYIWLAMKHGAGMNGTTCSCSGDSSDDMP
TTDFIPQYLRFLQEWVEHFCKQRQENVNAVIENCNSCKECGGTCNSDCEKKCKTECKNKCEAYKNFIEKF
CTADGGTSGYSWSKRWDQIYKRYSKYIEDAKRNRKAGTKSCGTSSTTSTAESKCVQS
>ghana2 745 amino acids | 667 aa
(SEQ ID NO: 41)
SYVKNNPYSKEYVTKLSFILNPSDANNPSETPSKYYDEVCNCNESGIACVGQAQTSGPSSNKT
CITHSFIGANKKKVCKDVKLGVREKDKDLKICVIEDTYLSGVDNCCFKDFLGMLQENCSDNKS
GSSSNGSCNNKNQDECEKNLDEALASLTNGYKCEKCKSGTSTVNKYWIWRKSSGNKEGLQKE
YANTIALPPRTHSLCLVVCLDEKEGKTQHKTISTNSELLKEWIIAAFHEGKNLKTSHEKKKGDD
GKKNADNNSKLCKALKYSFADYGDLIKGTSIWDNDFTKDLELNLQKIFGKLFRKYIKKNIASD
ENTSYSSLDELRESWWNTNKKYIWLAMKHGAGMNSTTCCGDGSVTGSSDSGSTTCCGDGSV
TGSGSSCDDMPTTDFIPQYLRFLQEWVEHFCKQRQENVNAVIENCNSCKECGGTCNSDCEKK
CKTECKGECDAYKEFIEKCNGGAAEGTSGSSWSKRWDQIYKRYSKYIEDAKRNRKAGTKNCG
TSSTTSTAESKCVQSDIDSFFKHLIDIGLTTPSSYLSIVLDENICGADNAPWTTYTTYTTYTTYT
TTEKCNKETDKSKLQQCNTSVVVNVPSPLGNTPHGYKYVCECRTPNKQETCDDRKEYMNQWISD
NTKNPKGSRSTNNDYELYTYNGVQIKPTTVRSNSTKLD
>gi|254952634|gb|ACT97147.1| VAR2CSA [Plasmodium falciparum] | 348 aa
(SEQ ID NO: 42)
KCDKCKSEQSKKNNKNWIWKKSSGNEKGLQKEYANTIGLPPRTQSLCLVVCLDEKEGKTQELKNIRTNS
ELLKEWIIAAFHEGKNLKTSHEKKKGDNNSKLCKDLKYSFADYGDLIKGTSIWDNEYTKDLELNLQNNFG
KLFRKYIKKNIASDENTSYSSLDELRESWWNTNKKYIWLAMKHGAGMNSTTCSSGSGSTTCSSGSGSTT
CSSGSGDSCDDMPTIDLIPQYLRFLQEWVEHFCKQRQEKVNAVIKNCNSCKESGGTCNGECKTECKNKC
EAYKTFIEEFCTADGGTSGSPWSKRWDQIYKMYSKHIEDAKRNRKAGTKNCGPSSTTNVSVSTDENKCV
QS
>ghana1 745 amino acids | 652 aa
(SEQ ID NO: 43)
DYIKDDPYFAEYVTKLSFILNSSDANNPSGETANHNDEVCNPNESGIASVEQAQTSDPSSNKT
CNTHSSIKANKKKVCKHVKLGVRENDKDLKICVIEHTSLSGVENCCCQDFLRILQENCSDNKS
GSSSNGSCNNKNQEACEKNLEKVLASLTNCYKCDKCKSEQSKKNNKNWIWKKSSGNEKGLQ
KEYANTIGLPPRTQSLCLVVCLDEKEGKTQELKNIRTNSELLKEWIIAAFHEGKNLKKRYPQNK
NDDNNSKLCKDLKYSFADYGDLIKGTSIWDNEYTKDLELNLQNNFGKLFRKYIKKNISTEQDT
LYSSLDELRESWWNTNKKYIWLAMKHGAGMNSTTCSSGSGSTTCSSGSGSTTCSSGSGDSCD
DMPTTDFIPQYLRFLQEWVEHFCKQRQEKVNAVIKNCNSCKESGGTCNGECKTECKNKCEAY
KTFIEEFCTADGGTSGSPWSKRWDQIYKMYSKHIEDAKRNRKAGTKNCGPSSTTNVSVSTDE
NKCVQSDIDSFFKHLIDIGLTTPSSYLSIVLDDNICGEDKAPWTTYTTYTTTKKCNKETDKSKS
QSCNTAVVVNVPSPLGNTPHGYKYACECKIPTTEETCDDRKEYMNQWIIDTSKKQKGSGSGKDDYE
LYTYNGVDVKPTTVRSNSTKLD
>V1S1 745 amino acids | 628 aa
(SEQ ID NO: 44)
DYIKDDPYSAQYTTKLSFILNPSDANTSSEKIQKNNDEACNCNESGISSVGQAQTSGPSSNKT
CITHSSIKANKKKVCKDVKLGINNNDKVLRVCVIEDTSLSGVDNCCCQDLLGILQENCSDNKR
GSSSNGSCNNNNEEACEKNLDEAPASLHNGYKNQKCKSGTSRSKKKWIWKKSSGNEKGLQE
EYANTIGLPPRTQSLCLVCLHEKEGKTQH KTISTNSELLKEWIIAAFHEGKNLKTSHEKKNDDN
GKKLCKALEYSFADYGDLIKGTSIWDNEYTKDLELNLQKAFGKLFRKYIKKNNTAEQDTSYSSL
DELRESWWNTNKKYIWIAMKHGAGMNGTTCSCSGDSSNDMPTIDLIPQYLRFLQEWVEHFC
EQRQAKVKDVITNCKSCKESGNKCKTECKTKCKDECEKYKTFIEDCNGGGTGTAGSSWVKRW
DQIYKRYSKHIEDAKRNRKAGTKNCGPSSITNAAASTDENKCVQSDIDSFFKHLIDIGLTTPSS
YLSNVLDENSCGDDKAPWTTYTTYTTTKNCDIQKDKSKSQPINTSVVVNVPSPLGNTPYRYKY
ACECKIPTTEESCDDRKEYMNQWSCGSARTMKRGYKNDNYELCKYNGVDVKPTTVRSNSSKLD
>raj116_var25 745 amino acids | 653 aa
(SEQ ID NO: 45)
DYIKGDPYFAEYATKLSFILNPSDTENASETPSKYYDEACNPNESEIASVEQAQTSGPSSNKTC
ITHSSIKTNKKKECKDVKLGVRENDKDLKICVIEDTSLSGVDNCCFKDLLGILQENCSDNKRGS
SSNDSCNNNNEEACEKNLDEALASLTNGYKCDKCKSGTSTVNKKWTWRKSSGNEEGLQKEYA
NTIGLPPRTQSLCLVCLHEKEGKTKHKTISTNSELLKEWIIAAFHEGKNLKTSHEKKNDDNGKK
LCKALEYSFADYGDLIKGTSIWDNEYTKDLELNLQKAFGKLFRKYIKKNNTAEQDTSYSSLDEL
RESWWNTNKKYIWTAMKHGAEMNGTTCSSGSGDNGDSSITGSSDSGSTTCSGDNGSISCDD
IPTTDFIPQYLRFLQEWVEHFCEQRQAKVKDVINSCNSCNESGGTCNGECKTKCKDECEKYKK
FIEDCNGGDGTAGSSWVKRWDQIYKRYSKHIEDAKRNRKAGTKNCGPSSITNAAASTDENKC
VQSDVDSFFKHLIDIGLTTPSSYLSIVLDENSCGDDKAPWTTYTTYTTTEKCNKERDKSKSQSS
DTLVVVNVPSPLGNTPHEYKYACECKIPTNEETCDDRKDYMNQWISDTSKKQKGSGSGKDYYELYTY
NGVQIKQAAGRSSSTKLD
>gi|31323048|gb|AAP37940.1| var2csa [Plasmodium falciparum] | 490 aa
(SEQ ID NO: 46)
KCDKCKSEQSKKNNNKWIWKKYSGNGEGLQKEYANTIGLPPRTQSLCLVCLHEKEGKTQHKTISTNSEL
LKEWIIAAFHEGKNLKKRYPQNKNDDNNSKLCKALEYSFADYGDLIKGTSIWDNEYTKDLELNLQKAFGK
LFRKYIKKNNTAEQDTSYSSLDELRESWWNTNKKYIWTAMKHGAEMNGTTCSSGSGDNGDSSCDDIPT
IDLIPQYLRFLQEWVEHFCKQRQAKVKDVINSCNSCKNTSGERKIGGTCNSDCEKKCKVACDAYKTFIEE
CRTAVGGTAGSSWVKRWDQIYKRYSKHIEDAKRNRKAGTKNCGPSSTTNAAENKCVQSDIDSFFKHLID
IGLTTPSSYLSNVLDENSCGADKAPWTTYTTYTTYTTYTTYTTTEKCNKERDKSKSQQSNTSVVVNVPSPL
GNTPHEYKYACECKIPTTEETCDDRKEYMNQWIIDNTKNPKGSGSTDNDYELYTYNGVQIKQAAGRSSST
KLD
>gi|254952620|gb|ACT97140.1| VAR2CSA [Plasmodium falciparum] | 335 aa
(SEQ ID NO: 47)
KCEKCKSGTSTVNNKWIWRKSSGKEGGLQKEYANTIGLPPRTQSLYLGNLPKLENVCKGVTDIIYDTKEK
FLSGCLIAAFHEGKNLKTTYLEKKNDDNGKKLCKALEYSFADYGDLIKGTSIWDNEYTKDLELNLQKIFGK
LFRKYIKKNNTAEQDTSYSSLDELRESWWNTNKKYIWIAMKHGAGMNGTTCSSGSGDSSNDIPTTDFIP
QYLRFLQEWVENFCEQRQAKVKPVIENCNSCKESGGTCNGECKTKCKVACDAYKKFIDGTGSGGGSRPT
GIAGSSWSKRWDQIYKRYSKHIEDAKRNRKAGTKNCGPSSITNVSVSTDENKCVQS
>T2C6 745 amino acids | 637 aa
(SEQ ID NO: 48)
NYIKDDPYSKEYVTKLSFIPNSSDANTSSEKIQKNNDEVCNPNESGISSVEQAQTSDPSSNKT
CITHSSIKANKKKECKDVKLGVRENDKDLKICVIEHTSLSGVDNCCFKDFLRMLQEPRIDKNQ
RGSSSNGSCDKNSEEACEKNLDEALASLTNGYKCDKCKSEQSKKNNNKWIWKKFPGKEGGLQ
EEYANTIGLPPRTQYLCLVVCLDEKEGKTQELKNIRTNSELLKEWIIAAFHEGKNLKTTYPQKK
NDDNGKKLCKDLKYSFADYGDLIKGTSIWDNEYTKNVELNLQNNFGKLFRKYIKKNNTAEQD
TSYSSLDELRESWWNTNKKYIWLAMKHGAEMNSTTCCGDGSVTGSGSSCDDIPTIDLIPQYL
RFLQEWVEHFCKQRQAKVKDVITNCNSCKESGNKCKTECKNKCKDECEKYKKFIEACGTAVG
GTGTAGSPWSKRWDQIYKRYSKHIEDAKRNRKAGTKNCGPSSTTNAAENKCVQSDIDSFFKH
LIDIGLTTPSSYLSIVLDDNICGADKAPWTTYTTYTTENCDIQKKTPKSQSCDTLVVVNVPSPL
GNTPHGYKYACQCRTPNKQESCDDRKEYMNQWIIDNTKNPKGSGSGKDYYELCKYNGVKETKPLGTL
KNSKLD
>gi|254952632|gb|ACT97146.1| VAR2CSA [Plasmodium falciparum] | 330 aa
(SEQ ID NO: 49)
KCDKCKSEQSKKNNNKWIWRKFPGKEGGLQKEYANTIGLPPRTQSLCLVCLHEKEGKTQHKTISTNSELL
KEWIIAAFHEGKNLKTTYLEKKNAENKKKLCKALKYSFADYGDLIKGTSIWDNEYTKDLELNLQKIFGKLF
RKYIKKNNTAEQDTSYSSLDELRESWWNTNKKYIWTAMKHGAGMNGTMCNADGSVTGSGSSCDDMPT
TDFIPQYLRFLQEWVEHFCKQRQAKVKDVIENCKSCKESGNKCKTECKNKCDAYKTFIEECGTAVGGTAG
SSWVKRWDQIYKRYSKHIEDAKRNRKAGTKNCGTSSTTNAAASTAENKCVQS
>gi|90193487|gb|ABD92339.1| erythrocyte membrane protein 1 [Plasmodium falciparum] |
269 aa
(SEQ ID NO: 50)
NYIKDDPYSKEYVTKLSFILNSSDAENASETPSKYYDEACNCNESGISSVEQASISDRSSQKACNTHSFIG
ANKKKVCKHVKLGVRENDKDLKICVIEDDSLRGVENCCFKDFLRMLQEPRIDKNQRGSSSNDSCNNNNE
EACEKNLDEALASLHNGYKNQKCKSEQSKKNNNKWIWKKSSGKEGGLQKEYANTIGLPPRTQSLCLVCL
HEKEGKTQHKTISTNSELLKEWIIDAFHEGKNLKTTYLEKKKGDNGKKLCKALKYSFADY
>gi|254952646|gb|ACT97153.1| VAR2CSA [Plasmodium falciparum] | 347 aa
(SEQ ID NO: 51)
KCDKCKSEQSKKNNKNWIWKKSSGKEGGLQKEYANTIALPPRTQSLCLVVCLHEKEGKTQHKTISTNSE
LLKEWIIDAFHEGKNLKTTYLEKQNADNGKKNADNNSKLCKDLKYSFADYGDLIKGTSIWDNEYTKDLEL
NLQQIFGKLFRKYIKKNIASDENTLYSSLDELRESWWNTNKKYIWTAMKHGAEMNGTTCSSGSGDSSSG
ENQTNSCDDIPTIDLIPQYLRFLQEWVEHFCEQRQAKVKDVITNCKSCKESGGTCNSDCKTKCKGECEKY
KKFIEKCKGGGTEGTSGSSWVKRWYQIYMRYSKYIEDAKRNRKAGTKSCGTSSGANSGVTTTESKCVQ
S
>gi|90193485|gb|ABD92338.1| erythrocyte membrane protein 1 [Plasmodium falciparum] |
269 aa
(SEQ ID NO: 52)
DYIKDDPYSKEYTTKLSFILNSSDANTSSEKIQKNNDEVCNPNESEISSVEQAQTSRPSSNKTCITHSSIK
ANKKKVCKDVKLGVRENDKVLRVCVIEHTSLSGVENCCCQDLLGILQENCSDNKRGSSSNGSCDKNSEE
ACEKNLDEALASLTNCYKNQKCKSEQSKKNNNKWIWKKSSGNEKGLQKEYANTIGLPPRTQSLCLVCLH
EKEGKTQELKNISTNSELLKEWIIAAFHEGKNLKTTYPQNKNDDNGKKLFKDLKYSFADY
>MTS1 745 amino acids | 646 aa
(SEQ ID NO: 53)
DYIKDDPYSKEYTTKLSFILNSSDANTSSEKIQKNNDEVCNPNESEISSVEQAQTSRPSSNKTC
ITHSSIKANKKKVCKDVKLGVRENDKVLRVCVIEHTSLSGVENCCCQDLLGILQENCSDNKRG
SSSNGSCDKNSEEACEKNLDEALASLTNCYKNQKCKSEQSKKNNNKWIWKKSSGKEGGLQKE
YANTIGLPPRTQSLYLGNLPKLENVCKGVTDINFDTKEKFLAGCLIAAFHEGKNLKTTYLEKKN
DDNGKKLCKALEYSFADYGDLIKGTSIWDNEYTKDLELNLQKAFGKLFRKYIKKNNTAEQDTS
YSSLDELRESWWNTNKKYIWTAMKHGAGMNGTTCSSGSGDSSNDIPTTDFIPQYLRFLQEW
VENFCEQRQAKVKDVIENCNSCKNTSGERKIGDTCNSDCEKKCKDECEKYKKFIEDCKGGDGT
AGSSWVKRWDQIYKRYSKHIEDAKRNRKAGTKNCGITTGTISGESSGATSGVTTTENKCVQS
DIDSFFKHLIDIGLTTPSSYLSNVLDDNICGEDNAPWTTYTTYTTEKCNKETDKSKSQQSNTAV
VVNVPSPLGNTPHGYKYACECKIPTTEETCDDRKEYMNQWSCGSAQTVRDRSGKDDYELCKYNGVQI
KQAAGTLKNSKLD
>Q8I639 (Q81639_PLAF7) Plasmodium falciparum (isolate 3D7), 632 aa extracellular part
(SEQ ID NO: 54)
NYIKGDPYFAEYATKLSFILNSSDANNPSEKIQKNNDEVCNCNESGIASVEQEQISDPSSNKTC
ITHSSIKANKKKVCKHVKLGVRENDKDLRVCVIEHTSLSGVENCCCQDFLRILQENCSDNKSG
SSSNGSCNNKNQEACEKNLEKVLASLTNCYKCDKCKSEQSKKNNKNWIWKKSSGKEGGLQK
EYANTIGLPPRTQSLCLVVCLDEKGKKTQELKNIRTNSELLKEWIIAAFHEGKNLKPSHEKKND
DNGKKLCKALEYSFADYGDLIKGTSIWDNEYTKDLELNLQKIFGKLFRKYIKKNNTAEQDTSYS
SLDELRESWWNTNKKYIWLAMKHGAGMNSTTCCGDGSVTGSGSSCDDIPTIDLIPQYLRFLQ
EWVEHFCKQRQEKVKPVIENCKSCKESGGTCNGECKTECKNKCEVYKKFIEDCKGGDGTAGSS
WVKRWDQIYKRYSKYIEDAKRNRKAGTKNCGPSSTTNAAENKCVQSDIDSFFKHLIDIGLTT
PSSYLSIVLDDNICGADKAPWTTYTTYTTTEKCNKETDKSKLQQCNTAVVVNVPSPLGNTPHG
YKYACQCKIPTNEETCDDRKEYMNQWSCGSARTMKRGYKNDNYELCKYNGVDVKPTTVRSNSSKLD
>Q8I639 (Q81639_PLAF7) Plasmodium falciparum (isolate 3D7), complete 2730 aa
extracellular part
(SEQ ID NO: 55)
MDKSSIANKIEAYLGAKSDDSKIDQSLKADPSEVQYYGSGGDGYYLRKNICKITVNHSDSGTNDPCDRIP
PPYGDNDQWKCAIILSKVSEKPENVFVPPRRQRMCINNLEKLNVDKIRDKHAFLADVLLTARNEGERIVQ
NHPDTNSSNVCNALERSFADIADIIRGTDLWKGTNSNLEQNLKQMFAKIRENDKVLQDKYPKDQNYRKL
REDWWNANRQKVWEVITCGARSNDLLIKRGWRTSGKSNGDNKLELCRKCGHYEEKVPTKLDYVPQFLR
WLTEWIEDFYREKQNLIDDMERHREECTSEDHKSKEGTSYCSTCKDKCKKYCECVKKWKSEWENQKNK
YTELYQQNKNETSQKNTSRYDDYVKDFFKKLEANYSSLENYIKGDPYFAEYATKLSFILNSSDANNPSEKI
QKNNDEVCNCNESGIASVEQEQISDPSSNKTCITHSSIKANKKKVCKHVKLGVRENDKDLRVCVIEHTSL
SGVENCCCQDFLRILQENCSDNKSGSSSNGSCNNKNQEACEKNLEKVLASLTNCYKCDKCKSEQSKKN
NKNWIWKKSSGKEGGLQKEYANTIGLPPRTQSLCLVVCLDEKGKKTQELKNIRTNSELLKEWIIAAFHEG
KNLKPSHEKKNDDNGKKLCKALEYSFADYGDLIKGTSIWDNEYTKDLELNLQKIFGKLFRKYIKKNNTAEQ
DTSYSSLDELRESWWNTNKKYIWLAMKHGAGMNSTTCCGDGSVTGSGSSCDDIPTIDLIPQYLRFLQE
WVEHFCKQRQEKVKPVIENCKSCKESGGTCNGECKTECKNKCEVYKKFIEDCKGGDGTAGSSWVKRW
DQIYKRYSKYIEDAKRNRKAGTKNCGPSSTTNAAENKCVQSDIDSFFKHLIDIGLTTPSSYLSIVLDDNIC
GADKAPWTTYTTYTTTEKCNKETDKSKLQQCNTAVVVNVPSPLGNTPHGYKYACQCKIPTNEETCDDRKE
YMNQWSCGSARTMKRGYKNDNYELCKYNGVDVKPTTVRSNSSKLDDKDVTFFNLFEQWNKEIQYQIEQ
YMTNTKISCNNEKNVLSRVSDEAAQPKFSDNERDRNSITHEDKNCKEKCKCYSLWIEKINDQWDKQKD
NYNKFQRKQIYDANKGSQNKKVVSLSNFLFFSCWEEYIQKYFNGDWSKIKNIGSDTFEFLIKKCGNDSG
DGETIFSEKLNNAEKKCKENESTNNKMKSSETSCDCSEPIYIRGCQPKIYDGKIFPGKGGEKQWICKDTII
HGDTNGACIPPRTQNLCVGELWDKRYGGRSNIKNDTKESLKQKIKNAIQKETELLYEYHDKGTAIISRNP
MKGQKEKEEKNNDSNGLPKGFCHAVQRSFIDYKNMILGTSVNIYEYIGKLQEDIKKIIEKGTTKQNGKTV
GSGAENVNAWWKGIEGEMWDAVRCAITKINKKQKKNGTFSIDECGIFPPTGNDEDQSVSWFKEWSEQF
CIERLQYEKNIRDACTNNGQGDKIQGDCKRKCEEYKKYISEKKQEWDKQKTKYENKYVGKSASDLLKEN
YPECISANFDFIFNDNIEYKTYYPYGDYSSICSCEQVKYYEYNNAEKKNNKSLCHEKGNDRTWSKKYIKKL
ENGRTLEGVYVPPRRQQLCLYELFPIIIKNKNDITNAKKELLETLQIVAEREAYYLWKQYHAHNDTTYLAHK
KACCAIRGSFYDLEDIIKGNDLVHDEYTKYIDSKLNEIFDSSNKNDIETKRARTDWWENEAIAVPNITGAN
KSDPKTIRQLVWDAMQSGVRKAIDEEKEKKKPNENFPPCMGVQHIGIAKPQFIRWLEEWTNEFCEKYTKY
FEDMKSNCNLRKGADDCDDNSNIECKKACANYTNWLNPKRIEWNGMSNYYNKIYRKSNKESEDGKDYS
MIMEPTVIDYLNKRCNGEINGNYICCSCKNIGENSTSGTVNKKLQKKETQCEDNKGPLDLMNKVLNKMD
PKYSEHKMKCTEVYLEHVEEQLKEIDNAIKDYKLYPLDRCFDDKSKMKVCDLIGDAIGCKHKTKLDELDE
WNDVDMRDPYNKYKGVLIPPRRRQLCFSRIVRGPANLRNLKEFKEEILKGAQSEGKFLGNYYNEDKDKEK
ALEAMKNSFYDYEYIIKGSDMLTNIQFKDIKRKLDRLLEKETNNTEKVDDWWETNKKSIWNAMLCGYKKS
GNKIIDPSWCTIPTTETPPQFLRWIKEWGTNVCIQKEENKEYVKSKCSNVTNLGAQESESKNCTSEIKKY
QEWSRKRSIQWEAISEGYKKYKGMDEFKNTFKNIKEPDANEPNANEYLKKHCSKCPCGFNDMQEITKYT
NIGNEAFKQIKEQVDIPAELEDVIYRLKHHEYDKGNDYICNKYKNINVNMKKNNDDTWTDLVKNSSDINK
GVLLPPRRKNLFLKIDESDICKYKRDPKLFKDFIYSSAISEVERLKKVYGEAKTKVVHAMKYSFADIGSIIKG
DDMMENNSSDKIGKILGDGVGQNEKRKKWWDMNKYHIWESMLCGYKHAYGNISENDRKMLDIPNND
DEHQFLRWFQEWTENFCTKRNELYENMVTACNSAKCNTSNGSVDKKECTEACKNYSNFILIKKKEYQSL
NSQYDMNYKETKAEKKESPEYFKDKCNGECSCLSEYFKDETRWKNPYETLDDTEVKNNCMCKPPPPASN
NTSDILQKTIPFGIALALGSIAFLFMKKKPKTPVDLLRVLDIPKGDYGIPTPKSSNRYIPYASDRYKGKTYIY
MEGDTSGDDDKYIWDL
>FCR3 complete 2734 aa extracellular part (577 aa highlighted corr. ID1-DBL2b)
(SEQ ID NO: 56)
MDSTSTIANKIEEYLGAKSDDSKIDELLKADPSEVEYYRSGGDGDYLKNNICKITVNHSDSGKYDPCEKKL
PPYDDNDQWKCQQNSSDGSGKPENICVPPRRERLCTYNLENLKFDKIRDNNAFLADVLLTARNEGEKIVQ
NHPDTNSSNVCNALERSFADLADIIRGTDQWKGTNSNLEKNLKQMFAKIRENDKVLQDKYPKDQKYTKL
REAWWNANRQKVWEVITCGARSNDLLIKRGWRTSGKSDRKKNFELCRKCGHYEKEVPTKLDYVPQFLR
WLTEWIEDFYREKQNLIDDMERHREECTREDHKSKEGTSYCSTCKDKCKKYCECVKKWKTEWENQENK
YKDLYEQNKNKTSQKNTSRYDDYVKDFFEKLEANYSSLENYIKGDPYFAEYATKLSFILNPSDANNP
SGETANHNDEACNCNESGISSVGQAQTSGPSSNKTCITHSSIKTNKKKECKDVKLGVRENDKD
LKICVIEDTSLSGVDNCCCQDLLGILQENCSDNKRGSSSNDSCDNKNQDECQKKLEKVFASLT
NGYKCDKCKSGTSRSKKKWIWKKSSGNEEGLQEEYANTIGLPPRTQSLYLGNLPKLENVCEDV
KDINFDTKEKFLAGCLIVSFHEGKNLKKRYPQNKNSGNKENLCKALEYSFADYGDLIKGTSIW
DNEYTKDLELNLQNNFGKLFGKYIKKNNTAEQDTSYSSLDELRESWWNTNKKYIWTAMKHG
AEMNITTCNADGSVTGSGSSCDDIPTIDLIPQYLRFLQEWVENFCEQRQAKVKDVITNCKSCK
ESGNKCKTECKTKCKDECEKYKKFIEACGTAGGGIGTAGSPWSKRWDQIYKRYSKHIEDAKR
NRKAGTKNCGTSSTTNAAASTDENKCVQSDIDSFFKHLIDIGLTTPSSYLSNVLDDNICGADK
APWTTYTTYTTTEKCNKERDKSKSQSSDTLVVVNVPSPLGNTPYRYKYACQCKIPTNEETCDDRK
EYMNQWSCGSARTMKRGYKNDNYELCKYNGVDVKPTTVRSNSSKLDGNDVTFFNLFEQWNKEIQYQIE
QYMTNANISCIDEKEVLDSVSDEGTPKVRGGYEDGRNNNTDQGTNCKEKCKCYKLWIEKINDQWGKQK
DNYNKFRSKQIYDANKGSQNKKVVSLSNFLFFSCWEEYIQKYFNGDWSKIKNIGSDTFEFLIKKCGNNSA
HGEEIFNEKLKNAEKKCKENESTDTNINKSETSCDLNATNYIRGCQSKTYDGKIFPGKGGEKQWICKDTII
HGDTNGACIPPRTQNLCVGELWDKSYGGRSNIKNDTKELLKEKIKNAIHKETELLYEYHDTGTAIISKNDK
KGQKGKNDPNGLPKGFCHAVQRSFIDYKNMILGTSVNIYEHIGKLQEDIKKIIEKGTPQQKDKIGGVGSS
TENVNAWWKGIEREMWDAVRCAITKINKKNNNSIFNGDECGVSPPTGNDEDQSVSWFKEWGEQFCIER
LRYEQNIREACTINGKNEKKCINSKSGQGDKIQGACKRKCEKYKKYISEKKQEWDKQKTKYENKYVGKS
ASDLLKENYPECISANFDFIFNDNIEYKTYYPYGDYSSICSCEQVKYYKYNNAEKKNNKSLCYEKDNDMTW
SKKYIKKLENGRSLEGVYVPPRRQQLCLYELFPIIIKNEEGMEKAKEELLETLQIVAEREAYYLWKQYNPTG
KGIDDANKKACCAIRGSFYDLEDIIKGNDLVHDEYTKYIDSKLNEIFGSSDTNDIDTKRARTDWWENETIT
NGTDRKTIRQLVWDAMQSGVRYAVEEKNENFPLCMGVEHIGIAKPQFIRWLEEWTNEFCEKYTKYFEDM
KSKCDPPKRADTCGDNSNIECKKACANYTNWLNPKRIEWNGMSNYYNKIYRKSNKESEGGKDYSMIMA
PTVIDYLNKRCHGEINGNYICCSCKNIGAYNTTSGTVNKKLQKKETECEEEKGPLDLMNEVLNKMDKKYS
AHKMKCTEVYLEHVEEQLNEIDNAIKDYKLYPLDRCFDDQTKMKVCDLIADAIGCKDKTKLDELDEWND
MDLRGTYNKHKGVLIPPRRRQLCFSRIVRGPANLRSLNEFKEEILKGAQSEGKFLGNYYKEHKDKEKALEA
MKNSFYDYEDIIKGTDMLTNIEFKDIKIKLDRLLEKETNNTKKAEDWWKTNKKSIWNAMLCGYKKSGNKI
IDPSWCTIPTTETPPQFLRWIKEWGTNVCIQKQEHKEYVKSKCSNVTNLGAQASESNNCTSEIKKYQEWS
RKRSIRWETISKRYKKYKRMDILKDVKEPDANTYLREHCSKCPCGFNDMEEMNNNEDNEKEAFKQIKEQ
VKIPAELEDVIYRIKHHEYDKGNDYICNKYKNIHDRMKKNNGNFVTDNFVKKSWEISNGVLIPPRRKNLFL
YIDPSKICEYKKDPKLFKDFIYWSAFTEVERLKKAYGGARAKVVHAMKYSFTDIGSIIKGDDMMEKNSSD
KIGKILGDTDGQNEKRKKWWDMNKYHIWESMLCGYREAEGDTETNENCRFPDIESVPQFLRWFQEWSE
NFCDRRQKLYDKLNSECISAECTNGSVDNSKCTHACVNYKNYILTKKTEYEIQTNKYDNEFKNKNSNDKD
APDYLKEKCNDNKCECLNKHIDDKNKTWKNPYETLEDTFKSKCDCPKPLPSPIKPDDLPPQADEPFDPTIL
QTTIPFGIALALGSIAFLFMKVIYIYIYVCCICMYVCMYVCMYVCMYVCMYVCMHVCMLCVYVIYVFKICIYI
EKEKRKK
>BPTI, protease inhibitor
(SEQ ID NO: 57)
RPDFCLEPPYTGPCKARIIRYFYNAKAGLCQTFVYGGCRAKRNNFKSAEDCMRTCGGA
>PE38, Pseudomonas exotoxin A, (underlining of KDEL represent a signal
sequence, which may be optional for the constructs according to the present
invention)
(SEQ ID NO: 58)
RHRQPRGWEQLEQCGYPVQRLVALYLAARLSWNQVDQVIRNALASPGSGGDLGEAIREQPEQARLALTL
AAAESERFVRQGTGNDEAGAANGPADSGDALLERNYPTGAEFLGDGGDISFSTRGTQNWTVERLLQAHR
QLEERGYVFVGYHGTFLEAAQSIVFGGVRARSQDLDAIWRGFYIAGDPALAYGYAQDQEPDARGRIRNG
ALLRVYVPRSSLPGFYRTSLTLAAPEAAGEVERLIGHPLPLRLDAITGPEEEGGRLETILGWPLAERTVVIPS
AIPTDPRNVGGDLDPSSIPDKEQAISALPDYASQPGKPPRKDEL
>PE38LR, variant of PE38 (underlining of KDEL represent a signal sequence, which may
be optional for the constructs according to the present invention)
(SEQ ID NO: 59)
RHRQPRGWEQLYPTGAEFLGDGGDISFSTRGTQNWTVERLLQAHRQLEERGYVFVGYHGTFLEAAQSIV
FGGVRARSQDLDAIWRGFYIAGDPALAYGYAQDQEPDARGRIRNGALLRVYVPRSSLPGFYRTSLTLAAP
EAAGEVERLIGHPLPLRLDAITGPEEEGGRLETILGWPLAERTVVIPSAIPTDPRNVGGDLDPSSIPDKEQAI
SALPDYASQPGKPPRKDEL

Sequences of VAR2CSA Polypeptides Fused with Truncated Fragments of Pseudomonas Exotoxin A (PE38)

Fused VAR2CSA-PE38 proteins may have modifications such as a protease inhibitor (BPTI) in the N-terminal and/or an optimized PE38 sequence that is less immunogenic (PE38LR)

>BPTI-ID1-ID2aFCR3-PE38LR, underlined sequence corresponds to the ID1
domain of FCR3, sequence in bold corresponds to DBL2Xb domain of FCR3, underlined
and bold sequence is ID2a
(SEQ ID NO: 60)
RPDFCLEPPYTGPCKARIIRYFYNAKAGLCQTFVYGGCRAKRNNFKSAEDCMRTCGGANYIKGDPYFAEYA
TKLSFILNPSDANNPSGETANHNDEACNCNESGISSVGQAQTSGPSSNKTCITHSSIKTNKKKECKDVKL
GVRENDKDLKICVIEDTSLSGVDNCCCQDLLGILQENCSDNKRGSSSNDSCDNKNQDECQKKLEKVFAS
LTNGYKCDKCKSGTSRSKKKWIWKKSSGNEEGLQEEYANTIGLPPRTQSLYLGNLPKLENVCE
DVKDINFDTKEKFLAGCLIVSFHEGKNLKKRYPQNKNSGNKENLCKALEYSFADYGDLIKGTSI
WDNEYTKDLELNLQNNFGKLFGKYIKKNNTAEQDTSYSSLDELRESWWNTNKKYIWTAMKH
GAEMNITTCNADGSVTGSGSSCDDIPTIDLIPQYLRFLQEWVENFCEQRQAKVKDVITNCKSC
KESGNKCKTECKTKCKDECEKYKKFIEACGTAGGGIGTAGSPWSKRWDQIYKRYSKHIEDAK
RNRKAGTKNCGTSSTTNAAASTDENKCVQSDIDSFFKHLIDIGLTTPSSYLSNVLDDNICGAD
KAPWTTYTTYTTTEKCNKERDKSKSQSSDTLVVVNVPSPLGNTPYRYKYACQCKIPTNEETCD
DRKEYMNQWSCGSARTMKRGYKNDNYELCKYNGVDVKPTTVRSNSSKLDRHRQPRGWEQLYP
TGAEFLGDGGDISFSTRGTQNWTVERLLQAHRQLEERGYVFVGYHGTFLEAAQSIVFGGVRARSQDLDAI
WRGFYIAGDPALAYGYAQDQEPDARGRIRNGALLRVYVPRSSLPGFYRTSLTLAAPEAAGEVERLIGHPLP
LRLDAITGPEEEGGRLETILGWPLAERTVVIPSAIPTDPRNVGGDLDPSSIPDKEQAISALPDYASQPGKPP
RKDEL
>BPTI-ID1-ID2aFCR3-PE38
(SEQ ID NO: 61)
RPDFCLEPPYTGPCKARIIRYFYNAKAGLCQTFVYGGCRAKRNNFKSAEDCMRTCGGANYIKGDPYFAEYA
TKLSFILNPSDANNPSGETANHNDEACNCNESGISSVGQAQTSGPSSNKTCITHSSIKTNKKKECKDVKL
GVRENDKDLKICVIEDTSLSGVDNCCCQDLLGILQENCSDNKRGSSSNDSCDNKNQDECQKKLEKVFAS
LTNGYKCDKCKSGTSRSKKKWIWKKSSGNEEGLQEEYANTIGLPPRTQSLYLGNLPKLENVCEDVKDINF
DTKEKFLAGCLIVSFHEGKNLKKRYPQNKNSGNKENLCKALEYSFADYGDLIKGTSIWDNEYTKDLELNL
QNNFGKLFGKYIKKNNTAEQDTSYSSLDELRESWWNTNKKYIWTAMKHGAEMNITTCNADGSVTGSGS
SCDDIPTIDLIPQYLRFLQEWVENFCEQRQAKVKDVITNCKSCKESGNKCKTECKTKCKDECEKYKKFIEA
CGTAGGGIGTAGSPWSKRWDQIYKRYSKHIEDAKRNRKAGTKNCGTSSTTNAAASTDENKCVQSDIDS
FFKHLIDIGLTTPSSYLSNVLDDNICGADKAPWTTYTTYTTTEKCNKERDKSKSQSSDTLVVVNVPSPLGN
TPYRYKYACQCKIPTNEETCDDRKEYMNQWSCGSARTMKRGYKNDNYELCKYNGVDVKPTTVRSNSSKL
DPEGGSLAALTAHQACHLPLETFTRHRQPRGWEQLEQCGYPVQRLVALYLAARLSWNQVDQVIRNALAS
PGSGGDLGEAIREQPEQARLALTLAAAESERFVRQGTGNDEAGAANGPADSGDALLERNYPTGAEFLGD
GGDISFSTRGTQNWTVERLLQAHRQLEERGYVFVGYHGTFLEAAQSIVFGGVRARSQDLDAIWRGFYIA
GDPALAYGYAQDQEPDARGRIRNGALLRVYVPRSSLPGFYRTSLTLAAPEAAGEVERLIGHPLPLRLDAITG
PEEEGGRLETILGWPLAERTVVIPSAIPTDPRNVGGDLDPSSIPDKEQAISALPDYASQPGKPPRKDEL
>ID1-ID2aFCR3-PE38
(SEQ ID NO: 62)
NYIKGDPYFAEYATKLSFILNPSDANNPSGETANHNDEACNCNESGISSVGQAQTSGPSSNKTCITHSSIK
TNKKKECKDVKLGVRENDKDLKICVIEDTSLSGVDNCCCQDLLGILQENCSDNKRGSSSNDSCDNKNQ
DECQKKLEKVFASLTNGYKCDKCKSGTSRSKKKWIWKKSSGNEEGLQEEYANTIGLPPRTQSLYLGNLPK
LENVCEDVKDINFDTKEKFLAGCLIVSFHEGKNLKKRYPQNKNSGNKENLCKALEYSFADYGDLIKGTSIW
DNEYTKDLELNLQNNFGKLFGKYIKKNNTAEQDTSYSSLDELRESWWNTNKKYIWTAMKHGAEMNITTC
NADGSVTGSGSSCDDIPTIDLIPQYLRFLQEWVENFCEQRQAKVKDVITNCKSCKESGNKCKTECKTKCK
DECEKYKKFIEACGTAGGGIGTAGSPWSKRWDQIYKRYSKHIEDAKRNRKAGTKNCGTSSTTNAAASTD
ENKCVQSDIDSFFKHLIDIGLTTPSSYLSNVLDDNICGADKAPWTTYTTYTTTEKCNKERDKSKSQSSDTL
VVVNVPSPLGNTPYRYKYACQCKIPTNEETCDDRKEYMNQWSCGSARTMKRGYKNDNYELCKYNGVDV
KPTTVRSNSSKLDPEGGSLAALTAHQACHLPLETFTRHRQPRGWEQLEQCGYPVQRLVALYLAARLSWN
QVDQVIRNALASPGSGGDLGEAIREQPEQARLALTLAAAESERFVRQGTGNDEAGAANGPADSGDALLE
RNYPTGAEFLGDGGDISFSTRGTQNWTVERLLQAHRQLEERGYVFVGYHGTFLEAAQSIVFGGVRARSQ
DLDAIWRGFYIAGDPALAYGYAQDQEPDARGRIRNGALLRVYVPRSSLPGFYRTSLTLAAPEAAGEVERLI
GHPLPLRLDAITGPEEEGGRLETILGWPLAERTVVIPSAIPTDPRNVGGDLDPSSIPDKEQAISALPDYASQ
PGKPPRKDEL
>ID1-ID2aFCR3-PE38LR
(SEQ ID NO: 63)
NYIKGDPYFAEYATKLSFILNPSDANNPSGETANHNDEACNCNESGISSVGQAQTSGPSSNKTCITHSSIK
TNKKKECKDVKLGVRENDKDLKICVIEDTSLSGVDNCCCQDLLGILQENCSDNKRGSSSNDSCDNKNQ
DECQKKLEKVFASLTNGYKCDKCKSGTSRSKKKWIWKKSSGNEEGLQEEYANTIGLPPRTQSLYLGNLPK
LENVCEDVKDINFDTKEKFLAGCLIVSFHEGKNLKKRYPQNKNSGNKENLCKALEYSFADYGDLIKGTSIW
DNEYTKDLELNLQNNFGKLFGKYIKKNNTAEQDTSYSSLDELRESWWNTNKKYIWTAMKHGAEMNITTC
NADGSVTGSGSSCDDIPTIDLIPQYLRFLQEWVENFCEQRQAKVKDVITNCKSCKESGNKCKTECKTKCK
DECEKYKKFIEACGTAGGGIGTAGSPWSKRWDQIYKRYSKHIEDAKRNRKAGTKNCGTSSTTNAAASTD
ENKCVQSDIDSFFKHLIDIGLTTPSSYLSNVLDDNICGADKAPWTTYTTYTTTEKCNKERDKSKSQSSDTL
VVVNVPSPLGNTPYRYKYACQCKIPTNEETCDDRKEYMNQWSCGSARTMKRGYKNDNYELCKYNGVDV
KPTTVRSNSSKLDRHRQPRGWEQLYPTGAEFLGDGGDISFSTRGTQNWTVERLLQAHRQLEERGYVFVG
YHGTFLEAAQSIVFGGVRARSQDLDAIWRGFYIAGDPALAYGYAQDQEPDARGRIRNGALLRVYVPRSSL
PGFYRTSLTLAAPEAAGEVERLIGHPLPLRLDAITGPEEEGGRLETILGWPLAERTVVIPSAIPTDPRNVGGD
LDPSSIPDKEQAISALPDYASQPGKPPRKDEL
>BPTI-DBL1-ID2aFCR3-PE38LR
(SEQ ID NO: 64)
RPDFCLEPPYTGPCKARIIRYFYNAKAGLCQTFVYGGCRAKRNNFKSAEDCMRTCGGANHSDSGKYDPCE
KKLPPYDDNDQWKCQQNSSDGSGKPENICVPPRRERLCTYNLENLKFDKIRDNNAFLADVLLTARNEGE
KIVQNHPDTNSSNVCNALERSFADLADIIRGTDQWKGTNSNLEKNLKQMFAKIRENDKVLQDKYPKDQK
YTKLREAWWNANRQKVWEVITCGARSNDLLIKRGWRTSGKSDRKKNFELCRKCGHYEKEVPTKLDYVP
QFLRWLTEWIEDFYREKQNLIDDMERHREECTREDHKSKEGTSYCSTCKDKCKKYCECVKKWKTEWEN
QENKYKDLYEQNKNKTSQKNTSRYDDYVKDFFEKLEANYSSLENYIKGDPYFAEYATKLSFILNPSDANNP
SGETANHNDEACNCNESGISSVGQAQTSGPSSNKTCITHSSIKTNKKKECKDVKLGVRENDKDLKICVIE
DTSLSGVDNCCCQDLLGILQENCSDNKRGSSSNDSCDNKNQDECQKKLEKVFASLTNGYKCDKCKSGT
SRSKKKWIWKKSSGNEEGLQEEYANTIGLPPRTQSLYLGNLPKLENVCEDVKDINFDTKEKFLAGCLIVSF
HEGKNLKKRYPQNKNSGNKENLCKALEYSFADYGDLIKGTSIWDNEYTKDLELNLQNNFGKLFGKYIKKN
NTAEQDTSYSSLDELRESWWNTNKKYIWTAMKHGAEMNITTCNADGSVTGSGSSCDDIPTIDLIPQYLR
FLQEWVENFCEQRQAKVKDVITNCKSCKESGNKCKTECKTKCKDECEKYKKFIEACGTAGGGIGTAGSP
WSKRWDQIYKRYSKHIEDAKRNRKAGTKNCGTSSTTNAAASTDENKCVQSDIDSFFKHLIDIGLTTPSSY
LSNVLDDNICGADKAPWTTYTTYTTTEKCNKERDKSKSQSSDTLVVVNVPSPLGNTPYRYKYACQCKIPT
NEETCDDRKEYMNQWSCGSARTMKRGYKNDNYELCKYNGVDVKPTTVRSNSSKLDRHRQPRGWEQLY
PTGAEFLGDGGDISFSTRGTQNWTVERLLQAHRQLEERGYVFVGYHGTFLEAAQSIVFGGVRARSQDLD
AIWRGFYIAGDPALAYGYAQDQEPDARGRIRNGALLRVYVPRSSLPGFYRTSLTLAAPEAAGEVERLIGHP
LPLRLDAITGPEEEGGRLETILGWPLAERTVVIPSAIPTDPRNVGGDLDPSSIPDKEQAISALPDYASQPGK
PPRKDEL
>BPTI-DBL1-ID2aFCR3-PE38
(SEQ ID NO: 65)
RPDFCLEPPYTGPCKARIIRYFYNAKAGLCQTFVYGGCRAKRNNFKSAEDCMRTCGGANHSDSGKYDPCE
KKLPPYDDNDQWKCQQNSSDGSGKPENICVPPRRERLCTYNLENLKFDKIRDNNAFLADVLLTARNEGE
KIVQNHPDTNSSNVCNALERSFADLADIIRGTDQWKGTNSNLEKNLKQMFAKIRENDKVLQDKYPKDQK
YTKLREAWWNANRQKVWEVITCGARSNDLLIKRGWRTSGKSDRKKNFELCRKCGHYEKEVPTKLDYVP
QFLRWLTEWIEDFYREKQNLIDDMERHREECTREDHKSKEGTSYCSTCKDKCKKYCECVKKWKTEWEN
QENKYKDLYEQNKNKTSQKNTSRYDDYVKDFFEKLEANYSSLENYIKGDPYFAEYATKLSFILNPSDANNP
SGETANHNDEACNCNESGISSVGQAQTSGPSSNKTCITHSSIKTNKKKECKDVKLGVRENDKDLKICVIE
DTSLSGVDNCCCQDLLGILQENCSDNKRGSSSNDSCDNKNQDECQKKLEKVFASLTNGYKCDKCKSGT
SRSKKKWIWKKSSGNEEGLQEEYANTIGLPPRTQSLYLGNLPKLENVCEDVKDINFDTKEKFLAGCLIVSF
HEGKNLKKRYPQNKNSGNKENLCKALEYSFADYGDLIKGTSIWDNEYTKDLELNLQNNFGKLFGKYIKKN
NTAEQDTSYSSLDELRESWWNTNKKYIWTAMKHGAEMNITTCNADGSVTGSGSSCDDIPTIDLIPQYLR
FLQEWVENFCEQRQAKVKDVITNCKSCKESGNKCKTECKTKCKDECEKYKKFIEACGTAGGGIGTAGSP
WSKRWDQIYKRYSKHIEDAKRNRKAGTKNCGTSSTTNAAASTDENKCVQSDIDSFFKHLIDIGLTTPSSY
LSNVLDDNICGADKAPWTTYTTYTTTEKCNKERDKSKSQSSDTLVVVNVPSPLGNTPYRYKYACQCKIPT
NEETCDDRKEYMNQWSCGSARTMKRGYKNDNYELCKYNGVDVKPTTVRSNSSKLDPEGGSLAALTAHQ
ACHLPLETFTRHRQPRGWEQLEQCGYPVQRLVALYLAARLSWNQVDQVIRNALASPGSGGDLG+AlREQ
PEQARLALTLAAAESERFVRQGTGNDEAGAAnGPADSGDALLERNYPTGAEFLGDGGDISFSTRGTQNW
TVERLLQAHRQLEERGYVFVGYHGTFLEAAQSIVFGGVRARSQDLDAIWRGFYIAGDPALAYGYAQDQEP
DARGRIRNGALLRVYVPRSSLPGFYRTsLTLAAPEAAGEVERLIGHPLPLRLDAITGPEEEGGRLETILGWPL
AERTVVIPSAIPTDPRNVGGDLDPSSIPDKEQAISALPDYASQPGKPPRKDEL
>DBL1-ID2aFCR3-PE38LR
(SEQ ID NO: 66)
NHSDSGKYDPCEKKLPPYDDNDQWKCQQNSSDGSGKPENICVPPRRERLCTYNLENLKFDKIRDNNAFL
ADVLLTARNEGEKIVQNHPDTNSSNVCNALERSFADLADIIRGTDQWKGTNSNLEKNLKQMFAKIREND
KVLQDKYPKDQKYTKLREAWWNANRQKVWEVITCGARSNDLLIKRGWRTSGKSDRKKNFELCRKCGHY
EKEVPTKLDYVPQFLRWLTEWIEDFYREKQNLIDDMERHREECTREDHKSKEGTSYCSTCKDKCKKYCEC
VKKWKTEWENQENKYKDLYEQNKNKTSQKNTSRYDDYVKDFFEKLEANYSSLENYIKGDPYFAEYATKL
SFILNPSDANNPSGETANHNDEACNCNESGISSVGQAQTSGPSSNKTCITHSSIKTNKKKECKDVKLGVR
ENDKDLKICVIEDTSLSGVDNCCCQDLLGILQENCSDNKRGSSSNDSCDNKNQDECQKKLEKVFASLTN
GYKCDKCKSGTSRSKKKWIWKKSSGNEEGLQEEYANTIGLPPRTQSLYLGNLPKLENVCEDVKDINFDTK
EKFLAGCLIVSFHEGKNLKKRYPQNKNSGNKENLCKALEYSFADYGDLIKGTSIWDNEYTKDLELNLQNN
FGKLFGKYIKKNNTAEQDTSYSSLDELRESWWNTNKKYIWTAMKHGAEMNITTCNADGSVTGSGSSCD
DIPTIDLIPQYLRFLQEWVENFCEQRQAKVKDVITNCKSCKESGNKCKTECKTKCKDECEKYKKFIEACGT
AGGGIGTAGSPWSKRWDQIYKRYSKHIEDAKRNRKAGTKNCGTSSTTNAAASTDENKCVQSDIDSFFK
HLIDIGLTTPSSYLSNVLDDNICGADKAPWTTYTTYTTTEKCNKERDKSKSQSSDTLVVVNVPSPLGNTPY
RYKYACQCKIPTNEETCDDRKEYMNQWSCGSARTMKRGYKNDNYELCKYNGVDVKPTTVRSNSSKLDR
HRQPRGWEQLYPTGAEFLGDGGDISFSTRGTQNWTVERLLQAHRQLEERGYVFVGYHGTFLEAAQSIVF
GGVRARSQDLDAIWRGFYIAGDPALAYGYAQDQEPDARGRIRNGALLRVYVPRSSLPGFYRTSLTLAAPE
AAGEVERLIGHPLPLRLDAITGPEEEGGRLETILGWPLAERTVVIPSAIPTDPRNVGGDLDPSSIPDKEQAIS
ALPDYASQPGKPPRKDEL
>DBL1-ID2aFCR3-PE38
(SEQ ID NO: 67)
NHSDSGKYDPCEKKLPPYDDNDQWKCQQNSSDGSGKPENICVPPRRERLCTYNLENLKFDKIRDNNAFL
ADVLLTARNEGEKIVQNHPDTNSSNVCNALERSFADLADIIRGTDQWKGTNSNLEKNLKQMFAKIREND
KVLQDKYPKDQKYTKLREAWWNANRQKVWEVITCGARSNDLLIKRGWRTSGKSDRKKNFELCRKCGHY
EKEVPTKLDYVPQFLRWLTEWIEDFYREKQNLIDDMERHREECTREDHKSKEGTSYCSTCKDKCKKYCEC
VKKWKTEWENQENKYKDLYEQNKNKTSQKNTSRYDDYVKDFFEKLEANYSSLENYIKGDPYFAEYATKL
SFILNPSDANNPSGETANHNDEACNCNESGISSVGQAQTSGPSSNKTCITHSSIKTNKKKECKDVKLGVR
ENDKDLKICVIEDTSLSGVDNCCCQDLLGILQENCSDNKRGSSSNDSCDNKNQDECQKKLEKVFASLTN
GYKCDKCKSGTSRSKKKWIWKKSSGNEEGLQEEYANTIGLPPRTQSLYLGNLPKLENVCEDVKDINFDTK
EKFLAGCLIVSFHEGKNLKKRYPQNKNSGNKENLCKALEYSFADYGDLIKGTSIWDNEYTKDLELNLQNN
FGKLFGKYIKKNNTAEQDTSYSSLDELRESWWNTNKKYIWTAMKHGAEMNITTCNADGSVTGSGSSCD
DIPTIDLIPQYLRFLQEWVENFCEQRQAKVKDVITNCKSCKESGNKCKTECKTKCKDECEKYKKFIEACGT
AGGGIGTAGSPWSKRWDQIYKRYSKHIEDAKRNRKAGTKNCGTSSTTNAAASTDENKCVQSDIDSFFK
HLIDIGLTTPSSYLSNVLDDNICGADKAPWTTYTTYTTTEKCNKERDKSKSQSSDTLVVVNVPSPLGNTPY
RYKYACQCKIPTNEETCDDRKEYMNQWSCGSARTMKRGYKNDNYELCKYNGVDVKPTTVRSNSSKLDP
EGGSLAALTAHQACHLPLETFTRHRQPRGWEQLEQCGYPVQRLVALYLAARLSWNQVDQVIRNALASPG
SGGDLGEAIREQPEQARLALTLAAAESERFVRQGTGNDEAGAANGPADSGDALLERNYPTGAEFLGDGG
DISFSTRGTQNWTVERLLQAHRQLEERGYVFVGYHGTFLEAAQSIVFGGVRARSQDLDAIWRGFYIAGDP
ALAYGYAQDQEPDARGRIRNGALLRVYVPRSSLPGFYRTSLTLAAPEAAGEVERLIGHPLPLRLDAITGPEE
EGGRLETILGWPLAERTVVIPSAIPTDPRNVGGDLDPSSIPDKEQAISALPDYASQPGKPPRKDEL
>ID1-ID2a3D7-PE38
(SEQ ID NO: 68)
LSFILNSSDANNPSEKIQKNNDEVCNCNESGIASVEQEQISDPSSNKTCITHSSIKANKKKVCKHVKLGV
RENDKDLRVCVIEHTSLSGVENCCCQDFLRILQENCSDNKSGSSSNGSCNNKNQEACEKNLEKVLASLT
NCYKCDKCKSEQSKKNNKNWIWKKSSGKEGGLQKEYANTIGLPPRTQSLCLVVCLDEKGKKTQELKNIR
TNSELLKEWIIAAFHEGKNLKPSHEKKNDDNGKKLCKALEYSFADYGDLIKGTSIWDNEYTKDLELNLQKI
FGKLFRKYIKKNNTAEQDTSYSSLDELRESWWNTNKKYIWLAMKHGAGMNSTTCCGDGSVTGSGSSCD
DIPTIDLIPQYLRFLQEWVEHFCKQRQEKVKPVIENCKSCKESGGTCNGECKTECKNKCEVYKKFIEDCKG
GDGTAGSSWVKRWDQIYKRYSKYIEDAKRNRKAGTKNCGPSSTTNAAENKCVQSDIDSFFKHLIDIGLT
TPSSYLSIVLDDNICGADKAPWTTYTTYTTTEKCNKETDKSKLQQCNTAVVVNVPSPLGNTPHGYKYACQ
CKIPTNEETCDDRKEYMNQWSCGSARTMKRGYKNDNYELCKYNGVDVKPTTVRSNSSKLDPEGGSLAA
LTAHQACHLPLETFTRHRQPRGWEQLEQCGYPVQRLVALYLAARLSWNQVDQVIRNALASPGSGGDLGE
AIREQPEQARLALTLAAAESERFVRQGTGNDEAGAANGPADSGDALLERNYPTGAEFLGDGGDISFSTRG
TQNWTVERLLQAHRQLEERGYVFVGYHGTFLEAAQSIVFGGVRARSQDLDAIWRGFYIAGDPALAYGYA
QDQEPDARGRIRNGALLRVYVPRSSLPGFYRTSLTLAAPEAAGEVERLIGHPLPLRLDAITGPEEEGGRLET
ILGWPLAERTVVIPSAIPTDPRNVGGDLDPSSIPDKEQAISALPDYASQPGKPPRKDEL
>ID1-ID2a3D7-PE38LR
(SEQ ID NO: 69)
LSFILNSSDANNPSEKIQKNNDEVCNCNESGIASVEQEQISDPSSNKTCITHSSIKANKKKVCKHVKLGV
RENDKDLRVCVIEHTSLSGVENCCCQDFLRILQENCSDNKSGSSSNGSCNNKNQEACEKNLEKVLASLT
NCYKCDKCKSEQSKKNNKNWIWKKSSGKEGGLQKEYANTIGLPPRTQSLCLVVCLDEKGKKTQELKNIR
TNSELLKEWIIAAFHEGKNLKPSHEKKNDDNGKKLCKALEYSFADYGDLIKGTSIWDNEYTKDLELNLQKI
FGKLFRKYIKKNNTAEQDTSYSSLDELRESWWNTNKKYIWLAMKHGAGMNSTTCCGDGSVTGSGSSCD
DIPTIDLIPQYLRFLQEWVEHFCKQRQEKVKPVIENCKSCKESGGTCNGECKTECKNKCEVYKKFIEDCKG
GDGTAGSSWVKRWDQIYKRYSKYIEDAKRNRKAGTKNCGPSSTTNAAENKCVQSDIDSFFKHLIDIGLT
TPSSYLSIVLDDNICGADKAPWTTYTTYTTTEKCNKETDKSKLQQCNTAVVVNVPSPLGNTPHGYKYACQ
CKIPTNEETCDDRKEYMNQWSCGSARTMKRGYKNDNYELCKYNGVDVKPTTVRSNSSKLDRHRQPRG
WEQLYPTGAEFLGDGGDISFSTRGTQNWTVERLLQAHRQLEERGYVFVGYHGTFLEAAQSIVFGGVRAR
SQDLDAIWRGFYIAGDPALAYGYAQDQEPDARGRIRNGALLRVYVPRSSLPGFYRTSLTLAAPEAAGEVER
LIGHPLPLRLDAITGPEEEGGRLETILGWPLAERTVVIPSAIPTDPRNVGGDLDPSSIPDKEQAISALPDYAS
QPGKPPRKDEL
>ID1-DBL2bFCR3-PE38LR
(SEQ ID NO: 70)
NYIKGDPYFAEYATKLSFILNPSDANNPSGETANHNDEACNCNESGISSVGQAQTSGPSSNKTCITHSSIK
TNKKKECKDVKLGVRENDKDLKICVIEDTSLSGVDNCCCQDLLGILQENCSDNKRGSSSNDSCDNKNQ
DECQKKLEKVFASLTNGYKCDKCKSGTSRSKKKWIWKKSSGNEEGLQEEYANTIGLPPRTQSLYLGNLPK
LENVCEDVKDINFDTKEKFLAGCLIVSFHEGKNLKKRYPQNKNSGNKENLCKALEYSFADYGDLIKGTSIW
DNEYTKDLELNLQNNFGKLFGKYIKKNNTAEQDTSYSSLDELRESWWNTNKKYIWTAMKHGAEMNITTC
NADGSVTGSGSSCDDIPTIDLIPQYLRFLQEWVENFCEQRQAKVKDVITNCKSCKESGNKCKTECKTKCK
DECEKYKKFIEACGTAGGGIGTAGSPWSKRWDQIYKRYSKHIEDAKRNRKAGTKNCGTSSTTNAAASTD
ENKCVQSDIDSFFKHLIDIGLTTPSSYLSNVLDDNICGADKAPWTTYTTYTTTEKCNKERDKSKSQSSDTL
VVVNVPSPLGNTPYRYKYRHRQPRGWEQLYPTGAEFLGDGGDISFSTRGTQNWTVERLLQAHRQLEERG
YVFVGYHGTFLEAAQSIVFGGVRARSQDLDAIWRGFYIAGDPALAYGYAQDQEPDARGRIRNGALLRVYV
PRSSLPGFYRTSLTLAAPEAAGEVERLIGHPLPLRLDAITGPEEEGGRLETILGWPLAERTVVIPSAIPTDPR
NVGGDLDPSSIPDKEQAISALPDYASQPGKPPRKDEL
>DT388, sequence of diphtheria toxin
(SEQ ID NO: 71)
MGADDVVDSSKSFVMENFSSYHGTKPGYVDSIQKGIQKPKSGTQGNYDDDWKGFYSTDNKYDAAGYS
VDNENPLSGKAGGVVKVTYPGLTKVLALKVDNAETIKKELGLSLTEPLMEQVGTEEFIKRFGDGASRVVLS
LPFAEGSSSVEYINNWEQAKALSVELEINFETRGKRGQDAMYEYMAQACAGNRVRRSVGSSLSCINLDW
DVIRDKTKTKIESLKEHGPIKNKMSESPNKTVSEEKAKQYLEEFHQTALEHPELSELKTVTGTNPVFAGAN
YAAWAVNVAQVIDSETADNLEKTTAALSILPGIGSVMGIADGAVHHNTEEIVAQSIALSSLMVAQAIPLVG
ELVDIGFAAYNFVESIINLFQVVHNSYNRPAYSPGHKTQPMHEF

Sequences of VAR2CSA Polypeptides Fused with Truncated Fragments of Diphtheria Toxin

>DT388-DBL1-ID2a 3D7
(SEQ ID NO: 72)
MGADDVVDSSKSFVMENFSSYHGTKPGYVDSIQKGIQKPKSGTQGNYDDDWKGFYSTDNKYDAAGYS
VDNENPLSGKAGGVVKVTYPGLTKVLALKVDNAETIKKELGLSLTEPLMEQVGTEEFIKRFGDGASRVVLS
LPFAEGSSSVEYINNWEQAKALSVELEINFETRGKRGQDAMYEYMAQACAGNRVRRSVGSSLSCINLDW
DVIRDKTKTKIESLKEHGPIKNKMSESPNKTVSEEKAKQYLEEFHQTALEHPELSELKTVTGTNPVFAGAN
YAAWAVNVAQVIDSETADNLEKTTAALSILPGIGSVMGIADGAVHHNTEEIVAQSIALSSLMVAQAIPLVG
ELVDIGFAAYNFVESIINLFQVVHNSYNRPAYSPGHKTQPMHEFHSDSGTNDPCDRIPPPYGDNDQWKCA
IILSKVSEKPENVFVPPRRQRMCINNLEKLNVDKIRDKHAFLADVLLTARNEGERIVQNHPDTNSSNVCNA
LERSFADIADIIRGTDLWKGTNSNLEQNLKQMFAKIRENDKVLQDKYPKDQNYRKLREDWWNANRQKV
WEVITCGARSNDLLIKRGWRTSGKSNGDNKLELCRKCGHYEEKVPTKLDYVPQFLRWLTEWIEDFYREK
QNLIDDMERHREECTSEDHKSKEGTSYCSTCKDKCKKYCECVKKWKSEWENQKNKYTELYQQNKNETS
QKNTSRYDDYVKDFFKKLEANYSSLENYIKGDPYFAEYATKLSFILNSSDANNPSEKIQKNNDEVCNCNES
GIASVEQEQISDPSSNKTCITHSSIKANKKKVCKHVKLGVRENDKDLRVCVIEHTSLSGVENCCCQDFLRI
LQENCSDNKSGSSSNGSCNNKNQEACEKNLEKVLASLTNCYKCDKCKSEQSKKNNKNWIWKKSSGKE
GGLQKEYANTIGLPPRTQSLCLVVCLDEKGKKTQELKNIRTNSELLKEWIIAAFHEGKNLKPSHEKKNDDN
GKKLCKALEYSFADYGDLIKGTSIWDNEYTKDLELNLQKIFGKLFRKYIKKNNTAEQDTSYSSLDELRESW
WNTNKKYIWLAMKHGAGMNSTTCCGDGSVTGSGSSCDDIPTIDLIPQYLRFLQEWVEHFCKQRQEKVK
PVIENCKSCKESGGTCNGECKTECKNKCEVYKKFIEDCKGGDGTAGSSWVKRWDQIYKRYSKYIEDAKR
NRKAGTKNCGPSSTTNAAENKCVQSDIDSFFKHLIDIGLTTPSSYLSIVLDDNICGADKAPWTTYTTYTTT
EKCNKETDKSKLQQCNTAVVVNVPSPLGNTPHGYKYACQCKIPTNEETCDDRKEYMNQWSCGSARTMK
RGYKNDNYELCKYNGVDVKPTTVRSNSSKLDSGR
>DT388-DBL1-ID2a FCR3
(SEQ ID NO: 73)
MGADDVVDSSKSFVMENFSSYHGTKPGYVDSIQKGIQKPKSGTQGNYDDDWKGFYSTDNKYDAAGYS
VDNENPLSGKAGGVVKVTYPGLTKVLALKVDNAETIKKELGLSLTEPLMEQVGTEEFIKRFGDGASRVVLS
LPFAEGSSSVEYINNWEQAKALSVELEINFETRGKRGQDAMYEYMAQACAGNRVRRSVGSSLSCINLDW
DVIRDKTKTKIESLKEHGPIKNKMSESPNKTVSEEKAKQYLEEFHQTALEHPELSELKTVTGTNPVFAGAN
YAAWAVNVAQVIDSETADNLEKTTAALSILPGIGSVMGIADGAVHHNTEEIVAQSIALSSLMVAQAIPLVG
ELVDIGFAAYNFVESIINLFQVVHNSYNRPAYSPGHKTQPMHEFHSDSGKYDPCEKKLPPYDDNDQWKC
QQNSSDGSGKPENICVPPRRERLCTYNLENLKFDKIRDNNAFLADVLLTARNEGEKIVQNHPDTNSSNVC
NALERSFADLADIIRGTDQWKGTNSNLEKNLKQMFAKIRENDKVLQDKYPKDQKYTKLREAWWNANRQ
KVWEVITCGARSNDLLIKRGWRTSGKSDRKKNFELCRKCGHYEKEVPTKLDYVPQFLRWLTEWIEDFYRE
KQNLIDDMERHREECTREDHKSKEGTSYCSTCKDKCKKYCECVKKWKTEWENQENKYKDLYEQNKNKT
SQKNTSRYDDYVKDFFEKLEANYSSLENYIKGDPYFAEYATKLSFILNPSDANNPSGETANHNDEACNCN
ESGISSVGQAQTSGPSSNKTCITHSSIKTNKKKECKDVKLGVRENDKDLKICVIEDTSLSGVDNCCCQDL
LGILQENCSDNKRGSSSNDSCDNKNQDECQKKLEKVFASLTNGYKCDKCKSGTSRSKKKWIWKKSSG
NEEGLQEEYANTIGLPPRTQSLYLGNLPKLENVCEDVKDINFDTKEKFLAGCLIVSFHEGKNLKKRYPQNK
NSGNKENLCKALEYSFADYGDLIKGTSIWDNEYTKDLELNLQNNFGKLFGKYIKKNNTAEQDTSYSSLDE
LRESWWNTNKKYIWTAMKHGAEMNITTCNADGSVTGSGSSCDDIPTIDLIPQYLRFLQEWVENFCEQRQ
AKVKDVITNCKSCKESGNKCKTECKTKCKDECEKYKKFIEACGTAGGGIGTAGSPWSKRWDQIYKRYSK
HIEDAKRNRKAGTKNCGTSSTTNAAASTDENKCVQSDIDSFFKHLIDIGLTTPSSYLSNVLDDNICGADK
APWTTYTTYTTTEKCNKERDKSKSQSSDTLVVVNVPSPLGNTPYRYKYACQCKIPTNEETCDDRKEYMNQ
WSCGSARTMKRGYKNDNYELCKYNGVDVKPTTVRSNSSKLDSGR
>DT388-ID1-ID2a 3D7
(SEQ ID NO: 74)
MGADDVVDSSKSFVMENFSSYHGTKPGYVDSIQKGIQKPKSGTQGNYDDDWKGFYSTDNKYDAAGYS
VDNENPLSGKAGGVVKVTYPGLTKVLALKVDNAETIKKELGLSLTEPLMEQVGTEEFIKRFGDGASRVVLS
LPFAEGSSSVEYINNWEQAKALSVELEINFETRGKRGQDAMYEYMAQACAGNRVRRSVGSSLSCINLDW
DVIRDKTKTKIESLKEHGPIKNKMSESPNKTVSEEKAKQYLEEFHQTALEHPELSELKTVTGTNPVFAGAN
YAAWAVNVAQVIDSETADNLEKTTAALSILPGIGSVMGIADGAVHHNTEEIVAQSIALSSLMVAQAIPLVG
ELVDIGFAAYNFVESIINLFQVVHNSYNRPAYSPGHKTQPMHEFLSFILNSSDANNPSEKIQKNNDEVCNC
NESGIASVEQEQISDPSSNKTCITHSSIKANKKKVCKHVKLGVRENDKDLRVCVIEHTSLSGVENCCCQD
FLRILQENCSDNKSGSSSNGSCNNKNQEACEKNLEKVLASLTNCYKCDKCKSEQSKKNNKNWIWKKSS
GKEGGLQKEYANTIGLPPRTQSLCLVVCLDEKGKKTQELKNIRTNSELLKEWIIAAFHEGKNLKPSHEKKN
DDNGKKLCKALEYSFADYGDLIKGTSIWDNEYTKDLELNLQKIFGKLFRKYIKKNNTAEQDTSYSSLDELR
ESWWNTNKKYIWLAMKHGAGMNSTTCCGDGSVTGSGSSCDDIPTIDLIPQYLRFLQEWVEHFCKQRQE
KVKPVIENCKSCKESGGTCNGECKTECKNKCEVYKKFIEDCKGGDGTAGSSWVKRWDQIYKRYSKYIED
AKRNRKAGTKNCGPSSTTNAAENKCVQSDIDSFFKHLIDIGLTTPSSYLSIVLDDNICGADKAPWTTYTTY
TTTEKCNKETDKSKLQQCNTAVVVNVPSPLGNTPHGYKYACQCKIPTNEETCDDRKEYMNQWSCGSART
MKRGYKNDNYELCKYNGVDVKPTTVRSNSSKLDSGR
>DT388-ID1-ID2a FCR3
(SEQ ID NO: 75)
MGADDVVDSSKSFVMENFSSYHGTKPGYVDSIQKGIQKPKSGTQGNYDDDWKGFYSTDNKYDAAGYS
VDNENPLSGKAGGVVKVTYPGLTKVLALKVDNAETIKKELGLSLTEPLMEQVGTEEFIKRFGDGASRVVLS
LPFAEGSSSVEYINNWEQAKALSVELEINFETRGKRGQDAMYEYMAQACAGNRVRRSVGSSLSCINLDW
DVIRDKTKTKIESLKEHGPIKNKMSESPNKTVSEEKAKQYLEEFHQTALEHPELSELKTVTGTNPVFAGAN
YAAWAVNVAQVIDSETADNLEKTTAALSILPGIGSVMGIADGAVHHNTEEIVAQSIALSSLMVAQAIPLVG
ELVDIGFAAYNFVESIINLFQVVHNSYNRPAYSPGHKTQPMHEFNYIKGDPYFAEYATKLSFILNPSDANNP
SGETANHNDEACNCNESGISSVGQAQTSGPSSNKTCITHSSIKTNKKKECKDVKLGVRENDKDLKICVIE
DTSLSGVDNCCCQDLLGILQENCSDNKRGSSSNDSCDNKNQDECQKKLEKVFASLTNGYKCDKCKSGT
SRSKKKWIWKKSSGNEEGLQEEYANTIGLPPRTQSLYLGNLPKLENVCEDVKDINFDTKEKFLAGCLIVSF
HEGKNLKKRYPQNKNSGNKENLCKALEYSFADYGDLIKGTSIWDNEYTKDLELNLQNNFGKLFGKYIKKN
NTAEQDTSYSSLDELRESWWNTNKKYIWTAMKHGAEMNITTCNADGSVTGSGSSCDDIPTIDLIPQYLR
FLQEWVENFCEQRQAKVKDVITNCKSCKESGNKCKTECKTKCKDECEKYKKFIEACGTAGGGIGTAGSP
WSKRWDQIYKRYSKHIEDAKRNRKAGTKNCGTSSTTNAAASTDENKCVQSDIDSFFKHLIDIGLTTPSSY
LSNVLDDNICGADKAPWTTYTTYTTTEKCNKERDKSKSQSSDTLVVVNVPSPLGNTPYRYKYACQCKIPT
NEETCDDRKEYMNQWSCGSARTMKRGYKNDNYELCKYNGVDVKPTTVRSNSSKLDSGR
>id1id2a FCR3 3x Nglyc mutant (asn/N to Gln/Q)
(SEQ ID NO: 128)
NYIKGDPYFAEYATKLSFILNPSDANNPSGETANHNDEACNCNESGISSVGQAQTSGPSSqKTCITHSSIK
TNKKKECKDVKLGVRENDKDLKICVIEDTSLSGVDNCCCQDLLGILQENCSDNKRGSSSNDSCDNKNQ
DECQKKLEKVFASLTNGYKCDKCKSGTSRSKKKWIWKKSSGNEEGLQEEYANTIGLPPRTQSLYLGNLPK
LENVCEDVKDINFDTKEKFLAGCLIVSFHEGKNLKKRYPQNKNSGNKENLCKALEYSFADYGDLIKGTSIW
DNEYTKDLELNLQNNFGKLFGKYIKKqNTAEQDTSYSSLDELRESWWNTNKKYIWTAMKHGAEMqITTC
NADGSVTGSGSSCDDIPTIDLIPQYLRFLQEWVENFCEQRQAKVKDVITNCKSCKESGNKCKTECKTKCK
DECEKYKKFIEACGTAGGGIGTAGSPWSKRWDQIYKRYSKHIEDAKRNRKAGTKNCGTSSTTNAAASTD
ENKCVQSDIDSFFKHLIDIGLTTPSSYLSNVLDDNICGADKAPWTTYTTYTTTEKCNKERDKSKSQSSDTL
VVVNVPSPLGNTPYRYKYACQCKIPTNEETCDDRKEYMNQWSCGSARTMKRGYKNDNYELCKYNGVDV
KPTTVRSNSSKLD
>SpyCatcher
(SEQ ID NO: 129)
GAMVDTLSGLSSEQGQSGDMTIEEDSATHIKFSKRDEDGKELAGATMELRDSSGKTISTWISDGQVKDF
YLYPGKYTFVETAAPDGYEVATAITFTVNEQGQVTVNGKATKGDAHI
>Spytag
(SEQ ID NO: 130)
AHIVMVDAYKPTK
>Minimal Spytag sequence
(SEQ ID NO: 131)
AHIVMVDA
>The β-strand of CnaB2 (K-Tag)
(SEQ ID NO: 132)
ATHIKFSKRD
SpyLigase
(SEQ ID NO: 133)
HHHHHHDYDGQSGDGKELAGATMELRDSSGKTISTWISDGQVKDFYLYPGKYTFVETAAPDGYEVATAI
TFTVNEQGQVTVNGKATKGGSGGSGGSGEDSATHI
>isopeptide Spy0128
(SEQ ID NO: 134)
TDKDMTITFTNKKDAE
>Split-Spy0128
(SEQ ID NO: 135)
ATTVHGETVVNGAKLTVTKNLDLVNSNALIPNTDFTFKIEPDTTVNEDGNKFKGVALNTPMTKVTYTNSDK
GGSNTKTAEFDFSEVTFEKPGVYYYKVTEEKIDKVPGVSYDTTSYTVQVHVLWNEEQQKPVATYIVGYKE
GSKVPIQFKNSLDSTTLTVKKKVSGTGGDRSKDFNFGLTLKANQYYKASEKVMIEKTTKGGQAPVQTEAS
IDQLYHFTLKDGESIKVTNLPVGVDYVVTEDDYKSEKYTTNVEVSPQDGAVKNIAGNSTEQETSTDKDMT
I
>SpyCatcher-ΔN
(SEQ ID NO: 136)
EDSATHIKFSKRDEDGKELAGATMELRDSSGKTISTWISDGQVKDFYLYPGKYTFVETAAPDGYEVATAIT
FTVNEQGQVTVNGKATKGDAHI
>Inversed Spytag sequence
(SEQ ID NO: 137)
KTPKYADVMVIHA

### EXAMPLES

Methods

Method 1—Cloning and Protein Expression

VAR2CSA sequence fragments were amplified from codon optimized FCR3 (GenBank accession no. GU249598) or 3D7 (GenBank accession no. JQ247428) VAR2CSA genes using specific primers (Table 2). Simple fragments were amplified in a one-step PCR. Amino acid substitution constructs were made in a two-step PCR. First PCR amplified two fragments from the codon optimized FCR3 template, containing overlapping complimentary ends. Second PCR amplified the total construct, using the two overlapping fragments as template with primers specific for the outer borders. All fragments were sequenced for verification. Fragments were cloned into the baculovirus vector pAcGP67-A (BD Biosciences), modified to contain a V5 and His tag at the C-terminal or a pEt coli expression vector. The proteins were expressed in baculovirus-infected insect cells as soluble protein secreted into the cell culture supernatant or in E. coli Shuffle cells. Synthetic genes are designed using standard algorithms for gene desing taking into account secondary DNA and RNA structures, repetitive regions and codon choice for a given expression cell system. Synthetic genes are made for Spy-tag and Spy-catcher fusions as well as CAR designs.

Method 2—Protein Purification and SDS-PAGE

The filtered supernatant containing the secreted recombinant protein was dialyzed using an ÄKTA cross-flow (GE Healthcare). The dialysis was performed in 10 mM NaH2PO4 (pH 7.4, Sigma-Aldrich) and 500 mM NaCl. The resulting solution was filtered (0.2 μm) and imidiazole was added to a final concentration of 15 mM. The protein was then purified on a 1-ml HisSelect column (H8286, Sigma-Aldrich). Bound protein was eluted with 10 mM NaH2PO4 (pH 7.4), 500 mM NaCl, and 500 mM imidiazole. Proteins needed for Quartz Crystal Microbalance measurements and SAXS were further purified to obtain monomers by size exclusion chromatography using a HiLoad 16/60 Superdex 200 column (GE Healthcare) in 20 mM Tris (pH 8) and 200 mM NaCl. The purity and structural integrity of the protein was verified by SDS-PAGE.

Method 3—ELISA

Falcon microtiter plates (351172, BD Biosciences) were incubated at a concentration of 3 μg/ml for CSPG (bovine) (D8428, Sigma) or HSPG (H4777, Sigma) and 100 μg/ml for CSA (C9819, Sigma), CSC (400675, Seikagaku), and CSB (C3788, Sigma) overnight at 4° C. The plates were then blocked with TSM binding buffer (20 mM Tris, 150 mM NaCl, 2 mM CaCl₂, 0.05% Tween-20, 1% BSA, PH7.4 at 25° C.) for 2 hours at 37° C. on a shaker. A 2-fold dilution series (1.56 mM-100 mM) of protein was prepared in TSM binding buffer and added to the plates, which was incubated 1 hr at 37° C. on a shaker. All measurements were performed in triplicates. The plates were washed three times in TSM washing buffer (20 mM Tris, 150 mM NaCl, 2 mM CaCl₂, 0.05% Tween-20, PH7.4 at 25° C.). The plates were then incubated with 1:3000 anti-V5-HRP antibody (R96125, Invitrogen) in TSM binding buffer 1 hr at 37° C. on a shaker. The plates were washed three times in TSM washing buffer. Finally the plates were developed with o-phenylenediamine substrate (DAKO) for 15 min. The reaction was quenched with 2.5M H₂SO₄. Absorbance was measured at 490 nm.

Method 4—Quartz Crystal Microbalance (Attana A100)

Experiments were performed on an Attana A100 (Attana AB), using gold plated 10 MHz, AT-cut quartz crystal, polystyrene chips (3611-3103 Attana AB). All buffers and reagents were filtered to 0.2 μm. The ligand was CSPG (Bovine) (D8428, Sigma) or HSPG (H4777, Sigma), coated at a concentration of 100 μg/ml. Coating was done in steady state by adding ligand solution and incubation 30 minutes at room temperature. This was followed by blocking the plate with PBS containing 0.1% Ig-free BSA (BSA-50, Rockland), 30 minutes at room temperature. The Attana A100 was washed with 1% SDS prior to every experiment, using the manufacturers predefined daily wash program. Following the wash, the running buffer was switched to PBS at a flow rate of 25 μL/min, at 25° C., and the machine was allowed to stabilize at a maximum change in frequency of 0.5 Hz/min. Once stabilized PBS was injected multiple times to show that the injection process minimally affected the baseline. Prior to sample injection PBS was injected as a blank. Analyte was injected in a 1:3 dilution series (0.25 μg/ml-60 μg/ml) starting with the lowest concentration. Association time was set to 84 seconds and disassociation time to 5 minutes. Due to high affinity of binding it was not possible to regenerate binding surface following injections. The data collected was processed in the Attester Evaluation software (Attana AB). Curves were fitted in a simple 1:1 model. kₒₙ and k_{off} were determined by curve fitting and K_{D} was calculated based K_{D}=k_{off}/kₒₙ.

Method 5—Cancer Cell Binding Assays

Flow-cytometry (FCM) was used to test the reactivity of the VAR2CSA minimal binding polypeptide to CSPG expressed on the surface of various cell lines. Cells were cultured in RPMI supplemented with 10% foetal calf serum (CHO cells, C32), Hams F12 (BeWo), kept in 5% carbon dioxide at 37° C. or purified from a human blood sample in CPD buffer (red blood cells). Aliquots of cells (1×105) were sequentially exposed to the VAR2CSA minimal binding polypeptide (150, 75 or 37 nM) and α-V5-FITC (1:800)(Invitrogen) diluted in FACS2 (PBS+2% FCS) for 30 minutes at +4 C in dark with smooth agitation. As negative controls a truncated version of the minimal binding polypeptide and FACS2 buffer were used. Intact cells were gated based on the forward and side scatter signal. Data were acquired using a FC500 flow-cytometer (Beckman Coulter) from a minimum of 5000 cells. All samples relating to a particular cell line were processed and analyzed in a single assay.

Method 6—Cancer Cell Binding Assays

As an alternative to the flow-cytometry assay above, cells were incubated with VAR2CSA minimal binding polypeptide and α-V5-FITC (1:500)(Invitrogen) diluted in HBSS. VAR2CSA polypeptide was used at the same concentrations as written above. Following α-V5-FITC staining cells were washed 3 times in HBSS, collected in Enzyme-free cell detachment buffer (Invitrogen) and analyzed on a FACS Calibur (BD Biosciences) for FL-1 signal intensity.

Abbreviations CIDR, cysteine-rich inter-domain region; CSA, chondroitin sulfate A; CSPG, chondroitin sulfate proteoglycan; DBL, Duffy binding-like domain; FCM, flow-cytometry; FV2, full-length ecto-domain of the VAR2CSA protein without N-terminal segment; HSPG, heparan sulfate proteoglycan; ID, inter-domain; IE, P. falciparum-infected erythrocyte; NTS, N-terminal segment; PM, placental malaria; PfEMP1, Plasmodium falciparum erythrocyte membrane protein 1; PM, placental malaria.

Method 7—Staining of Paraffin-Embedded Human Tissue Samples

The binding of recombinant VAR2CSA to primary cancer tissue obtained from human patients is investigated using immunohistochemistry (IHC). Paraffin embedded tissue spotted on glass slides subjected no antigen retrieval was incubated with 0.1-500 nM V5-VAR2CSA variants or V5-Control protein (DBL4) for 1 h in room temperature, washed for 8 minutes, incubated with 1:700 mouse anti-V5 antibody for 30 minutes, washed for 8 minutes. Bound anti-V5 was subsequently detected using UltraMap anti-mouse HRP using the Ventana Discovery XT platform.

### Example 1 Production of Truncated Recombinant VAR2CSA Proteins

All protein truncations were produced according to previously defined domain borders (Dahlbäck M, Jorgensen L M, Nielsen M A, Clausen T M, Ditlev S B, et al. J Biol Chem 286: 15908-15917). For the purpose of simplification we have divided the CIDR_{PAM} domain into two domains ID2a and ID2b, where ID2a is the N-terminal part of CIDR_{PAM} not containing the CIDR-like sequence and ID2b corresponds to the CIDR-like sequence. We also used a new DBL2X border incorporating 93 amino acids of ID2a. For simplification we call this border DBL2Xb, while the old border will be referred to as DBL2Xa. Primers used in cloning are listed in Table 2. Fragments were expressed in baculovirus-infected insect cells as soluble proteins as described in Method 1. Most proteins were produced based on the FCR3 genotype. Some FCR3 fragments did not express and these were instead made based on the 3D7 genotype. The proteins were used interchangeably in the analysis since we show that recombinant VAR2CSA from both genotypes bind equally to CSA. All proteins showed a shift in gel mobility when comparing reduced and non-reduced samples by SDS-PAGE (Method 2). This is consistent with the formation of intra-molecular disulfide bridges. Some proteins formed high-molecular weight complexes detected by non-reduced SDS-PAGE. This is probably due to the formation of inter-molecular disulfide bridges between unpaired cysteines. This was confirmed by reducing the complexes to monomeric protein using DTT.

TABLE 2
Cloning Primers
FCR3 Primers

| Protein | Forward Primer | Reverse Primer |
|---|---|---|
| ID1-ID2b | AACTACATCAAGGGCGAC (SEQ ID NO: 76) | CTTGTTGATATTGGTGTCGGT (SEQ ID NO: 77) |
| DBL1X-ID2a | CACAGCGATAGCGGCAAG (SEQ ID NO: 78) | GTCCAGCTTGCTGGAGTT (SEQ ID NO: 79) |
| ID1-ID2a | AACTACATCAAGGGCGAC (SEQ ID NO: 80) | GTCCAGCTTGCTGGAGTT (SEQ ID NO: 81) |
| ID1-DBL2Xa | AACTACATCAAGGGCGAC (SEQ ID NO: 82) | AGCGGCGTTGGTGGTGGA (SEQ ID NO: 83) |
| ID1-DBL2Xb | AACTACATCAAGGGCGAC (SEQ ID NO: 84) | GTACTTGTACCGGTAGGG (SEQ ID NO: 85) |
| DBL1X-DBL2Xb | CACAGCGATAGCGGCAAG (SEQ ID NO: 86) | GTACTTGTACCGGTAGGG (SEQ ID NO: 87) |

3d7 Primers

| Protein | Forward Primer | Reverse Primer |
|---|---|---|
| DBL2X-DBL4E | CTGACCAACTGCTACAAG (SEQ ID NO: 88) | GGTCCAGAGGGTACAGCTT (SEQ ID NO: 89) |
| ID1-DBL3E | CTGTCCTTCATCCTGAAC (SEQ ID NO: 90) | TTCAGCGTTGTTGTACTCGTA (SEQ ID NO: 91) |
| ID1-DBL4E | CTGTCCTTCATCCTGAAC (SEQ ID NO: 92) | GTCCAGAGGGTACAGCTT (SEQ ID NO: 93) |
| DBL1X-ID2b | CACTCTGACTCTGGCACC (SEQ ID NO: 94) | AGAGGACTTCATCTTGTTGTTGGT (SEQ ID NO: 95) |
| ID1-ID2b | CTGTCCTTCATCCTGAAC (SEQ ID NO: 96) | AGAGGACTTCATCTTGTTGTTGGT (SEQ ID NO: 97) |
| DBL1X-ID2a | CACTCTGACTCTGGCACC (SEQ ID NO: 98) | GTCCAGCTTAGAGGAGTT (SEQ ID NO: 99) |
| ID1-ID2a | CTGTCCTTCATCCTGAAC (SEQ ID NO: 100) | GTCCAGCTTAGAGGAGTT (SEQ ID NO: 101) |
| DBL1X-DBL2Xa | CACTCTGACTCTGGCACC (SEQ ID NO: 102) | GGCGGCGTTGGTGGTAGA (SEQ ID NO: 103) |
| ID1-DBL2Xa | CTGTCCTTCATCCTGAAC (SEQ ID NO: 104) | GGCGGCGTTGGTGGTAGA (SEQ ID NO: 105) |
| DBL1X-DBL2Xb | CACTCTGACTCTGGCACC (SEQ ID NO: 106) | GTACTTGTATCCGTGGGG (SEQ ID NO: 107) |
| ID1-DBL2Xb | CTGTCCTTCATCCTGAAC (SEQ ID NO: 108) | GTACTTGTATCCGTGGGG (SEQ ID NO: 109) |
| | Mutating Putative CSA Binding Sites | |
| | PCR1 | |
| | Fragment 1 | |
| Protein | Forward | Reverse |
| DBL1X-ID2a (DSM | CACAGCGATAGCGGCAAG (SEQ ID NO: 110) | GGTGTCGAAGTTGATGTCGGGCAGATTGCCCAGGTA |
| Deletion) | | (SEQ ID NO: 111) |
| Alanine sub. | CACAGCGATAGCGGCAAG (SEQ ID NO: 112) | AGCTGCGGCCAGATTAGCGCCCTCGTGGAAGGACAC |
| K(626,629,630), R(631) | | (SEQ ID NO: 113) |
| Alanine sub. | | AGCGCATTCAGCTGCGGCGTTGGTCTTGATGGAGCT |
| K(459,460,461,464) | CACAGCGATAGCGGCAAG (SEQ ID NO: 114) | (SEQ ID NO: 115) |
| | Fragment 2 | |
| Protein | Forward | Reverse |
| DBL1X-ID2a (DSM | CACAGCGATAGCGGCAAG (SEQ ID NO: 116) | GTCCAGCTTGCTGGAGTT (SEQ ID NO: 117) |
| Deletion) | | |
| Alanine sub. | GCTAATCTGGCCGCAGCTTACCCCCAGAATAAGAAC | GTCCAGCTTGCTGGAGTT (SEQ ID NO: 119) |
| K(626,629,630), R(631) | (SEQ ID NO: 118) | |
| Alanine sub. | GCCGCAGCTGAATGCGCTGACGTGAAGCTGGGCGTG | GTCCAGCTTGCTGGAGTT (SEQ ID NO: 121) |
| K(459,460,461,464) | (SEQ ID NO: 120) | |
| | PCR2 | |
| | Final Constuct | |
| Protein | Forward | Reverse |
| DBL1X-ID2a (DSM | CACAGCGATAGCGGCAAG (SEQ ID NO: 122) | GTCCAGCTTGCTGGAGTT (SEQ ID NO: 123) |
| Deletion) | | |
| Alanine sub. | CACAGCGATAGCGGCAAG (SEQ ID NO: 124) | GTCCAGCTTGCTGGAGTT (SEQ ID NO: 125) |
| K(626,629,630), R(631) | | |
| Alanine sub. | | |
| K(459,460,461,464) | CACAGCGATAGCGGCAAG (SEQ ID NO: 126) | GTCCAGCTTGCTGGAGTT (SEQ ID NO: 127) |

### Example 2 VAR2CSA from FCR3 and 3D7 Binds CSA with Similar Affinity and Specificity

FCR3 infected erythrocytes (IE) adhere much stronger to CSA in vitro compared to 3D7 or NF54 (IE). If the differences are related to the sequence differences of the expressed VAR2CSA the information could be used to define residues involved in the adhesion process. To test this, we produced a series of overlapping 3D7 VAR2CSA fragments, identical to the ones we have previously tested for FCR3.

The proteins were first screened for specific CSPG binding in a solid phase binding assay (ELISA) (described in Method 3). Proteins binding specifically to CSPG were then further purified by size exclusion chromatography, to obtain pure monomeric protein, and subjected to kinetic analysis on a Quartz Crystal Microbalance (Attana A100) (Method 2 and 4, respectively). The 3D7 VAR2CSA fragments showed binding characteristics very similar to their FCR3 counterparts in the solid state binding assay. The same is true in the kinetic analysis (Table 3). The sensorgrams show association and dissociation data collected at different protein concentrations. This allows determination of the association rate constant (kₒₙ), disassociation rate constant (k_{off}), and the equilibrium constant (K_{D}). Together with the peak response levels these parameters give estimation for the CSPG binding affinity. There is no apparent difference between 3D7 and FCR3 fragments. Some fragments show lower affinity, but this characteristic is maintained in the fragments counterpart. This indicates that the 3D7 and FCR3 VAR2CSA proteins fold and function in the same way.

TABLE 3
CSA binding affinity of produced VAR2CSA proteins.

| | FCR3 | | 3D7 |
|---|---|---|---|
| VAR2CSA Fragment | Baculo | E. coli | Baculo |
| FV2 | 5.2* | | 8.2 |
| ID1-DBL4ε | 8.6* | | 9.4 |
| ID1-DBL3ε | 0.3* | | 8.5 |
| DBL2X-DBL4ε | 2.4* | | 1.2 |
| DBL1-ID2b | 1.5* | | |
| DBL1-ID2a | 8.0 | 3.5 | 29.5 |
| ID1-ID2a | 7.6 | 18.3 | 5.7 |
| DBL1X-DBL2Xb | | 14.6 | |
| DBL1X-DBL2Xa | N/A | | |
| ID1-DBL2Xb | | | 21.8 |
| ID1-DBL2Xa | | | N/A |

*Proteins published in (Dahlbäck et al, JBC, 2011)
Affinity is given as a K_{D} (nM) value determined in kinetics experiments using a quartz crystal microbalance biosensor (Attana A100).
N/A: proteins for which no K_{D} value could be determined, due to a lack of binding to CSA.

### Example 3—the Core-CSA Binding Site Lies within the DBL2X Domain

It has been suggested that the minimal CSA binding region in VAR2CSA lies within DBL2X-ID2b, with the need of flanking domains for full affinity binding (Dahlbäck M, Jorgensen L M, Nielsen M A, Clausen T M, Ditlev S B, et al. J Biol Chem 286: 15908-15917). Here we have analyzed shorter fragments of VAR2CSA to further map the regions required for CSA binding.

The truncated proteins were first screened for binding to a CSA proteoglycan (CSPG) in ELISA and then further purified to obtain monomers for examination on the Quartz Crystal Microbalance (Methods 3, 2 and 4, respectively). The minimal binding region is ID1-DBL2Xb (Table 3). This region showed a binding affinity of 21.8 nM, which is comparable to that of full-length VAR2CSA.

Placental IEs are highly selective for low-sulfated placental CSPG. They do not adhere to any other glycosaminoglycans (GAG), such as heparan sulfate (HS). The same is true for the full-length recombinant VAR2CSA protein. The solid state binding assay showed that the VAR2CSA fragments, containing the minimal CSA binding region, bound specifically to CSA. To confirm this the minimal binding fragments were further tested for binding to a heparan sulfate proteoglycan (HSPG) on the Quartz Crystal Microbalance (Method 4). None of the fragments bound HSPG.

### Example 4

VAR2CSA Minimal CSA Binding Region Binds Specifically to a Wide Panel of Cancer Cells

Many different cancer cells have been associated with high expression of the proteoglycan CSPG4. This molecule was initially described as a marker for melanoma but it has recently been found in many cancer forms, including cancer stem cells. The CS chain(s) attached to CSPG4 is known to be primarily CSA. One of the smallest VAR2CSA fragments (ID1-ID2a) was analyzed for binding to a large panel of various cancer cell lines by flow-cytometry (Method 5 and 6). The non-CSA binding protein ID1-DBL2Xa was used as a negative control. The VAR2CSA recombinant protein (ID1-ID2a) binds strongly at 75 nM to all cancer cell lines transcribing CSPG4 (microarray data) including cutaneous Melanoma (C32, MeWo), Lung carcinoma (A549), Breast carcinoma (HCC1395), Osterosarcoma (U205, MNNG/HOS), Rhabdomyosarcoma (RH30) (Table 4 and 5). This protein also binds strongly to cutaneous T-cell lymphoma, which does not express CSPG4 (Table 4). The negative control protein ID1-DBL2Xa did not bind to any of the cell lines tested (Table 4). In addition, ID1-ID2a did not interact with human red blood cells, which were used as control cells. Wild type and GAG-deficient Chinese hamster ovary (CHO) cells were also analyzed for ID1-ID2a interaction. The strong interaction seen for ID1-ID2a with wild-type CHO cells was completely abolished when analyzing the CHO-745 cell line, in which the GAG-synthesis is disrupted. The CSA specificity of the interaction was also verified by inhibiting VAR2CSA binding to cells by pre-mixing VAR2CSA with CSA, CSC or HS. CSC and HS did not have any effect on the binding, whereas CSA efficiently abrogated binding of VAR2CSA to the cancer cells.

Following these results, a larger panel of cancer cells were screened by flow cytometry (Table 6 and 7) using the DBL1-ID2a or ID1-ID2a fragment of VAR2CSA. The main purpose of this screening is to identify cell lines suitable for xenograft modeling in vivo.

TABLE 4
Staining of cancer cell lines and negative control cells
using the minimal binding domain of VAR2CSA (ID1-ID2a).

| Cell type | Blank | ID1-DBL2Xa | ID1-ID2a |
|---|---|---|---|
| C32 | 5.77 | 6.94 | 63.81 |
| MyLa 2059 | 5.61 | 5.61 | 145.35 |
| MyLa 1850 | 5.87 | 5.6 | 137.86 |
| Cho WT | 3.09 | 4.35 | 34.79 |
| Cho 745 | 4.24 | 4.29 | 4.38 |
| PBMC | 1.34 | 1.36 | 1.67 |
| Erythrocytes | 1.11 | 1.17 | 1.07 |

Cells were incubated with medium alone (blank) or recombinant proteins (ID1-DBL2 or ID1-ID2a) at 75 nM for 30 minutes, followed by incubation with anti-V5-FITC (Invitrogen) at 1:800, cells were washed thrice between each incubation. Shown are the mean FITC fluorescence values recorded from a minimum of 5000 cells using a FC500 flowcytometer (Becton Dickinson).

TABLE 5
Staining of cancer cell lines using recombinant VAR2CSA

| Cell type | Blank | DBL1-ID2a |
|---|---|---|
| U2OS | NS | +++ |
| MG63 | NS | ++++ |
| MDA-MB-231 | NS | +++ |
| TC32 | NS | + |
| TC71 | NS | ++ |
| MNNG | NS | +++ |
| CHLA9 | NS | ++ |
| CHLA10 | NS | ++ |
| RH30 | NS | +++ |
| RH18 | NS | ++ |
| PC3 | NS | +++ |

Cells were incubated with medium alone (blank) or recombinant proteins (DBL1-ID2a or ID1-ID2a) at 75 nM for 30 minutes, followed by incubation with anti-V5-FITC (Invitrogen) at 1:800, cells were washed thrice between each incubation. Shown are the medium score of FITC fluorescence intensity recorded from a minimum of 4 high power field images using a HAL100 Zeiss microscope.
NS: No staining;
+: weak; ++: medium; +++: strong; ++++: Very strong.

TABLE 6
Screening of diverse human cancer cell lines for binding
of recombinant VAR2CSA (using DBL1-ID2a or ID1-ID2a).

| | | 75 nM | 150 nM | |
|---|---|---|---|---|
| Cell line | Control | VAR2CSA | VAR2CSA | Comments |
| MeWo | NS | +++ | ++++ | Melanoma (Fibroblast morphology, derived |
| | | | | from lymphnode) |
| A549 | NS | +++ | +++ | Lung Adenocarcinoma (K-RasG12S) |
| HCC1395 | NS | +++ | ++++ | Invasive ductal breast carcinoma TNM |
| | | | | stage 1 grade 3; no lymphnode |
| | | | | metastasis; Her2-neg, ER-neg, PR-neg |
| | | | | (Triple-negative) |
| RH30 | NS | +++ | ++++ | Rhabdomyosarcoma (TPp53 negativ; |
| | | | | PAX7-FOXO1A fusion positive; highly |
| | | | | genomic instable (>50 chromosome |
| | | | | rearangements)) |
| MNNG | NS | +++ | +++ | Osteosarcoma from 13 year old female |
| | | | | Caucasian (TPR-Met positive) |
| U2OS | NS | +++ | +++ | Osterosarcoma from 15 year old female |
| | | | | caucasian (IGF-R1 and IGFR-II positive; |
| | | | | TPp53 wt, pRb wt, p16-neg; highly |
| | | | | aneuploid) |
| H1792 | NS | ++ | ++ | Lung Adenocarcinoma (K-RasG12S: |
| | | | | TPp53het)) |
| MDA-MD-435 | NS | ++ | +++ | Breast carcinoma of melanocytic origin |
| | | | | (ER-neg, Her2-pos, PR-pos) |
| MG63 | NS | +++ | ++++ | Osteosarcoma |
| TC32 | NS | ++ | ++ | Ewing's sarcoma |
| CHLA9 | NS | ++ | ++ | Ewing's sarcoma |
| CHLA10 | NS | ++ | ++ | Ewing's sarcoma |
| TC71 | NS | ++ | ++ | Ewing's sarcoma |
| HOS | NS | +++ | ++++ | Osteosarcoma |
| PC3 | NS | ++ | ++ | Prostate carcinoma |
| SKNMC | NS | ++ | +++ | Ewing's sarcoma |
| MCF-7 | NS | + | ++ | Breast carcinoma |

Binding was measured by flow cytometry as described in METHOD 5 and 6.
NS: No staining;
+: weak; ++: medium; +++ : strong; ++++ : Very strong.

TABLE 7
Screening of more human cell cancer cell lines for binding
of recombinant VAR2CSA (using DBL1-ID2a or ID1-ID2a)

| | Negative | ID1- | |
|---|---|---|---|
| Cell type | control | ID2a | Comments |
| GP202 | 21.63 | 111.37 | Gastric Carcinoma |
| NCI-N87 | 7.18 | 207.72 | Gastric Carcinoma |
| MKN45 | 4.22 | 55.4 | Gastric Carcinoma |
| MKN28 | 6.9 | 103.84 | Gastric Carcinoma |
| AGS | 7.25 | 18.21 | Gastric Carcinoma |
| KatoIII | 7.33 | 18.76 | Gastric Carcinoma |
| SNU-1 | 4.33 | 155.79 | Gastric Carcinoma |
| SNU-638 | 8.47 | 8.49 | Gastric Carcinoma |
| IPA220 | 7.72 | 13.67 | Gastric Carcinoma |
| MDA-231 | 3.39 | 63.43 | Triple negative Breast |
| T47D | 3.63 | 48.13 | Luminal Breast |
| LNCap | 6.58 | 24.86 | Prostate |
| PC3 | 5.2 | 29.82 | Prostate |
| Ovc316 | 1.89 | 7.24 | Ovarian cancer stem cells |
| | | DBL1- | |
| Cell type | Blank | ID2a | |
| NALM-6 | 6.19 | 8.22 | Acute lymphatic leukaemia (ALL) |
| 697 | 3.23 | 30.36 | ALL |
| AMO-1 | 2.68 | 35.22 | Myelomatosis |
| KMM-1 | 2.82 | 16.1 | Myelomatosis |
| MOLP-8 | 2.44 | 19.24 | Myelomatosis |
| KMS-12-PE | 3.02 | 7.14 | Myelomatosis |
| KMS-12-BM | 2.2 | 3.25 | Myelomatosis |
| U2932 | 4.24 | 16.83 | Diffuse Large B-cell lymphoma |
| | | | (DLBCL) |
| SU-DHL8 | ND | 3.75 | DLBCL |
| SU-DHL5 | 2.19 | 10.28 | DLBCL |
| Oci_Ly19 | 3.38 | 18.96 | DLBCL |
| HBL1 | 6.53 | 39.53 | DLBCL |
| Farage | 2.8 | 3.28 | DLBCL |
| RIVA | 2.26 | 3.32 | DLBCL |
| WSU-FSCCL | 4.89 | 22.32 | Low-grade follicular small cleaved |
| | | | cell lymphoma |
| U-698-M | 2.24 | 2.85 | Lymphoblastic lymphoma |
| | | | del(6)(q15q22) |

Binding was measured by flow cytometry as described in METHOD 5 and 6.
Values shown are mean fluorescence intensity using protein concentration of 200 nM.

### Example 5

Recombinant VAR2CSA Binds to Cancer Cells with High Affinity

The binding affinity of the recombinant VAR2CSA fragment DBL1-ID2a to the cancer cell lines, C32 melanoma and two Cho cell lines (described in example 8) was analysed using a Quartz Crystal Microbalance biosensor (Attana Cell200). A 2-fold dilution series (25-400 nM) of the protein was analysed for binding to the cell surface, with regeneration of the binding surface in between each new protein injection. The binding affinity was estimated to lie in the nano-molar range (Table 8), which is similar to the binding affinity to pure receptor (Table 3).

TABLE 8
Estimated binding affinity (K_{D}) of recombinant DBL1-ID2a
(E. coli) to cancer cells expressing CSA (C32 and Cho WT)
and lack of binding to a CSA-negative cell line (Cho 745)

| Cell type | K_{D} (nM) |
|---|---|
| C32 melanoma cells | 13 |
| Cho WT | 1.4 |
| Cho 745 | N/A |

N/A: K_{D} could not be determined due to lack of binding to the cells

### Example 6

Recombinant VAR2CSA Protein Binds to Cancer Tissue with High Specificity

The binding of recombinant VAR2CSA to primary cancer tissue obtained from human patients is investigated using immunohistochemistry (IHC). The method was developed using human placenta tissue as positive control and Tonsil and liver tissue as negative control. The staining protocol was optimized on the Ventana Discovery XT platform with no epitope retrieval. Paraffin embedded tissue spotted on glass slides was incubated with 0.1-500 nM V5-VAR2CSA (ID1-ID2a) or V5-Control protein (DBL4) for 1 h in room temperature, washed for 8 minutes, incubated with 1:700 mouse anti-V5 antibody for 30 minutes, washed for 8 minutes. Bound anti-V5 was subsequently detected using UltraMap anti-mouse HRP. V5-VAR2CSA stains human placenta in 0.5 nM concentrations with no staining in Tonsil or normal liver. The staining can be completely blocked by adding 200 μg/μl CSA to the reaction buffer. V5-control protein does not stain human placenta tissue at any concentrations tested. A multi-organ tissue micro-array (TMA) representing 24 normal organs showed low or absent staining when stained with 1 nM V5-VAR2CSA, while cancer specimens of breast, colon, Rectum, Prostate, kidney, liver, bladder, pancreas, squamous cell, Lung, Gall bladder, Stomach, Testis, Ovary, Uterus, Adrenal gland, Thyroid and Thymus, hematopoietic system, and the connective tissue (sarcomas) stained positive with intensities equal or higher than human placenta positive control tissue (Table 9).

TABLE 9
Detection of CSA on primary human tumor specimens using recombinant
VAR2CSA. Table shows number of positive/total number of cases
stained as described in Example 10 for main cancer groups.
Positive staining is defined as intensity equal or higher
than that observed in placenta tissue.

| Cancer group | Positive ratio |
|---|---|
| Bladder carcinoma | 44/56 |
| Prostate carcinoma | 71/76 |
| Breast carcinoma | 64/75 |
| Melanoma | 5/6 |
| Sarcoma | 23/25 |
| Esophagus Squamous cell carcinoma | 2/3 |
| Stomach Adenocarcinoma | 3/3 |
| Colon carcinoma | 2/3 |
| Rectal Adenocarcinoma | 3/3 |
| Liver carcinoma | 3/3 |
| Renal carcinoma | 3/3 |
| Lung carcinoma | 2/3 |
| Cervix carcinoma | 3/3 |
| Ovarian carcinoma | 2/3 |
| Diffuse B-cell lymphoma | 1/3 |
| Astrocytoma | 3/3 |
| Pancreatic carcinoma | 3/3 |

### Example 7

Identification of Potential CSPG Molecules that were Targeted by VAR2CSA

Recombinant VAR2CSA protein (DBL1-ID2a) with a V5-tag was screened for binding to a panel of transfected HEK293 cells expressing >3000 human membrane receptors. A set of 25 receptors have been identified as potential targets of VAR2CSA (Table 10). The interaction between VAR2CSA and these receptors will be further verified by analysis of the binding specificity through inhibition with CSA and HS, both in the HEK293 system and in ELISA.

TABLE 10
Receptors that were experimentally identified
as potential targets of VAR2CSA.

| Gene ID | Name | UniProt/SwissProt |
|---|---|---|
| BCAN | Brevican | PGCB HUMAN, |
| | | Q96GW7 |
| BDKRB2 | Bradykinin receptor B2 | BKRB2 HUMAN, |
| | | P30411 |
| CA9 | Carbonic anhydrase IX | CAH9 HUMAN, |
| | | Q16790 |
| CCR10 | chemokine (C-C motif) receptor 10 | CCR10 HUMAN, |
| | | P46092 |
| CD44 | CD44 molecule (Indian blood group) | CD44 HUMAN, |
| | | P16070 |
| CDH8 | Cadherin 8, type 2 | CADH8 HUMAN, |
| | | P55286 |
| CFB | Complement factor B | CFAB HUMAN, |
| | | P00751 |
| GABBR2 | gamma-aminobutyric acid (GABA) B | GABR2 HUMAN, |
| | receptor, 2 | O75899 |
| GPC3 | Glypican 3 | GPC3 HUMAN, |
| | | P51654 |
| GPC5 | Glypican 5 | GPC5 HUMAN, |
| | | P78333 |
| GPR65 | G-protein coupled receptor 65 | PSYR HUMAN, |
| | | Q8IYL9 |
| GPRC5B | G protein-coupled receptor, family | GPC5B HUMAN, |
| | C, group 5, member B | Q9NZH0 |
| KCNA2 | potassium voltage-gated channel, | KCNA2 HUMAN, |
| | shaker-related subfamily, member 2 | P16389 |
| PKD2 | polycystic kidney disease 2 | PKD2 HUMAN, |
| | (autosomal dominant) | Q13563 |
| PODXL2 | podocalyxin-like 2 | PDXL2 HUMAN, |
| | | Q9NZ53 |
| PTPRG | protein tyrosine phosphatase, | PTPRG HUMAN, |
| | receptor type, G | P23470 |
| S100A9 | S100 calcium binding protein A9 | S10A9 HUMAN, |
| | | P06702 |
| SDC1 | Syndecan 1 | SDC1 HUMAN, |
| | | P18827 |
| SDC4 | Syndecan 4 | SDC4 HUMAN, |
| | | P31431 |
| STX2 | Syntaxin 2 | STX2 HUMAN, |
| | | P32856 |
| STXBP5 | syntaxin binding protein 5 (tomosyn) | STXB5 HUMAN, |
| | | Q5T5C0 |
| TGFBR3 | transforming growth factor, beta | TGBR3 HUMAN, |
| | receptor III | Q03167 |
| TMEFF1 | transmembrane protein with EGF-like | TEFF1 HUMAN, |
| | and two follistatin-like domains 1 | Q8IYR6 |
| TMEFF2/TE | transmembrane protein with EGF-like | TEFF2 HUMAN, |
| NB2 | and two follistatin-like domains 2 | Q9UIK5 |
| TMEM154 | Transmembrane protein 154 | (None) |
| THBD | Thrombomodulin | TRBM HUMAN, |
| | | P07204 |
| CSPG5 | chondroitin sulfate proteoglycan 5 | CSPG5 HUMAN, |
| | (neuroglycan C) | O95196 |
| STXBP5 | syntaxin binding protein 5 (tomosyn) | STXB5 HUMAN, |
| | | Q5T5C0 |

### Example 8

Bi-Specific VAR2CSA-scFv-Anti-CD3 Protein

DBL1-ID2a and ID1-ID2a VAR2CSA gene fragments have been fused to scFv sequences encoding single chain antibodies against CD3. These VAR2CSA-scFv proteins are expressed in E. coli or in insect cells. These VAR2CSA-scFv proteins are bi-specific towards CSA-expressing cancer cells and CD3-expressing T cells. As such, these proteins are able to bind CSA in the tumor environment through the VAR2CSA domain while recruiting CD3 positive T cells via the scFv-anti-CD3 domain.

### Example 9

VAR2CSA-Spy Fusion Proteins

DBL1-ID2a and ID1-ID2a VAR2CSA gene fragments have been fused to various variants of SpyTag, K-tag, SpyCatcher and SpyCather-ΔN (SEQ ID NO: 130, 129, 136). These VAR2CSA-Spy proteins are expressed in E. coli or in insect cells. The recombinant VAR2CSA-Spy protein can form an isopeptide bond with the corresponding split-protein binding partner. For example, VAR2CSA-SpyTag can bind to any molecule that has a SpyCatcher or ΔN-SpyCather sequence; VAR2CSA-SpyCatcher or VAR2CSA-ΔN-SpyCather can bind to any molecule that has a SpyTag sequence; VAR2CSA-SpyTag can bind to any molecule that has a K-Tag sequence in the presence of SpyLigase; or VAR2CSA-K-Tag can bind to any molecule that has a K-Tag sequence in the presence of SpyLigase. Furthermore, a VAR2CSA-K-Tag can be polymerized (attached to each other) through the K-Tag in the presence of SpyLigase, and subsequently combined with any protein expression a SpyCatcher, such as a Spy-CAR. As such, these proteins are able to bind any protein sequence having the corresponding sequence of the split-protein binding system. By simple mixing of the Spy-tagged VAR2CSA protein with Spy-catcher or Spy-catcher tagged VAR2CSA with Spy-tag we observed a rapid and effective coupling between the proteins as measured by size exclusion chromatography or SDS page. IV injection of Spy-tagged VAR2CSA into mice with established tumors followed by IV injection of fluorescence labeled Spy-catcher demonstrate by IVIS in vivo imaging and post mortem analyses that the isopeptide bond and pairing of Spy-tag and Spy-catcher does happen in vivo.

### Example 10

Bi-Specific VAR2CSA-Spy-scFv-Anti-CD3 Fusion Proteins

DBL1-ID2a and ID1-ID2a VAR2CSA gene fragments have been fused to various variants of SpyTag, K-tag, SpyCatcher and SpyCather-ΔN (SEQ ID NO: 130, 129, 136) and scFv sequences encoding single chain antibodies against CD3. These VAR2CSA-Spy proteins are expressed in E. coli or in insect cells. The recombinant VAR2CSA-Spy-scFv-anti-CD3 fusion proteins can form an isopeptide bond with the corresponding split-protein binding partner. For example, VAR2CSA-Spy-scFv-anti-CD3 can bind to any molecule that has a SpyCatcher or ΔN-SpyCather sequence; VAR2CSA-SpyCatcher-scFv-anti-CD3 or VAR2CSA-ΔN-SpyCather-scFv-anti-CD3 can bind to any molecule that has a SpyTag sequence; VAR2CSA-Spy-scFv-anti-CD3 can bind to any molecule that has a K-Tag sequence in the presence of SpyLigase; or VAR2CSA-K-Tag-scFv-anti-CD3 can bind to any molecule that has a K-Tag sequence in the presence of SpyLigase. Furthermore, a VAR2CSA-K-Tag-scFv-anti-CD3 can be polymerized (attached to each other) through the K-Tag in the presence of SpyLigase, and subsequently combined with any protein expression a SpyCatcher, such as a Spy-CAR. The VAR2CSA-Spy-scFv-anti-CD3 proteins are synthesized and validated as described in example 9.

### Example 11

Chimeric Antigen Receptors Expressing VAR2CSA (VAR2-CAR)

CAR-encoding sequences are synthesized using codon-optimization algorithms (Optimizer, PMID: 17439967) and subcloned into “destination” vectors encoding second generation (CD28 transmembrane and intracellular T cell signaling domains and CD3-zeta chain intracellular T cell signaling domain); or third generation (CD8 transmembrane domain linked to CD28, 4-1BB, and CD3-zeta intracellular T cell signaling domains). Retroviral vector supernatants are created by transfecting 293GP packaging cells (Clonetech) with plasmids encoding CAR retroviral vectors and the RD114 envelope glycoprotein, collecting culture supernatants (s/n) 48-72 hours later. The culture supernatants are frozen or used immediately to transduce OKT3 and IL-2 activated human PBMC for 2 consecutive days (culture of lymphocytes on plates coated with RECTRONECTIN (Clonetech) pre-exposed to dilutions of vector containing s/n). CAR expression on transduced T cells is determined by flow cytometry. (Table 11).

TABLE 11
Expression of VAR2-CAR on the plasma membrane of
transduced T cells as detected by flow cytometry.

| | Percentage of positive cell | Percentage of Negative cell |
|---|---|---|
| VAR2-CAR | 61.40% | 38.60% |

### Example 12

Chimeric Antigen Receptors Expressing Spy-CAR

CAR-encoding sequences SpyTag, SpyCatcher, ΔN-SpyCatcher or K-Tag (SEQ ID NO: 130, 129, 136, or 132) are synthesized using codon-optimization algorithms (Optimizer, PMID: 17439967) and subcloned into “destination” vectors encoding second generation (CD28 transmembrane and intracellular T cell signaling domains and CD3-zeta chain intracellular T cell signaling domain); or third generation (CD8 transmembrane domain linked to CD28, 4-1BB, and CD3-zeta intracellular T cell signaling domains). Retroviral vector supernatants are created by transfecting 293GP packaging cells (Clonetech) with plasmids encoding CAR retroviral vectors and the RD114 envelope glycoprotein, collecting culture supernatants (s/n) 48-72 hours later. The culture supernatants are frozen or used immediately to transduce OKT3 and IL-2 activated human PBMC for 2 consecutive days (culture of lymphocytes on plates coated with RECTRONECTIN (Clonetech) pre-exposed to dilutions of vector containing s/n). CAR expression on transduced T cells is determined by flow cytometry (Table 12).

TABLE 12
Expression of Spy-CAR on the plasma membrane of
transduced T cells as detected by flow cytometry.

| | Percentage of positive cell | Percentage of Negative cell |
|---|---|---|
| Spy-CAR | 64.82% | 35.18% |

### Example 13

Chimeric Antigen Receptors Expressing Spy-CAR and Subsequently Linked to a VAR2-Spy Fusion Protein: VAR2-Spy-Spy-CARs

CAR-encoding sequences SpyTag, SpyCatcher, ΔN-SpyCatcher or K-tag (SEQ ID NO: 130, 129, 136, or 132) are synthesized using codon-optimization algorithms (Optimizer, PMID: 17439967 and subcloned into “destination” vectors encoding second generation (CD28 transmembrane and intracellular T cell signaling domains and CD3-zeta chain intracellular T cell signaling domain); or third generation (CD8 transmembrane domain linked to CD28, 4-1BB, and CD3-zeta intracellular T cell signaling domains). Retroviral vector supernatants are created by transfecting 293GP packaging cells (Clonetech) with plasmids encoding CAR retroviral vectors and the RD114 envelope glycoprotein, collecting culture supernatants (s/n) 48-72 hours later. The culture supernatants are frozen or used immediately to transduce OKT3 and IL-2 activated human PBMC for 2 consecutive days (culture of lymphocytes on plates coated with RECTRONECTIN (Clonetech) pre-exposed to dilutions of vector containing s/n). Spy-CAR transgenic T cells are subsequently incubated with at a corresponding VAR2CSA-Spy fusion protein (corresponding to the other half of the split-protein binding system of the Spy-CAR) to produce a fully functional VAR2CSA-Spy-Spy-CAR receptor. CAR expression on transduced T cells is determined by flow cytometry and/or co-immunofluorescence (Table 13).

TABLE 13
Spy-CAR transgenic T cells are subsequently incubated with at a
corresponding VAR2CSA-Spy fusion protein (corresponding to the
other half of the split-protein binding system of the Spy-CAR)
to produce an armed VAR2CSA-Spy-Spy-CAR receptor as measured by
flow cytometry and immunofluorescence (IF) co-localization.

| | Armed | Un-armed | VAR2-Spy/ |
|---|---|---|---|
| VAR2-SPY | cells | cells | Spy-CAR |
| attachment | (Flow | (Flow | (IF co- |
| to SPY-CAR | positive) | negative) | localization) |
| Over expression | 93.19% | 6.81% | 71.30% |
| CTRL | 6.93% | 93.07% | 1.38% |

### Example 14

Comparing and Selecting VAR2-CARs

To normalize for the transduction efficiency of VAR2-CARs and VAR2-Spy-Spy-CARs, effector T cells are analyzed for percent CAR expression (transduction percentage). Based on this, an effector cell-to-target cell ratio (E:T ratio) is calculated. This is compared to % lysis. One Lytic Unit is defined as 30% lysis of target cells at an E:T ratio of 10:1 (as defined in patent application WO 2013059593 A1). This functional “unit” definition quantifies the amount of lytic activity in each transduced effector cell population being compared. Lytic units represents normalized E:T ratios for the differences in transduction efficiency between constructs.

### Example 15

Lytic Activity of Different VAR2-CARs

To compare the lytic potential of different VAR2-CAR constructs, CARs encoding forward and reverse translated VAR2CSA are compared in ⁵¹Cr release CTL assays or colorimetric cytotoxicity assay using five different CSA-expressing tumor cell lines (e.g. Colo205 colorectal cancer, REH leukemia, UM-UC13 bladder cancer, PC-3 prostate cancer, and RH30 pediatric rhabdomyosarcoma). As a control VAR2-CARs are also tested on non-CSA expressing non-tumorigenic HEK293 cells. The E:T ratios are normalized according to the percent transduction of each individual CAR construct as described in Example 14, thus lytic values are directly comparable. An example of lytic activity of a VAR2-CAR is shown in Table 14.

### Example 16

Lytic Activity of Different VAR2-Spy-Spy-CARs

To compare the lytic potential of different VAR2-Spy-Spy-CAR constructs, CARs encoding forward and reverse translated VAR2-Spy-Spy-CAR constructs are compared in ⁵¹Cr release CTL assays or colorimetric cytotoxicity assay using five different CSA-expressing tumor cell lines (e.g. Colo205 colorectal cancer, REH leukemia, UM-UC13 bladder cancer, PC-3 prostate cancer, and RH30 pediatric rhabdomyosarcoma). As a control VAR2-Spy-Spy-CARs are also tested on non-CSA expressing non-tumorigenic HEK293 cells. The E:T ratios are normalized according to the percent transduction of each individual CAR construct as described in Example 14, thus lytic values are directly comparable. An example of lytic activity of a VAR2-Spy armed Spy-CAR is shown in Table 14.

TABLE 14
T cells purified and transduced with either a VAR2-CAR
or a Spy-CAR subsequently armed with VAR2-Spy as in
Example 13. CARs were added to UC13 cells in the designated
E:T ratios and T-cell mediated cytotoxicity was measured
as described in Example 15 and 16.

| E:T ratio | 40;1 | 20;1 | 10;1 | 5;1 | 2.5;1 |
|---|---|---|---|---|---|
| VAR2-Spy- | 99.82% | 53.47% | 21.37% | 11.65% | 9.02% |
| Spy-CAR | | | | | |
| (cytotoxicity) | | | | | |
| VAR2-CAR | 74.43% | −19.05% | 2.87% | 5.21% | 3.55% |
| (cytotoxicity) | | | | | |
| CTRL | 27.33% | −24.95% | −11.41% | 0% | 11.75% |
| (cytotoxicity) | | | | | |

### Example 17

Lytic Activity of Different VAR2-CARs and VAR2-Spy-Spy-CARs In Vivo

NSG (NOD scid gamma), immune deficient mice are injected on day 0 with a CSA-positive human cancer cell line (e.g. Colo205 colorectal cancer, REH leukemia, UM-UC13 bladder cancer, PC-3 prostate cancer, and RH30 pediatric rhabdomyosarcoma) engineered to express luciferase. On Day 3, mice are treated with 1×10⁷ control T cells (“mock,” untransduced) or 1×10⁷ T cells transduced with VAR2-CARs and VAR2-Spy-Spy-CARs. Tumor burden was measured over a time period of 30 days with bioluminescent imaging using IVIS. Mice are injected intraperitoneally (i.p.) with 3 mg D-luciferin (Caliper Life Sciences, Inc.) and 4 minutes post injection anesthetized mice are imaged with an exposure time of 10-60 seconds. LIVING IMAGE software is used to analyze the bioluminescent signals per each mouse as photons/s/cm/sr.

### ADDITIONAL REMARKS

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A polypeptide comprising i) a first polypeptide domain being a transmembrane domain and an endodomain of a chimeric antigen receptor (CAR), and ii) a second extracellular polypeptide domain selected from a VAR2CSA polypeptide or a peptide part of a split-protein binding system.

2. The polypeptide according to claim 1, which polypeptide comprising said CAR and a peptide part of a split-protein binding system may be combined with a split-protein tagged tumor-targeting agent in vitro or in vivo, such as an antibody.

3. A polypeptide comprising i) a first polypeptide domain being a VAR2CSA polypeptide or other tumor targeting agent, such as an single chain scFv, and ii) a second polypeptide domain selected from one or more of a peptide part of a split-protein binding system, a single-domain antibody, such as an scFv or nanobody.

4. The polypeptide according to any one of claim 1 or 2, wherein said one peptide part of a split-protein binding system is selected from K-Tag (SEQ ID NO:132), SpyCatcher (SEQ ID NO:129), SpyCatcher-ΔN (SEQ ID NO:136), SpyTag (SEQ ID NO:130), Minimal Spytag sequence (SEQ ID NO:131), split-Spy0128 (SEQ ID NO:135), isopeptide Spy0128 (SEQ ID NO:134), or any inverse sequence thereof, or a variant thereof with sequence identity of at least about 80%, such as at least about 82, 84, 86, 88, 90, 92, 94, 96, 98, or 99%.

5. The polypeptide according to any one of claims 1-3, wherein said polypeptide further comprises one or more linker or hinge sequences, such as a GC linker.

6. The polypeptide according to any one of claims 1-4, wherein said VAR2CSA polypeptide consist of SEQ ID NO:55 or SEQ ID NO:56 or fragments or variants thereof with the ability to bind chondroitin sulfate A (CSA) that could be presented on a proteoglycans (CSPG).

7. The polypeptide according to any one of claims 1-5, wherein said VAR2CSA polypeptide is a fragment of VAR2CSA that consist of a sequential amino acid sequence of
a) ID1, and
b) DBL2Xb, and optionally
c) ID2a.

8. The polypeptide according to any one of claims 1-6, wherein said VAR2CSA polypeptide binds chondroitin sulfate A (CSA) on proteoglycans (CSPG) with an affinity as measured by a K_{D} lower than 100 nM, such as lower than 80 nM, such as lower than 70 nM, such as lower than 60 nM, such as lower than 50 nM, such as lower than 40 nM, such as lower than 30 nM, such as lower than 26 nM, such as lower than 24 nM, such as lower than 22 nM, such as lower than 20 nM, such as lower than 18 nM, such as lower than 16 nM, such as lower than 14 nM, such as lower than 12 nM, such as lower than 10 nM, such as lower than 9 nM, such as lower than 8 nM, such as lower than 7 nM, such as lower than 6 nM, or lower than 4 nM.

9. The polypeptide according to any one of claims 1-7, wherein said VAR2CSA polypeptide comprises an amino acid sequence having at least 70% sequence identity with any one amino acid sequence of 1-577 of SEQ ID NO:1, 1-592 of SEQ ID NO:3, 1-579 of SEQ ID NO:4, 1-576 of SEQ ID NO:5, 1-586 of SEQ ID NO:10, 1-579 of SEQ ID NO:11, 1-565 of SEQ ID NO:29, 1-584 of SEQ ID NO:34, 1-569 of SEQ ID NO:36, 1-575 of SEQ ID NO:37, 1-592 of SEQ ID NO:38, 1-603 of SEQ ID NO:41, 1-588 of SEQ ID NO:43, 1-565 of SEQ ID NO:44, 1-589 of SEQ ID NO:45, 1-573 of SEQ ID NO:48, 1-583 of SEQ ID NO:53, 1-569 of SEQ ID NO:54, or 1-640 of SEQ ID NO:128.

10. The polypeptide according to any one of claims 1-8, wherein said VAR2CSA polypeptide comprises an amino acid sequence having at least 70% sequence identity with an amino acid sequence of 578-640 of SEQ ID NO:1, 593-656 of SEQ ID NO:3, 580-643 of SEQ ID NO:4, 577-640 of SEQ ID NO:5, 587-650 of SEQ ID NO:10, 580-643 of SEQ ID NO:11, 566-628 of SEQ ID NO:29, 585-647 of SEQ ID NO:34, 570-632 of SEQ ID NO:36, 576-639 of SEQ ID NO:37, 593-655 of SEQ ID NO:38, 604-667 of SEQ ID NO:41, 589-652 of SEQ ID NO:43, 566-628 of SEQ ID NO:44, 590-653 of SEQ ID NO:45, 574-637 of SEQ ID NO:48, 584-646 of SEQ ID NO:53, or 570-632 of SEQ ID NO:54.

11. The polypeptide according to any one of claims 1-9, wherein said VAR2CSA polypeptide comprises an amino acid sequence having at least 70% sequence identity with an amino acid sequence of SEQ ID NO:2, 6, 8, 9, 12, 13, 14, 15, 16, 17, 18, 19, 20, 21, 22, 23, 24, 25, 26, 27, 28, 30, 31, 32, 33, 35, 39, 40, 42, 46, 47, 49, 50, 51, 52, or 128.

12. The polypeptide according to any one of claims 1-10, wherein said VAR2CSA polypeptide consists of an amino acid sequence having at least 70% sequence identity with any one amino acid sequence of 1-577 of SEQ ID NO:1, 1-592 of SEQ ID NO:3, 1-579 of SEQ ID NO:4, 1-576 of SEQ ID NO:5, 1-586 of SEQ ID NO:10, 1-579 of SEQ ID NO:11, 1-565 of SEQ ID NO:29, 1-584 of SEQ ID NO:34, 1-569 of SEQ ID NO:36, 1-575 of SEQ ID NO:37, 1-592 of SEQ ID NO:38, 1-603 of SEQ ID NO:41, 1-588 of SEQ ID NO:43, 1-565 of SEQ ID NO:44, 1-589 of SEQ ID NO:45, 1-573 of SEQ ID NO:48, 1-583 of SEQ ID NO:53, 1-569 of SEQ ID NO:54, or 1-640 of SEQ ID NO:128.

13. The polypeptide according to any one of claims 1-11, wherein said VAR2CSA polypeptide consists of an amino acid sequence selected from the list consisting of SEQ ID NO:1, 3-5, 10, 11, 29, 34, 36-38, 41, 43-45, 48, 53, 54, and 128.

14. The polypeptide according to any one of claims 1-12, wherein said VAR2CSA polypeptide consists of an amino acid sequence having a length of less than 700 amino acids, such as less than 690 amino acids, such as less than 680 amino acids, such as less than 670 amino acids, such as less than 660 amino acids, such as less than 650 amino acids, such as less than 640 amino acids, such as less than 630 amino acids, such as less than 620 amino acids, such as less than 610 amino acids, such as less than 600 amino acids, such as less than 590 amino acids, such as less than 580 amino acids, such as less than 570 amino acids.

15. The polypeptide according to any one of claims 1-13, wherein said VAR2CSA polypeptide has a molecular mass of less than about 100 kDa under non-reducing conditions on an SDS-PAGE.

16. The polypeptide according to any one of claims 1-14, wherein said VAR2CSA polypeptide is non-glycosylated.

17. The polypeptide according to any one of claims 1-15, wherein said transmembrane domain and/or endodomain of a chimeric antigen receptor (CAR) is derived from a from a 1^{st}, 2^{nd}, or 3^{rd} generation CAR.

18. The polypeptide according to any one of claims 1-15, wherein said polypeptide comprising a first polypeptide domain being a transmembrane domain and an endodomain of a chimeric antigen receptor (CAR), further comprises other regulatory elements or suicide gene systems, such as inducible Caspase-9 (iCASP9) or Sleeping Beauty (SB) transposon system.

19. The polypeptide of any one of claims 1-16, wherein said transmembrane domain and/or endodomain of a chimeric antigen receptor (CAR) comprising a CD28 and/or CD3 zeta signaling domain.

20. The polypeptide according to any one of claims 1-3, wherein said transmembrane domain and/or endodomain of a chimeric antigen receptor (CAR) is as described in any one of WO2014127261, WO13126729, WO13126733, WO14055442, WO14055771, WO2014039523, WO14179759 and WO14138704.

21. An isolated nucleic acid molecule comprising a nucleotide sequences encoding the polypeptide of claims 1-19.

22. An expression vector comprising an isolated nucleic acid molecule according to claim

23. A cell comprising the vector of claim 21.

24. The cell of claim 21, wherein the cell is selected from the group consisting of an αβT cell, γδT cell, a Natural Killer (NK) cell, a cytotoxic T lymphocyte (CTL), a regulatory T cell, or any combination thereof.

25. A two-component therapeutic comprising
a) a first component being a polypeptide comprising i) a first polypeptide domain being a transmembrane domain and an endodomain of a chimeric antigen receptor (CAR), and ii) a second polypeptide domain being a peptide part of a split-protein binding system attached to the extracellular part of said CAR; and
b) a second component being a polypeptide comprising i) a first polypeptide domain being a VAR2CSA polypeptide or any other tumor targeting agent, such as an antibody or scFv, and ii) a second polypeptide domain being a peptide part of a split-protein binding system; optionally further comprising iii) a single-domain antibody, such as an scFv or nanobody,
wherein said peptide parts of a split-protein binding system defined by components a) and b) spontaneously form an irreversible isopeptide bond.

26. The two-component therapeutic, wherein said polypeptides are as defined in any one of claims 1-19.

27. A method of treating or preventing the occurrence of any indication or condition associated with expression, such as inappropriate expression of CSA, such as in cancer, in a subject; the method comprising: (a) culturing a population of cytotoxic lymphocytes under conditions promoting activation; (b) transfecting the lymphocyte population of (a) with a vector encoding a polypeptide comprising i) a first polypeptide domain being the transmembrane domain and the endodomain of a chimeric antigen receptor (CAR), and ii) a second polypeptide domain selected from a VAR2CSA polypeptide, or any other targeting agent, such as an antibody; (c) administering a therapeutically or prophylactically effective number of the transfected lymphocytes of (b) to a subject having or in risk at said indication; wherein the polypeptide comprising the domains of a CAR has binding specificity for the targeted moiety or can be bound by the targeted moiety; thereby treating or preventing said indication in said subject.

28. A method of treating or preventing the occurrence of any indication or condition associated with expression, such as inappropriate expression of CSA, such as in cancer, in a subject; the method comprising: (a) culturing a population of cytotoxic lymphocytes under conditions promoting activation; (b) transfecting the lymphocyte population of (a) with a vector encoding a polypeptide comprising i) a first polypeptide domain being the transmembrane domain and the endodomain of a chimeric antigen receptor (CAR), and ii) a second polypeptide domain a peptide part of a split-protein binding system attached to the extracellular part of said CAR; (c) administering a therapeutically or prophylactically effective number of the transfected lymphocytes of (b) to a subject having or in risk at said indication; and (d) administering a polypeptide comprising i) a first polypeptide domain being a VAR2CSA polypeptide or other targeting agent, such as an antibody, and ii) a second polypeptide domain being a peptide part of a split-protein binding system, wherein said peptide parts of a split-protein binding system defined in the two different polypeptides used spontaneously form an irreversible isopeptide bond; thereby treating or preventing said indication in said subject.

29. The polypeptide according to any one of claims 1-19, or two-component therapeutic according to any one of claims 24-25 for use as a medicament.

30. A pharmaceutical composition comprising the polypeptide according to any one of claims 1-19, or two-component therapeutic according to any one of claims 24-25.

31. Use of a polypeptide according to any one of claims 1-19, or two-component therapeutic according to any one of claims 24-25; for the preparation of a medicament.

32. A polypeptide according to any one of claims 1-19, or two-component therapeutic according to any one of claims 24-25, or pharmaceutical composition according to claim 29 for the treatment of any indications associated with a condition involving expression, such as inappropriate expression of CSA, such as in cancer.

33. The polypeptide or two-component therapeutic according to claim 31, or methods according to claim 26 or 27, wherein said cancer is selected from all CSA-expressing malignancies including carcinomas (including but not limited to Breast carcinoma, Pancreatic carcinoma, Ovarian carcinoma, Endometrial carcinoma, Hepatocellular carcinoma, Lung carcinoma, Colon carcinoma, Prostate carcinoma, Cervix carcinoma, Testis carcinoma, Basal cell skin carcinoma, Clear cell renal cell carcinoma, Head and neck squamous cell carcinoma, Skin squamous cell carcinoma, Vulvar kreatinized squamous cell carcinoma and Vulvar basal cell carcinoma), sarcomas (including but not limited to Fibrosarcoma, Dedifferentiated chondro- and liposarcoma, Leiomyosarcoma, Liposarcoma, Myxoid liposarcoma, Uterine corpus leiomyosarcoma, Osteosarcoma, Ewing sarcoma and Rhabdomyosarcoma, Synovial sarcoma, Solitary Fibrous tumor), hematopoietic cancers (including but not limited to Chronic lymphatic leukaemia (CLL), Acute lymphatic leukaemia (ALL), Acute myeloid leukaemia (AML), B-cell, T-cell and large granular lymphoma), tumours of neuroepithelial tissue, such but not limited to Astrocytomas (Pleomorphic Xanthoastrocytoma, Fibrillary Astrocytomas, Anaplastic astrocytoma, Glioblastoma Multiforme), Oligodrendroglioma, Ependymoma, Choroid plexus turmor, Oligoastrocytoma, gliosarcoma, Ganglioglioma, Retinoblastoma, Neurocytoma, Neuroblastomas (Esthesioneuroblastoma and Ganglioneuroblastoma), Medulloblastoma, Atypical Teratoid Rhabdoid tumors and all types of neuroendocrine cancer, and any other CSA-expressing cancer.

34. A polypeptide according to any one of claims 1-19, or two-component therapeutic according to any one of claims 24-25, or pharmaceutical composition according to claim 29 in combination with any other suitable anticancer agent.
